SEQUENCE LISTING (155 sequences)

SEQ ID NO: 1 - protein, 300 aa, Homo sapiens
  misc_feature (1)..(300): CD38 protein  Human (UniProt P28907)
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 300 aa, Macaca fascicularis
  misc_feature (1)..(300): CD38 protein  Cynomolgus monkey (UniProt Q5VAN0)
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSG

SEQ ID NO: 3 - protein, 304 aa, Mus musculus
  misc_feature (1)..(304): CD38 protein  Mouse
MANYEFSQVSGDRPGCRLSRKAQIGLGVGLLVLIALVVGIVVILLRPRSLLVWTGEPTTKHFSDIFLGRCLIYTQILRPEMRDQNCQEILSTFKGAFVSKNPCNITREDYAPLVKLVTQTIPCNKTLFWSKSKHLAHQYTWIQGKMFTLEDTLLGYIADDLRWCGDPSTSDMNYVSCPHWSENCPNNPITVFWKVISQKFAEDACGVVQVMLNGSLREPFYKNSTFGSVEVFSLDPNKVHKLQAWVMHDIEGASSNACSSSSLNELKMIVQKRNMIFACVDNYRPARFLQCVKNPEHPSCRLNT

SEQ ID NO: 4 - protein, 303 aa, Rattus sp.
  misc_feature (1)..(303): CD38 protein  Rat
MANYEFSQVSEDRPGCRLTRKAQIGLGVGLLLLVALVVVVVIVLWPRSPLVWKGKPTTKHFADIILGRCLIYTQILRPEMRDQDCKKILSTFKRGFISKNPCNITNEDYAPLVKLVTQTIPCNKTLFWSKSKHLAHQYTWIQGKMFTLEDTLLGYIADDLRWCGDPSTSDMNYDSCPHWSENCPNNPVAVFWNVISQKFAEDACGVVQVMLNGSLSEPFYRNSTFGSVEVFNLDPNKVHKLQAWVMHDIKGTSSNACSSPSINELKSIVNKRNMIFACQDNYRPVRFLQCVKNPEHPSCRLNV

SEQ ID NO: 5 - protein, 330 aa, Artificial Sequence
  Synthetic Anti-CD38 heavy chain constant (IgG1 CPPC)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 330 aa, Artificial Sequence
  Synthetic Anti-CD38 heavy chain constant (IgG1 SPPC)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 106 aa, Artificial Sequence
  Synthetic Anti-CD38 light chain constant (lambda)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 107 aa, Artificial Sequence [sequence omitted]

SEQ ID NO: 9 - protein, 118 aa, Artificial Sequence
  Synthetic Anti-CD38 A2 heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS

SEQ ID NO: 10 - protein, 110 aa, Artificial Sequence
  Synthetic Anti-CD38 A2 light chain variable region
QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 11 - protein, 118 aa, Artificial Sequence; identical to SEQ ID NO: 9
  Synthetic Anti-CD38 A2-SV heavy chain variable region

SEQ ID NO: 12 - protein, 110 aa, Artificial Sequence
  Synthetic Anti-CD38 A2-SV light chain variable region
QSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 13 - protein, 118 aa, Artificial Sequence
  Synthetic Anti-CD38 3H10 heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS

SEQ ID NO: 14 - protein, 110 aa, Artificial Sequence; identical to SEQ ID NO: 10
  Synthetic Anti-CD38 3H10 light chain variable region

SEQ ID NO: 15 - protein, 118 aa, Artificial Sequence; identical to SEQ ID NO: 13
  Synthetic Anti-CD38 3H10m1 heavy chain variable region

SEQ ID NO: 16 - protein, 110 aa, Artificial Sequence
  Synthetic Anti-CD38 3H10m1 light chain variable region
QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 17 - protein, 118 aa, Artificial Sequence; identical to SEQ ID NO: 13
  Synthetic Anti-CD38 3H10m1g heavy chain variable region

SEQ ID NO: 18 - protein, 110 aa, Artificial Sequence
  Synthetic Anti-CD38 3H10m1g light chain variable region
QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 19 - protein, 118 aa, Artificial Sequence
  Synthetic Anti-CD38 3G8m1 heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTNWGQGTLVTVSS

SEQ ID NO: 20 - protein, 110 aa, Artificial Sequence; identical to SEQ ID NO: 16
  Synthetic Anti-CD38 3G8m1 light chain variable region

SEQ ID NO: 21 - protein, 118 aa, Artificial Sequence
  Synthetic Anti-CD38 3E3m1 heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTDWGQGTLVTVSS

SEQ ID NO: 22 - protein, 110 aa, Artificial Sequence; identical to SEQ ID NO: 16
  Synthetic Anti-CD38 3E3m1 light chain variable region

SEQ ID NO: 23 - protein, 118 aa, Artificial Sequence
  Synthetic Anti-CD38 3G3 heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWSGEFTDWGQGTLVTVSS

SEQ ID NO: 24 - protein, 110 aa, Artificial Sequence; identical to SEQ ID NO: 10
  Synthetic Anti-CD38 3G3 light chain variable region

SEQ ID NO: 25 - protein, 118 aa, Artificial Sequence; identical to SEQ ID NO: 9
  Synthetic Anti-CD38 3E10 heavy chain variable region

SEQ ID NO: 26 - protein, 110 aa, Artificial Sequence; identical to SEQ ID NO: 16
  Synthetic Anti-CD38 3E10 light chain variable region

SEQ ID NO: 27 - protein, 118 aa, Artificial Sequence
  Synthetic Anti-CD38 3E10N heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSS

SEQ ID NO: 28 - protein, 110 aa, Artificial Sequence; identical to SEQ ID NO: 18
  Synthetic Anti-CD38 3E10N light chain variable region

SEQ ID NO: 29 - protein, 118 aa, Artificial Sequence
  Synthetic Anti-CD38 3E10NS heavy chain variable region
QVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREDWGGEFTDWGQGTLVTVSS

SEQ ID NO: 30 - protein, 110 aa, Artificial Sequence; identical to SEQ ID NO: 18
  Synthetic Anti-CD38 3E10NS light chain variable region

SEQ ID NO: 31 - protein, 118 aa, Artificial Sequence
  Synthetic Anti-CD38 3E11 heavy chain variable region
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWGGEFTDWGQGTLVTVSS

SEQ ID NO: 32 - protein, 110 aa, Artificial Sequence; identical to SEQ ID NO: 10
  Synthetic Anti-CD38 3E11 light chain variable region

SEQ ID NO: 33 - protein, 48 aa, Artificial Sequence
  Synthetic CD8 hinge
AKPTTTPAPRPPTPAPTIASQPLSLRPEACRPAAGGAVHTRGLDFAPR

SEQ ID NO: 34 - protein, 40 aa, Artificial Sequence
  Synthetic CD28 hinge
KIEVMYPPPYLDNEKSNGTIIHVKGKHLCPSPLFPGPSKP

SEQ ID NO: 35 - protein, 88 aa, Artificial Sequence
  Synthetic CD8 + CD28 hinge (long hinge)
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 36 - protein, 27 aa, Artificial Sequence
  Synthetic CD28 transmembrane
FWVLVVVGGVLACYSLLVTVAFIIFWV

SEQ ID NO: 37 - protein, 23 aa, Artificial Sequence
  Synthetic CD8 transmembrane
IYIWAPLAGTCGVLLLSLVITLY

SEQ ID NO: 38 - protein, 27 aa, Artificial Sequence
  Synthetic 4-1BB transmembrane
IISFFLALTSTALLFLLFFLTLRFSVV

SEQ ID NO: 39 - protein, 21 aa, Artificial Sequence
  Synthetic CD3zeta transmembrane
LCYLLDGILFIYGVILTALFL

SEQ ID NO: 40 - protein, 42 aa, Artificial Sequence
  Synthetic 4-1BB costimulatory
KRGRKKLLYIFKQPFMRPVQTTQEEDGCSCRFPEEEEGGCEL

SEQ ID NO: 41 - protein, 41 aa, Artificial Sequence
  Synthetic CD28 costimulatory
RSKRSRLLHSDYMNMTPRRPGPTRKHYQPYAPPRDFAAYRS

SEQ ID NO: 42 - protein, 42 aa, Artificial Sequence
  Synthetic OX40 costimulatory
ALYLLRRDQRLPPDAHKPPGGGSFRTPIQEEQADAHSTLAKI

SEQ ID NO: 43 - protein, 94 aa, Artificial Sequence
  Synthetic Human IL2Rbeta intracellular (with Box 1 and Box 2 motifs)
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 44 - protein, 9 aa, Artificial Sequence
  Synthetic Box 1 from IL2Rbeta intracellular region
LKCNTPDPS

SEQ ID NO: 45 - protein, 10 aa, Artificial Sequence
  Synthetic Box 2 from IL2Rbeta intracellular region
PLEVLERDKV

SEQ ID NO: 46 - protein, 112 aa, Artificial Sequence
  Synthetic CD3zeta ITAM 1, 2, 3 (w/BRR1,2,3; no STAT binding motif YXXQ)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 112 aa, Artificial Sequence
  Synthetic CD3zeta ITAM 1, 2, 3 (w/BRR1,2,3; w/ STAT binding motif YXXQ; BRR1,2,3)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 48 - protein, 96 aa, Artificial Sequence
  Synthetic CD3zeta ITAM 1, 3 (w/ BRR1,2,3; no STAT binding motif YXXQ)
RVKFSRSADAPAYQQGQNQLYNELNLGRREEYDVLDKRRGRDPEMGGKPRRKNPQEGLGMKGERRRGKGHDGLYQGLSTATKDTYDALHMQALPPR

SEQ ID NO: 49 - protein, 96 aa, Artificial Sequence
  Synthetic CD3zeta ITAM 1, 3 (w/ BRR1,2,3; w/ STAT binding motif YXXQ)
RVKFSRSADAPAYQQGQNQLYNELNLGRREEYDVLDKRRGRDPEMGGKPRRKNPQEGLGMKGERRRGKGHDGLYQGLSTATKDTYDAYRHQALPPR

SEQ ID NO: 50 - protein, 73 aa, Artificial Sequence
  Synthetic CD3zeta ITAM 1, d3 (w/ BRR3; no STAT binding motif YXXQ)
RVKFSRSADAPAYQQGQNQLYNELNLGRREEYDVLGMKGERRRGKGHDGLYQGLSTATKDTYDALHMQALPPR

SEQ ID NO: 51 - protein, 73 aa, Artificial Sequence
  Synthetic CD3zeta ITAM 1, d3 (w/ BRR3; w/ STAT binding motif YXXQ)
RVKFSRSADAPAYQQGQNQLYNELNLGRREEYDVLGMKGERRRGKGHDGLYQGLSTATKDTYDAYRHQALPPR

SEQ ID NO: 52 - protein, 35 aa, Artificial Sequence
  Synthetic CD3zeta ITAM 1 (no BRR1,2,3)
RVKFSRSADAPAYQQGQNQLYNELNLGRREEYDVL

SEQ ID NO: 53 - protein, 40 aa, Artificial Sequence
  Synthetic CD3zeta ITAM 1 (w/ BRR1)
RVKFSRSADAPAYQQGQNQLYNELNLGRREEYDVLDKRRG

SEQ ID NO: 54 - protein, 58 aa, Artificial Sequence
  Synthetic CD3zeta ITAM 1 (w/ BRR1,2)
RVKFSRSADAPAYQQGQNQLYNELNLGRREEYDVLDKRRGRDPEMGGKPRRKNPQEGL

SEQ ID NO: 55 - protein, 38 aa, Artificial Sequence
  Synthetic CD3zeta ITAM d3 (w/ BRR3; no STAT binding motif YXXQ)
GMKGERRRGKGHDGLYQGLSTATKDTYDALHMQALPPR

SEQ ID NO: 56 - protein, 38 aa, Artificial Sequence
  Synthetic CD3zeta ITAM d3 (w/ BRR3; w/ STAT binding motif YXXQ)
GMKGERRRGKGHDGLYQGLSTATKDTYDAYRHQALPPR

SEQ ID NO: 57 - protein, 74 aa, Artificial Sequence
  Synthetic CD3zeta ITAM 3 (w/ BRR2,3; no STAT binding motif YXXQ)
RGRDPEMGGKPRRKNPQEGLYNELQKDKMAEAYSEIGMKGERRRGKGHDGLYQGLSTATKDTYDALHMQALPPR

SEQ ID NO: 58 - protein, 74 aa, Artificial Sequence
  Synthetic CD3zeta ITAM 3 (w/ BRR2,3; w/ STAT binding motif; YXXQ)
RGRDPEMGGKPRRKNPQEGLYNELQKDKMAEAYSEIGMKGERRRGKGHDGLYQGLSTATKDTYDAYRHQALPPR

SEQ ID NO: 59 - protein, 43 aa, Artificial Sequence
  Synthetic CD3zeta ITAM 3 (w/ BRR3; no STAT binding motif YXXQ)
AYSEIGMKGERRRGKGHDGLYQGLSTATKDTYDALHMQALPPR

SEQ ID NO: 60 - protein, 43 aa, Artificial Sequence
  Synthetic CD3zeta ITAM 3 (w/ BRR3; w/ STAT binding motif YXXQ)
AYSEIGMKGERRRGKGHDGLYQGLSTATKDTYDAYRHQALPPR

SEQ ID NO: 61 - protein, 15 aa, Artificial Sequence
  Synthetic CD3zeta ITAM 1 motif
YNELNLGRREEYDVL

SEQ ID NO: 62 - protein, 11 aa, Artificial Sequence
  Synthetic CD3zeta ITAM 2 motif
YNELQKDKMAE

SEQ ID NO: 63 - protein, 15 aa, Artificial Sequence
  Synthetic CD3zeta ITAM 3 motif
YQGLSTATKDTYDAL

SEQ ID NO: 64 - protein, 5 aa, Artificial Sequence
  Synthetic BRR1 motif
DKRRG

SEQ ID NO: 65 - protein, 5 aa, Artificial Sequence
  Synthetic BRR2 motif
KPRRK

SEQ ID NO: 66 - protein, 8 aa, Artificial Sequence
  Synthetic BRR3 motif
KGERRRGK

SEQ ID NO: 67 - protein, 4 aa, Artificial Sequence
  Synthetic STAT binding motif (general sequence)
  misc_feature (2)..(3): Xaa can be any naturally occurring amino acid
YXXQ

SEQ ID NO: 68 - protein, 4 aa, Artificial Sequence
  Synthetic STAT binding motif (general sequence)
  misc_feature (2)..(3): Xaa can be any naturally occurring amino acid
YXXF

SEQ ID NO: 69 - protein, 4 aa, Artificial Sequence
  Synthetic STAT binding motif (general sequence)
  misc_feature (2)..(3): Xaa can be any naturally occurring amino acid
YXXL

SEQ ID NO: 70 - protein, 4 aa, Artificial Sequence
  Synthetic STAT binding motif
YRHQ

SEQ ID NO: 71 - protein, 4 aa, Artificial Sequence
  Synthetic STAT binding motif
YLRQ

SEQ ID NO: 72 - protein, 4 aa, Artificial Sequence
  Synthetic STAT binding motif
YFKQ

SEQ ID NO: 73 - protein, 4 aa, Artificial Sequence
  Synthetic STAT binding motif
YLPQ

SEQ ID NO: 74 - protein, 4 aa, Artificial Sequence
  Synthetic STAT binding motif
YMPQ

SEQ ID NO: 75 - protein, 4 aa, Artificial Sequence
  Synthetic STAT binding motif
YVLQ

SEQ ID NO: 76 - protein, 4 aa, Artificial Sequence
  Synthetic STAT binding motif
YQPQ

SEQ ID NO: 77 - protein, 4 aa, Artificial Sequence
  Synthetic STAT binding motif
YKPQ

SEQ ID NO: 78 - protein, 4 aa, Artificial Sequence
  Synthetic STAT binding motif
YRPQ

SEQ ID NO: 79 - protein, 4 aa, Artificial Sequence
  Synthetic STAT binding motif
YTHQ

SEQ ID NO: 80 - protein, 4 aa, Artificial Sequence
  Synthetic STAT binding motif
YLKQ

SEQ ID NO: 81 - protein, 4 aa, Artificial Sequence
  Synthetic STAT binding motif
YHNQ

SEQ ID NO: 82 - protein, 4 aa, Artificial Sequence
  Synthetic STAT binding motif
YFFF

SEQ ID NO: 83 - protein, 4 aa, Artificial Sequence
  Synthetic STAT binding motif
YCTF

SEQ ID NO: 84 - protein, 4 aa, Artificial Sequence
  Synthetic STAT binding motif
YLSL

SEQ ID NO: 85 - protein, 5 aa, Artificial Sequence
  Synthetic STAT binding motif
YLSLQ

SEQ ID NO: 86 - protein, 5 aa, Artificial Sequence
  Synthetic STAT binding motif
YCTFP

SEQ ID NO: 87 - protein, 5 aa, Artificial Sequence
  Synthetic STAT binding motif
YFFFH

SEQ ID NO: 88 - protein, 19 aa, Artificial Sequence
  Synthetic Heavy chain leader sequence
MEWSWVFLFFLSVTTGVHS

SEQ ID NO: 89 - protein, 20 aa, Artificial Sequence
  Synthetic Light chain leader sequence
MSVPTQVLGLLLLWLTDARC

SEQ ID NO: 90 - protein, 21 aa, Artificial Sequence
  Synthetic Alternative leader sequence
MALPVTALLLPLALLLHAARP

SEQ ID NO: 91 - protein, 21 aa, Artificial Sequence
  Synthetic T2A self-cleaving sequence
GSGEGRGSLLTCGDVEENPGP

SEQ ID NO: 92 - protein, 22 aa, Artificial Sequence
  Synthetic P2A self-cleaving sequence
GSGATNFSLLKQAGDVEENPGP

SEQ ID NO: 93 - protein, 23 aa, Artificial Sequence
  Synthetic E2A self-cleaving sequence
GSGQCTNYALLKLAGDVESNPGP

SEQ ID NO: 94 - protein, 25 aa, Artificial Sequence
  Synthetic F2A self-cleaving sequence
GSGVKQTLNFDLLKLAGDVESNPGP

SEQ ID NO: 95 - protein, 817 aa, Artificial Sequence
  Synthetic Precursor memory DAR (V1)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 96 - protein, 560 aa, Artificial Sequence
  Synthetic Memory DAR (V1) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 97 - protein, 236 aa, Artificial Sequence
  Synthetic Memory DAR (V1) light chain (2nd polypeptide)
MSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 98 - protein, 801 aa, Artificial Sequence
  Synthetic Precursor memory DAR (V2)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - protein, 544 aa, Artificial Sequence
  Synthetic Memory DAR (V2) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPR

SEQ ID NO: 100 - protein, 236 aa, Artificial Sequence; identical to SEQ ID NO: 97
  Synthetic Memory DAR (V2) light chain (2nd polypeptide)

SEQ ID NO: 101 - protein, 778 aa, Artificial Sequence
  Synthetic Precursor memory DAR (V3)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 102 - protein, 521 aa, Artificial Sequence
  Synthetic Memory DAR (V3) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLGMKGERRRGKGHDGLYQGLSTATKDTYDAYRHQALPPR

SEQ ID NO: 103 - protein, 236 aa, Artificial Sequence; identical to SEQ ID NO: 97
  Synthetic Memory DAR (V3) light chain (2nd polypeptide)

SEQ ID NO: 104 - protein, 723 aa, Artificial Sequence
  Synthetic Precursor traditional DAR (V10)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 105 - protein, 466 aa, Artificial Sequence
  Synthetic Traditional anti-CD38 DAR (V10) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 236 aa, Artificial Sequence; identical to SEQ ID NO: 97
  Synthetic Traditional anti-CD38 DAR (V10) light chain (2nd polypeptide)

SEQ ID NO: 107 - protein, 707 aa, Artificial Sequence
  Synthetic Precursor traditional DAR (V11)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLGMKGERRRGKGHDGLYQGLSTATKDTYDALHMQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 108 - protein, 450 aa, Artificial Sequence
  Synthetic Traditional DAR (V11) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLGMKGERRRGKGHDGLYQGLSTATKDTYDALHMQALPPR

SEQ ID NO: 109 - protein, 236 aa, Artificial Sequence; identical to SEQ ID NO: 97
  Synthetic Traditional DAR (V11) light chain (2nd polypeptide)

SEQ ID NO: 110 - protein, 684 aa, Artificial Sequence
  Synthetic Precursor traditional DAR (V12)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLGMKGERRRGKGHDGLYQGLSTATKDTYDALHMQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 427 aa, Artificial Sequence
  Synthetic Traditional DAR (V12) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLGMKGERRRGKGHDGLYQGLSTATKDTYDALHMQALPPR

SEQ ID NO: 112 - protein, 236 aa, Artificial Sequence; identical to SEQ ID NO: 97
  Synthetic Traditional DAR (V12) light chain (2nd polypeptide)

SEQ ID NO: 113 - protein, 775 aa, Artificial Sequence
  Synthetic Precursor memory DAR (V4)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - protein, 518 aa, Artificial Sequence
  Synthetic Memory DAR (V4) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 115 - protein, 236 aa, Artificial Sequence; identical to SEQ ID NO: 97
  Synthetic Memory DAR (V4) light chain (2nd polypeptide)

SEQ ID NO: 116 - protein, 759 aa, Artificial Sequence
  Synthetic Precursor memory DAR (V5)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 117 - protein, 502 aa, Artificial Sequence
  Synthetic Memory DAR (V5) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPR

SEQ ID NO: 118 - protein, 236 aa, Artificial Sequence; identical to SEQ ID NO: 97
  Synthetic Memory DAR (V5) light chain (2nd polypeptide)

SEQ ID NO: 119 - protein, 736 aa, Artificial Sequence
  Synthetic Precursor memory DAR (V6)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 120 - protein, 479 aa, Artificial Sequence
  Synthetic Memory DAR (V6) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLGMKGERRRGKGHDGLYQGLSTATKDTYDAYRHQALPPR

SEQ ID NO: 121 - protein, 236 aa, Artificial Sequence; identical to SEQ ID NO: 97
  Synthetic Memory DAR (V6) light chain (2nd polypeptide)

SEQ ID NO: 122 - protein, 759 aa, Artificial Sequence
  Synthetic Precursor memory DAR (V7)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRDPEMGGKPRRKNPQEGLGMKGERRRGKGHDGLYQGLSTATKDTYDAYRHQALPPRGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 123 - protein, 502 aa, Artificial Sequence
  Synthetic Memory DAR (V7) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRDPEMGGKPRRKNPQEGLGMKGERRRGKGHDGLYQGLSTATKDTYDAYRHQALPPR

SEQ ID NO: 124 - protein, 236 aa, Artificial Sequence; identical to SEQ ID NO: 97
  Synthetic Memory DAR (V7) light chain (2nd polypeptide)

SEQ ID NO: 125 - protein, 759 aa, Artificial Sequence
  Synthetic Precursor memory DAR (V8)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPRNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 502 aa, Artificial Sequence
  Synthetic Memory DAR (V8) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPRNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 127 - protein, 236 aa, Artificial Sequence; identical to SEQ ID NO: 97
  Synthetic Memory DAR (V8) light chain (2nd polypeptide)

SEQ ID NO: 128 - protein, 775 aa, Artificial Sequence
  Synthetic Precursor memory DAR (V9)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVGSGEGRGSLLTCGDVEENPGPMSVPTQVLGLLLLWLTDARCQSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 129 - protein, 518 aa, Artificial Sequence
  Synthetic Memory DAR (V9) heavy chain (1st polypeptide)
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 130 - protein, 236 aa, Artificial Sequence; identical to SEQ ID NO: 97
  Synthetic Memory DAR (V9) light chain (2nd polypeptide)

SEQ ID NO: 131 - protein, 248 aa, Artificial Sequence
  Synthetic Memory DAR (V1) intracellular region
KRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 132 - protein, 232 aa, Artificial Sequence
  Synthetic Memory DAR (V2) intracellular region
KRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPR

SEQ ID NO: 133 - protein, 209 aa, Artificial Sequence
  Synthetic Memory DAR (V3) intracellular region
KRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLGMKGERRRGKGHDGLYQGLSTATKDTYDAYRHQALPPR

SEQ ID NO: 134 - protein, 206 aa, Artificial Sequence
  Synthetic Memory DAR (V4) intracellular region
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 135 - protein, 190 aa, Artificial Sequence
  Synthetic Memory DAR (V5) intracellular region
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLGMKGERRRGKGHDGLYQGLSTATKDTYDAYRHQALPPR

SEQ ID NO: 136 - protein, 167 aa, Artificial Sequence
  Synthetic Memory DAR (V6) intracellular region
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLGMKGERRRGKGHDGLYQGLSTATKDTYDAYRHQALPPR

SEQ ID NO: 137 - protein, 190 aa, Artificial Sequence
  Synthetic Memory DAR (V7) intracellular region
RVKFSRSADAPAYQQGQNQLYNELNLGRREEYDVLDKRRGNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRDPEMGGKPRRKNPQEGLGMKGERRRGKGHDGLYQGLSTATKDTYDAYRHQALPPR

SEQ ID NO: 138 - protein, 190 aa, Artificial Sequence
  Synthetic Memory DAR (V8) intracellular region
RVKFSRSADAPAYQQGQNQLYNELNLGRREEYDVLDKRRGRDPEMGGKPRRKNPQEGLGMKGERRRGKGHDGLYQGLSTATKDTYDAYRHQALPPRNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 139 - protein, 206 aa, Artificial Sequence
  Synthetic Memory DAR (V9) intracellular region
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 140 - protein, 154 aa, Artificial Sequence
  Synthetic Traditional DAR (V10) intracellular region
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 138 aa, Artificial Sequence
  Synthetic Traditional DAR (V11) intracellular region
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLGMKGERRRGKGHDGLYQGLSTATKDTYDALHMQALPPR

SEQ ID NO: 142 - protein, 115 aa, Artificial Sequence
  Synthetic Traditional DAR (V12) intracellular region
KRGRKKLLYIFKQPFMRPVQTTQEEDGCSCRFPEEEEGGCELRVKFSRSADAPAYQQGQNQLYNELNLGRREEYDVLGMKGERRRGKGHDGLYQGLSTATKDTYDALHMQALPPR

SEQ ID NO: 143 - protein, 551 aa, Homo sapiens
  misc_feature (1)..(551): Human IL2Rbeta full-length (UniProtKB P14784)
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 144 - protein, 29 aa, Homo sapiens
  misc_feature (1)..(29): ITAM from human CD3 gamma (UniProtKB P09693)
DKQTLLPNDQLYQPLKDREDDQYSHLQGN

SEQ ID NO: 145 - protein, 29 aa, Homo sapiens
  misc_feature (1)..(29): ITAM from human CD3 delta (UniProtKB P04234)
DTQALLRNDQVYQPLRDRDDAQYSHLGGN

SEQ ID NO: 146 - protein, 28 aa, Homo sapiens
  misc_feature (1)..(28): ITAM from human CD3 epsilon (UniProtKB P07766)
ERPPPVPNPDYEPIRKGQRDLYSGLNQR

SEQ ID NO: 147 - protein, 10 aa, Artificial Sequence
  Synthetic IgG1 upper hinge sequence
EPKSCDKTHT

SEQ ID NO: 148 - protein, 9 aa, Artificial Sequence
  Synthetic lower hinge/CH2 sequence
PAPELLGGP

SEQ ID NO: 149 - protein, 12 aa, Artificial Sequence
  Synthetic Fc region (CH2)
SVFLFPPKPKDT

SEQ ID NO: 150 - protein, 23 aa, Artificial Sequence
  Synthetic hinge region
EPKSCDKTHTCPPCPAPELLGGP

SEQ ID NO: 151 - protein, 4 aa, Artificial Sequence
  Synthetic ITAM motif
YNEL

SEQ ID NO: 152 - protein, 4 aa, Artificial Sequence
  Synthetic ITAM motif
YDVL

SEQ ID NO: 153 - protein, 4 aa, Artificial Sequence
  Synthetic ITAM motif
YSEI

SEQ ID NO: 154 - protein, 4 aa, Artificial Sequence
  Synthetic ITAM motif
YQGL

SEQ ID NO: 155 - protein, 4 aa, Artificial Sequence
  Synthetic ITAM motif
YDAL

(19) United States
(12) Patent Application Publication
(10) Pub. No.: US 2023/0167191 A1
(43) Pub. Date: Jun. 1, 2023

(54) Memory Dimeric Antigen Receptors (mDARs)

(71) Applicant: Sorrento Therapeutics, Inc., San Diego, CA (US)

(72) Inventors: Henry Hongjun Ji, Rancho Santa Fe, CA (US); Runqiang Chen, San Diego, CA (US); Xiaomei Yuan, San Diego, CA (US)

(73) Assignee: Sorrento Therapeutics, Inc., San Diego, CA (US)

(21) Appl. No.: 17/920,724

(22) PCT Filed: Apr. 23, 2021
(86) PCT No.: PCT/US2021/028968

§ 371 Date: Oct. 21, 2022

Related U.S. Application Data
(60) Provisional application No. 63/014,964, filed on Apr. 24, 2020.

(51) Int. Cl. C07K 16/28; C07K 14/725; C07K 14/715; C07K 14/73; C07K 14/705; A61K 48/00; A61K 35/17

(52) U.S. Cl. CPC C07K 16/2896; C07K 14/7051; C07K 14/7155; C07K 14/70514; C07K 14/70521; A61K 48/005; A61K 35/17; C07K 2319/00; A61K 38/00

(57) ABSTRACT

The present disclosure provides transgenic cells that express memory dimeric antigen receptors (mDARs), where the mDAR constructs comprise a JAK-STAT intracellular region having a cytokine receptor intracellular region which includes Box 1 and Box 2 motifs for binding a Janus kinase (JAK) which can play a role in JAK-STAT cellular signaling pathway to induce effector cell activation and proliferation. In one embodiment, the JAK-STAT intracellular region further comprises a CD3zeta intracellular signaling region having an intact ITAM region, or having ITAM 1 and 3, or having only ITAM 3 with a partial deletion. Transgenic cells expressing the mDAR constructs exhibit potent cytotoxicity, and release reduced levels of cytokines, compared to traditional DARs that lack a cytokine receptor intracellular region. The mDAR constructs have antibody-like properties as they bind specifically to a target antigen. Transgenic cells expressing the mDAR constructs can be used for directed cell therapy.

### CROSS REFERENCE TO RELATED APPLICATIONS

This application is a national phase entry pursuant to 35 U.S.C. § 371 of International Application No. PCT/US2021/028968, filed Apr. 23, 2021, which claims the benefit of priority to U.S. provisional application No. 63/014,964, filed Apr. 24, 2020, the contents of which are incorporated herein by reference in their entireties.

### SEQUENCE LISTING

The instant application contains a Sequence Listing which has been submitted electronically in ASCII format via EFS-Web and is hereby incorporated by reference in its entirety. Said ASCII copy, created on Apr. 23, 2021, is named “01223-0072-00PCT_ST25” and is 251 kilobytes in size.

### TECHNICAL FIELD

The present disclosure provides memory dimeric antigen receptors (mDARs) and related polypeptide constructs that bind specifically to a target antigen, nucleic acids that encode the mDARs and related polypeptide constructs, vectors comprising the nucleic acids, and transgenic host cells (e.g., host T cells) harboring the vectors that can express the mDAR constructs. The mDAR constructs described herein include Janus kinase (JAK) binding motifs and Signal Transducer and Activator of Transcription proteins (STAT) binding motifs for improved host T cell activation upon binding to a target antigen.

### BACKGROUND

Chimeric antigen receptors (CARs) have been developed to target antigens associated, in particular, with cancer. The first-generation CAR was engineered to contain a signaling domain (TCR) that delivers an activation stimulus (signal 1) only (Geiger et al., J. Immunol. 162(10): 5931-5939, 1999; Haynes et al., J. Immunol. 166(1): 182-187, 2001) (Hombach et al. Cancer Res. 61(5): 1976-1982, 2001; Hombach et al., J. Immunol. 167(11): 6123-6131, 2001; Maher et al., Nat. Biotechnol. 20(1): 70-75, 2002). T cells grafted with the first-generation CARs exhibited limited anti-tumor efficacy due to suboptimal activation (Beecham et al., J. Immunother. 23(6): 631-642, 2000). The second-generation CAR, immunoglobulin-CD28-T cell receptor (IgCD28TCR), incorporated a costimulatory CD28 domain (signal 2) into the first-generation receptor (Gerstmayer et al., J. Immunol. 158(10): 4584-4590, 1997; Emtage et al., Clin. Cancer Res. 14(24): 8112-8122, 2008; Lo, Ma et al., Clin. Cancer Res. 16(10): 2769-2780, 2010) that resulted in CAR-T cells with a greater anti-tumor capacity (Finney et al., J. Immunol. 161(6): 2791-2797, 1998; Hombach et al., Cancer Res. 61(5): 1976-1982, 2001, Maher et al., Nat. Biotechnol. 20(1): 70-75, 2002). Various CAR variants have been developed by replacing the signal domains of TCR or CD28 with molecules with similar functions, such as FcRγ, 4-1BB and OX40 (Eshhar et al., Proc. Natl. Acad. Sci. USA 90(2): 720-724, 1993). TCR CAR-T cells against various tumor antigens have been developed (Ma et al., Cancer Gene Ther. 11(4): 297-306, 2004; Ma et al., Prostate 61(1): 12-25, 2004; Lo et al., Clin. Cancer Res. 16(10): 2769-2780, 2010; Kong et al., Clin. Cancer Res. 18(21): 5949-5960, 2012; Ma et al., Prostate 74(3): 286-296, 2014; Katz et al., Clin. Cancer Res. 21(14): 3149-3159, 2015; Junghans et al., 2016 The Prostate, 76(14): 1257-1270).

Adoptive immunotherapy by infusion of T cells engineered with chimeric antigen receptors (CARs) for redirected tumoricidal activity represents a potentially highly specific modality for the treatment of metastatic cancer. CAR-T cells targeting CD19, a molecule expressed on B cells, have shown success in treatment of B cell malignancies and have received FDA approval, with some trials showing a response rate of up to 70%, including sustained complete responses. Nonetheless, CAR-T cells may show nonspecific activation, which may result in potentially serious adverse events through inappropriate immune activity.

Dimeric antigen receptors (DARs) have two polypeptides that when produced by a transgenic host cell associate with one another to form an antigen binding receptor. The first polypeptide is a transmembrane polypeptide with an extracellular domain that includes a binding region and a region for associating with the second (non-transmembrane) polypeptide of the DAR, a transmembrane domain, and an intracellular domain that can include a signaling domain and optionally one or more co-stimulatory domains. The second polypeptide is engineered to be secreted from a host cell engineered to express the DAR and includes a binding domain and a domain for association with the first (transmembrane) polypeptide. In various DAR configurations, the first polypeptide of the DAR includes a variable region of a heavy chain of an antibody and the second polypeptide of the DAR includes a variable region of a light chain of the antibody, such that association of the first and second polypeptides of the at the exterior of the host cell expressing the DAR allows formation of a binding site having the structure of a Fab fragment, where the Fab fragment is attached, via the transmembrane domain of the first polypeptide, to an intracellular signaling domain (which is also part of the first polypeptide). (In alternative configurations, the first polypeptide of the DAR includes a variable region of a light chain of an antibody and the second polypeptide of the DAR includes a variable region of a heavy chain of the antibody.) DARs and their various configurations and domains, constructs encoding DARs, and cells expressing DARs and their uses in cell therapy are disclosed, for example, in WO 2019/173837 and WO 2021 046445, both of which are incorporated by reference herein in their entireties.

Cytokine receptors expressed on certain T lymphocytes control T lymphocyte differentiation and function by regulating the activity of intracellular tyrosine kinases. Type I and II cytokine receptors employ the JAK-STAT pathway to direct innate and adaptive immune responses. The Janus kinase-signal transducer and activator of transcription (JAK-STAT) signaling pathway plays a pivotal role in transferring signals from outside the cell via cell membrane receptors to the cell's nucleus to induce transcription or suppression of numerous target genes. The JAK protein family includes different types of tyrosine kinases that constitutively bind to cytokine receptors at JAK binding sites having conserved sequences known as Box 1 and Box 2 motifs. Multimerization of the cytokine receptors occurs upon receptor binding to its cognate ligand (cytokine). The multimerization of the cytokine receptors brings the JAK proteins (which are bound to the cytokine receptors) into close proximity leading to trans-phosphorylation of the Box 1 and 2 motifs on the multimerized cytokine receptors, which in turn leads to phosphorylation of STAT binding sites on the multimerized cytokine receptors. STAT proteins are recruited to bind the cytokine receptors at the phosphorylated STAT binding sites leading to dimerization of STAT proteins. The dimerized STAT complexes dissociate from the cytokine receptor and translocate to the nucleus to activate or suppress transcription of certain genes. The human JAK family includes four types of JAK proteins: JAK1, JAK2, JAK3 and TYK2. The amino acid sequences of Box 1 and 2 motifs of different human cytokine receptors are known. The human STAT family includes seven types of STAT proteins: STAT1, STAT2, STAT3, STAT4, STAT5A, STAT5B and STAT6. It is known that JAK1 and STAT3 play a role in activating signaling for IL-2Rbeta cytokine receptors.

The stimulation effects of IL-2 cytokine on IL-2 receptor signaling is complex and involves at least JAK1, JAK3 and STAT5A/B. It is known that IL-2 cytokine stimulates proliferation of CD8+ and CD4+ T cells. It has been previously shown that high levels of IL-2 cytokine interaction on IL-2 receptors stimulates immune-activated CD8+ T cells to become terminallydifferentiated short-lived cytolytic effector T cells. By contrast, low levels of IL-2 cytokine drive development of long-lived CD8+ and CD4+ memory T cells.

It has been previously demonstrated by others that cytokines, such as IL-2 and IL-15, mediate intracellular signaling via a common gamma chain (γc) to promote development of CD8+ short-lived effector memory cells by activating the JAK-STAT intracellular signaling pathway (Mathieu 2015 European Journal of Immunology 45:3324-3338). By contrast, IL-7 cytokine, which also mediates signaling via the common gamma chain (γc), promotes development of long-lived memory cells.

### SUMMARY

Memory dimeric antigen receptors (mDAR) comprising both an antibody heavy chain binding region and an antibody light chain binding region in separate polypeptide chains that form a Fab fragment joined to transmembrane and intracellular signaling regions, and transgenic host cells (e.g., transgenic host T cells) expressing such mDARs that provide improved activation of transgenic host T cells are provided. The intracellular signaling regions of the mDAR constructs include JAK and STAT binding motifs. T cells expressing the mDARs can exhibit improved target-specific expansion, cytotoxicity, in vivo expansion, and in vivo persistence, e.g., in comparison to T cells expressing a traditional DAR. Embodiments according to this disclosure are set forth in the claims and the detailed description.

The present disclosure provides mDARs that include Box 1 and Box 2 motifs from IL2Rbeta (IL2Rβ) and STAT binding motifs in their intracellular regions which are demonstrated herein to mediate phosphorylation of STAT3 and STAT5. and increase T cell activation and proliferation, compared to traditional mDAR constructs. The intracellular region of traditional DAR constructs include a CD3zeta (CD3ζ) ITAM signaling region and costimulatory region (4-1BB and/or CD28) but lack Box 1, Box 2, and STAT binding motifs. The activity levels of the traditional DAR (which lacks JAK and STAT binding motifs) and mDAR constructs described herein are directly comparable because the hinge regions, transmembrane regions and intracellular regions are the same in both types of constructs. Without wishing to be bound by theory, it is postulated that transgenic T cells expressing the mDAR constructs stimulate three intracellular signals upon target antigen binding: (1) T cell receptor (TCR) engagement signal mediated by binding the target antigen; (2) costimulatory signal mediated by 4-1BB and/or CD28 intracellular regions; and (3) cytokine engagement signal mediated by the Box 1 and 2 motifs for JAK binding, and by STAT binding motif.

The intracellular region of the mDAR constructs described herein have a chimeric arrangement which includes (i) an IL2Rβ intracellular region with Box 1 and Box 2 motifs for binding JAK proteins, and (ii) at least one CD3ζ intracellular domain with a heterologous STAT motif for binding STAT proteins. It is demonstrated herein that transgenic T cells expressing the mDAR constructs stimulate CD8+ memory T cell development upon binding to a target antigen by utilizing the extracellular antigen binding domain and JAK-STAT intracellular signaling pathway. The mDAR construct can provide superior results relative to the corresponding traditional DAR format in binding to cells expressing the target antigen, antigen-induced cytokine release, and/or antigen-induced cytotoxicity.

In some embodiments, transgenic host cells express mDAR constructs comprising an intracellular region that can bind a Janus kinase (JAK). In some embodiments, transgenic host cells express mDAR constructs comprising a cytokine receptor intracellular region with Box 1 and Box 2 motifs for mediating the JAK-STAT intracellular signaling pathway.

In some embodiments, the intracellular region comprises a cytokine receptor intracellular region such as an intracellular region derived from IL2Rβ (e.g., amino acids 266-551 of SEQ ID NO:143, or a sequence having at least 95% identity thereto). In some embodiments, the cytokine receptor intracellular region comprises an IL2Rβ intracellular region having a partial deletion of any portion within the region comprising amino acids 338-529 of SEQ ID NO:143. In some embodiments, the cytokine receptor intracellular region comprises an IL2Rβ intracellular region having a partial deletion of amino acids 338-529 of SEQ ID NO:143, where the cytokine receptor intracellular region comprises the amino acid sequence of SEQ ID NO:43 or an amino acid sequence having at least 95% identity thereto. In some embodiments, the Box 1 and 2 motifs comprise the amino acid sequence of SEQ ID NO:44 and 45, respectively.

In some embodiments, the intracellular region further comprises an intracellular signaling region comprising an immunoreceptor tyrosine-based activation motif (ITAM). In some embodiments, the intracellular signaling region comprises an intracellular region from CD3 gamma, delta, or epsilon, each having a single ITAM domain (e.g., SEQ ID NOS:144, 145 and 146, respectively, or amino acid sequences having at least 95% identity thereto). In some embodiments, the intracellular region comprises at least one ITAM domain from a CD3ζ intracellular signaling region (e.g., any one of SEQ ID NO:46-60, or amino acid sequences having at least 95% identity thereto). In some embodiment, the intracellular region comprises any one or any combination of two or three ITAMs 1, 2 and/or 3 domain(s) from a CD3ζ intracellular signaling region. In one embodiment, the intracellular region comprises CD3ζ intracellular signaling region with ITAMs 1 and 2, or ITAMs 2 and 3, or ITAMs 1 and 3, or ITAM 1 only, or ITAM 2 only, or ITAM 3 only. In some embodiments, the intracellular region comprises a CD3ζ intracellular signaling region having at least one ITAM domain with at least two ITAM motifs (e.g., at least two of SEQ ID NOS:61, 62 and 63, or amino acid sequences having at least 95% identity thereto). In some embodiments, the CD3ζ intracellular signaling region further comprises a heterologous binding motif for Signal Transducer and Activator of Transcription proteins (STAT) (e.g., any one of SEQ ID NO:67-87, or amino acid sequences having at least 95% identity thereto).

In some embodiments, the intracellular region further comprises an optional intracellular costimulatory region for example from 4-1BB (SEQ ID NO:40, or an amino acid sequence having at least 95% identity thereto), CD28 (SEQ ID NO:41, or an amino acid sequence having at least 95% identity thereto) and/or OX40 (SEQ ID NO:42, or an amino acid sequence having at least 95% identity thereto.

In some embodiments, mDAR constructs lack a hinge region. In other embodiments, an mDAR includes a hinge region between the Fab fragment and the transmembrane region.

Also included herein are nucleic acid molecules encoding precursor mDAR polypeptides such as any disclosed herein. The nucleic acid molecules can encode the first and second polypeptides can be fused, for example, via a 2A sequence such that an encoding nucleic acid molecule can encode both component polypeptides as a single transcriptional unit. Alternatively a nucleic acid molecule as provided herein can encode one or both of a first or second mDAR precursor polypeptide. As nonlimiting examples, a nucleic acid molecule can encode a precursor polypeptide of any of SEQ ID NOs:95, 98, 101, 113, 116, 119, 122, 125 or 128, or a polypeptide having at least 95%, at least 96%, at least 97%, at least 98%, or at least 99% identity to any of SEQ ID NO:95, 98, 101, 113, 116, 119, 122, 125 or 128. In some examples a nucleic acid molecule encodes an mDAR precursor polypeptide having at least 95%, at least 96%, at least 97%, at least 98%, or at least 99% identity to any of SEQ ID NO:95, SEQ ID NO:98, or SEQ ID NO:101. A nucleic acid molecule as provided herein can be an expression construct where a promoter is operably linked to a DAR polypeptide-encoding sequence. The nucleic acid molecule can be a vector, e.g., a retroviral, AAV, or adenoviral vector, or can be, for example, a DNA fragment.

The present disclosure also provides transgenic host cells (e.g., transgenic T cells) expressing the mDARs, where the transgenic host cells exhibit target antigen binding-dependent JAK/STAT signaling pathway activation. In various embodiments a host cell is transfected with one or more nucleic acid molecules encoding the first and second DAR polypeptides. The transgenic host can be a T cell that is knocked out for the T cell receptor, i.e., expresses an mDAR and does not express the T cell receptor. The cells may be provided as a pharmaceutical composition and may be frozen.

In various embodiments, host cells provided herein can exhibit enhance target cell killing, reduced cytokine release in response to target cells, and/or enhanced expansion in vivo with respect to host cells that express a traditional DAR that does not include intracellular regions that bind JAK or STAT proteins.

### DESCRIPTION OF THE DRAWINGS

FIG. 1A) is a schematic showing an exemplary memory dimeric antigen receptor (mDAR) comprising an antibody heavy chain carrying an intracellular JAK-STAT signaling region; B) is a schematic showing an exemplary memory dimeric antigen receptor (mDAR) comprising an antibody light chain carrying an intracellular JAK-STAT signaling region; C) is a schematic showing an exemplary traditional dimeric antigen receptor (DAR) comprising an antibody heavy chain carrying an intracellular signal region that lacks JAK-STAT motifs; and D) is a schematic showing an exemplary traditional dimeric antigen receptor (DAR) comprising an antibody light chain carrying an intracellular signal region that lacks JAK-STAT motifs.

FIG. 2A) is a schematic showing exemplary embodiments (Versions 1-3) of different intracellular JAK-STAT signaling regions that include a costimulatory region designed for various memory dimeric antigen receptors (mDAR) described herein;) B is a schematic showing exemplary embodiments (Versions 4-6) of different intracellular JAK-STAT signaling regions that lack a costimulatory region designed for various memory dimeric antigen receptors (mDAR) described herein; C) is a schematic showing exemplary embodiments (Versions 7-9) of different intracellular signaling regions that lack a costimulatory region designed for various memory dimeric antigen receptors (mDAR) described herein; D) is a schematic showing exemplary embodiments (Versions 10-12) of different traditional dimeric antigen receptors that lack an intracellular JAK-STAT region described herein.

FIG. 3A is a schematic showing an exemplary precursor polypeptide molecule comprising a heavy chain antibody region with an intracellular JAK-STAT signaling region, a self-cleaving sequence, and a light chain antibody region; B) is a schematic showing an exemplary precursor polypeptide molecule comprising a light chain antibody region with an intracellular JAK-STAT signaling region, a self-cleaving sequence, and a heavy chain antibody region.

FIG. 4 shows the results of a flow cytometry study for detecting expression of CD38 DAR and CD3 in negative control cells at day 7, 10 or 13 post-transfection. The negative control cells included non-transgenic activated T cells (ATC) and T cells having knocked-out TRAC locus (KO TRAC) (see Example 5).

FIG. 5A shows the results of a flow cytometry study for detecting expression of CD62L and CD45RA, or CD45RA and CCR7, in CD4+ negative control cells at day 7 post-transfection. The negative control cells included non-transgenic activated T cells (ATC) and T cells having knocked-out TRAC locus (KO TRAC) (see Example 5).

FIG. 5B shows the results of a flow cytometry study for detecting expression of CD62L and CD45RA, or CD45RA and CCR7, in CD4+ negative control cells at day 10 post-transfection. The negative control cells included non-transgenic activated T cells (ATC) and T cells having knocked-out TRAC locus (KO TRAC) (see Example 5).

FIG. 5C shows the results of a flow cytometry study for detecting expression of CD62L and CD45RA, or CD45RA and CCR7, in CD4+ negative control cells at day 13 post-transfection. The negative control cells included non-transgenic activated T cells (ATC) and T cells having knocked-out TRAC locus (KO TRAC) (see Example 5).

FIG. 6A shows the results of a flow cytometry study for detecting expression of CD62L and CD45RA, or CD45RA and CCR7, in CD8+ negative control cells at day 7 post-transfection. The negative control cells included non-transgenic activated T cells (ATC) and T cells having knocked-out TRAC locus (KO TRAC) (see Example 5).

FIG. 6B shows the results of a flow cytometry study for detecting expression of CD62L and CD45RA, or CD45RA and CCR7, in CD8+ negative control cells at day 10 post-transfection. The negative control cells included non-transgenic activated T cells (ATC) and T cells having knocked-out TRAC locus (KO TRAC) (see Example 5).

FIG. 6C shows the results of a flow cytometry study for detecting expression of CD62L and CD45RA, or CD45RA and CCR7, in CD8+ negative control cells at day 13 post-transfection. The negative control cells included non-transgenic activated T cells (ATC) and T cells having knocked-out TRAC locus (KO TRAC) (see Example 5).

FIG. 7 shows the results of a flow cytometry study for detecting expression of CD38 DAR and CD3 in traditional DAR (V10) T cells or memory DAR (V1) T cells at day 7, 10 or 13 post-transfection (Example 6).

FIG. 8A shows the results of a flow cytometry study for detecting the fraction of memory T cell subsets in transgenic CD4+ T cells expressing CD38 traditional DAR (V10) or CD38 mDAR (V1). The CD4+ memory T cell subsets expressing CD62L+ and CD45RA+, or CD45RA+ and CCR7+ are shown. The data was collected at day 7 post-transfection. The memory T cells study is described in Example 6.

FIG. 8B shows the results of a flow cytometry study for detecting the fraction of memory T cell subsets in transgenic CD4+ T cells expressing CD38 traditional DAR (V10) or CD38 mDAR (V1). The CD4+ memory T cell subsets expressing CD62L+ and CD45RA+, or CD45RA+ and CCR7+ are shown. The data was collected at day 10 post-transfection. The memory T cells study is described in Example 6.

FIG. 8C shows the results of a flow cytometry study for detecting the fraction of memory T cell subsets in transgenic CD4+ T cells expressing CD38 traditional DAR (V10) or CD38 mDAR (V1). The CD4+ memory T cell subsets expressing CD62L+ and CD45RA+, or CD45RA+ and CCR7+ are shown. The data was collected at day 13 post-transfection. The memory T cells study is described in Example 6.

FIG. 9A shows the results of a flow cytometry study for detecting the fraction of memory T cell subsets in transgenic CD8+ T cells expressing CD38 traditional DAR (V10) or CD38 mDAR (V1). The CD8+ memory T cell subsets expressing CD62L+ and CD45RA+, or CD45RA+ and CCR7+ are shown. The data was collected at day 7 post-transfection. The memory T cells study is described in Example 6.

FIG. 9B shows the results of a flow cytometry study for detecting the fraction of memory T cell subsets in transgenic CD8+ T cells expressing CD38 traditional DAR (V10) or CD38 mDAR (V1). The CD8+ memory T cell subsets expressing CD62L+ and CD45RA+, or CD45RA+ and CCR7+ are shown. The data was collected at day 10 post-transfection. The memory T cells study is described in Example 6.

FIG. 9C shows the results of a flow cytometry study for detecting the fraction of memory T cell subsets in transgenic CD8+ T cells expressing CD38 traditional DAR (V10) or CD38 mDAR (V1). The CD8+ memory T cell subsets expressing CD62L+ and CD45RA+, or CD45RA+ and CCR7+ are shown. The data was collected at day 13 post-transfection. The memory T cells study is described in Example 6.

FIG. 10 shows the results of a flow cytometry study for determining viability of transgenic T cells expressing either CD38 traditional DAR (V10) or CD38 memory DAR (V1). The data was collected at day 23 post-transfection. The viability flow cytometry study is described in Example 7.

FIG. 11A is a graph showing a comparison of cell counts of transgenic T cells (donor 34) express either CD38 traditional DAR (V10) or CD38 mDAR (V1) (Example 8).

FIG. 11B is a graph showing a comparison of cell viability of transgenic T cells (donor 36) expressing either CD38 traditional DAR (V10) or CD38 mDAR (V1) (Example 8).

FIG. 12A is a graph showing a comparison of cell counts of transgenic T cells (donor 34) expressing either CD38 traditional DAR (V10) or CD38 mDAR (V1) (Example 8).

FIG. 12B is a graph showing a comparison of cell viability of transgenic T cells (donor 36) expressing either CD38 traditional DAR (V10) or CD38 mDAR (V1) (Example 8).

FIG. 13A shows the results of an assay for detecting phosphorylated STAT5 in CD4+ transgenic T cells expressing CD38 traditional DAR (V10) (designated (C)) compared to transgenic T cells expressing CD38 mDAR (V1) (designated (B)) and compared to non-transgenic T cells activated with IL-2 (control effector cells designated (A)). The transgenic T cells were reacted with RPMI8226 cells (CD38+ expressing cell line) or K562 (CD38-minus cell line) at an effector/target cell ratio of 2:1. Non-transgenic activated T cells were used as control effector cells (designated (A)). Flow cytometry was gated on CD4+ cells to detect phosphorylated STAT5. The detection of phosphorylated STAT5 indicates JAK-STAT pathway activation in response to target antigen binding by the mDAR T cells. The STAT5 assay is described in Example 9.

FIG. 13B shows the results of an assay for detecting phosphorylated STAT5 in CD8+ transgenic T cells expressing CD38 traditional DAR (V10) (designated (C)) compared to transgenic T cells expressing CD38 mDAR (V1) (designated (B)) and compared to non-transgenic T cells activated with IL-2 (control effector cells designated (A)). The transgenic T cells were reacted with RPMI8226 cells (CD38+ expressing cell line) or K562 (CD38-minus cell line) at an effector/target cell ratio of 2:1. Non-transgenic activated T cells were used as control effector cells (designated (A)). Flow cytometry was gated on CD8+ cells to detect phosphorylated STAT5. The detection of phosphorylated STAT5 indicates JAK-STAT pathway activation in response to target antigen binding by the mDAR T cells. The STAT5 assay is described in Example 9.

FIG. 14A shows the results of an assay for detecting phosphorylated STAT3 in CD4+ transgenic T cells expressing CD38 traditional DAR (V10) (designated (C)) compared to transgenic T cells expressing CD38 mDAR (V1) (designated (B) and compared to non-transgenic T cells activated with IL-2 (control effector cells designated (A)). The transgenic T cells were reacted with RPMI8226 cells (CD38+ expressing cell line) or K562 (CD38-minus cell line) at an effector/target cell ratio of 2:1. Non-transgenic activated T cells were used as control effector cells (designated (A)). Flow cytometry was gated on CD4+ cells to detect phosphorylated STAT3. The detection of phosphorylated STAT3 indicates JAK-STAT pathway activation in response to target antigen binding by the mDAR T cells. The STAT3 assay is described in Example 9.

FIG. 14B shows the results of an assay for detecting phosphorylated STAT3 in CD8+ transgenic T cells expressing CD38 traditional DAR (V10) (designated (C)) compared to transgenic T cells expressing CD38 mDAR (V1) (designated (B)) and compared to non-transgenic T cells activated with IL-2 (control effector cells designated (A)). The transgenic T cells were reacted with RPMI8226 cells (CD38+ expressing cell line) or K562 (CD38-minus cell line) at an effector/target cell ratio of 2:1. Non-transgenic activated T cells were used as control effector cells (designated (A)). Flow cytometry was gated on CD8+ cells to detect phosphorylated STAT3. The detection of phosphorylated STAT3 indicates JAK-STAT pathway activation in response to target antigen binding by the mDAR T cells. The STAT3 assay is described in Example 9.

FIG. 15A is a bar graph showing the level of cytotoxicity of transgenic T cells expressing either CD38 traditional DAR (V10) or CD38 mDAR (V1) on target RPMI 8226 cells that express CD38. Activated T cells (ATC) and knocked-out TRAC T cells (TCR KO) were used as controls. The cytotoxicity study was conducted using transgenic T cells at day 14 post-transfection. Viability of the target cells were detected via flow cytometry using Annexin V as an index for cytotoxicity. The effector-to-target ratios testing included 2:1, 1:1, 0.5:1 and 0:1. The cytotoxicity study is described in Example 10.

FIG. 15B is a bar graph showing the level of cytotoxicity of transgenic T cells expressing either CD38 traditional DAR (V10) or CD38 mDAR (V1) on target Raji cells that express CD38. Activated T cells (ATC) and knocked-out TRAC T cells (TCR KO) were used as controls. The cytotoxicity study was conducted using transgenic T cells at day 14 post-transfection. Viability of the target cells were detected via flow cytometry using Annexin V as an index for cytotoxicity. The effector-to-target ratios testing included 2:1, 1:1, 0.5:1 and 0:1. The cytotoxicity study is described in Example 10.

FIG. 15C is a bar graph showing the level of cytotoxicity of transgenic T cells expressing either CD38 traditional DAR (V10) or CD38 mDAR (V1) on target K562 cells (CD38-minus cell line). Activated T cells (ATC) and knocked-out TRAC T cells (TCR KO) were used as controls. The cytotoxicity study was conducted using transgenic T cells at day 14 post-transfection. Viability of the target cells were detected via flow cytometry using Annexin V as an index for cytotoxicity. The effector-to-target ratios testing included 2:1, 1:1, 0.5:1 and 0:1. The cytotoxicity study is described in Example 10.

FIG. 16A is a bar graph showing the level of tumor necrosis factor alpha (TNFa) released by transgenic CD4+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 16B is a bar graph showing the level of granulocyte-macrophage colony-stimulating factor (GM-CSF) released by transgenic CD4+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 16C is a bar graph showing the level of interleukin-2 (IL-2) released by transgenic CD4+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 16D is a bar graph showing the level of interferon gamma (IFNg) released by transgenic CD4+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 17A is a bar graph showing the level of tumor necrosis factor alpha (TNFa) released by transgenic CD8+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 17B is a bar graph showing the level of granulocyte-macrophage colony-stimulating factor (GM-CSF) released by transgenic CD8+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 17C is a bar graph showing the level of interleukin-2 (IL-2) released by transgenic CD8+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 17D is a bar graph showing the level of interferon gamma (IFNg) released by transgenic CD8+ T cells expressing either CD38 traditional DAR (V10) or CD38 mDAR (V1) when contacted with target cells. The target cells include K562 (CD38-minus cell line), and two cell lines that express CD38 antigen (RPMI 8226 and Raji). Activated T cells and knocked-out TRAC T cells (KO) were used as controls. The effector-to-target cell ratio was 1:1. The cytokine release study was conducted at day 14 post-transfection. The cytokine release study is described in Example 11.

FIG. 18A shows bioluminescent imaging of tumoricidal activity of transgenic T cells expressing either CD38 traditional DAR (V10) (designated T) or CD38 mDAR (V1) (designated M) in a xenograft mouse model at days −1, 6, 9, 13, 20 and 36 post-treatment with the transgenic T cells. The mice were inoculated with RPMI8226-Fluc tumor cells. The xenograft mouse study is described in Example 12.

FIG. 18B is a graph showing detection of circulating CD45+ human T cells in blood samples from mice treated with either CD38 traditional DAR (V10) (designated DAR-T) or CD38 mDAR (V1) (designated mDAR-T) in the xenograft mouse model described in FIG. 18A. The detection of circulating CD45+ cells in the xenograft mouse study is described in Example 12.

FIG. 19A shows bioluminescent imaging of a tumor re-challenge study in mice inoculated with RPMI8226-Fluc tumor cells and previously treated with transgenic T cells expressing either CD38 traditional DAR (V10) (designated T) or CD38 mDAR (V1) (designated M). The tumor imaging data shows days 8, 11, 14 and 21 post-re-challenge. The xenograft mouse study is described in Example 12.

FIG. 19B is a graph showing detection of circulating CD45+ human T cells in blood samples from the xenograft mice that were re-challenged with RPMI8226-Fluc tumor cells as in described in FIG. 19A. The detection of circulating CD45+ cells in the xenograft mouse tumor re-challenge study is described in Example 12.

FIG. 19C shows bioluminescent imaging of a dose study in xenograft mice inoculated with RPMI8226-Fluc tumor cells and then treated with four different doses of transgenic T cells expressing CD38 memory DAR (V1) or control T cells having knocked-out TRAC locus (KO). The bioluminescent images were taken at days −1, 6, 22, 26 and 33 post treatment. The mouse dose study is described in Example 12.

Example 19D is a graph showing detection of circulating CD45+ human T cells in blood samples from the xenograft mice that were treated with different doses of transgenic T cells expressing CD38 memory DAR (V1) as in described in FIG. 19C. The detection of circulating CD45+ cells in the xenograft mouse dose study is described in Example 12.

FIG. 20A shows the results of in vitro an assay for detecting phosphorylated STAT5 in CD4+ transgenic T cells expressing CD38 traditional DAR (V12) (designated (C)) compared to transgenic T cells expressing CD38 mDAR (V3) (designated (B)) and compared to non-transgenic T cells activated with IL-2 (control effector cells designated (A)). The STAT5 assay is described in Example 9.

FIG. 20B shows the results of an in vitro assay for detecting phosphorylated STAT5 in CD8+ transgenic T cells expressing CD38 traditional DAR (V11) (designated (C)) compared to transgenic T cells expressing CD38 mDAR (V2) (designated (B)) and compared to non-transgenic T cells activated with IL-2 (control effector cells designated (A)). The STAT5 assay is described in Example 9.

FIG. 21A shows the results of a flow cytometry study for detecting expression of CD38 DAR and CD3 in transgenic T cells expressing traditional DAR, either V10, V11 or V12 traditional DAR constructs, at day 7, 10, 14 or 17 post-transfection (Example 6).

FIG. 21B shows the results of a flow cytometry study for detecting expression of CD38 DAR and CD3 in transgenic T cells expressing memory DAR, either V1, V2 or V3 mDAR constructs, at day 7, 10, 14 or 17 post-transfection (Example 6).

FIG. 22A shows the results of a flow cytometry study for detecting the fraction of memory T cell subsets in transgenic CD4+ T cells expressing CD38 traditional DAR constructs, either V11 or V12. The CD4+ memory T cell subsets expressing CD62L+ and CD45RA+, or CD45RA+ and CCR7+ are shown. The data was collected at day 7 post-transfection. The memory T cells study is described in Example 6.

FIG. 22B shows the results of a flow cytometry study for detecting the fraction of memory T cell subsets in transgenic CD4+ T cells expressing CD38 traditional DAR constructs, either V10, V11 or V12. The CD4+ memory T cell subsets expressing CD62L+ and CD45RA+, or CD45RA+ and CCR7+ are shown. The data was collected at day 10 post-transfection. The memory T cells study is described in Example 6.

FIG. 22C shows the results of a flow cytometry study for detecting the fraction of memory T cell subsets in transgenic CD4+ T cells expressing CD38 traditional DAR constructs, either V10, V11 or V12. The CD4+ memory T cell subsets expressing CD62L+ and CD45RA+, or CD45RA+ and CCR7+ are shown. The data was collected at day 14 post-transfection. The memory T cells study is described in Example 6.

FIG. 22D shows the results of a flow cytometry study for detecting the fraction of memory T cell subsets in transgenic CD4+ T cells expressing CD38 traditional DAR constructs, either V10, V11 or V12. The CD4+ memory T cell subsets expressing CD62L+ and CD45RA+, or CD45RA+ and CCR7+ are shown. The data was collected at day 17 post-transfection. The memory T cells study is described in Example 6.

FIG. 23A shows the results of a flow cytometry study for detecting the fraction of memory T cell subsets in transgenic CD4+ T cells expressing CD38 memory DAR constructs, either V2 or V3. The CD4+ memory T cell subsets expressing CD62L+ and CD45RA+, or CD45RA+ and CCR7+ are shown. The data was collected at day 7 post-transfection. The memory T cells study is described in Example 6.

FIG. 23B shows the results of a flow cytometry study for detecting the fraction of memory T cell subsets in transgenic CD4+ T cells expressing CD38 memory DAR constructs, either V1, V2 or V3. The CD4+ memory T cell subsets expressing CD62L+ and CD45RA+, or CD45RA+ and CCR7+ are shown. The data was collected at day 10 post-transfection. The memory T cells study is described in Example 6.

FIG. 23C shows the results of a flow cytometry study for detecting the fraction of memory T cell subsets in transgenic CD4+ T cells expressing CD38 memory DAR constructs, either V1, V2 or V3. The CD4+ memory T cell subsets expressing CD62L+ and CD45RA+, or CD45RA+ and CCR7+ are shown. The data was collected at day 14 post-transfection. The memory T cells study is described in Example 6.

FIG. 23D shows the results of a flow cytometry study for detecting the fraction of memory T cell subsets in transgenic CD4+ T cells expressing CD38 memory DAR constructs, either V1, V2 or V3. The CD4+ memory T cell subsets expressing CD62L+ and CD45RA+, or CD45RA+ and CCR7+ are shown. The data was collected at day 17 post-transfection. The memory T cells study is described in Example 6.

FIG. 24A shows the results of a flow cytometry study for detecting the fraction of memory T cell subsets in transgenic CD8+ T cells expressing CD38 traditional DAR constructs, either V11 or V12. The CD8+ memory T cell subsets expressing CD62L+ and CD45RA+, or CD45RA+ and CCR7+ are shown. The data was collected at day 7 post-transfection. The memory T cells study is described in Example 6.

FIG. 24B shows the results of a flow cytometry study for detecting the fraction of memory T cell subsets in transgenic CD8+ T cells expressing CD38 traditional DAR constructs, either V10, V11 or V12. The CD8+ memory T cell subsets expressing CD62L+ and CD45RA+, or CD45RA+ and CCR7+ are shown. The data was collected at day 10 post-transfection. The memory T cells study is described in Example 6.

FIG. 24C shows the results of a flow cytometry study for detecting the fraction of memory T cell subsets in transgenic CD8+ T cells expressing CD38 traditional DAR constructs, either V10, V11 or V12. The CD8+ memory T cell subsets expressing CD62L+ and CD45RA+, or CD45RA+ and CCR7+ are shown. The data was collected at day 14 post-transfection. The memory T cells study is described in Example 6.

FIG. 24D shows the results of a flow cytometry study for detecting the fraction of memory T cell subsets in transgenic CD8+ T cells expressing CD38 traditional DAR constructs, either V10, V11 or V12. The CD8+ memory T cell subsets expressing CD62L+ and CD45RA+, or CD45RA+ and CCR7+ are shown. The data was collected at day 17 post-transfection. The memory T cells study is described in Example 6.

FIG. 25A shows the results of a flow cytometry study for detecting the fraction of memory T cell subsets in transgenic CD8+ T cells expressing CD38 memory DAR constructs, either V2 or V3. The CD8+ memory T cell subsets expressing CD62L+ and CD45RA+, or CD45RA+ and CCR7+ are shown. The data was collected at day 7 post-transfection. The memory T cells study is described in Example 6.

FIG. 25B shows the results of a flow cytometry study for detecting the fraction of memory T cell subsets in transgenic CD8+ T cells expressing CD38 memory DAR constructs, either V1, V2 or V3. The CD8+ memory T cell subsets expressing CD62L+ and CD45RA+, or CD45RA+ and CCR7+ are shown. The data was collected at day 10 post-transfection. The memory T cells study is described in Example 6.

FIG. 25C shows the results of a flow cytometry study for detecting the fraction of memory T cell subsets in transgenic CD8+ T cells expressing CD38 memory DAR constructs, either V1, V2 or V3. The CD8+ memory T cell subsets expressing CD62L+ and CD45RA+, or CD45RA+ and CCR7+ are shown. The data was collected at day 14 post-transfection. The memory T cells study is described in Example 6.

FIG. 25D shows the results of a flow cytometry study for detecting the fraction of memory T cell subsets in transgenic CD8+ T cells expressing CD38 memory DAR constructs, either V1, V2 or V3. The CD8+ memory T cell subsets expressing CD62L+ and CD45RA+, or CD45RA+ and CCR7+ are shown. The data was collected at day 17 post-transfection. The memory T cells study is described in Example 6.

FIG. 26A shows the results of a flow cytometry study for determining percentage of viable transgenic T cells expressing CD38 traditional DAR (V10). The data was collected at day 14 post-transfection. The viability flow cytometry study is described in Example 7.

FIG. 26B shows the results of a flow cytometry study for determining percentage of viable transgenic T cells expressing CD38 traditional DAR (V11). The data was collected at day 14 post-transfection. The viability flow cytometry study is described in Example 7.

FIG. 26C shows the results of a flow cytometry study for determining percentage of viable transgenic T cells expressing CD38 traditional DAR (V12). The data was collected at day 14 post-transfection. The viability flow cytometry study is described in Example 7.

FIG. 27A shows the results of a flow cytometry study for determining percentage of viable transgenic T cells expressing CD38 memory DAR (V1). The data was collected at day 14 post-transfection. The viability flow cytometry study is described in Example 7.

FIG. 27B shows the results of a flow cytometry study for determining percentage of viable transgenic T cells expressing CD38 memory DAR (V2). The data was collected at day 14 post-transfection. The viability flow cytometry study is described in Example 7.

FIG. 27C shows the results of a flow cytometry study for determining percentage of viable transgenic T cells expressing CD38 memory DAR (V3). The data was collected at day 14 post-transfection. The viability flow cytometry study is described in Example 7.

FIG. 28A is a bar graph showing the level of cytotoxicity of transgenic T cells expressing either CD38 traditional DAR (V10, V11 or V12) or CD38 mDAR (V1, V2 or V3) on target RPMI 8226 cells that express CD38. Activated T cells (ATC) and knocked-out TRAC T cells (KO) were used as controls. The cytotoxicity study was conducted using transgenic T cells at day 15 post-transfection. Viability of the target cells were detected via flow cytometry using Annexin V as an index for cytotoxicity. The effector-to-target ratios testing included 2:1, 1:1, 0.5:1 and 0:1. The cytotoxicity study is described in Example 10.

FIG. 28B is a bar graph showing the level of cytotoxicity of transgenic T cells expressing either CD38 traditional DAR (V10, V11 or V12) or CD38 mDAR (V1, V2 or V3) on target Raji cells that express CD38. Activated T cells (ATC) and knocked-out TRAC T cells (KO) were used as controls. The cytotoxicity study was conducted using transgenic T cells at day 15 post-transfection. Viability of the target cells were detected via flow cytometry using Annexin V as an index for cytotoxicity. The effector-to-target ratios testing included 2:1, 1:1, 0.5:1 and 0:1. The cytotoxicity study is described in Example 10.

FIG. 29A is a bar graph showing the level of interleukin-2 (IL-2) released by transgenic CD4+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 29B is a bar graph showing the level of interferon gamma (IFNγ) released by transgenic CD4+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 29C is a bar graph showing the level of tumor necrosis factor alpha (TNFα) released by transgenic CD4+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 29D is a bar graph showing the level of granulocyte-macrophage colony stimulating factor (GS-CSF) released by transgenic CD4+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 30A is a bar graph showing the level of interleukin-2 (IL-2) released by transgenic CD8+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 30B is a bar graph showing the level of interferon gamma (IFNγ) released by transgenic CD8+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 30C is a bar graph showing the level of tumor necrosis factor alpha (TNFα) released by transgenic CD8+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 30D is a bar graph showing the level of granulocyte-macrophage colony stimulating factor (GM-CSF) released by transgenic CD8+ T cells expressing either CD38 traditional DAR (V10, V11 or V12) or CD38 mDAR (V1, V2 or V3) when contacted with target RPMI cells. Activated T cells (ATC) and knocked-out TRAC T cells (KO) were used as controls. The effector-to-target cell ratio was 2:1. The cytokine release study was conducted at day 15 post-transfection. The cytokine release study is described in Example 11.

FIG. 31 is a graph showing the results of an in vitro tumor re-challenge assay. Transgenic T cells expressing either CD38 traditional DAR (V10, V11 or V12) or CD38 mDAR (V1, V2 or V3) were challenged, and then re-challenged, with CD38-expressing tumor cell lines RPMI or Raji. The re-challenge assay was conducted with transgenic DAR and mDAR T cells at day 15 post-transfection. The re-challenge assay is described in Example 13.

FIG. 32A is a graph showing the ratio of CD8+/CD4+ transgenic T cells expressing one of three different CD38 traditional DAR constructs (V10, V11 or V12) compared to the ratio of CD8+/CD4+ transgenic T cells expressing one of three different CD38 mDAR constructs (V10, V11 or V12). The data was collected at day 7, 10, 14, 17, 28 and 35 post-transfection, from PBMCs from donor 36. The assay is described in Example 14.

FIG. 32B is a graph showing the ratio of CD8+/CD4+ transgenic T cells expressing one of three different CD38 traditional DAR constructs (V10, V11 or V12) compared to the ratio of CD8+/CD4+ transgenic T cells expressing one of three different CD38 mDAR constructs (V10, V11 or V12). The data was collected at day 7, 10, 14, 17, 28 and 35 post-transfection, from PBMCs from donor 37. The assay is described in Example 14.

FIG. 33 lists amino acid sequences of exemplary embodiments of intracellular costimulatory regions, and intracellular region from human IL2Rβ and amino acid sequences of exemplary embodiments of intracellular regions from CD3ζ.

FIG. 34 lists amino acid sequences of exemplary embodiments of intracellular regions from CD3ζ.

FIG. 35 lists amino acid sequences of exemplary embodiments of ITAM 1, 2 and 3 regions from CD3ζ, and BRR motifs 1, 2 and 3.

FIG. 36 lists amino acid sequences of precursor, first polypeptide and second polypeptide of memory DAR (V1).

FIG. 37 lists amino acid sequences of precursor, first polypeptide and second polypeptide of memory DAR (V2).

FIG. 38 lists amino acid sequences of precursor, first polypeptide and second polypeptide of memory DAR (V3).

FIG. 39 lists amino acid sequences of precursor, first polypeptide and second polypeptide of traditional DAR (V10).

FIG. 40 lists amino acid sequences of precursor, first polypeptide and second polypeptide of traditional DAR (V11).

FIG. 41 lists amino acid sequences of precursor, first polypeptide and second polypeptide of traditional DAR (V12).

FIG. 42 lists amino acid sequences of precursor, first polypeptide and second polypeptide of memory DAR (V4).

FIG. 43 lists amino acid sequences of precursor, first polypeptide and second polypeptide of memory DAR (V5).

FIG. 44 lists amino acid sequences of precursor, first polypeptide and second polypeptide of memory DAR (V6).

FIG. 45 lists amino acid sequences of precursor, first polypeptide and second polypeptide of memory DAR (V7).

FIG. 46 lists amino acid sequences of precursor, first polypeptide and second polypeptide of memory DAR (V8).

FIG. 47 lists amino acid sequences of precursor, first polypeptide and second polypeptide of memory DAR (V9).

FIG. 48 lists amino acid sequences of intracellular regions of memory DARs V1, V2, V3, V4, V5 and V6.

FIG. 49 lists amino acid sequences of intracellular regions of memory DARs V7, V8 and V9, and intracellular regions of traditional DARs V10, V11 and V12.

FIG. 50 lists amino acid sequences of full-length human IL2Rβ including the extracellular region (amino acids 1-290), transmembrane region (amino acids 241-265), and intracellular region (amino acids 387-551). The underlined portions comprise the intracellular IL2Rβ used in the mDAR constructs described herein. FIG. 50 also lists the amino acid sequences of ITAM regions from human CD3 gamma, delta and epsilon.

FIG. 51 provides the in vivo images up to 8 weeks after treatment of mice inoculated with RPMI8826-Fluc tumor cells and then treated with PBS only, TRAC knockout T cells (“KO”), and, proceeding from left to right: 10⁶ CD38 mDAR(V1)-T cells, 10⁵ CD38 mDAR(V1)-T cells, 10⁴ CD38 mDAR(V1)-T cells, 10⁷ CD38 DAR(V10)-T cells, and 10⁶ CD38 DAR(V10)-T cells.

FIG. 52A is a graph of the average total flux over time of the tumors of treatment groups of RPMI8826 tumor-bearing mice shown in FIG. 51: PBS control, TRAC knockout T cells (“KO”), 10⁶ CD38 mDAR(V1)-T cells (“1e6 m”), 10⁵ CD38 mDAR(V1)-T cells (“1e5 m”), 10⁴ CD38 mDAR(V1)-T cells (“1e4 m”), 10⁷ CD38 DAR(V10)-T cells (“1e7 DAR”), and 10⁶ CD38 DAR(V10)-T cells (“1e6 DAR”).

FIG. 52A is a graph of the average body weights of RPMI8826 tumor-inoculated mice in the treatment groups as shown in FIG. 51 over the course of the study: PBS control, TRAC knockout T cells (“KO”), 10⁶ CD38 mDAR(V1)-T cells (“1e6 m”), 10⁵ CD38 mDAR(V1)-T cells (“1e5 m”), 10⁴ CD38 mDAR(V1)-T cells (“1e4 m”), 10⁷ CD38 DAR(V10)-T cells (“1e7 DAR”), and 10⁶ CD38 DAR(V10)-T cells (“1e6 DAR”).

FIG. 53 provides a graph depicting average numbers of human T cells in the peripheral blood of RPMI8826 tumor-inoculated mice of the treatment groups depicted in FIG. 51 over the course of the study: PBS control, TRAC knockout T cells (“KO”), 10⁶ CD38 mDAR(V1)-T cells (“1e6 m”), 10⁵ CD38 mDAR(V1)-T cells (“1e5 m”), 10⁴ CD38 mDAR(V1)-T cells (“1e4 m”), 10⁷ CD38 DAR(V10)-T cells (“1e7 DAR”), and 10⁶ CD38 DAR(V10)-T cells (“1e6 DAR”).

FIG. 54 provides the in vivo images up to 8 weeks after treatment of mice inoculated with RPMI8826-Fluc tumor cells and then treated with PBS only, TRAC knockout T cells (“KO”), and, proceeding from left to right: 10⁶ CD38 mDAR(V1)-T cells, 10⁵ CD38 mDAR(V1)-T cells, 10⁴ CD38 mDAR(V1)-T cells, 10⁶ CD38 mDAR(V3)-T cells, 10⁵ CD38 mDAR(V3)-T cells, 10⁴ CD38 mDAR(V3)-T cells, 10⁶ CD38 mDAR(V2)-T cells, 10⁵ CD38 mDAR(V2)-T cells, and 10⁴ CD38 mDAR(V2)-T cells, with seven mice per treatment group.

FIG. 55A is a graph of the average total flux over time of the tumors of treatment groups of RPMI8826 tumor-bearing mice shown in FIG. 54: PBS control, TRAC knockout T cells (“KO”), 10⁶ CD38 mDAR(V1)-T cells (“DAR JS 1e6”), 10⁵ CD38 mDAR(V1)-T cells (“DAR JS 1e5”), 10⁴ CD38 mDAR(V1)-T cells (“DAR JS 1e4”), 10⁶ CD38 mDAR(V3)-T cells (“BRR3 JS 1e6”), 10⁵ CD38 mDAR(V3)-T cells (“BRR3 JS 1e5”), 10⁴ CD38 mDAR(V3)-T cells (“BRR3 JS 1e4”), 10⁶ CD38 mDAR(V2)-T cells (“BRR123 JS 1e6”), 10⁵ CD38 mDAR(V2)-T cells (“BRR123 JS 1e5”), and 10⁴ CD38 mDAR(V2)-T cells (“BRR123 JS 1e4”).

FIG. 55B is a graph of the average body weights of RPMI8826 tumor-inoculated mice in the treatment groups as shown in FIG. 54 over the course of the study: PBS control, TRAC knockout T cells (“KO”), 10⁶ CD38 mDAR(V1)-T cells (“DAR JS 1e6”), 10⁵ CD38 mDAR(V1)-T cells (“DAR JS 1e5”), 10⁴ CD38 mDAR(V1)-T cells (“DAR JS 1e4”), 10⁶ CD38 mDAR(V3)-T cells (“BRR3 JS 1e6”), 10⁵ CD38 mDAR(V3)-T cells (“BRR3 JS 1e5”), 10⁴ CD38 mDAR(V3)-T cells (“BRR3 JS 1e4”), 10⁶ CD38 mDAR(V2)-T cells (“BRR123 JS 1e6”), 10⁵ CD38 mDAR(V2)-T cells (“BRR123 JS 1e5”), and 10⁴ CD38 mDAR(V2)-T cells (“BRR123 JS 1e4”).

FIG. 56 provides a graph depicting the average number of human T cells in the peripheral blood of RPMI8826 tumor-inoculated mice of the treatment groups depicted in FIG. 54 over the course of the study: PBS control, TRAC knockout T cells (“KO”), 10⁶ CD38 mDAR(V1)-T cells (“DAR JS 1e6”), 10⁵ CD38 mDAR(V1)-T cells (“DAR JS 1e5”), 10⁴ CD38 mDAR(V1)-T cells (“DAR JS 1e4”), 10⁶ CD38 mDAR(V3)-T cells (“BRR3 JS 1e6”), 10⁵ CD38 mDAR(V3)-T cells (“BRR3 JS 1e5”), 10⁴ CD38 mDAR(V3)-T cells (“BRR3 JS 1e4”), 10⁶ CD38 mDAR(V2)-T cells (“BRR123 JS 1e6”), 10⁵ CD38 mDAR(V2)-T cells (“BRR123 JS 1e5”), and 10⁴ CD38 mDAR(V2)-T cells (“BRR123 JS 1e4”).

FIG. 57 provides the in vivo images up to 8 weeks after treatment of mice inoculated with Daudi-Fluc tumor cells and then treated with PBS only, TRAC knockout T cells (“KO”), and, proceeding from left to right: 10⁷ CD38 DAR(V10)-T cells, 10⁶ CD38 DAR(V10)-T cells, 10⁵ CD38 DAR(V10)-T cells, 10⁷ CD38 mDAR(V3)-T cells, 10⁶ CD38 mDAR(V3)-T cells, and 10⁵ CD38 mDAR(V3)-T cells (eight mice per group).

FIG. 58A is a graph of the average total flux over time of the tumors of treatment groups of Daudi tumor-bearing mice shown in FIG. 57: PBS control, TRAC knockout T cells (“KO”), 10⁷ CD38 DAR(V10)-T cells (“CD38 DAR 1e7”), 10⁶ CD38 DAR(V10)-T cells (“CD38 DAR 1e6”), 10⁵ CD38 DAR(V10)-T cells (“CD38 DAR 1e5”), 10⁷ CD38 mDAR(V3)-T cells (“BRR3 JS 1e7”), 10⁶ CD38 mDAR(V3)-T cells (“BRR3 JS 1e6”), and 10⁵ CD38 mDAR(V3)-T cells (“BRR3 JS 1e5”).

FIG. 58B is a graph of the average body weights of Daudi tumor-inoculated mice in the treatment groups as shown in FIG. 57 over the course of the study: PBS control, TRAC knockout T cells (“KO”), 10⁷ CD38 DAR(V10)-T cells (“CD38 DAR 1e7”), 10⁶ CD38 DAR(V10)-T cells (“CD38 DAR 1e6”), 10⁵ CD38 DAR(V10)-T cells (“CD38 DAR 1e5”), 10⁷ CD38 mDAR(V3)-T cells (“BRR3 JS 1e7”), 10⁶ CD38 mDAR(V3)-T cells (“BRR3 JS 1e6”), and 10⁵ CD38 mDAR(V3)-T cells (“BRR3 JS 1e5”).

FIG. 59A provides a graph depicting average numbers of human T cells in the peripheral blood of Daudi tumor-inoculated mice of the treatment groups depicted in FIG. 57 over the course of the study: PBS control, TRAC knockout T cells (“KO”), PBS control, TRAC knockout T cells (“KO”), 10⁷ CD38 DAR(V10)-T cells (“CD38 DAR 1e7”), 10⁶ CD38 DAR(V10)-T cells (“CD38 DAR 1e6”), 10⁵ CD38 DAR(V10)-T cells (“CD38 DAR 1e5”), 10⁷ CD38 mDAR(V3)-T cells (“BRR3 JS 1e7”), 10⁶ CD38 mDAR(V3)-T cells (“BRR3 JS 1e6”), and 10⁵ CD38 mDAR(V3)-T cells (“BRR3 JS 1e5”).

FIG. 59B provides a graph depicting the average number of human T cells in the peripheral blood of Daudi tumor-inoculated mice of the mice treated with 10⁷ CD38 mDAR(V3)-T cells (“BRR3 JS 1e7”) up to seven weeks after treatment.

FIG. 60 provides in vivo images of mice inoculated with RPMI-8226 cells and treated with 3.5×10⁶ T cells expressing either CD38 mDAR(V3 (left 2 columns) or CD38 DAR (V10) (right 2 columns). Mice were rechallenge with tumor at the timepoints designated with arrows.

### DETAILED DESCRIPTION

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The headings provided herein are not limitations of the various aspects of the disclosure, which aspects can be understood by reference to the specification as a whole.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, N.Y.; Harlow and Lane (1989) Antibodies: A Laboratory Manual Cold Spring Harbor Press, N.Y.; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “peptide chain”, “polypeptide”, “polypeptide chain” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecule. Precursor molecules include those that have not yet been subjected to post-translation modification such as proteolytic cleavage, cleavage due to ribosomal skipping (e.g., mediated by a self-cleaving cleaving sequence such as for example T2A, P2A, E2A or F2A), hydroxylation, methylation, lipidation, acetylation, SUMOylation, ubiquitination, glycosylation, fucosylation, phosphorylation, disulfide bond formation, processing of a secretory signal peptide or non-enzymatic cleavage at certain amino acid residues. Polypeptides include mature molecules that have undergone any one or any combination of the post-translation modifications described above. These terms encompass native proteins, recombinant proteins and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 2-6 or more polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex. Dimeric antigen receptors (mDAR) comprising two polypeptide chains are described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an mDAR, or a fragment, derivative, mutein, or variant thereof. In one embodiment, nucleic acids comprise one type of polynucleotide or a mixture of two or more different types of polynucleotides. Nucleic acids encoding dimeric antigen receptors (mDAR) or antigen-binding portions thereof, are described herein. With respect to embodiments involving a first nucleic acid (e.g., encoding a first polypeptide) and a second nucleic acid (e.g., encoding a second polypeptide), the first nucleic acid and second nucleic acid may be provided either as separate molecules or within the same continuous molecule (e.g., a plasmid or other construct containing first and second coding sequences).

As used herein, the term “heterologous” refers to a foreign nucleic acid sequence (or a fragment thereof) that is inserted into, or appended to, a nucleic acid gene sequence where the foreign nucleic acid sequence is not native to the nucleic acid gene sequence. The foreign nucleic acid sequence may have a function not found in the nucleic acid gene sequence. The function of the foreign nucleic acid sequence may be conferred upon the nucleic acid gene sequence by inserting or appending the foreign nucleic acid sequence to the nucleic acid gene sequence. The term “heterologous” also refers to a foreign polypeptide sequence (or a fragment thereof) that is inserted into, or appended to, a polypeptide element where the foreign polypeptide sequence is not native to the polypeptide element. The foreign polypeptide sequence may have a function not found in the polypeptide element. The function of the foreign polypeptide sequence may be conferred upon the polypeptide element by inserting or appending the foreign polypeptide sequence to the polypeptide sequence. In one embodiment, a STAT binding motif is not found in native CD3ζ intracellular sequence. In one embodiment, native CD3ζ intracellular signaling regions lack a STAT binding motif and therefore do not bind STAT (Signal Transducer and Activator of Transcription) proteins. Appending a foreign/heterologous STAT binding sequence to the CD3ζ intracellular signaling region confers STAT protein binding capability to the CD3ζ intracellular signaling region.

The term “recover” or “recovery” or “recovering”, and other related terms, refers to obtaining a protein (e.g., an mDAR or a precursor or antigen binding portion thereof), from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an mDAR or a precursor or antigen binding portion thereof) or polynucleotide that is substantially free of other cellular material. A protein may be rendered substantially free of naturally associated components (or components associated with a cellular expression system or chemical synthesis methods used to produce the mDAR) by isolation, using protein purification techniques well known in the art. The term isolated also refers in some embodiment to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrometry. In one embodiment, isolated precursor polypeptides, and first and second polypeptide chains, of the memory dimeric antigen receptor (mDAR) or antigen-binding portions thereof, of the present disclosure are isolated.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8α, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS (SEQ ID NO: 88).

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include memory dimeric antigen receptors (mDARs), antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins comprising memory dimeric antigen receptors (mDAR) are described herein.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having memory dimeric antigen receptor (mDAR) structures with immunoglobulin-like properties that bind specifically to a target antigen (e.g., CD38 antigen) are described herein.

The variable regions of immunoglobulin chains, and more generally, any polypeptide (e.g., mDAR or precursor thereof) comprising a light chain variable region and/or heavy chain variable region, exhibit the same general structure of relatively conserved framework regions (FR) joined by three hypervariable regions, also called complementarity determining regions or CDRs. From N-terminus to C-terminus, both light and heavy chains comprise the segments FRE CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Antibodies-like molecules comprising memory dimeric antigen receptors (mDAR) are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Memory dimeric antigen receptors (mDARs) having antibody heavy chain variable regions and antibody light chain variable regions that form antigen binding domains are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹M or less, or 10⁻¹⁰ M or less, or 10⁻¹¹M or less. In one embodiment, memory dimeric antigen receptors (mDAR) that bind specifically to their target antigen (e.g., CD38 antigen) are described herein.

In one embodiment, binding specificity of an antibody or antigen binding protein or antibody fragment can be measure by ELISA, radioimmune assay (RIA), electrochemiluminescence assays (ECL), immunoradiometric assay (IRMA), or enzyme immune assay (EIA).

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. In one embodiment, memory dimeric antigen receptors (mDAR) or antigen-binding portions thereof that bind an epitope of CD38 antigen are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. In one embodiment, dimeric antigen receptors comprising a Fab fragment joined to a hinge, transmembrane and intracellular regions are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). In one embodiment, dimeric antigen receptors comprising a Fab fragment joined to a hinge, transmembrane and intracellular regions are described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Memory dimeric antigen receptors (mDAR) comprising fully human antibody heavy chain variable region and fully human antibody light chain variable regions are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293. In some embodiments of an mDAR or precursor thereof described herein, the heavy chain variable domain and light chain variable domain of the mDAR or precursor thereof are humanized.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the memory dimeric antigen receptor (mDAR) antigen-binding portions thereof are described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In one embodiment, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from is a hinge region of a naturally-occurring protein, such as a CD8 hinge region or a fragment thereof, a CD8α hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In one embodiment, the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO: 147). In one embodiment, the hinge region comprises an IgG1 core hinge sequence CPXC, wherein X is P, R or S. In one embodiment, the hinge region comprises a lower hinge/CH2 sequence PAPELLGGP (SEQ ID NO: 148). In one embodiment, the hinge is joined to an Fc region (CH2) having the amino acid sequence SVFLFPPKPKDT (SEQ ID NO: 149). In one embodiment, the hinge region includes the amino acid sequence of an upper, core and lower hinge and comprises EPKSCDKTHTCPPCPAP ELLGGP (SEQ ID NO: 150). In one embodiment, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions, and may or may not include a portion of the hinge region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. In one embodiment, the Fc region can include a mutation that increases or decreases any one or any combination of these functions. In one embodiment, the Fc domain comprises a LALA-PG mutation (L234A, L235A, P329G) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex. An Fc region can bind an Fc receptor, including FcγRI (e.g., CD64), FcγRII (e.g., CD32) and/or FcγRIII (e.g., CD16a). An Fc region can bind a complement component C1q.

The term “labeled” or related terms as used herein with respect to a polypeptide refers to joinder thereof to a detectable label or moiety for detection. Exemplary detectable labels or moieties include radioactive, colorimetric, antigenic, or enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the memory dimeric antigen receptors (mDAR) or antigen-binding portions thereof that described herein can be unlabeled or can be joined to a detectable label or detectable moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test construct (e.g., mDAR) may be similar but not necessarily identical to any of the amino acid sequences of the polypeptides that make up a given memory dimeric antigen receptor (mDAR) or antigen-binding portions thereof that are described herein. The similarities between the test construct and the polypeptides can be at least 95%, or at or at least 96% identical, or at least 97% identical, or at least 98% identical, or at least 99% identical, to any of the polypeptides that make up the memory dimeric antigen receptor (mDAR) or antigen-binding portions thereof that are described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies, including the memory dimeric antigen receptors (mDAR) described herein can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “Chimeric Antigen Receptor” or “CAR” refers to a single chain fusion protein comprising an extracellular antigen-binding protein that is fused to an intracellular domain. The CAR extracellular binding domain is a single chain variable fragment (scFv or sFv) derived from fusing the variable heavy and light regions of a monoclonal antibody, such as a human monoclonal antibody. In one embodiment, a CAR comprises (i) an antigen binding protein comprising a heavy chain variable (VH) domain and a light chain variable (VL) domain wherein the VH and VL domains are joined together by a peptide linker; (ii) a hinge domain, (iii) a transmembrane domain; and (iv) an intracellular domain comprising an intracellular signaling sequence. The disclosed constructs are mDARs which are distinct from CARs in that mDARs do not use a single chain antibody for targeting but instead use separate heavy and light chain variable domain regions.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the memory dimeric antigen receptors (mDAR) or antigen-binding portions thereof that are described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one into which an exogenous nucleic acid (e.g., comprising a transgene) has been introduced. The host cell includes the primary subject cell and its progeny. Exogenous nucleic acids encoding at least a portion of any of the memory dimeric antigen receptors (mDAR) or antigen-binding portions thereof that are described herein can be introduced into a host cell. Expression vectors comprising at least a portion of any of the memory dimeric antigen receptors (mDAR) or antigen-binding portions thereof that are described herein can be introduced into a host cell, and the host cell can express polypeptides comprising at least a portion of the memory dimeric antigen receptor (mDAR) or antigen-binding portions thereof that are described herein.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. The host cell (or a population thereof) includes immortalized cell lines. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome, or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS or CRISPR/Cas. A transgene can be introduced into a host cell's genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or traditional DAR or mDAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Patent Application Publication No. US 2018/0346927. In one embodiment, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a traditional DAR or mDAR construct described herein. Electroporation, nucleofection or lipofection can be used to co-deliver into the host cell the donor DNA with the zinc finger nuclease, TALEN or CRISPR/Cas system.

Transgenic host cells can be prepared by transducing host cells (e.g., T cells) with a retroviral vector carrying a nucleic acid encoding a traditional DAR or mDAR construct. The transduction can be performed essentially as described in Ma et al., 2004 The Prostate 61:12-25; and Ma et al., The Prostate 74(3):286-296, 2014 (the disclosures of which are incorporated by reference herein in their entireties). The retroviral vector can be transfected into a Phoenix-Eco cell line (ATCC) using FuGene reagent (Promega, Madison, Wis.) to produce Ecotropic retrovirus, then harvest transient viral supernatant (Ecotropic virus) can be used to transduce PG13 packaging cells with Gal-V envelope to produce retrovirus to infect human cells. Viral supernatant from the PG13 cells can be used to transduce activated T cells (or PBMCs) two to three days after CD3 or CD3/CD28 activation. Activated human T cells can be prepared by activating normal healthy donor peripheral blood mononuclear cells (PBMC) with 100 ng/ml mouse anti-human CD3 antibody OKT3 (Orth Biotech, Rartian, N.J.) or anti-CD3, anti-CD28 TransAct (Miltenyi Biotech, German) as manufacturer's manual and 300-1000 U/ml IL-2 in AIM-V growth medium (GIBCO-Thermo Fisher scientific, Waltham, Mass.) supplemented with 5% FBS for two days. Approximately 5×10⁶ activated human T cells can be transduced in a 10 ug/ml retronectin (Takara Bio USA) pre-coated 6-well plate with 3 ml viral supernatant and centrifuged at 1000 g for about 1 hour at approximately 32° C. After transduction, the transduced T cells can be expanded in AIM-V growth medium supplemented with 5% FBS and 300-1000 U/ml IL-2.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed or transfected) with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more polypeptides that comprise a memory dimeric antigen receptor (mDAR) or antigen-binding portions thereof are described herein.

The host cell or the population of host cells comprise T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

Polypeptides of the present disclosure (e.g., memory dimeric antigen receptors (mDAR)) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. The protein is then purified from culture media or cell extracts. Any of the polypeptide chains that comprise the memory dimeric antigen receptors (mDAR) or antigen-binding portions thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are at least 65% pure, at least 75% pure, at least 85% pure, at least 95% pure, or at least 98% pure. Regardless of the exact numerical value of the purity, the polypeptide is sufficiently pure for use as a pharmaceutical product. Any of the memory dimeric antigen receptors (mDAR) or antigen-binding portions thereof that are described herein can be expressed by transgenic host cells and then purified to about 65-98% purity or high level of purity using any art-known method.

In certain embodiments, the antibodies and antigen binding proteins described herein (e.g., mDAR) can further comprise post-translational modifications. Exemplary post-translational protein modifications include phosphorylation, acetylation, methylation, ADP-ribosylation, ubiquitination, glycosylation, afucosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, glycosylation can be sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

The present disclosure provides therapeutic compositions comprising any of the memory dimeric antigen receptors (mDAR) that are described herein, or cells described herein (e.g., expressing an mDAR described herein) in an admixture with a pharmaceutically-acceptable excipient. Excipients encompass, for example, carriers, stabilizers, diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers. Where a therapeutic composition comprises cells, the pharmaceutically-acceptable excipients will be chosen so as not to interfere with the viability or activity of the cells.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the memory dimeric antigen receptors (mDAR) or antigen-binding portions thereof described herein may be administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the mDAR (or antigen binding portions thereof) is formulated in the presence of sodium acetate to increase thermal stability.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the memory dimeric antigen receptors (mDAR) or antigen-binding portions thereof described herein can be administered to a subject using art-known methods and delivery routes.

The terms “effective amount”, “therapeutically effective amount” or “effective dose” or related terms may be used interchangeably and refer to an amount of any of the transgenic T cells expressing memory dimeric antigen receptors (mDAR) described herein that when administered to a subject, is sufficient to effect a measurable improvement or prevention of a disease or disorder associated with tumor or cancer antigen expression. Therapeutically effective amounts of transgenic T cells expressing mDAR provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered at about 0.01 mg/kg to about 50 mg/kg per day, preferably 0.01 mg/kg to about 30 mg/kg per day, most preferably 0.1 mg/kg to about 20 mg/kg per day. The polypeptide may be administered daily (e.g., once, twice, three times, or four times daily) or preferably less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

In one embodiment, a therapeutically effective amount comprises a dose of about 10³-10¹² transgenic host cells administered to the subject. In one embodiment, the transgenic host cells harbor one or more expression vectors that express the polypeptide chains that comprise any of the mDARs described herein. The therapeutically effective amount can be determined by considering the subject to receive the therapeutically effective amount and the disease/disorder to be treated which may be ascertained by one skilled in the art using known techniques. The therapeutically effective amount may consider factors pertaining to the subject such as age, body weight, general health, sex, diet, time of administration, drug interaction, and the severity of the disease/disorder. The therapeutically effective amount may consider the purity of the transgenic host cells, which can be about 65%-98% or higher levels of purity. The therapeutically effective amount of the transgenic host cells can be administered to the subject at least once, or twice, three times, 4 times, 5 times, or more over a period of time. The period of time can be per day, per week, per month, or per year. The therapeutically effective amount of the transgenic cells administered to the subject can be same each time or can be increased or decreased at each administration event. The therapeutically effective amount of the transgenic cells can be administered to the subject until the tumor size or number of cancer cells is reduced by 5%-90% or more, compared to the tumor size or number of cancer cells prior to administration of the transgenic host cells.

The present disclosure provides methods for treating a subject having a disease/disorder associated with expression or over-expression of one or more tumor-associated antigens. The disease comprises cancer or tumor cells expressing the tumor-associated antigens, such as for example CD38 antigen. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the cancer comprises hematological cancers, including leukemias, lymphomas, myelomas and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

Memory Dimeric Antigen Receptors (mDARs)

The present disclosure provides memory dimeric antigen receptors (mDARs) comprising a Fab fragment joined to a transmembrane region and an intracellular JAK-STAT signaling region.

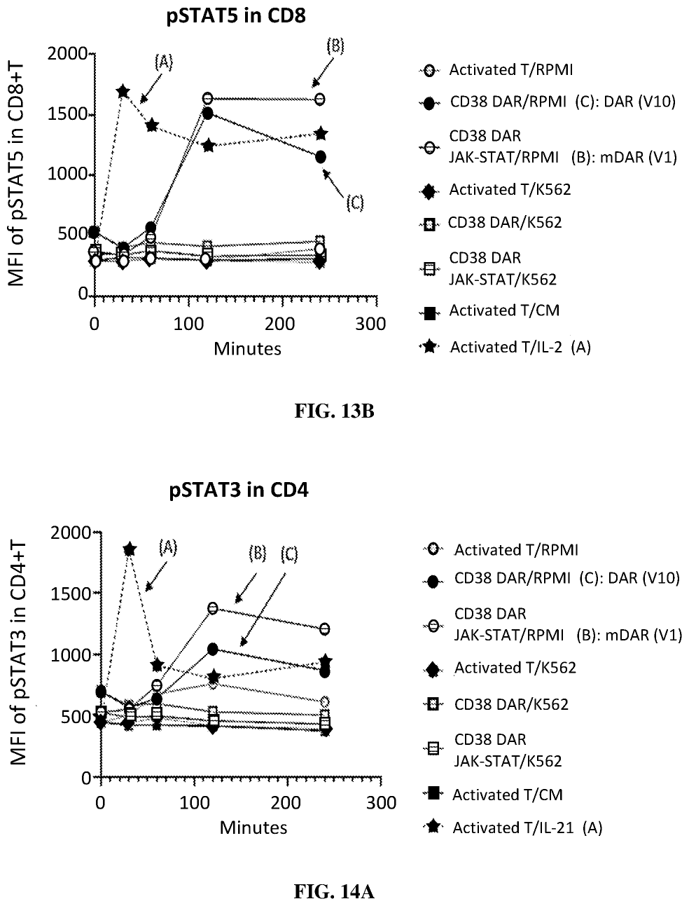

The mDAR-expressing transgenic host cells (e.g., T cells) described herein are designed to induce a JAK-STAT T-cell intracellular activation pathway in response to engaging a target antigen. The CD38 mDAR-expressing transgenic T cells described herein are demonstrated to induce higher phosphorylation activity of STAT3 and STAT5 in a target-specific manner compared to transgenic T cells expressing traditional DAR constructs (e.g., FIGS. 13A-B, 14A-B and 20A-B). Traditional DARs, such as those described in WO 2019/173837 and WO 2021 046445 (incorporated herein by reference), do not include an intracellular JAK-STAT region described herein, for example, do not include regions that bind JAK or STAT proteins. These results demonstrate that the mDAR-expressing transgenic T cells undergo enhanced intracellular JAK-STAT activation in response to target-specific binding with respect to transgenic T cells expressing traditional DARs.

The present disclosure provides mDAR constructs comprising a heavy chain binding region on one polypeptide chain and a light chain binding region on a separate polypeptide chain. One of the two polypeptide chains has a transmembrane domain and an intracellular domain that can include one or more regions (or domains) that mediate intracellular signaling. The other polypeptide chain is not membrane bound or anchored. The two polypeptide chains that make up the memory dimeric antigen receptors can dimerize to form a protein complex. The memory dimeric antigen receptors have antibody-like properties as they bind specifically to a target antigen. The mDARs can be used for directed cell therapy.

In various embodiments described herein, a general design of an mDAR includes a first polypeptide chain and a second polypeptide chain, wherein the first polypeptide chain comprises an antigen binding region connected to a dimerization domain, which is connected (proceeding from the N-terminus to the C-terminus of the polypeptide) to a hinge region, which is connected to a transmembrane region, which is then connected to an intracellular signaling domain that includes JAK-STAT signaling domains or regions, and wherein the second polypeptide chain comprises an antigen binding domain and a dimerization domain. In various embodiments, the antigen binding domain on one or both of the first and the second polypeptide chains comprises a heavy chain variable region or a light chain variable region. For example, the first polypeptide can include a heavy chain variable region and the second polypeptide can include a light chain variable region or alternatively the first polypeptide can include a light chain variable region and the second polypeptide can include a heavy chain variable region. The heavy and light chain variable regions on the first and second polypeptides of a DAR are preferably derived from the same antibody, for example, the same monoclonal antibody.

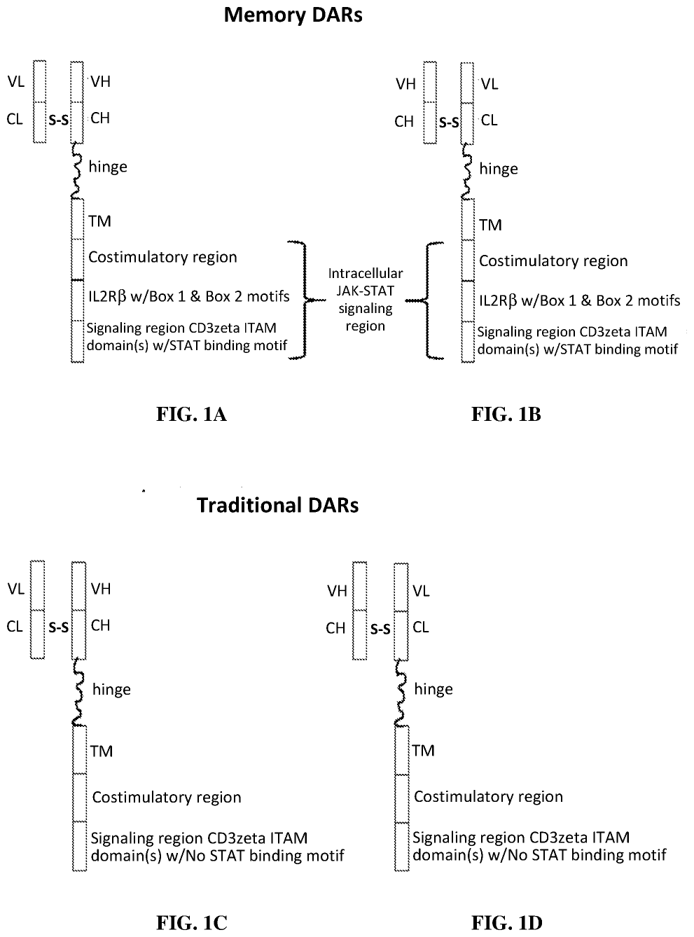

In various embodiments, the dimerization domain on one or both of the first and second polypeptide chains can be selected from the group consisting of a kappa light chain constant region, a lambda light chain constant region, a CH1 constant region of an antibody heavy chain (e.g., from IgG, IgA, IgM, IgE, or IgD, or from an IgG1, IgG2, IgG3, or IgG4), a leucine zipper, myc-max components, and combinations thereof. For example, the dimerization may be a CH1 constant region of an antibody heavy chain such as for example an IgG1 or IgG4, where the two polypeptides of the DAR associate by one or more disulfide bonds. In FIGS. 1A-B and 2A-B, “S-S” represents a disulfide bond or any chemical bond or association that results in dimerization of the first and second polypeptide chains, including a a leucine zipper or myc-max components.

The mDARs described herein, such as the CD38 mDARs described herein, have an antigen-binding extracellular portion and an intracellular signaling portion that includes regions that mediate JAK-STAT signaling. When expressed on the cell membrane of transgenic cells (e.g., transgenic T cells) that are engineered to express the mDARs, the extracellular portion can exhibit high affinity and avidity binding of CD38-overexpressing diseased hematopoietic cells, leading to T cell activation and diseased-cell killing. The intracellular portion of the mDAR comprises signaling regions that stimulate T cell activation via JAK-STAT signaling pathway upon binding of the extracellular antigen binding portion of the mDAR to antigen, which can lead to enhanced T cell expansion and/or formation of memory T cells that express the CD38 mDAR constructs. It is postulated that formation of memory T cells is important to prevent disease relapse in a subject suffering from a hematologic disease involving CD38-overexpression. Described herein are multiple configurations of mDAR constructs that differ in the type and number of intracellular signaling regions, with optional costimulatory regions, providing flexibility in designing mDAR constructs.

The present disclosure provides in some embodiments an mDAR having a first polypeptide chain and a second polypeptide chain, where the first polypeptide chain comprises a heavy chain variable region of an antibody and the second polypeptide chain comprises a light chain variable region of an antibody, or alternatively where the first polypeptide chain comprises a light chain variable region of an antibody and the second polypeptide chain comprises a heavy chain variable region of an antibody, where the first polypeptide chain is linked to the second polypeptide chain by one or a plurality of disulfide bonds at regions outside of a transgenic host cell when both the first polypeptide chain and the second polypeptide chain are expressed by the same cell. The first polypeptide chain can comprise, in order (N-terminus to C-terminus), the antibody heavy or light chain variable domain region and a corresponding antibody constant region, an optional hinge region, a transmembrane region, and an intracellular region that includes JAK-STAT signaling domains. The second polypeptide chain comprises an antibody light or heavy chain variable domain region with a corresponding constant region, where the constant regions in each first and second polypeptide chains are linked with one or more disulfide bonds (e.g., see FIGS. 1A and B).

The mDARs comprise two polypeptide chains, a first polypeptide having an antigen-binding domain, e.g., an antibody heavy or light chain variable domain plus a constant region domain, a transmembrane domain, and intracellular signaling regions, and a second polypeptide having an antigen binding domain, e.g., an antibody heavy or light chain domain plus a constant region domain, without any transmembrane domain or intracellular signaling domains. When the first polypeptide of the mDAR comprises a heavy chain variable domain, the second polypeptide comprises a light chain variable domain, and when the first polypeptide of the mDAR comprises a light chain variable domain, the second polypeptide comprises a heavy chain variable domain. The heavy and light chain variable domains of a particular mDAR are preferably derived from the same antibody, for example, may be derived from the same monoclonal antibody. In various embodiments a (first or second) mDAR polypeptide having a heavy chain variable domain further comprises, as a dimerization domain, a CH1 region and a (first or second) mDAR polypeptide having a heavy chain variable domain further comprises, as a dimerization domain, a light chain constant region (CL or CK).

The mDAR construct thus comprises an antibody heavy chain variable region and an antibody light chain variable region on separate polypeptide chains, where the heavy chain variable region and the light chain variable region form an antigen binding domain of the assembled mDAR.

In these and all other embodiments described herein in which a polypeptide chain comprises elements set forth in order, the polypeptide chain may also comprise additional elements before, between, or after the explicitly recited elements as long as the explicitly recited elements occur in the specified order, unless explicitly indicated to the contrary.

In various embodiments the present disclosure provides memory dimeric antigen receptors (mDARs) where the first polypeptide chain carries the heavy chain variable (VH) and heavy chain constant regions (CH), and the second polypeptide chain carries the light chain variable (VL) and light chain constant regions (CL) (e.g., FIGS. 1A and B). In various examples, an mDAR can comprise: (a) a first polypeptide chain comprising a plurality of polypeptide regions, the plurality comprising, in order from the amino terminus to the carboxyl terminus: (i) an antibody heavy chain variable region (VH), (ii) an antibody heavy chain constant region (CH), (iii) an optional hinge region, (iv) a transmembrane region (TM), and (v) an intracellular signaling region comprising JAK-STAT signaling domains, where the intracellular signaling region includes, in any order (1) a cytokine receptor intracellular region having a Box 1 motif and a Box 2 motif that bind a Janus kinase (JAK), (2) a CD3ζ intracellular signaling region that includes at least one CD3ζ ITAM domain that has two ITAM motifs and further including a heterologous STAT3 binding motif, and (3) an optional intracellular costimulatory region (e.g., CD28 or 4-1BB or OX40); and (b) a second polypeptide chain comprising a plurality of polypeptide regions, the plurality comprising, in order from the amino terminus to the carboxyl terminus: (vi) an antibody light chain variable region (VL) (e.g., kappa or lambda), and (vii) an antibody light chain constant region (CL), wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain (FIG. 1A).

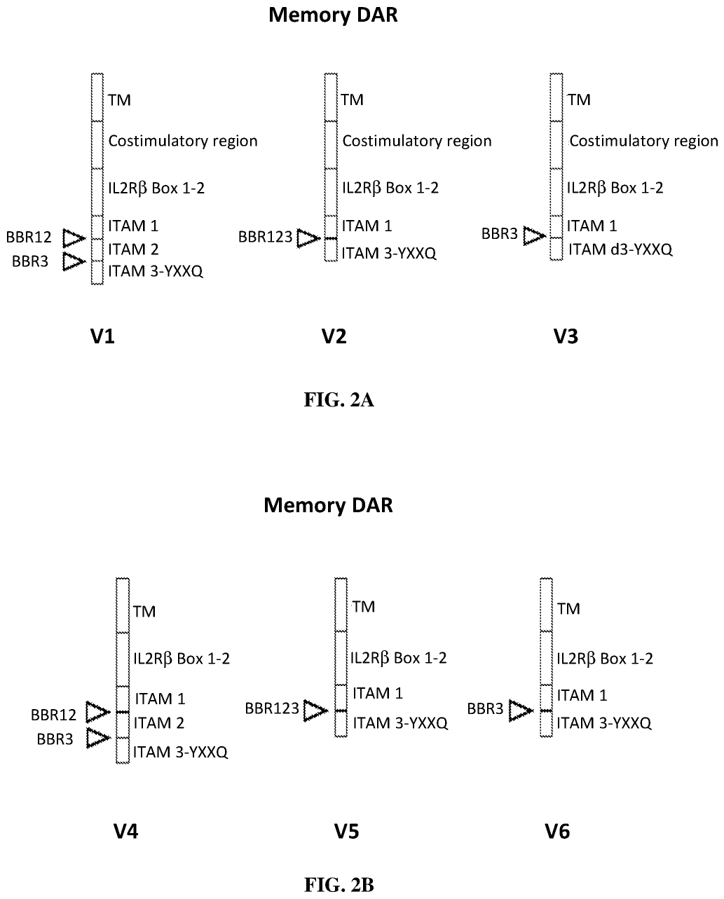

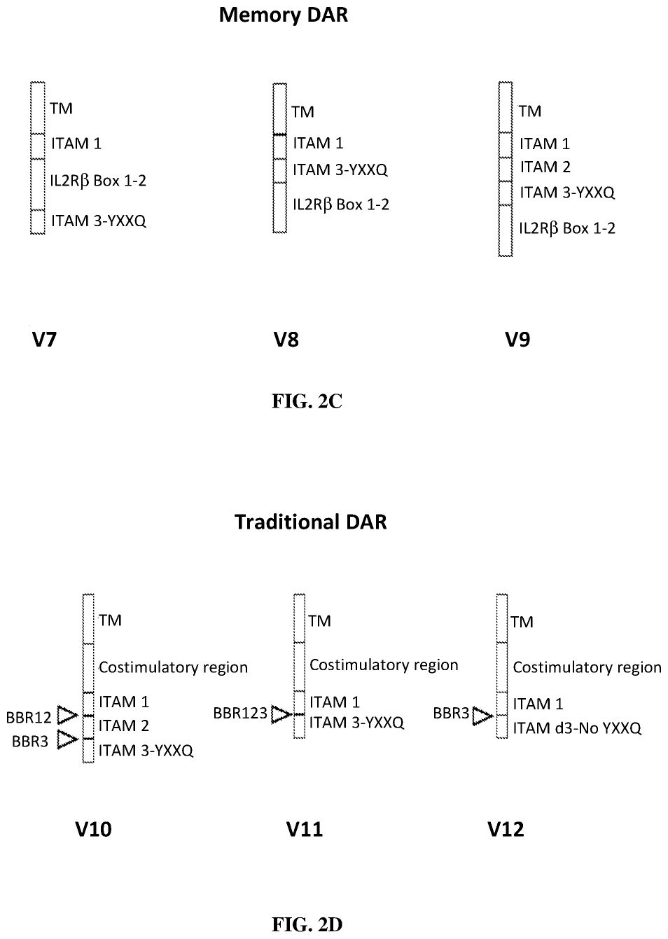

The present disclosure also provides memory dimeric antigen receptors (mDAR) constructs where the first polypeptide chain carries the light chain variable (VL) and light chain constant regions (CL), and the second polypeptide chain carries the heavy chain variable (VH) and heavy chain constant regions (CH) (e.g., FIGS. 2A and B). In one embodiment, the memory dimeric antigen receptors (mDAR) constructs comprises (a) a first polypeptide chain comprising a plurality of polypeptide regions, the plurality comprising, in order from the amino terminus to the carboxyl terminus: (i) an antibody light chain variable region (VL), (ii) an antibody light chain constant region (CL), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) a second polypeptide chain comprising a plurality of polypeptide regions, the plurality comprising, in order from the amino terminus to the carboxyl terminus: (vi) an antibody heavy chain variable region (VH), and (vii) an antibody heavy chain constant region (CH), wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain (FIG. 1B).

In various embodiments, the antibody heavy chain constant region (CH) and the antibody light chain constant region (CL) of an mDAR dimerize when the two polypeptide chains are produced by a host cell. In various embodiments, the antibody heavy chain constant region and the antibody light chain constant region dimerize via one or two disulfide bonds that are external to the host cell.

In preferred embodiments, the antibody heavy chain variable region (VH) and the antibody light chain variable region (VL) associate with each other to form an antigen binding domain external to the host cell. For example, the antibody heavy chain variable region and the antibody light chain variable region can associate with each other when the antibody heavy chain constant region and the antibody light chain constant region dimerize. The antigen binding domain, which is formed from the antibody heavy chain variable region and the antibody light chain variable region, can bind a target antigen.

In some embodiments, the antibody heavy chain variable region and the antibody light chain variable region are fully human antibody regions, humanized antibody region, or chimeric antibody regions.

mDARs such as those shown in FIGS. 1A and 1B, and FIGS. 2A and 2B, may include a hinge region which is about 10 to about 100 amino acids in length. In some embodiments, the hinge region comprises a hinge region or a fragment thereof from an antibody (e.g., IgG, IgA, IgM, IgE, or IgD). The hinge region can be joined to a constant domain (e.g., CH1 and/or CH2) of an antibody. A hinge region in a DAR can comprises any one or any combination of two or more of a CD8, CD8α, and a CD28 hinge region, or a fragment of any thereof. In some embodiments, the hinge region comprises a CPPC or SPPC amino acid sequence. In some embodiments, the hinge region comprises both CD8 and CD28 hinge sequences (e.g., a long hinge region), only CD8 sequences (short hinge) or only CD28 hinge sequences (e.g., short hinge region). In some alternative embodiments, any of the dimeric antigen receptors shown in FIG. 1A or 1B, or FIG. 2A or 2B, can lack a hinge region.

In various embodiments, for example, the mDARs as illustrated in FIGS. 1A and 1B, and FIGS. 2A and 2B, the transmembrane regions of the first and second polypeptide chains can be independently derived from CD8α, CD8β, 4-1BB(CD137), CD28, CD34, CD4, FcεRIγ, CD16, OX40(CD134), CD3ζ, CD3ε, CD3γ, CD3δ, TCRα, TCRβ, TCRζ, CD32, CD64, CD64, CD45, CD5, CD9, CD22, CD33, CD38, CD64, CD80, CD86, CD137, CD154, LFA-1 T cell co-receptor, CD2 T cell co-receptor/adhesion molecule, CD40, CD4OL/CD154, VEGFR2, FAS, and FGFR2B.

In some embodiments, for mDARs as illustrated in FIGS. 1A and 1B, and 2A and 2B, the intracellular JAK-STAT signaling region comprises an IL2Rβ intracellular region (SEQ ID NO:43) with Box 1 and Box 2 motifs for binding JAK proteins, such as a JAK1 and/or JAK3 proteins. For example, the Box 1 motif can comprise the amino acid sequence LKCNTPDPS (SEQ ID NO:44). In another example, the Box 2 motif comprises the amino acid sequence PLEVLE (SEQ ID NO:45).

In some embodiments, mDARs as shown in FIGS. 1A and 1B, and 2A and 2B, include an intracellular JAK-STAT signaling region comprising a CD3ζ intracellular signaling region which comprises at least one CD3ζ ITAM domain having two ITAM motifs. In some embodiments, the CD3ζ intracellular signaling region comprises any one or any combination of two or more of ITAM 1, 2 and/or 3 domains, where each ITAM domain includes two ITAM motifs (e.g., having the core amino acid sequence YXX(L/I)). For example, the ITAM motif can comprise the sequence YNEL (SEQ ID NO: 151), YDVL (SEQ ID NO: 152), YSEI (SEQ ID NO: 153), YQGL (SEQ ID NO: 154) or YDAL (SEQ ID NO: 155). In some embodiments, the CD3ζ intracellular signaling region comprises a heterologous STAT binding motif that is 4-6 amino acids in length and having the general amino acid sequence YXXQ, YXPQ, YXXL, or YXXF (where X is any amino acid). In some embodiments, the heterologous STAT binding motif is derived from the intracellular region of gp130 subunit (e.g., UniProt Q17RAO) from an IL-6 receptor family. For example, the heterologous STAT binding motif can bind STAT1, STAT3, STAT5A and/or STAT5B. In some examples, the heterologous STAT binding motif comprises the sequence YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively).

In some embodiments, an mDAR as shown in FIGS. 1A and 1B, and 2A and 2B comprises an intracellular JAK-STAT signaling region of the first polypeptide comprising an intracellular costimulatory region having any combination in any order of 1-5 intracellular sequences from 4-1BB, CD28, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2, CD226, and combinations thereof. In one embodiment, the intracellular region comprises CD28, and/or 4-1BB costimulatory intracellular sequences. In some embodiments, the memory dimeric antigen receptors lack an intracellular costimulatory region.

The present disclosure provides memory dimeric antigen receptors (mDAR) constructs having first and second polypeptide chains that associate with each other to form an antigen binding domain that binds a CD38 protein (e.g., target antigen) or a fragment thereof. In one embodiment, the CD38 protein is from human, ape (e.g., chimpanzee), monkey (e.g., cynomolgus), murine (e.g., mouse and/or rat), canine (e.g., dog) and/or feline (e.g., cat). In one embodiment, the CD38 protein comprises human CD38 protein (e.g., UniProt P28907; SEQ ID NO:1), cynomolgus monkey CD38 protein (UniProt Q5VAN0; SEQ ID NO:2), mouse CD38 protein (UniProt P56528; SEQ ID NO:3), or rat CD38 protein (UniProt Q64244; SEQ ID NO:4).

In one embodiment, the first polypeptide chain of the memory dimeric antigen receptor (FIG. 1A) comprises an antibody heavy chain variable region having the heavy chain CDR1, CDR2, and CDR3 of a heavy chain variable region comprising the sequence of any one of SEQ ID NO: 9, 11, 13, 15, 17, 19, 21, 23, 25, 27, 29 or 31. In one embodiment, the first polypeptide chain of the memory dimeric antigen receptor (FIG. 1A) comprises an antibody heavy chain variable region comprising an amino acid sequence having at least 95% sequence identity to the amino acid sequence of any one of SEQ ID NOS:9, 11, 13, 15, 17, 19, 21, 23, 25, 27, 29 or 31. In one embodiment, the antibody heavy chain constant region comprises sequences derived from a human antibody constant region, e.g., a human CH1 domain. In one embodiment, the antibody heavy chain constant region can be derived from an IgM, IgA, IgG, IgE or IgD antibody. In one embodiment, the antibody heavy chain constant region comprises the amino acid sequence of SEQ ID NO:5 or 6. In one embodiment, the hinge region comprises a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the first polypeptide lacks a hinge region. In one embodiment, the transmembrane region comprises the amino acid sequence of SEQ ID NO:36 (from CD28), SEQ ID NO:37 (from CD8), SEQ ID NO:38 (from 4-1BB), or SEQ ID NO:39 (from CD3). In one embodiment, the intracellular JAK-STAT signaling region comprises an IL2Rβ intracellular region (SEQ ID NO:43) having Box 1 and Box 2 motifs (SEQ ID NOS:44 and 45, respectively). In one embodiment, the intracellular JAK-STAT signaling region comprises the amino acid sequence from at least one CD3ζITAM domain which includes a heterologous STAT binding motif, for example: CD3ζ ITAM 1, 2 and 3 (SEQ ID NO:47); CD3ζ ITAM 1 and 3 (SEQ ID NO:49); CD3ζ ITAM 1 and d3 (where ITAM 3 is partially deleted) (SEQ ID NO:51); CD3ζ ITAM 1 (SEQ ID NO:52, 53 or 54); CD3ζ ITAM d3 (wherein ITAM 3 is partially deleted) (SEQ ID NO:55 or 56); or CD3ζITAM 3 (SEQ ID NO:57, 58, 59 or 60). In one embodiment, the STAT binding motif in the CD3ζ ITAM domain comprises the amino acid sequence YXXQ, or can be substituted with YXXF or YXXL, wherein “X” is any amino acid (SEQ ID NOS:67, 68 and 69, respectively). In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the intracellular JAK-STAT signaling region optionally includes a costimulatory region comprising an intracellular region from CD28 (SEQ ID NO:41), 4-1BB (SEQ ID NO:40) or OX40 (SEQ ID NO:42). In one embodiment, the first polypeptide chain comprises leader sequence comprising the amino acid sequence of SEQ ID NO:88 or 90, or the first polypeptide lacks a leader sequence.

In one embodiment, the second polypeptide chain of the memory dimeric antigen receptor (FIG. 1A) comprises an antibody light chain variable region having the light chain CDR1, CDR2, and CDR3 of a light chain variable region comprising the sequence of any one of SEQ ID NO: 10, 12, 14, 16, 18, 20, 22, 24, 26, 28, 30 or 32. In one embodiment, the second polypeptide chain of the memory dimeric antigen receptor (FIG. 1A) comprises an antibody light chain variable region comprising an amino acid sequence having at least 95% sequence identity to the amino acid sequence of any one of SEQ ID NO:10, 12, 14, 16, 18, 20, 22, 24, 26, 28, 30 or 32. In one embodiment, the antibody light chain constant region comprises a sequence from a human light chain constant region. In one embodiment, the antibody light chain constant region comprises a sequence from a kappa or lambda light chain constant region. In one embodiment, the antibody light chain constant region comprises an amino acid sequence having at least 95% sequence identity to the amino acid sequence of SEQ ID NO:7 (CL lambda) or SEQ ID NO:8 (CL kappa). In one embodiment, the second polypeptide chain comprises leader sequence comprising the amino acid sequence of SEQ ID NO:89 or 90, or the second polypeptide lacks a leader sequence.

In one embodiment, the first polypeptide chain of the memory dimeric antigen receptor (FIG. 1B) comprises an antibody light chain variable region having the light chain CDR1, CDR2, and CDR3 of a light chain variable region comprising the sequence of any one of SEQ ID NO: 10, 12, 14, 16, 18, 20, 22, 24, 26, 28, 30 or 32. In one embodiment, the first polypeptide chain of the memory dimeric antigen receptor (FIG. 1B) comprises an antibody light chain variable region comprising an amino acid sequence having at least 95% sequence identity to the amino acid sequence of any one of SEQ ID NO: 10, 12, 14, 16, 18, 20, 22, 24, 26, 28, 30 or 32. In one embodiment, the antibody light chain constant region comprises a sequence from a human light chain constant region. In one embodiment, the antibody light chain constant region comprises a sequence from a kappa or lambda light chain constant region. In one embodiment, the antibody light chain constant region comprises an amino acid sequence having at least 95% sequence identity to the amino acid sequence of SEQ ID NO:7 (CL lambda) or SEQ ID NO:8 (CL kappa). In one embodiment, the hinge region comprises a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the first polypeptide lacks a hinge region. In one embodiment, the transmembrane region comprises the amino acid sequence of SEQ ID NO:36 (from CD28), SEQ ID NO:37 (from CD8), SEQ ID NO:38 (from 4-1BB), or SEQ ID NO:39 (from CD3). In one embodiment, the intracellular JAK-STAT signaling region comprises an IL2Rβ intracellular region (SEQ ID NO:43) having Box 1 and Box 2 motifs (SEQ ID NOS:44 and 45, respectively). In one embodiment, the intracellular JAK-STAT signaling region comprises the amino acid sequence from at least one CD3ζITAM domain which includes a heterologous STAT binding motif, for example: CD3ζITAM 1, 2 and 3 (SEQ ID NO:47); CD3ζITAM 1 and 3 (SEQ ID NO:49); CD3ζITAM 1 and d3 (where ITAM 3 is partially deleted) (SEQ ID NO:51); CD3ζITAM 1 (SEQ ID NO:52, 53 or 54); CD3ζITAM d3 (wherein ITAM 3 is partially deleted) (SEQ ID NO:55 or 56); or CD3ζ ITAM 3 (SEQ ID NO:57, 58, 59 or 60). In one embodiment, the STAT binding motif in the CD3ζ ITAM domain comprises the amino acid sequence YXXQ, or can be substituted with YXXF or YXXL, wherein “X” is any amino acid (SEQ ID NOS:67, 68 and 69, respectively). In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the intracellular JAK-STAT signaling region optionally includes a costimulatory region comprising an intracellular region from CD28 (SEQ ID NO:41), 4-1BB (SEQ ID NO:40) or OX40 (SEQ ID NO:42). In one embodiment, the first polypeptide chain comprises leader sequence comprising the amino acid sequence of SEQ ID NO:89 or 90, or the first polypeptide lacks a leader sequence.

In one embodiment, the second polypeptide chain of the memory dimeric antigen receptor (FIG. 1B) comprises an antibody heavy chain variable region having the heavy chain CDR1, CDR2, and CDR3 of a heavy chain variable region comprising the sequence of any one of SEQ ID NO: 9, 11, 13, 15, 17, 19, 21, 23, 25, 27, 29 or 31. In one embodiment, the second polypeptide chain of the memory dimeric antigen receptor (FIG. 1B) comprises an antibody heavy chain variable region comprising an amino acid sequence having at least 95% sequence identity to the amino acid sequence of any one of SEQ ID NO: 9, 11, 13, 15, 17, 19, 21, 23, 25, 27, 29 or 31. In one embodiment, the antibody heavy chain constant region comprises sequences derived from a human antibody constant region, e.g., a human CH1 domain. In one embodiment, the antibody heavy chain constant region can be derived from an IgM, IgA, IgG, IgE or IgD antibody. In one embodiment, the antibody heavy chain constant region comprises the amino acid sequence of SEQ ID NO:5 or 6. In one embodiment, the second polypeptide chain comprises leader sequence comprising the amino acid sequence of SEQ ID NO:88 or 90, or the second polypeptide lacks a leader sequence.

In some embodiments, the antibody heavy chain variable region of the mDAR comprises the heavy chain CDR1, CDR2, and CDR3 of a heavy chain variable region and the antibody light chain variable region of the mDAR comprises the light chain CDR1, CDR2, and CDR3 of a light chain variable region, and the heavy and light chain regions comprise the sequence of SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively. In some embodiments, the antibody heavy chain variable region of the mDAR comprises a sequence having at least 95%, 97%, 98%, or 99% identity to a first sequence and the antibody light chain variable region of the mDAR comprises a sequence having at least 95%, 97%, 98%, or 99% identity to a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively. In some embodiments, the antibody heavy chain variable region of the mDAR comprises a first sequence and the antibody light chain variable region of the mDAR comprises a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

In some embodiments, any of the first polypeptide chains exemplified in FIG. 1A or 1B include an intracellular JAK-STAT signaling region (e.g., Version 1) comprising in order, from N-terminus to C-terminus, an intracellular JAK-STAT signaling region comprising: a 4-1BB costimulatory region; an IL2Rβ intracellular region having Box 1 and Box 2 motifs; and a CD3-zeta intracellular region having ITAMs 1, 2 and 3 domains wherein the ITAM 3 domain includes a STAT binding motif YXXQ (SEQ ID NO:67) (e.g., YRHQ, SEQ ID NO:70). In one embodiment, the first polypeptide of Version 1 comprises an IL2Rβ intracellular region having a deletion region. In some embodiments, the first polypeptide chain of Version 1 includes a STAT binding motif where YRHQ (SEQ ID NO:70) is replaced with a STAT binding motif of any one of SEQ ID NOS:70-87. In one embodiment, the intracellular JAK-STAT intracellular signaling region (e.g., Version 1) includes BRR1, BRR2 and BRR3 motifs, SEQ ID NOS:64, 65 and 65, respectively. In one embodiment, the intracellular JAK-STAT intracellular signaling region (e.g., Version 1) includes any one or any combination of two or three of the BRR1, BRR2 and/or BRR3 motifs. In one embodiment, any of the first polypeptide chains exemplified in FIG. 1A or 1B include an intracellular JAK-STAT signaling region (e.g., Version 1) comprising the amino acid sequence of SEQ ID NO:131.

In some embodiments, any of the first polypeptide chains exemplified in FIG. 1A or 1B include an intracellular JAK-STAT signaling region (e.g., Version 2) comprising in order, from N-terminus to C-terminus, an intracellular JAK-STAT signaling region comprising: a 4-1BB costimulatory region; an IL2Rβ intracellular region having Box 1 and Box 2 motifs; and a CD3-zeta intracellular region having ITAMs 1 and 3 domains wherein the ITAM 3 domain includes a STAT binding motif (e.g., YRHQ, SEQ ID NO:70). In some embodiments, the first polypeptide of Version 2 comprises an IL2Rβ intracellular region having a deletion region. In one embodiment, the first polypeptide chain of Version 2 includes a STAT binding motif where YRHQ (SEQ ID NO:70) is replaced with a STAT binding motif of any one of SEQ ID NOS:70-87. In some embodiments, the intracellular JAK-STAT intracellular signaling region (e.g., Version 2) includes BRR1, BRR2 and BRR3 motifs, SEQ ID NOS:64, 65 and 66, respectively. In one embodiment, the intracellular JAK-STAT intracellular signaling region (e.g., Version 2) includes any one or any combination of two or three of the BRR1, BRR2 and/or BRR3 motifs. In one embodiment, any of the first polypeptide chains exemplified in FIG. 1A or 1B include an intracellular JAK-STAT signaling region (e.g., Version 2) comprising the amino acid sequence of SEQ ID NO:132.

In one embodiment, any of the first polypeptide chains exemplified in FIG. 1A or 1B include an intracellular JAK-STAT signaling region (e.g., Version 3) comprising in order, from N-terminus to C-terminus, an intracellular JAK-STAT signaling region comprising: a 4-1BB costimulatory region; an IL2Rβ intracellular region having Box 1 and Box 2 motifs; and a CD3-zeta intracellular region having ITAMs 1 and 3 domains, wherein the ITAM 3 domain has a portion that is deleted (designated ITAM d3), and wherein the ITAM d3 domain includes a STAT binding motif (e.g., YRHQ). In one embodiment, the first polypeptide of Version 3 comprises an IL2Rβ intracellular region having a deletion region. In one embodiment, the first polypeptide chain of Version 3 includes a STAT binding motif where YRHQ (SEQ ID NO:70) is replaced with a STAT binding motif of any one of SEQ ID NOS:70-87. In one embodiment, the intracellular JAK-STAT intracellular signaling region (e.g., Version 3) includes a BRR3 motif (SEQ ID NO:66). In one embodiment, the intracellular JAK-STAT intracellular signaling region (e.g., Version 3) includes any one or any combination of two or three of the BRR1, BRR2 and/or BRR3 motifs. In one embodiment, any of the first polypeptide chains exemplified in FIG. 1A or 1B include an intracellular JAK-STAT signaling region (e.g., Version 3) comprising the amino acid sequence of SEQ ID NO:133.

In one embodiment, any of the first polypeptide chains exemplified in FIG. 1A or 1B include an intracellular JAK-STAT signaling region (e.g., Version 4) comprising in order, from N-terminus to C-terminus, an intracellular JAK-STAT signaling region comprising: an IL2Rβ intracellular region having Box 1 and Box 2 motifs; and a CD3-zeta intracellular region having ITAMs 1, 2 and 3 domains wherein the ITAM 3 domain includes a STAT binding motif (e.g., YRHQ, SEQ ID NO:70). In one embodiment, the first polypeptide of Version 4 comprises an IL2Rβ intracellular region having a deletion region. In one embodiment, the first polypeptide chain of Version 4 includes a STAT binding motif where YRHQ (SEQ ID NO:70) is replaced with a STAT binding motif of any one of SEQ ID NOS:70-87. In one embodiment, the intracellular JAK-STAT intracellular signaling region (e.g., Version 4) includes BRR1, BRR2 and BRR3 motifs, SEQ ID NOS:64, 65 and 66, respectively. In one embodiment, the intracellular JAK-STAT intracellular signaling region (e.g., Version 4) includes any one or any combination of two or three of the BRR1, BRR2 and/or BRR3 motifs. In one embodiment, any of the first polypeptide chains exemplified in FIG. 1A or 1B include an intracellular JAK-STAT signaling region (e.g., Version 4) comprising the amino acid sequence of SEQ ID NO: 134.

In one embodiment, any of the first polypeptide chains exemplified in FIG. 1A or 1B include an intracellular JAK-STAT signaling region (e.g., Version 5) comprising in order, from N-terminus to C-terminus, an intracellular JAK-STAT signaling region comprising: an IL2Rβ intracellular region having Box 1 and Box 2 motifs; and a CD3-zeta intracellular region having ITAMs 1 and 3 domains wherein the ITAM 3 domain includes a STAT binding motif (e.g., YRHQ). In one embodiment, the first polypeptide of Version 5 comprises an IL2Rβ intracellular region having a deletion region. In one embodiment, the first polypeptide chain of Version 5 includes a STAT binding motif where YRHQ (SEQ ID NO:70) is replaced with a STAT binding motif of any one of SEQ ID NOS:70-87. In one embodiment, the intracellular JAK-STAT intracellular signaling region (e.g., Version 5) includes BRR1, BRR2 and BRR3 motifs, SEQ ID NOS:64, 65 and 66, respectively. In one embodiment, the intracellular JAK-STAT intracellular signaling region (e.g., Version 5) includes any one or any combination of two or three of the BRR1, BRR2 and/or BRR3 motifs. In one embodiment, any of the first polypeptide chains exemplified in FIG. 1A or 1B include an intracellular JAK-STAT signaling region (e.g., Version 5) comprising the amino acid sequence of SEQ ID NO: 135.

In one embodiment, any of the first polypeptide chains exemplified in FIG. 1A or 1B include an intracellular JAK-STAT signaling region (e.g., Version 6) comprising in order, from N-terminus to C-terminus, an intracellular JAK-STAT signaling region comprising: an IL2Rβ intracellular region having Box 1 and Box 2 motifs; and a CD3-zeta intracellular region having ITAMs 1 and 3 domains wherein the ITAM 3 domain includes a STAT binding motif (e.g., YRHQ, SEQ ID NO:70). In one embodiment, the first polypeptide of Version 6 comprises an IL2Rβ intracellular region having a deletion region. In one embodiment, the first polypeptide chain of Version 6 includes a STAT binding motif where YRHQ is replaced with a STAT binding motif of any one of SEQ ID NOS:70-87. In one embodiment, the intracellular JAK-STAT intracellular signaling region (e.g., Version 6) includes a BRR3 motif, SEQ ID NOS:66. In one embodiment, the intracellular JAK-STAT intracellular signaling region (e.g., Version 6) includes any one or any combination of two or three of the BRR1, BRR2 and/or BRR3 motifs. In one embodiment, any of the first polypeptide chains exemplified in FIG. 1A or 1B include an intracellular JAK-STAT signaling region (e.g., Version 6) comprising the amino acid sequence of SEQ ID NO:136.

In one embodiment, any of the first polypeptide chains exemplified in FIG. 1A or 1B include an intracellular JAK-STAT signaling region (e.g., Version 7) comprising in order, from N-terminus to C-terminus, an intracellular JAK-STAT signaling region comprising: a CD3ζITAM 1 domain; an IL2Rβ intracellular region having Box 1 and Box 2 motifs; and a CD3-zeta ITAM 3 domain which includes a STAT binding motif (e.g., YRHQ, SEQ ID NO:70). In one embodiment, the first polypeptide of Version 7 comprises an IL2Rβ intracellular region having a deletion region. In one embodiment, the first polypeptide chain of Version 7 includes a STAT binding motif where YRHQ is replaced with a STAT binding motif of any one of SEQ ID NOS:70-87. In one embodiment, the intracellular JAK-STAT intracellular signaling region (e.g., Version 7) includes any one or any combination of two or three of the BRR1, BRR2 and/or BRR3 motifs (SEQ ID NOS:64, 65 and 66, respectively). In one embodiment, any of the first polypeptide chains exemplified in FIG. 1A or 1B include an intracellular JAK-STAT signaling region (e.g., Version 7) comprising the amino acid sequence of SEQ ID NO:137.

In one embodiment, any of the first polypeptide chains exemplified in FIG. 1A or 1B include an intracellular JAK-STAT signaling region (e.g., Version 8) comprising in order, from N-terminus to C-terminus, an intracellular JAK-STAT signaling region comprising: a CD3-zeta intracellular region having ITAMs 1 and 3 domains wherein the ITAM 3 domain includes a STAT binding motif (e.g., YRHQ, SEQ ID NO:70); and an IL2Rβ intracellular region having Box 1 and Box 2 motifs. In one embodiment, the first polypeptide of Version 8 comprises an IL2Rβ intracellular region having a deletion region. In one embodiment, the first polypeptide chain of Version 8 includes a STAT binding motif where YRHQ is replaced with a STAT binding motif of any one of SEQ ID NOS:70-87. In one embodiment, the intracellular JAK-STAT intracellular signaling region (e.g., Version 8) includes any one or any combination of two or three of the BRR1, BRR2 and/or BRR3 motifs (SEQ ID NOS:64, 65 and 66, respectively). In one embodiment, any of the first polypeptide chains exemplified in FIG. 1A or 1B include an intracellular JAK-STAT signaling region (e.g., Version 8) comprising the amino acid sequence of SEQ ID NO:138.

In one embodiment, any of the first polypeptide chains exemplified in FIG. 1A or 1B include an intracellular JAK-STAT signaling region (e.g., Version 9) comprising in order, from N-terminus to C-terminus, an intracellular JAK-STAT signaling region comprising: a CD3-zeta intracellular region having ITAMs 1, 2 and 3 domains wherein the ITAM 3 domain includes a STAT binding motif (e.g., YRHQ, SEQ ID NO:70); and an IL2Rβ intracellular region having Box 1 and Box 2 motifs. In one embodiment, the first polypeptide of Version 9 comprises an IL2Rβ intracellular region having a deletion region. In one embodiment, the first polypeptide chain of Version 9 includes a STAT binding motif where YRHQ (SEQ ID NO:70) is replaced with a STAT binding motif of any one of SEQ ID NOS:70-87. In one embodiment, the intracellular JAK-STAT intracellular signaling region (e.g., Version 9) includes any one or any combination of two or three of the BRR1, BRR2 and/or BRR3 motifs (SEQ ID NOS:64, 65 and 66, respectively). In one embodiment, any of the first polypeptide chains exemplified in FIG. 1A or 1B include an intracellular JAK-STAT signaling region (e.g., Version 9) comprising the amino acid sequence of SEQ ID NO:139.

In one embodiment, any of the first polypeptide chains exemplified in FIG. 1C or 1D lack an intracellular JAK-STAT signaling region (e.g., Version 10) comprising in order, from N-terminus to C-terminus, an intracellular signaling region comprising: a 4-1BB costimulatory region; and a CD3-zeta intracellular region having ITAMs 1, 2 and 3 domains wherein the ITAM 3 domain lacks a STAT binding motif YXXQ (SEQ ID NO:67) (e.g., YRHQ, SEQ ID NO:70). In one embodiment, the intracellular signaling region (e.g., Version 10) includes BRR1, BRR2 and BRR3 motifs, SEQ ID NOS:64, 65 and 66, respectively. In one embodiment, any of the first polypeptide chains exemplified in FIG. 1C or 1D include an intracellular signaling region (e.g., Version 10) comprising the amino acid sequence of SEQ ID NO:140.

In one embodiment, any of the first polypeptide chains exemplified in FIG. 1C or 1D lack an intracellular JAK-STAT signaling region (e.g., Version 11) comprising in order, from N-terminus to C-terminus, an intracellular signaling region comprising: a 4-1BB costimulatory region; and a CD3-zeta intracellular region having ITAMs 1 and 3 domains wherein the ITAM 3 domain lacks a STAT binding motif YXXQ (SEQ ID NO:67) (e.g., YRHQ, SEQ ID NO:70). In one embodiment, the intracellular signaling region (e.g., Version 11) includes BRR1, BRR2 and BRR3 motifs, SEQ ID NOS:64, 65 and 66, respectively. In one embodiment, any of the first polypeptide chains exemplified in FIG. 1C or 1D include an intracellular signaling region (e.g., Version 11) comprising the amino acid sequence of SEQ ID NO:141.

In one embodiment, any of the first polypeptide chains exemplified in FIG. 1C or 1D lack an intracellular JAK-STAT signaling region (e.g., Version 12) comprising in order, from N-terminus to C-terminus, an intracellular signaling region comprising: a 4-1BB costimulatory region; and a CD3-zeta intracellular region having ITAMs 1 and 3 domains wherein the ITAM 3 domain has a portion that is deleted (designated ITAM d3), and wherein the ITAM d3 domain lacks a STAT binding motif (e.g., YRHQ, SEQ ID NO:70). In one embodiment, the intracellular signaling region (e.g., Version 12) includes a BRR3 motif, SEQ ID NOS:66. In one embodiment, any of the first polypeptide chains exemplified in FIG. 1C or 1D include an intracellular signaling region (e.g., Version 12) comprising the amino acid sequence of SEQ ID NO:142.

The present disclosure provides a Version 1 (V1) memory dimeric antigen receptors (mDAR) construct comprising a first polypeptide chain carrying heavy chain variable (VH) and heavy chain constant regions (CH), and a second polypeptide chain carrying light chain variable (VL) and light chain constant regions (CL) (e.g., FIGS. 1A and 2A), wherein (a) the first polypeptide chain comprising a plurality of polypeptide regions, the plurality comprising, in order from the amino terminus to the carboxyl terminus: (i) an anti-CD38 antibody heavy chain variable region (VH), (ii) an antibody heavy chain constant region (CH), (iii) a CD28 hinge region, (iv) a CD28 transmembrane region (TM), and (v) an intracellular JAK-STAT signaling region comprising a 4-1BB costimulatory region, an IL2Rβ intracellular region having Box 1 and Box 2 motifs, and a CD3-zeta intracellular region having ITAMs 1, 2 and 3 domains wherein the ITAM 3 domain includes a STAT binding motif, wherein the CD3-zeta intracellular region includes BRR1, BRR2 and BRR3 motifs, and wherein the first polypeptide chain comprises the amino acid sequence of SEQ ID NO:96; (b) a second polypeptide chain comprising a plurality of polypeptide regions, the plurality comprising, in order from the amino terminus to the carboxyl terminus: (i) an antibody anti-CD38 light chain variable region (VL) (e.g., kappa or lambda), and (ii) an antibody light chain constant region (CL), wherein the second polypeptide chain comprises the amino acid sequence of SEQ ID NO:97, wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain. The intracellular JAK-STAT signaling region of an mDAR construct Version 1 (V1) is shown in the schematic of FIG. 2A. In one embodiment, the first polypeptide chain of an mDAR Version 1 construct includes a STAT binding motif where YRHQ (SEQ ID NO:70) (bolded and double-underlined in SEQ ID NO:96) can replaced with any one of the STAT binding motifs of SEQ ID NOS:70-87.

The present disclosure provides a Version 2 (V2) memory dimeric antigen receptors (mDAR) construct comprising a first polypeptide chain carrying heavy chain variable (VH) and heavy chain constant regions (CH), and a second polypeptide chain carrying light chain variable (VL) and light chain constant regions (CL) (e.g., FIGS. 1A and 2A), wherein (a) the first polypeptide chain comprising a plurality of polypeptide regions, the plurality comprising, in order from the amino terminus to the carboxyl terminus: (i) an anti-CD38 antibody heavy chain variable region (VH), (ii) an antibody heavy chain constant region (CH), (iii) a CD28 hinge region, (iv) a CD28 transmembrane region (TM), and (v) an intracellular JAK-STAT signaling region comprising a 4-1BB costimulatory region, an IL2Rβ intracellular region having Box 1 and Box 2 motifs, and a CD3-zeta intracellular region having ITAMs 1 and 3 domains wherein the ITAM 3 domain includes a STAT binding motif, wherein the CD3-zeta intracellular region includes BRR1, BRR2 and BRR3 motifs, and wherein the first polypeptide chain comprises the amino acid sequence of SEQ ID NO:99; (b) a second polypeptide chain comprising a plurality of polypeptide regions, the plurality comprising, in order from the amino terminus to the carboxyl terminus: (i) an antibody anti-CD38 light chain variable region (VL) (e.g., kappa or lambda), and (ii) an antibody light chain constant region (CL), wherein the second polypeptide chain comprises the amino acid sequence of SEQ ID NO:100, wherein the antibody heavy chain constant region and the antibody light chain constant region form a dimerization domain for formation of the mDAR, and wherein the antibody heavy chain variable region and the antibody light chain variable region form an antigen binding domain. The intracellular JAK-STAT signaling region of an mDAR construct Version 2 (V2) is shown in the schematic of FIG. 2A. In one embodiment, the first polypeptide chain of an mDAR Version 2 construct includes a STAT binding motif where YRHQ (SEQ ID NO:70) (bolded and double-underlined in SEQ ID NO:99) can replaced with any one of the STAT binding motifs of SEQ ID NOS: 70-87

The present disclosure provides a Version 3 (V3) mDAR comprising a first polypeptide chain carrying heavy chain variable (VH) and heavy chain constant regions (CH), and a second polypeptide chain carrying light chain variable (VL) and light chain constant regions (CL) (e.g., FIGS. 1A and 2A), wherein (a) the first polypeptide chain comprising a plurality of polypeptide regions, the plurality comprising, in order from the amino terminus to the carboxyl terminus: (i) an anti-CD38 antibody heavy chain variable region (VH), (ii) an antibody heavy chain constant region (CH), (iii) a CD28 hinge region, (iv) a CD28 transmembrane region (TM), and (v) an intracellular JAK-STAT signaling region comprising a 4-1BB costimulatory region, an IL2Rβ intracellular region having Box 1 and Box 2 motifs, and a CD3-zeta intracellular region having ITAMs 1 domain and an ITAM 3 domain having deleted region (ITAM d3) wherein the ITAM d3 domain includes a STAT binding motif, wherein the CD3-zeta intracellular region includes a BRR3 motif, and wherein the first polypeptide chain comprises the amino acid sequence of SEQ ID NO:102; (b) a second polypeptide chain comprising a plurality of polypeptide regions, the plurality comprising, in order from the amino terminus to the carboxyl terminus: (i) an antibody anti-CD38 light chain variable region (VL) (e.g., kappa or lambda), and (ii) an antibody light chain constant region (CL), wherein the second polypeptide chain comprises the amino acid sequence of SEQ ID NO:103, wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain. The intracellular JAK-STAT signaling region of an mDAR construct Version 3 (V3) is shown in the schematic of FIG. 2A. In one embodiment, the first polypeptide chain of an mDAR Version 3 construct includes a STAT binding motif where YRHQ (SEQ ID NO:70) (bolded and double-underlined in SEQ ID NO:102) can replaced with any one of the STAT binding motifs of SEQ ID NOS:70-87.

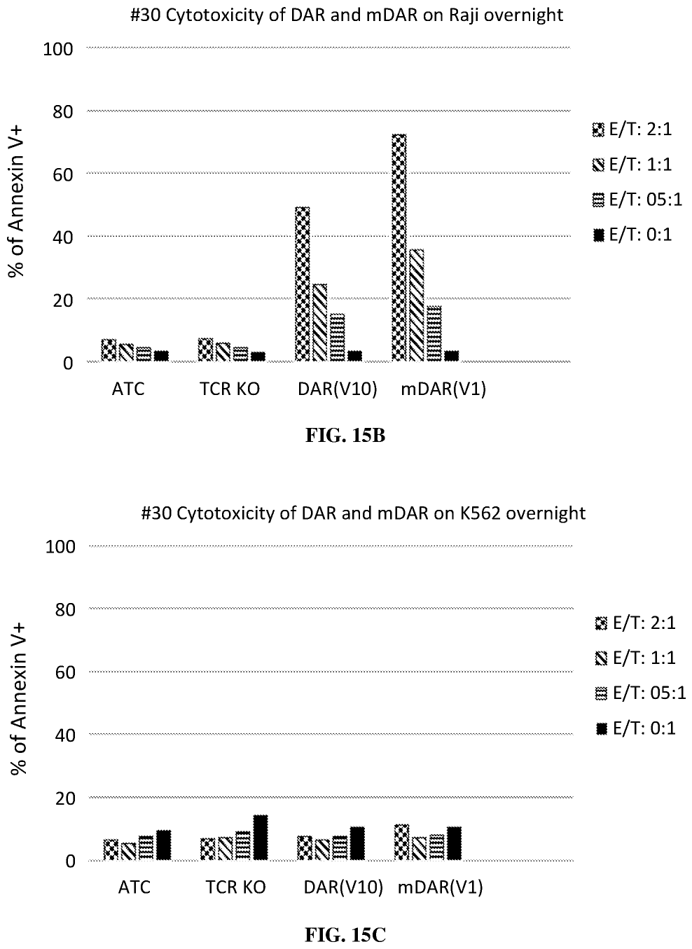

Transgenic T cells expressing CD38 mDARs can exhibit potent cytotoxicity, and in some cases exhibit higher levels of cytotoxicity than is exhibited by transgenic T cells expressing traditional DAR constructs (e.g., FIGS. 15A-C, 28A-B). In particular, transgenic T cells expressing CD38 mDARs exhibited target-specific killing, having a high level of cytotoxicity against CD38-expressing tumor cell lines (e.g., RPMI 8226 and Raji) but little or no cytotoxicity toward a low-CD38-expressing tumor cell line (K562).

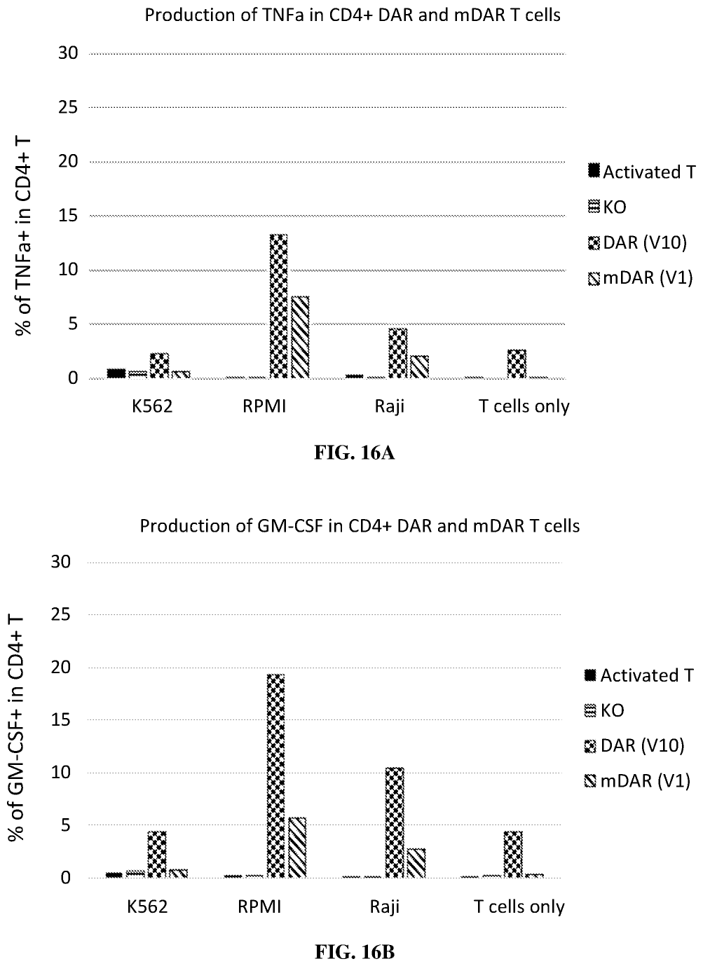

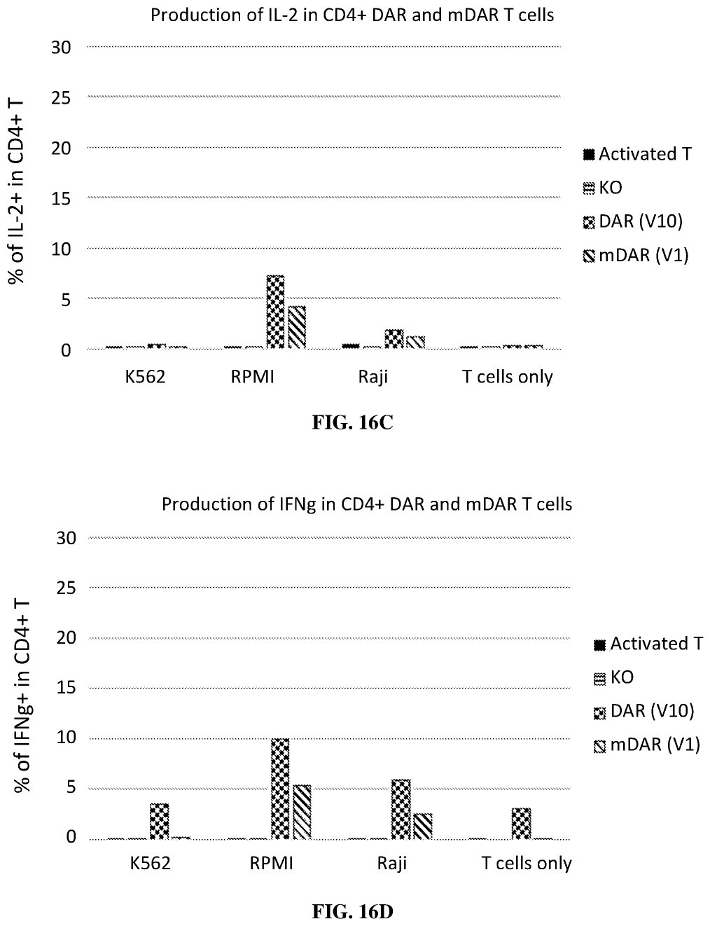

In an in vitro assay, transgenic T cells expressing CD38 mDARs release lower levels of cytokines (e.g., TNFalpha, GM-CSF, IL-2 and IFNgamma) compared to transgenic T cells expressing traditional DARs (FIGS. 16A-D, 17A-D, 29A-D, 30A-D). The mDAR-expressing T cells may induce a reduced level of cytokine storm when administered to a subject and thus may be safer compared to traditional DAR-expressing T cells.

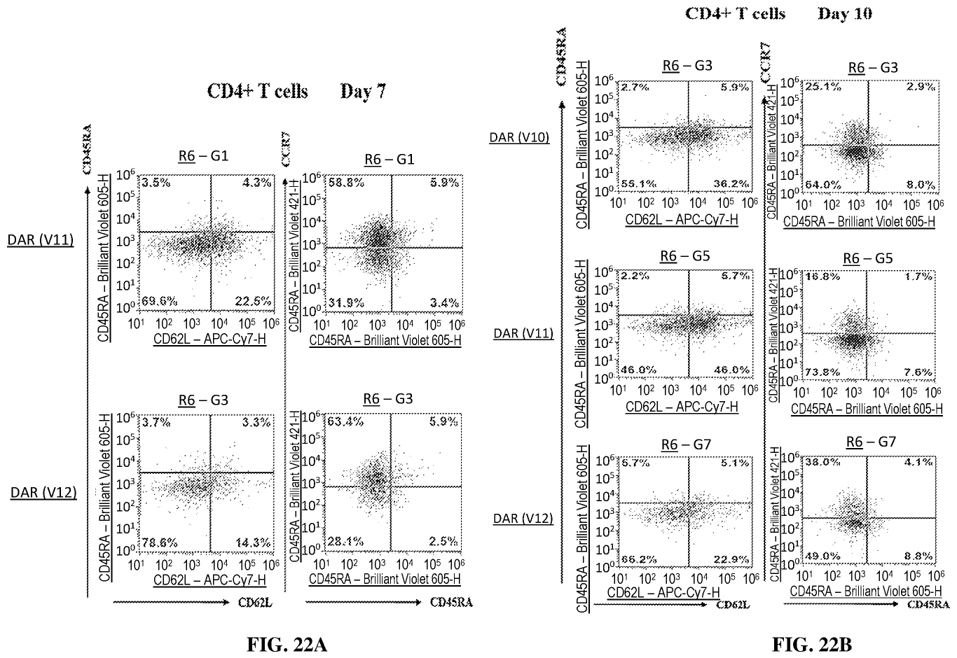

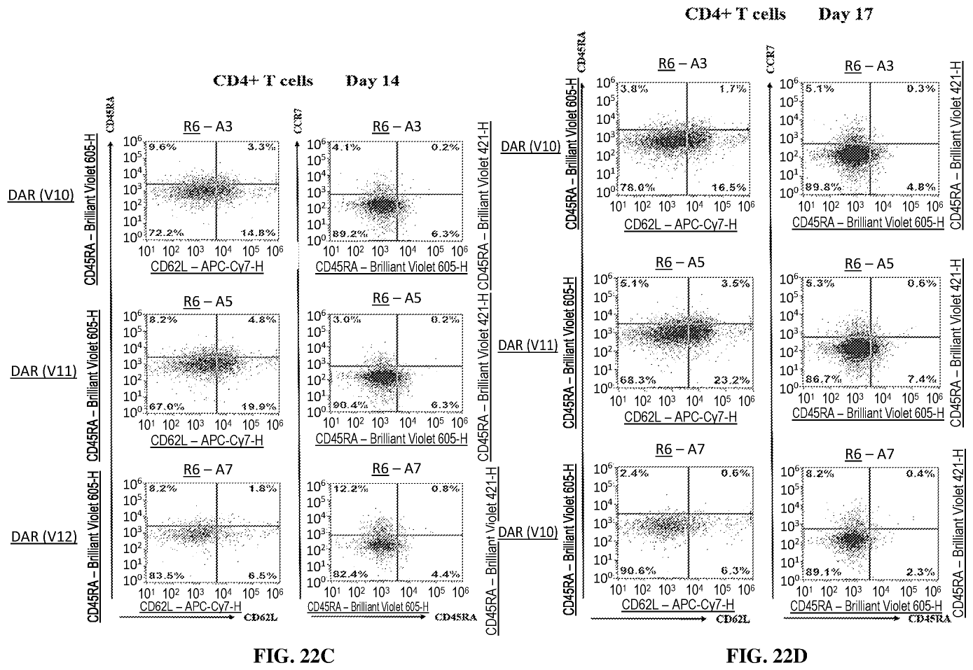

In an in vitro assay, a higher percent of transgenic T cells expressing CD38 mDARs develop into memory T cell (e.g., central memory T cell) compared to transgenic T cells expressing traditional DAR constructs (FIGS. 22A-D, 23A-D, 24A-D, 25A-D). The mDAR-expressing T cells may develop into long-lived memory T cells after administered to a subject which may be effective in reducing relapse of hematologic cancers.

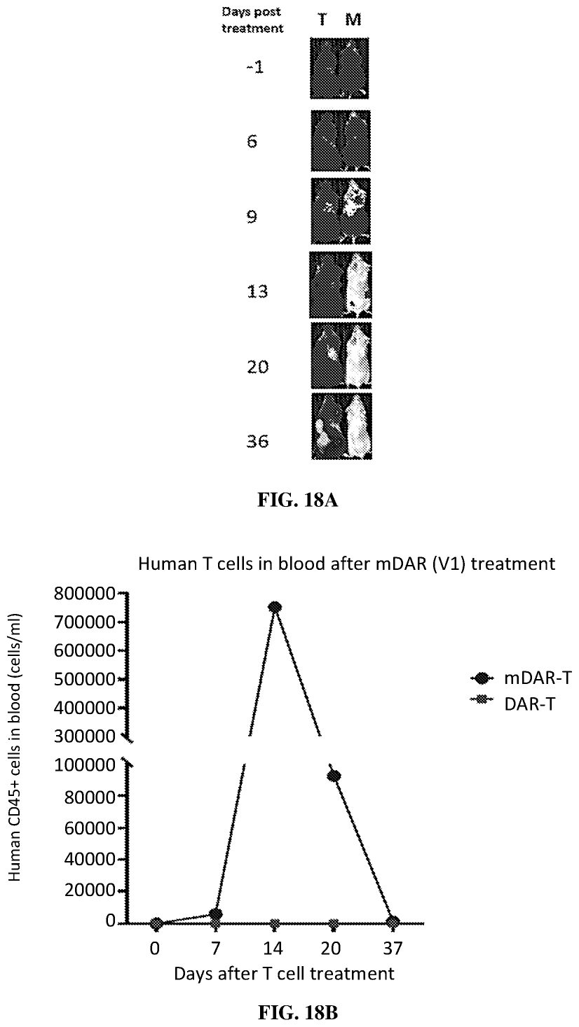

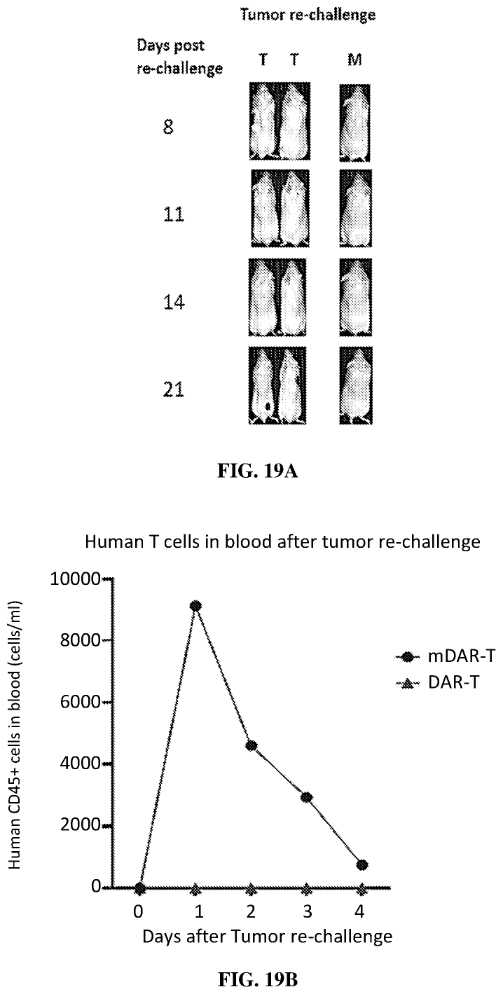

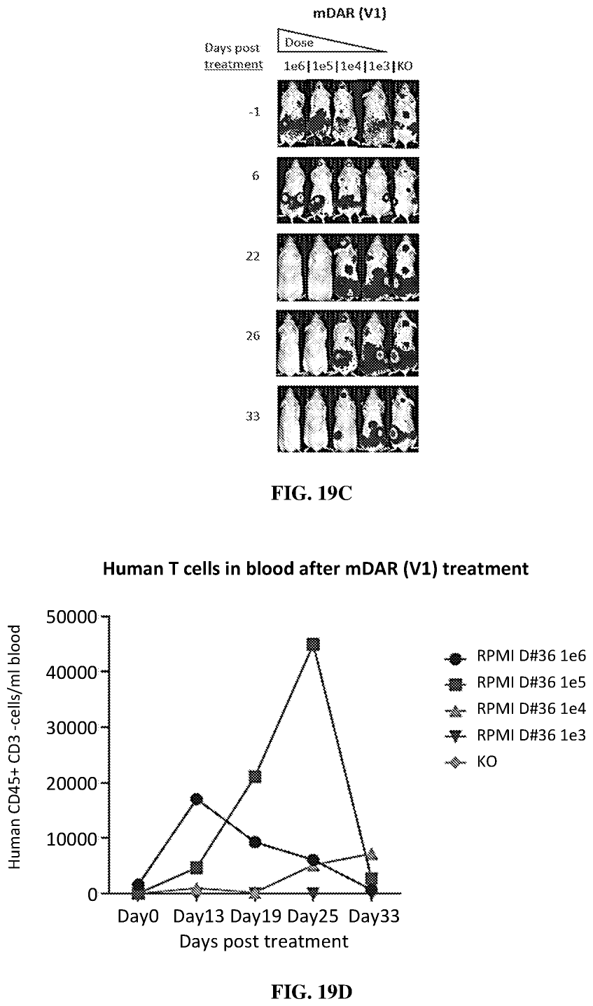

In an animal xenograft model, transgenic T cells expressing CD38 mDARs exhibited marked anti-tumorigenic activity (FIG. 18A) and undergo higher levels of proliferation which is detectable as circulating CD38 mDAR-expressing T cells (FIG. 18B) compared to transgenic T cells expressing traditional DAR constructs. In an animal tumor re-challenge assay, a significantly higher level of circulating CD38 mDAR-expressing T cells are detectable in animal serum compared to re-challenged animals treated with transgenic T cells expressing traditional DAR constructs (FIG. 19B).

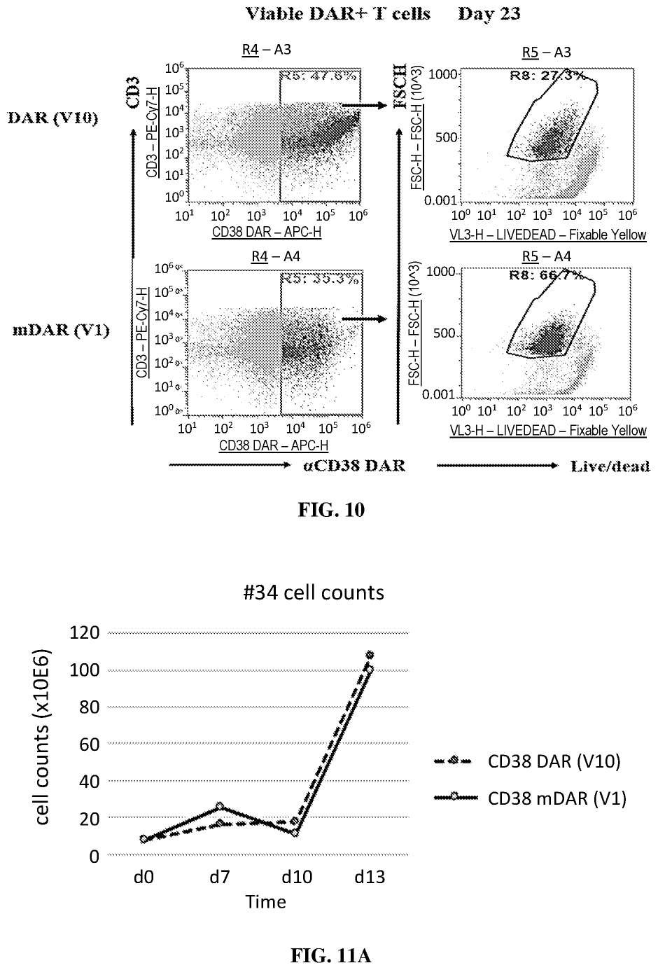

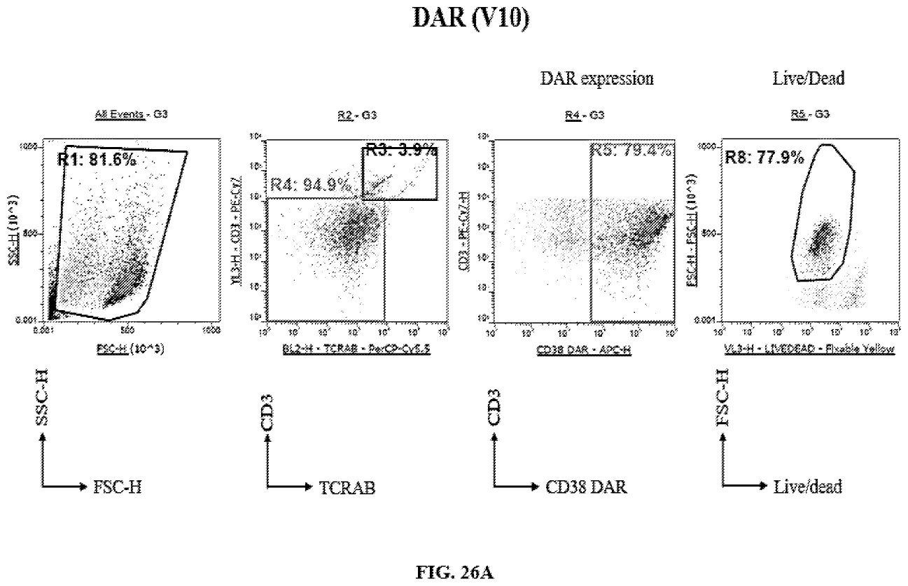

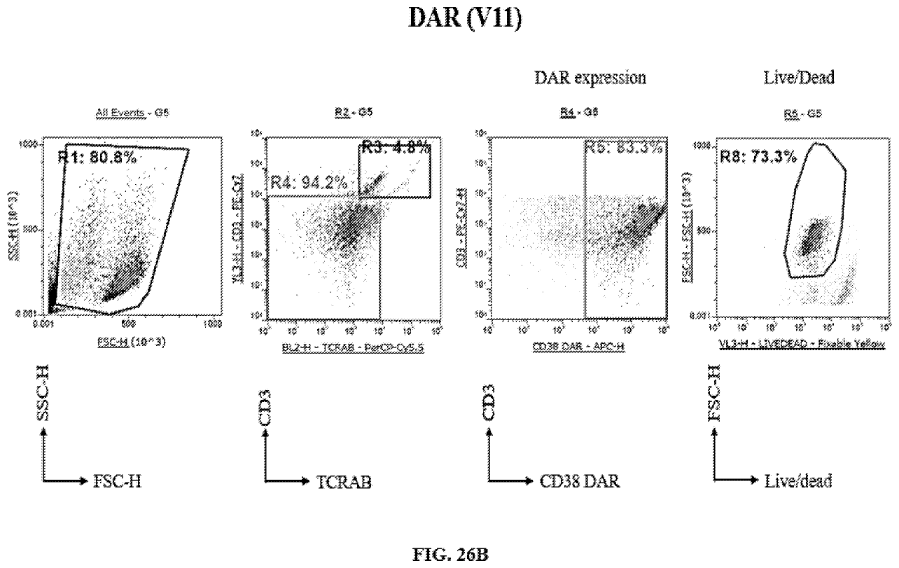

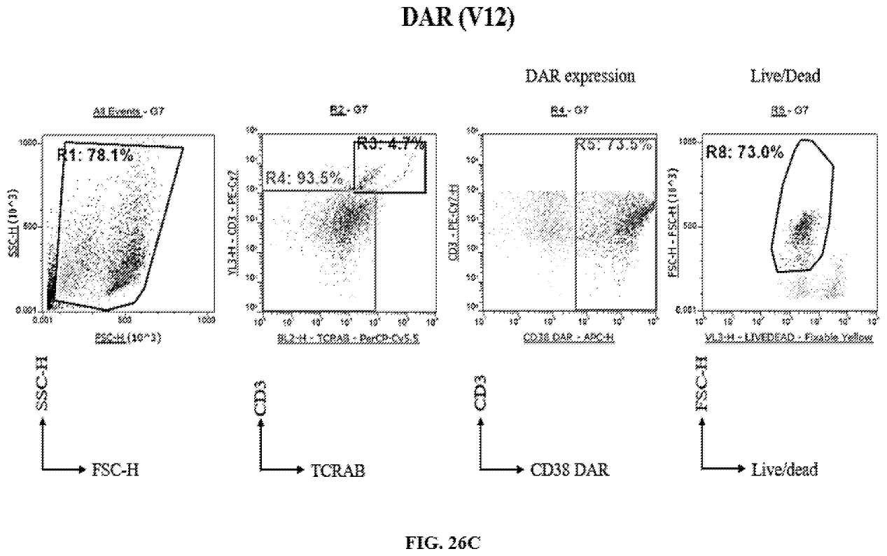

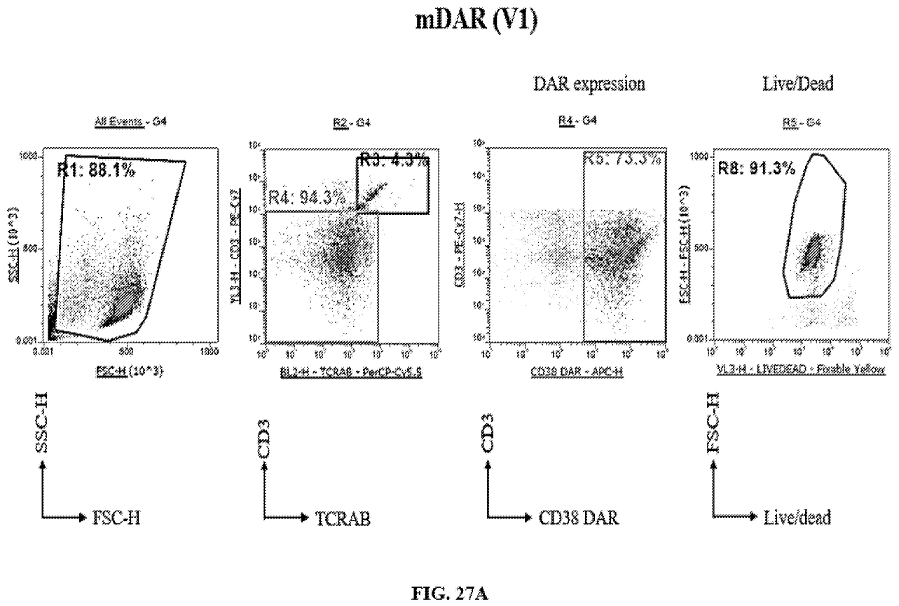

A higher percentage of transgenic T cells expressing CD38 mDARs are viable several weeks post-transfection compared to transgenic T cells expressing traditional DAR constructs (FIGS. 10, 26A-C, 27A-C). The mDAR-expressing transgenic T cells expand well in vitro and have improved viability compared to transgenic T cells expressing traditional DAR constructs.

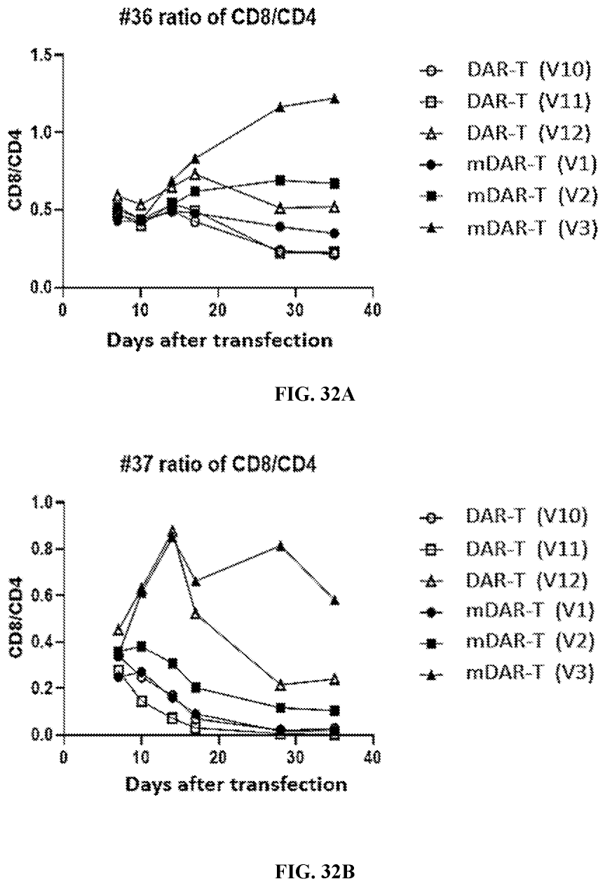

Transgenic T cells expressing CD38 mDARs develop an increase in the frequency of CD8+ T cells, while the frequency of CD4+ T cells decreases. Transgenic T cells expressing either V2 or V3 mDAR constructs develop an increased proportion of CD8+ T cells with a decrease in CD4+ T cells up to 35 days post-transfection, compared to transgenic T cells expressing traditional DAR constructs with comparable intracellular signaling regions (FIGS. 32A-B). Furthermore, the ratio of CD8+/CD4+ T cells in mDAR V3 transgenic T cells increases with time, compared to transgenic T cells expressing either traditional DAR V10 construct or mDAR V2 construct (FIGS. 32A-B). The traditional DAR V10 construct has CD3ζ ITAMs 1, 2 and 3 regions and carries BRR 1, 2 and 3 sequences. The mDAR V2 construct has CD3ζ ITAMs 1 and 3 regions but lacks a CD3ζITAM2 region, and carries BRR 1, 2 and 3 sequences. By contrast, the mDAR V3 construct has CD3ζ ITAMs 1 and a deleted version of ITAM 3 region and lacks a CD3ζ ITAM 2 region but carries only BRR 3 sequence. The lack of the CD3ζ ITAM 2 region may promote development of CD8+ T cells, and the lack of BRR 1 and 2 regions may further promote development of CD8+ T cells. CD8+ T cells are known to have potent anti-tumor cytotoxicity activity both in vitro and in vivo. Thus, transgenic T cells that express the mDAR V2 or V3 constructs can be potent in tumor killing in vivo.

An unexpected characteristic of transgenic T cells expressing the mDAR V2 or V3 constructs is that removal of the intracellular CD3ζITAM 1 and/or 2, and/or removal of BRR 1 and 2 motifs, boosts intracellular signaling which promotes increased phosphorylation of STAT5, potent cytotoxicity, reduced cytokine release, and increased memory T cell development, and enhanced in vivo expansion compared to transgenic T cells expressing mDAR V1 which contains intracellular CD3ζITAMs 1, 2 and 3.

### Precursor Polypeptides of Memory Dimeric Antigen Receptors

The present disclosure provides precursor polypeptides of memory dimeric antigen receptors (mDARs). In one embodiment, the precursor polypeptide can be expressed by a host cell and processed by the cell to become first and second polypeptide chains that associate/assemble to form memory dimeric antigen receptors (mDAR) constructs. In one embodiment, host cell processing includes cleaving the precursor polypeptide at the self-cleaving sequence to release the first and second polypeptide chains, secreting the first and second polypeptides, and/or anchoring the mDAR construct in the host cell's cellular membrane to become first and second polypeptide chains that associate/assemble to form memory dimeric antigen receptors (mDAR) constructs. In any of the precursor polypeptide embodiments described herein that comprise a self-cleaving sequence, the self-cleaving sequence may be a T2A, P2A, E2A, or F2A sequence. In one embodiment, the self-cleaving sequence is other than a T2A sequence, e.g., the self-cleaving sequence is a P2A, E2A, or F2A sequence.

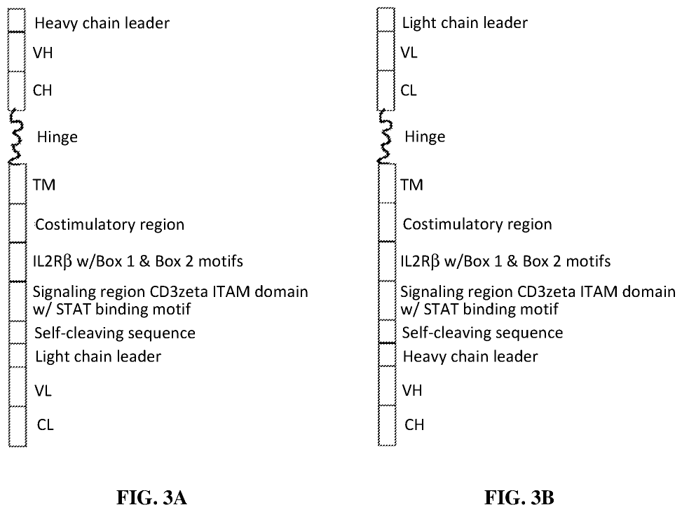

The present disclosure provides an mDAR precursor polypeptide comprising a single polypeptide chain having the amino acid sequences of the antibody heavy chain and antibody light chain sequences with an intervening self-cleaving sequence (e.g., FIG. 3A). In one embodiment, an mDAR precursor polypeptide comprises a plurality of polypeptide regions, including: a heavy chain leader sequence, an antibody heavy chain region with a variable domain region and a CH1 region, an optional hinge region, a transmembrane region, an intracellular costimulatory region, an intracellular JAK-STAT signaling region, a self-cleaving sequence, a light chain leader sequence, and an antibody light chain variable domain region (kappa (K) or lambda (L)) with a corresponding CL/CK region.

The present disclosure provides mDAR precursor polypeptides comprising a plurality of polypeptide regions, the plurality comprising, in order from the amino terminus to the carboxyl terminus: (i) a heavy chain leader sequence, (ii) an antibody heavy chain variable region, (iii) an antibody heavy chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζITAM domain with two ITAM motifs and a STAT3 binding motif, and (3) an optional intracellular costimulatory region (e.g., CD28 or 4-1BB or OX40), (vii) a self-cleaving sequence, (viii) a light chain leader sequence, (ix) an antibody light chain variable region, and (x) an antibody light chain constant region, wherein the self-cleaving sequence permits cleaving of the precursor polypeptide and release of the first and second polypeptide chains (e.g., FIG. 3A).

In one embodiment, a precursor polypeptide comprising the amino acid sequence of SEQ ID NO:95 is cleaved at the self-cleaving sequence to release the first and second polypeptides that assemble to become mDAR Version 1. In one embodiment, a precursor polypeptide comprising the amino acid sequence of SEQ ID NO:98 is cleaved at the self-cleaving sequence to release the first and second polypeptides that assemble to become mDAR Version 2. In one embodiment, a precursor polypeptide comprising the amino acid sequence of SEQ ID NO:101 is cleaved at the self-cleaving sequence to release the first and second polypeptides that assemble to become mDAR Version 3.

In one embodiment, upon release of the first and second polypeptide chains from the precursor polypeptide, the antibody heavy chain constant region and the antibody light chain constant region can dimerize to form a dimerization domain. In one embodiment, the antibody heavy chain constant region and the antibody light chain constant region dimerize via one or two disulfide bonds.

In one embodiment, upon release of the first and second polypeptide chains from the precursor polypeptide, the antibody heavy chain variable region and the antibody light chain variable region associate with each other to form an antigen binding domain.

In one embodiment, the antigen binding domain, which is formed from the antibody heavy chain variable region and the antibody light chain variable region, binds a target antigen.

In one embodiment, the antibody heavy chain variable region and the antibody light chain variable region are fully human antibody regions, humanized antibody regions, or chimeric antibody regions.

The present disclosure provides an mDAR precursor polypeptide comprising a single polypeptide chain having the amino acid sequences of the antibody light chain and antibody heavy chain sequences with an intervening self-cleaving sequence (e.g., FIG. 3B). In one embodiment, an mDAR precursor polypeptide comprises a plurality of polypeptide regions, including: a light chain leader sequence, an antibody light chain variable domain region (kappa (K) or lambda (L)) with a corresponding CL/CK region, an optional hinge region, a transmembrane region, an intracellular costimulatory region, an intracellular JAK-STAT signaling region, a self-cleaving sequence, a heavy chain leader sequence, and an antibody heavy chain region with a variable domain region and a CH1 region.

The present disclosure provides mDAR precursor polypeptides comprising a plurality of polypeptide regions, the plurality comprising, in order from the amino terminus to the carboxyl terminus: (i) a light chain leader sequence, (ii) an antibody light chain variable region, (iii) an antibody light chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζITAM domain with two ITAM motifs and a STAT3 binding motif, and (3) an optional intracellular costimulatory region (e.g., CD28 or 4-1BB or OX40), (vii) a self-cleaving sequence, (viii) a heavy chain leader sequence, (ix) an antibody heavy chain variable region, and (x) an antibody heavy chain constant region, wherein the self-cleaving sequence permits cleaving of the precursor polypeptide and release of the first and second polypeptide chains (e.g., FIG. 3B).

In one embodiment, upon release of the first and second polypeptide chains from the precursor polypeptide, the antibody light chain constant region and the antibody heavy chain constant region can dimerize to form a dimerization domain. In one embodiment, the antibody light chain constant region and the antibody heavy chain constant region dimerize via one or two disulfide bonds.

In one embodiment, upon release of the first and second polypeptide chains from the precursor polypeptide, the antibody light chain variable region and the antibody heavy chain variable region associate with each other to form an antigen binding domain.

In one embodiment, the antigen binding domain, which is formed from the antibody light chain variable region and the antibody heavy chain variable region, binds a target antigen.

In one embodiment, the antibody light chain variable region and the antibody heavy chain variable region are fully human antibody regions, humanized antibody region, or chimeric antibody regions.

In one embodiment, the precursor polypeptide (e.g., FIGS. 3A and 3B) includes a self-cleaving sequence comprising an amino acid sequence that promotes ribosomal skipping and recommencement of protein translation which generates two separate polypeptides (e.g., release the first and second polypeptide chains). In any of the precursor polypeptide embodiments described herein that comprise a self-cleaving sequence, the self-cleaving sequence may be a T2A, P2A, E2A, or F2A sequence, comprising the amino acid sequence of SEQ ID NO:91, 92, 93 and 94, respectively. In one embodiment, a population of precursor polypeptides includes a mixture of polypeptides that have been cleaved at the self-cleaving sequence or not.

In one embodiment, the precursor polypeptide (e.g., FIGS. 3A and 3B) includes heavy chain and light chain leader sequences comprising peptide signal sequences that target a polypeptide chain (e.g., first and second polypeptide chains) to the secretory pathway of a host cell and allows for integration and anchoring of the polypeptide into the lipid bilayer of the cellular membrane. The heavy and light chain leader sequences can direct transport of the precursor polypeptide from the cytosol to the endoplasmic reticulum of a host cell. The heavy and light chain leader sequences can direct transport of the precursor polypeptide from endoplasmic reticulum to the lipid bilayer of the cellular membrane. The heavy chain and light chain leader sequences include signal sequences comprising CD8α, CD28 or CD16 leader sequences. In one embodiment, for the precursor polypeptides shown in FIGS. 3A and 3B, the N-terminal end of a precursor polypeptide includes a first peptide signal sequence (e.g., heavy chain or light chain leader sequence). In one embodiment, for the precursor polypeptides shown in FIGS. 3A and 3B, the precursor polypeptide can include a second peptide signal sequence (e.g., heavy chain or light chain leader sequence) located after a cleavage sequence.

In one embodiment, the precursor polypeptide can be cleaved at the self-cleaving sequence thereby generating first and second polypeptide chains each having a peptide signal sequence at their N-terminal ends. In one embodiment, for the precursor polypeptides shown in FIGS. 3A and 3B, the processing of the precursor polypeptide includes cleaving the precursor into first and second polypeptide chains, secreting the first and second polypeptide chains via the heavy and light chain leader sequences, and/or anchoring the mDAR construct in the host cell's cellular membrane. In one embodiment, the heavy chain leader sequence and/or the light chain leader sequence is cleaved off to generate a new N-terminal end. In one embodiment, the precursor polypeptide includes a heavy chain leader sequence having the amino acid sequence of SEQ ID NO:88 or 90. In one embodiment, the precursor polypeptide includes a light chain leader sequence having the amino acid sequence of SEQ ID NO:89 or 90. In one embodiment, a population of precursor polypeptides includes a mixture of polypeptides that have been cleaved at the heavy chain and/or light chain leader sequences or not.

In one embodiment, for the precursor polypeptides shown in FIGS. 3A and 3B, the hinge region is about 10 to about 100 amino acids in length. In one embodiment, the hinge region comprises a hinge region or a fragment thereof from an antibody (e.g., IgG, IgA, IgM, IgE, or IgD). In one embodiment, the hinge region is joined to a constant domain (e.g., CH1 and/or CH2) of an antibody. In one embodiment, the hinge region comprises any one or any combination of two or more of a CD8, CD8α) and/or CD28 hinge region, or a fragment thereof. In one embodiment, the hinge region comprises a CPPC or SPPC amino acid sequence. In one embodiment, the hinge region comprises both CD8 and CD28 hinge sequences (e.g., long hinge region), only CD8 sequence (short hinge) or only CD28 hinge sequence (e.g., short hinge region). In one embodiment, any of the precursor polypeptides shown in FIGS. 3A and 3B, lack a hinge region. In one embodiment, the precursor polypeptide includes a hinge sequence having the amino acid sequence of SEQ ID NO:33, 34 or 35.

In one embodiment, for the precursor polypeptides shown in FIGS. 3A and 3B, the transmembrane regions of the precursor polypeptide chain can be derived from CD8α, CD8β, 4-1BB(CD137), CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD38, CD64, CD80, CD86, CD137, CD154, LFA-1 T cell co-receptor, CD2 T cell co-receptor/adhesion molecule, CD40, CD4OL/CD154, VEGFR2, FAS, and FGFR2B. In one embodiment, the precursor polypeptide includes a transmembrane region having the amino acid sequence of SEQ ID NO:36, 37, 38 or 39.

In one embodiment, for the precursor polypeptides shown in FIGS. 3A and 3B, the intracellular JAK-STAT signaling region comprises an IL2Rβ intracellular region (SEQ ID NO:43) with Box 1 and Box 2 motifs for binding JAK proteins, such as a JAK1 and/or JAK3 proteins. For example, the Box 1 motif comprises the amino acid sequence LKCNTPDPS (SEQ ID NO:44). In another example, the Box 2 motif comprise the amino acid sequence PLEVLE (SEQ ID NO:45).

In one embodiment, for the precursor polypeptides shown in FIGS. 3A and 3B, the intracellular JAK-STAT signaling region comprises a CD3ζ intracellular signaling region which comprises at least one CD3ζ ITAM domain having two ITAM motifs. In one embodiment, the CD3ζ intracellular signaling region comprises any one or any combination of two or more of ITAM 1, 2 and/or 3 domain, where each ITAM domain includes two ITAM motifs (e.g., having the amino acid sequence YXX(L or I)). In one embodiment, the ITAM motif comprises the sequence YNEL (SEQ ID NO: 151), YDVL (SEQ ID NO: 152), YSEI (SEQ ID NO: 153), YQGL (SEQ ID NO: 154) or YDAL (SEQ ID NO: 155). In one embodiment, the precursor polypeptide includes a CD3ζ ITAM domain comprising: CD3ζ ITAM 1, 2 and 3 (SEQ ID NO:47); CD3ζ ITAM 1 and 3 (SEQ ID NO:49); CD3ζ ITAM 1 and d3 (where ITAM 3 is partially deleted) (SEQ ID NO:51); CD3ζ ITAM 1 (SEQ ID NO:52, 53 or 54); CD3ζ ITAM d3 (wherein ITAM 3 is partially deleted) (SEQ ID NO:55 or 56); or CD3ζ ITAM 3 (SEQ ID NO:57, 58, 59 or 60). In one embodiment, the CD3ζ intracellular signaling region comprises a heterologous STAT binding motif having the general amino acid sequence YXXQ, YXPQ, YXXL, or YXXF (where X is any amino acid) (SEQ ID NO:67, 68 and 69, respectively). In one embodiment, the heterologous STAT binding motif is derived from the intracellular region of gp130 subunit (e.g., UniProt Q17RAO) from an IL-6 receptor family. In one embodiment, the heterologous STAT binding motif binds STAT1, STAT3, STAT5A and/or STAT5B. In one embodiment, the heterologous STAT binding motif comprises the sequence YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively).

In one embodiment, for the precursor polypeptides shown in FIGS. 3A and 3B, the CD3ζ intracellular signaling region includes at least one or any combination of two or three of a BRR1, BRR2 and/or BRR3 motif. For example, the CD3ζ intracellular signaling region comprises BRR1, BRR2 and BRR3 motifs (SEQ ID NOS:64, 65 and 66, respectively). The CD3ζ intracellular signaling region comprises BRR1 and BRR3 motifs (SEQ ID NOS:64 and 66, respectively). The CD3ζ intracellular signaling region comprises BRR1 and BRR2 motifs (SEQ ID NOS:64 and 65, respectively). The CD3ζ intracellular signaling region comprises BRR2 and BRR3 motifs (SEQ ID NOS:65 and 66, respectively). The CD3ζ intracellular signaling region comprises only one of a BRR1 motif (SEQ ID NO:64), BRR2 motif (SEQ ID NO:65), or BRR3 motif (SEQ ID NO:66).

In one embodiment, for the precursor polypeptides shown in FIGS. 3A and 3B, the intracellular JAK-STAT signaling region of the precursor polypeptide comprises intracellular costimulatory region in any order and any combination of 1-5 intracellular sequences from CD28, 4-1BB, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2, CD226, and combinations thereof. In one embodiment, the intracellular costimulatory region comprises any one, or any combination of two or three, of intracellular costimulatory sequence(s) from 4-1BB (SEQ ID NO:40), CD28 (SEQ ID NO:41) and/or OX40 (SEQ ID NO:42). In one embodiment, the precursor polypeptide lacks an intracellular costimulatory region.

In one embodiment, the precursor polypeptides shown in FIGS. 3A and 3B include an intracellular JAK-STAT signaling region comprising the amino acid sequence of Version 1 (SEQ ID NO:131), Version 2 (SEQ ID NO:132), Version 3 (SEQ ID NO:133), Version 4 (SEQ ID NO:134), Version 5 (SEQ ID NO:135), Version 6 (SEQ ID NO:136), Version 7 (SEQ ID NO:137), Version 8 (SEQ ID NO:138), or Version 9 (SEQ ID NO:139).

In one embodiment, the precursor polypeptides (e.g., shown in FIG. 3A) comprise an antibody heavy chain variable region having the heavy chain CDR1, CDR2, and CDR3 of a heavy chain variable region comprising the sequence of any one of SEQ ID NO: 9, 11, 13, 15, 17, 19, 21, 23, 25, 27, 29 or 31. In one embodiment, the precursor polypeptides (e.g., shown in FIG. 3A) comprise a heavy chain leader sequence comprising the amino acid sequence of SEQ ID NO:88 or 90, or lacks a heavy chain leader sequence. In one embodiment, the precursor polypeptides comprise an antibody heavy chain variable region having an amino acid sequence having at least 95% sequence identity to the amino acid sequence of any one of SEQ ID NOS: 9, 11, 13, 15, 17, 19, 21, 23, 25, 27, 29 or 31. In one embodiment, the precursor polypeptides comprise an antibody heavy chain constant region having sequences derived from a human antibody constant region, e.g., a human CH1 domain. In one embodiment, the precursor polypeptides comprise an antibody heavy chain constant region which is derived from an IgM, IgA, IgG, IgE or IgD antibody. In one embodiment, the precursor polypeptides comprise an antibody heavy chain constant region having the amino acid sequence of SEQ ID NO:5 (CPPC) or 6 (SPPC). In one embodiment, the precursor polypeptides comprise a hinge region having a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the precursor polypeptides lacks a hinge region. In one embodiment, the precursor polypeptides comprise a transmembrane region having the amino acid sequence of SEQ ID NO:36 (from CD28), SEQ ID NO:37 (from CD8), SEQ ID NO:38 (from 4-1BB), or SEQ ID NO:39 (from CD3). In one embodiment, the precursor polypeptides comprise an intracellular JAK-STAT signaling region having an IL2Rf3 intracellular region with Box 1 and Box 2 motifs (SEQ ID NO:43). In one embodiment, the precursor polypeptides comprise an intracellular JAK-STAT signaling region having the amino acid sequence of at least one CD3ζ ITAM domain which includes a heterologous STAT binding motif, for example: CD3ζ ITAM 1 (SEQ ID NO:52, 53 or 54); CD3ζ ITAM 2; CD3ζ ITAM 3 (SEQ ID NO:49, 58 or 60); CD3ζ ITAM3 having a deleted portion (designated ITAM d3, SEQ ID NO:51 or 55); CD3ζ ITAM 1 and 2; CD3ζ ITAM 1, 2 and 3 (SEQ ID NO:47); CD3ζ ITAM 1 and 3 (SEQ ID NO:49); CD3ζITAM 2 and 3; or CD3ζITAM 1 and ITAM d3 (SEQ ID NO:51 or 56). In one embodiment, the STAT binding motif in the CD3ζ ITAM domain comprises the amino acid sequence YXXQ (SEQ ID NO:70), or can be substituted with YXXF (SEQ ID NO:68) or YXXL (SEQ ID NO:69), wherein “X” is any amino acid. In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the precursor polypeptides comprise an intracellular JAK-STAT signaling region which optionally includes a costimulatory region having an intracellular region from CD28 (SEQ ID NO:41), 4-1BB (SEQ ID NO:40) or OX40 (SEQ ID NO:42). In one embodiment, the precursor polypeptides comprise a self-cleaving sequence having the sequence of a T2A, P2A, E2A, or F2A sequence (SEQ ID NO:91, 92, 93 and 94, respectively). In one embodiment, the precursor polypeptides comprise a light chain leader sequence comprising the amino acid sequence of SEQ ID NO:89 or 90, or the precursor polypeptide lacks a light chain leader sequence. In one embodiment, the precursor polypeptides comprise an antibody light chain variable region having the light chain CDR1, CDR2, and CDR3 of a light chain variable region comprising the sequence of any one of SEQ ID NO: 10, 12, 14, 16, 18, 20, 22, 24, 26, 28, 30 or 32. In one embodiment, the precursor polypeptides comprise an antibody light chain variable region having an amino acid sequence having at least 95% sequence identity to the amino acid sequence of any one of SEQ ID NO: 10, 12, 14, 16, 18, 20, 22, 24, 26, 28, 30 or 32. In one embodiment, the precursor polypeptides comprise an antibody light chain constant region having a sequence from a human light chain constant region. In one embodiment, the precursor polypeptides comprise an antibody light chain constant region having a sequence from a kappa or lambda light chain constant region. In one embodiment, the precursor polypeptides comprise an antibody light chain constant region having an amino acid sequence having at least 95% sequence identity to the amino acid sequence of SEQ ID NO:7 (lambda) or 8 (kappa). In one embodiment, the precursor polypeptides (e.g., FIG. 3A) comprise the amino acid sequence of SEQ ID NO:95, 98, 101, 113, 116, 119, 122, 125 or 128, or comprise an amino acid sequence having at least 95%, at least 96%, at least 97%, at least 98%, or at least 99% identity to any of SEQ ID NO:95, 98, 101, 113, 116, 119, 122, 125 or 128.

In one embodiment, the precursor polypeptides (e.g., shown in FIG. 3B) comprise a light chain leader sequence comprising the amino acid sequence of SEQ ID NO:89 or 90, or lacks a light chain leader sequence. In one embodiment, the precursor polypeptides comprise an antibody light chain variable region having an amino acid sequence having at least 95% sequence identity to the amino acid sequence of any one of SEQ ID NO: 10, 12, 14, 16, 18, 20, 22, 24, 26, 28, 30 or 32. In one embodiment, the precursor polypeptides comprise an antibody light chain constant region having a sequence from a human light chain constant region. In one embodiment, the precursor polypeptides comprise an antibody light chain constant region having a sequence from a kappa or lambda light chain constant region. In one embodiment, the precursor polypeptides comprise an antibody light chain constant region having an amino acid sequence having at least 95% sequence identity to the amino acid sequence of SEQ ID NO:7 (lambda) or 8 (kappa). In one embodiment, the precursor polypeptides comprise a hinge region having a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the precursor polypeptides lacks a hinge region. In one embodiment, the precursor polypeptides comprise a transmembrane region having the amino acid sequence of SEQ ID NO:36 (from CD28), SEQ ID NO:37 (from CD8), SEQ ID NO:38 (from 4-1BB), or SEQ ID NO:39 (from CD3). In one embodiment, the precursor polypeptides comprise an intracellular JAK-STAT signaling region having an IL2Rβ intracellular region with Box 1 and Box 2 motifs (SEQ ID NO:43). In one embodiment, the precursor polypeptides comprise an intracellular JAK-STAT signaling region having the amino acid sequence from at least one CD3ζ ITAM domain which includes a heterologous STAT binding motif, for example: CD3ζ ITAM 1 (SEQ ID NO:52, 53 or 54); CD3ζ ITAM 2; CD3ζ ITAM 3 (SEQ ID NO:49, 58 or 60); CD3ζ ITAM3 having a deleted portion (designated ITAM d3, SEQ ID NO:51 or 55); CD3ζ ITAM 1 and 2; CD3ζ ITAM 1, 2 and 3 (SEQ ID NO:47); CD3ζ ITAM 1 and 3 (SEQ ID NO:49); CD3ζ ITAM 2 and 3; or CD3ζ ITAM 1 and ITAM d3 (SEQ ID NO:51 or 56). In one embodiment, the STAT binding motif in the CD3ζ ITAM domain comprises the amino acid sequence YXXQ (SEQ ID NO:70), or can be substituted with YXXF (SEQ ID NO:68) or YXXL (SEQ ID NO:69), wherein “X” is any amino acid. In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the precursor polypeptides comprise an intracellular JAK-STAT signaling region which optionally includes a costimulatory region having an intracellular region from CD28 (SEQ ID NO:41), 4-1BB (SEQ ID NO:40) or OX40 (SEQ ID NO:42). In one embodiment, the precursor polypeptides comprise a self-cleaving sequence having the sequence of a T2A, P2A, E2A, or F2A sequence (SEQ ID NO:91, 92, 93 and 94, respectively). In one embodiment, the precursor polypeptides comprise a heavy chain leader sequence comprising the amino acid sequence of SEQ ID NO:88 or 90, or the precursor polypeptide lacks a heavy chain leader sequence. In one embodiment, the precursor polypeptides comprise an antibody heavy chain variable region having an amino acid sequence having at least 95% sequence identity to the amino acid sequence of any one of SEQ ID NOS: 9, 11, 13, 15, 17, 19, 21, 23, 25, 27, 29 or 31. In one embodiment, the precursor polypeptides comprise an antibody heavy chain constant region having sequences derived from a human antibody constant region, e.g., a human CH1 domain. In one embodiment, the precursor polypeptides comprise an antibody heavy chain constant region which is derived from an IgM, IgA, IgG, IgE or IgD antibody. In one embodiment, the precursor polypeptides comprise an antibody heavy chain constant region having the amino acid sequence of SEQ ID NO:5 (CPPC) or 6 (SPPC).

In some embodiments, the antibody heavy chain variable region of the precursor polypeptide comprises the heavy chain CDR1, CDR2, and CDR3 of a heavy chain variable region and the antibody light chain variable region of the precursor polypeptide comprises the light chain CDR1, CDR2, and CDR3 of a light chain variable region, and the heavy and light chain regions comprise the sequence of SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively. In some embodiments, the antibody heavy chain variable region of the precursor polypeptide comprises a sequence having at least 95%, 97%, 98%, or 99% identity to a first sequence and the antibody light chain variable region of the precursor polypeptide comprises a sequence having at least 95%, 97%, 98%, or 99% identity to a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively. In some embodiments, the antibody heavy chain variable region of the precursor polypeptide comprises a first sequence and the antibody light chain variable region of the precursor polypeptide comprises a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

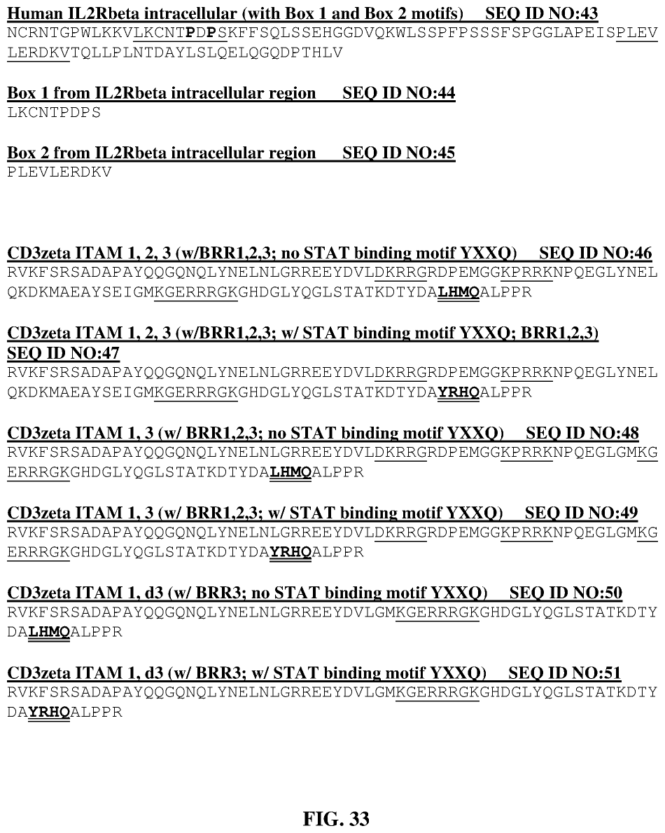

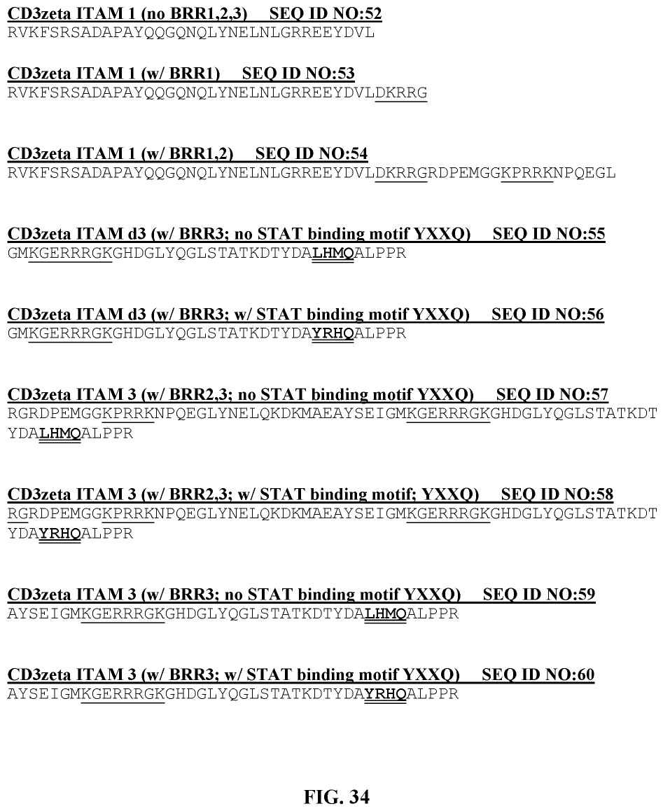

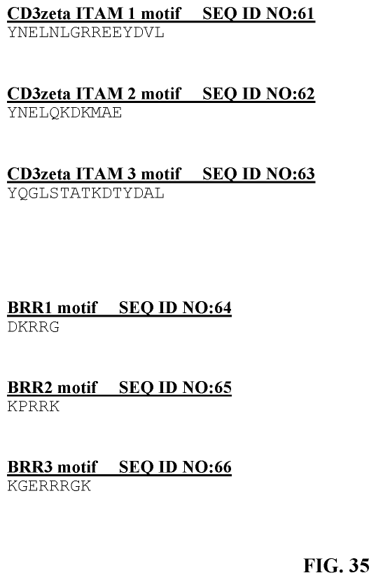

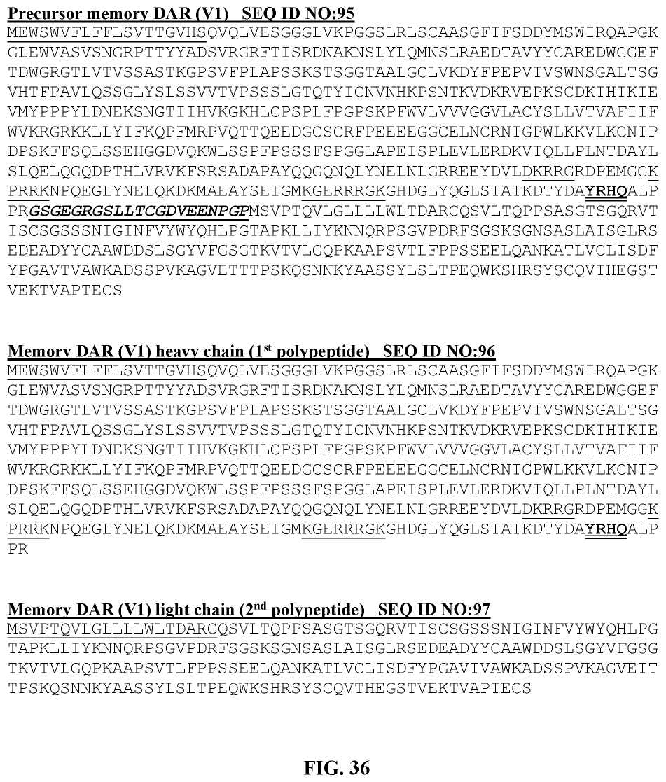

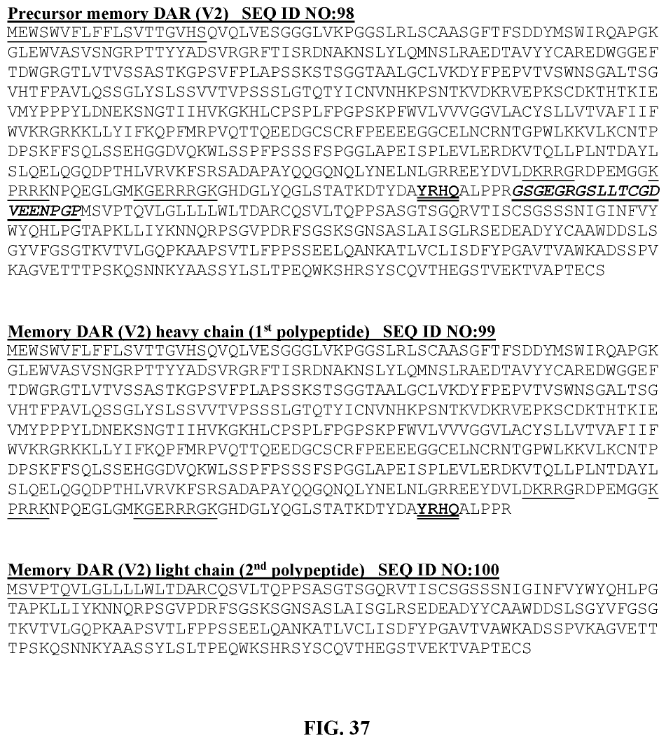

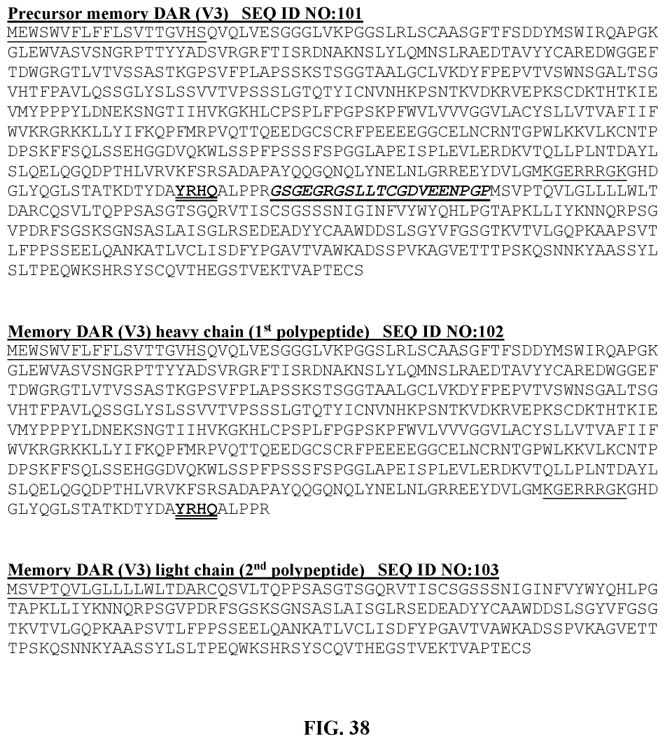

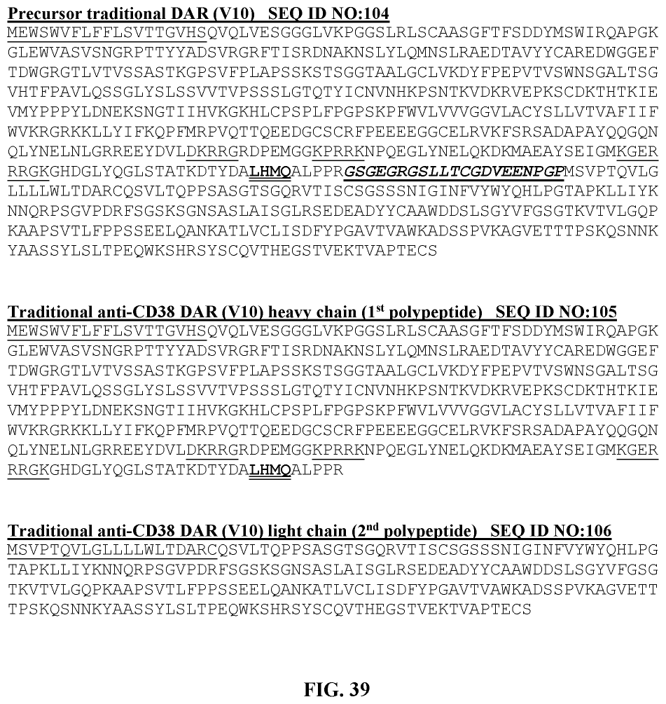

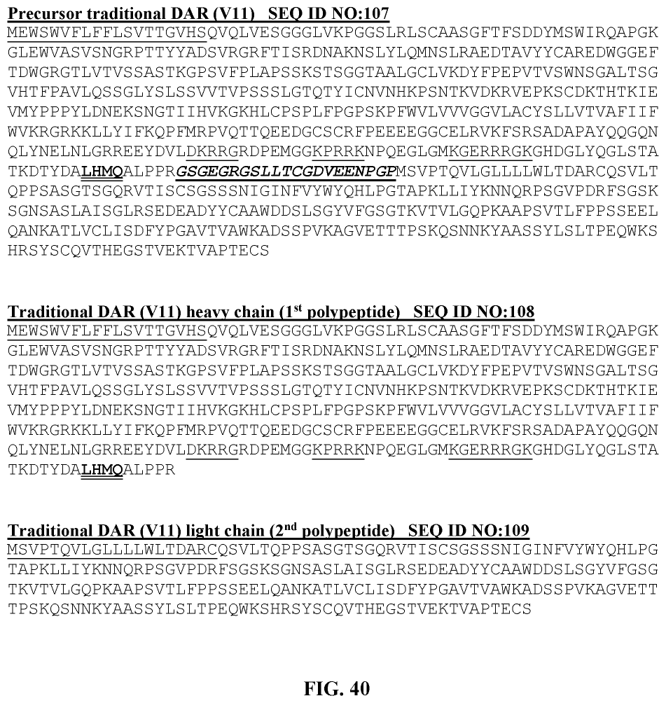

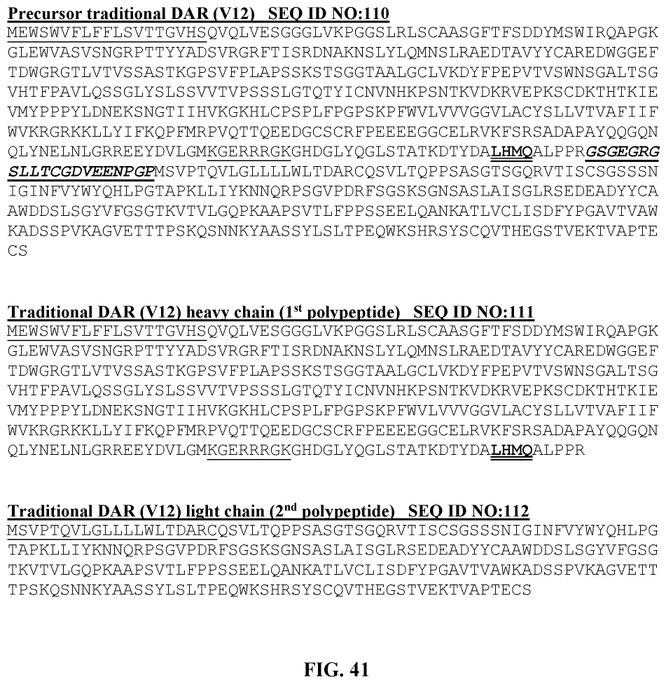

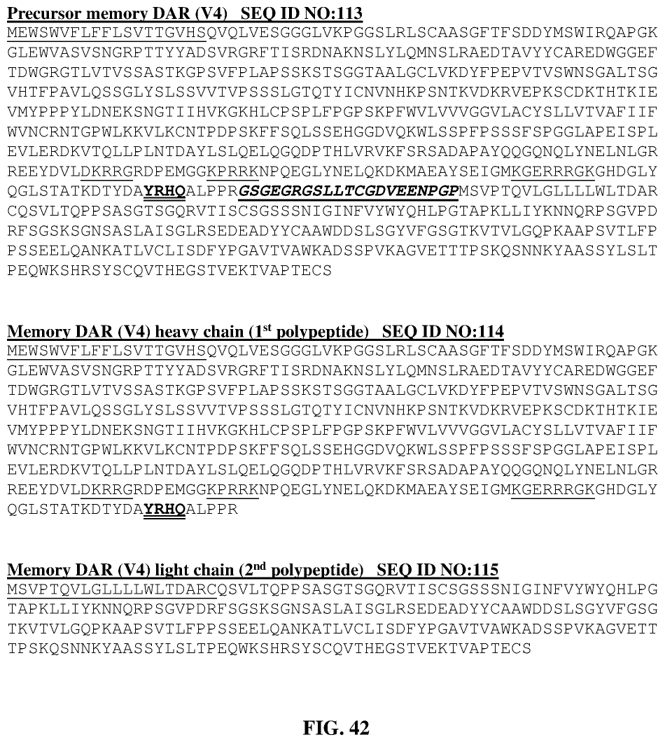

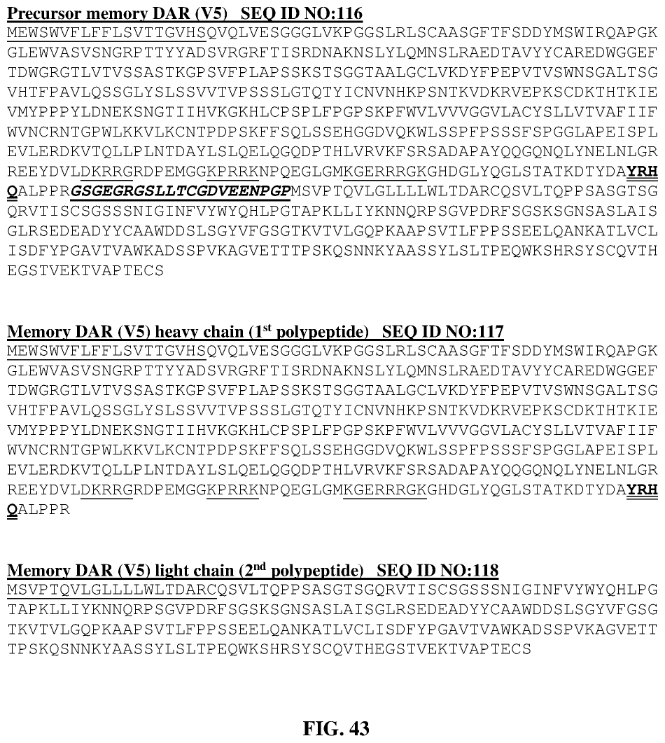

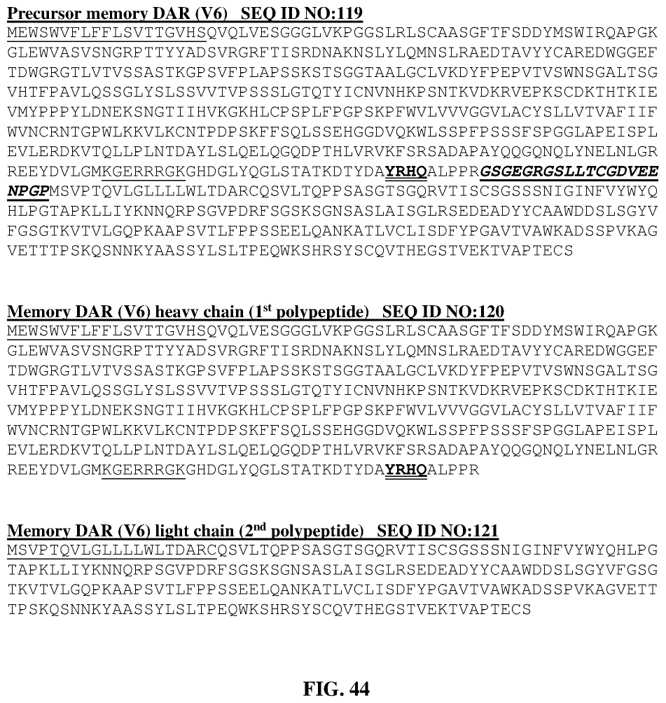

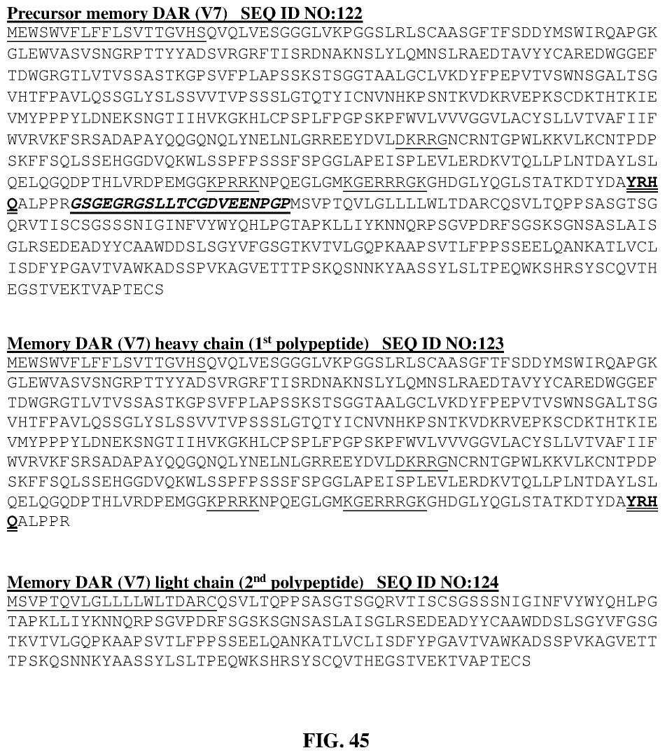

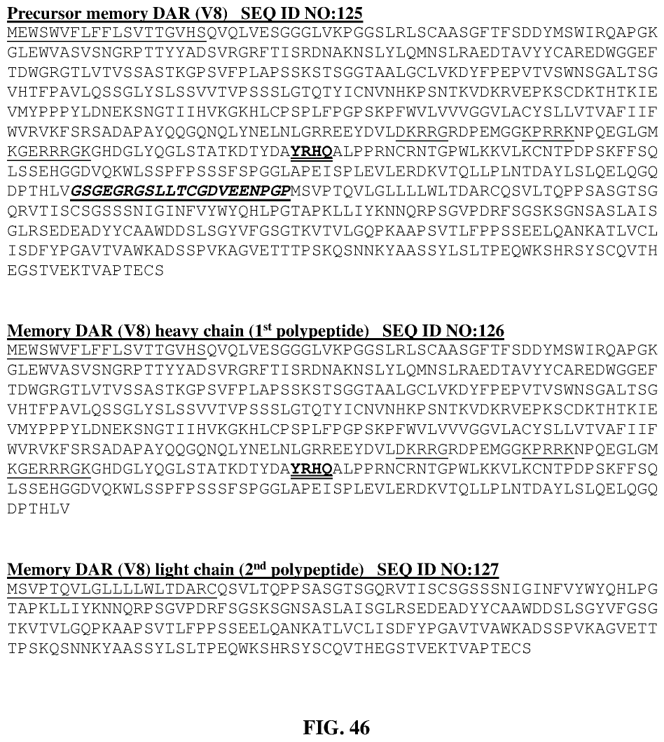

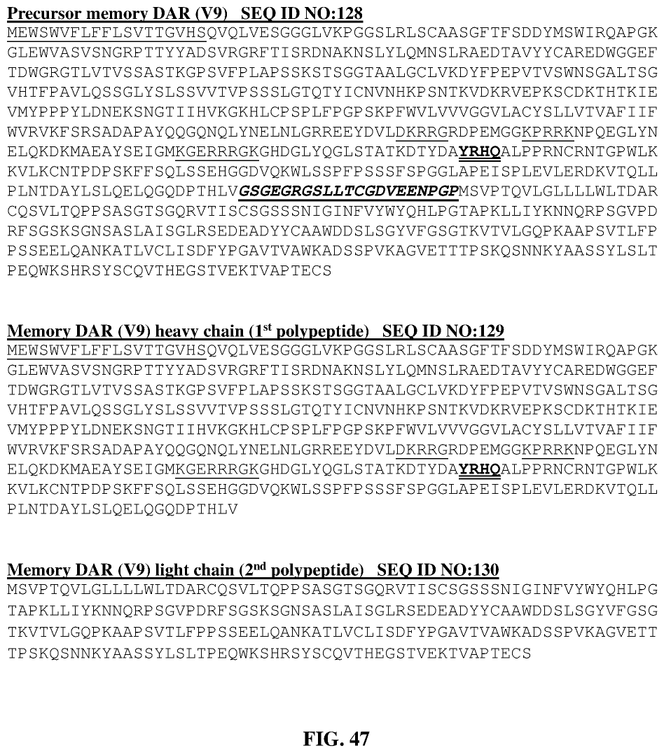

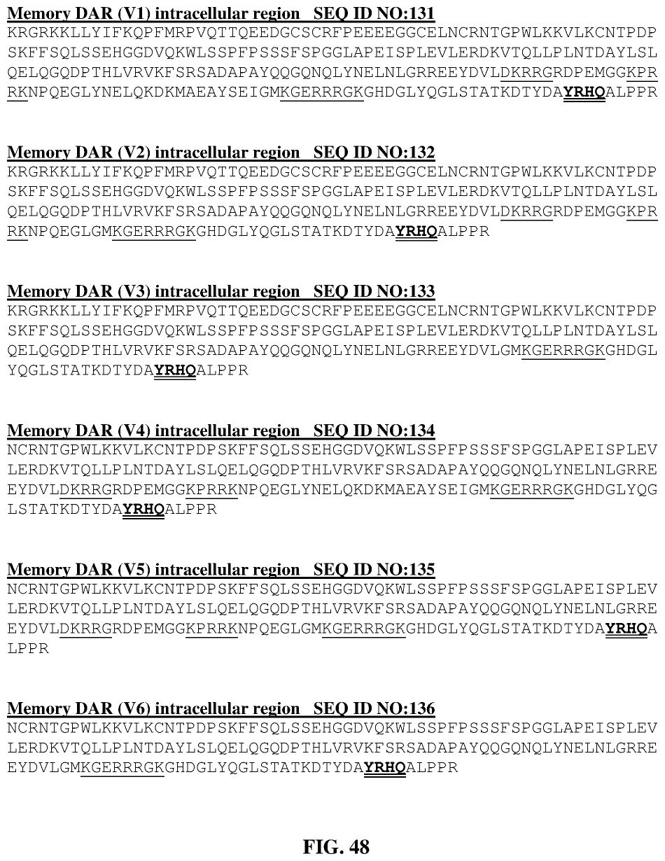

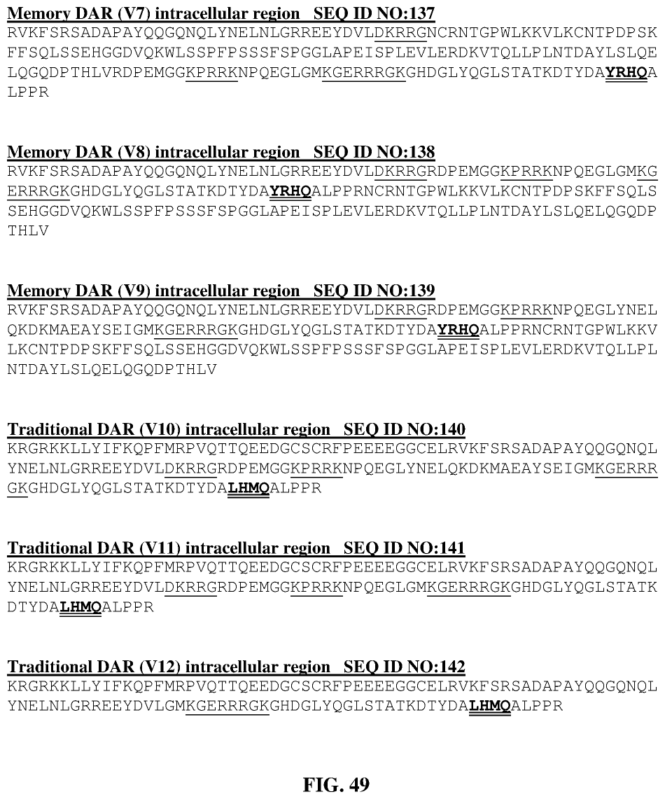

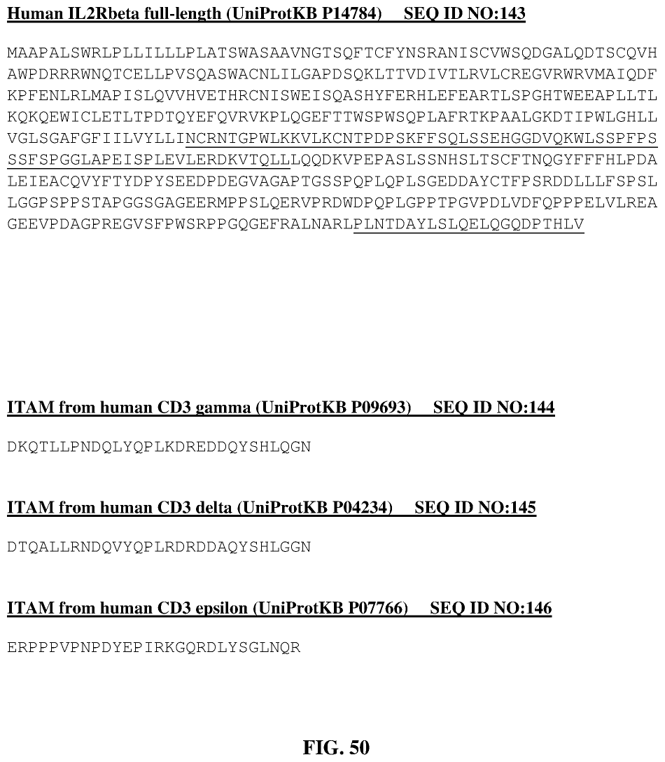

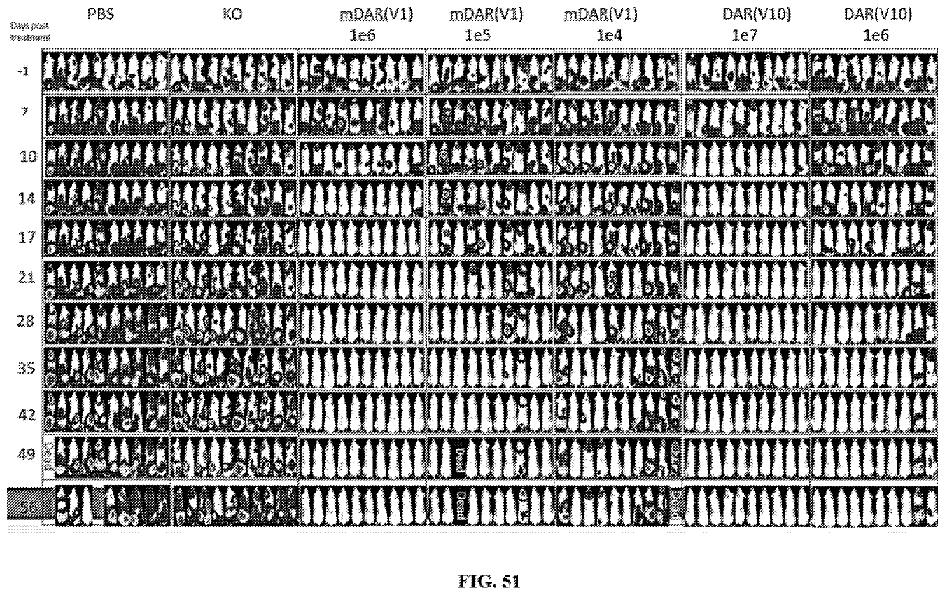

FIGS. 33-35 provide sequences of polypeptide domains and regions as well as amino acid sequence motifs and regions that can be found in mDARs as disclosed herein. FIGS. 36-38 provide examples of mDARs disclosed herein. For example, the exemplary mDAR V1 precursor protein of SEQ ID NO:95 and first and second precursor mDAR(V1) polypeptides as SEQ ID NO:96 and SEQ ID NO:97 are provided in FIG. 36, where the signal peptides of the precursors are underlined. The exemplary mDAR V2 precursor protein of SEQ ID NO:98 and first and second precursor mDAR(V2) polypeptides as SEQ ID NO:99 and SEQ ID NO:100 are provided in FIG. 37. The exemplary mDAR V3 precursor protein of SEQ ID NO:101 and first and second precursor mDAR(V3) polypeptides as SEQ ID NO:102 and SEQ ID NO:103 are provided in FIG. 38. Additional DAR polypeptides are set forth in FIGS. 42-47. In these figures, the signal peptides of the precursors are underlined, and other domains, motifs, or binding regions of these sequences (that may be identified in FIGS. 33-35) may also be underlined or in bold type. While not limiting the mDARs disclosed herein to the embodiments of FIG. 36-38 or 42-47, specifically considered herein are mDAR precursor polypeptides having the amino acid sequences of the polypeptides disclosed therein, mature mDAR polypeptides derived therefrom, as well as mDAR precursors having at least 95%, at least 96%, at least 97%, at least 98%, or at least 99% identity thereto; i.e., precursor polypeptides having at least 95%, at least 96%, at least 97%, at least 98%, or at least 99% amino acid sequence identity to any of SEQ ID NO:98-103, and precursor polypeptides having at least 95%, at least 96%, at least 97%, at least 98%, or at least 99% amino acid sequence identity to any of SEQ ID NOs:113-130.

### Nucleic Acids Encoding Dimeric Antigen Receptors and Related Molecules

The present disclosure provides nucleic acids that encode any of the precursor polypeptides, first polypeptide chains or second polypeptide chains described herein. In any of the nucleic acid embodiments described herein that encode a precursor polypeptide comprising a self-cleaving sequence, the self-cleaving sequence may be a T2A, P2A, E2A, or F2A sequence. In one embodiment, the self-cleaving sequence is other than a T2A sequence, e.g., the self-cleaving sequence is a P2A, E2A, or F2A sequence.

The present disclosure provides a nucleic acid encoding an mDAR precursor polypeptide (FIG. 3A) comprising a plurality of polypeptide regions, the plurality comprising, in order from the amino terminus to the carboxyl terminus: (i) a heavy chain leader sequence, (ii) an antibody heavy chain variable region, (iii) an antibody heavy chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40), (vii) a self-cleaving sequence, (viii) a light chain leader sequence, (ix) an antibody light chain variable region, and (x) an antibody light chain constant region, wherein the self-cleaving sequence permits cleaving of the precursor polypeptide and release of the encoded first and second polypeptide chains.

The present disclosure provides a nucleic acid encoding an mDAR precursor polypeptide (FIG. 3A), comprising a heavy chain leader sequence having the amino acid sequence of SEQ ID NO:88 or 90, or lacks a heavy chain leader sequence. In one embodiment, the nucleic acid encodes a precursor polypeptide comprising an antibody heavy chain variable region having an amino acid sequence having at least 95% sequence identity to the amino acid sequence of any one of SEQ ID NOS: 9, 11, 13, 15, 17, 19, 21, 23, 25, 27, 29 or 31. In one embodiment, the nucleic acid encodes a precursor polypeptide comprising an antibody heavy chain constant region having sequences derived from a human antibody constant region, e.g., a human CH1 domain. In one embodiment, the nucleic acid encodes a precursor polypeptide comprising an antibody heavy chain constant region which is derived from an IgM, IgA, IgG, IgE or IgD antibody. In one embodiment, the nucleic acid encodes a precursor polypeptide comprising an antibody heavy chain constant region having the amino acid sequence of SEQ ID NO:5 (CPPC) or 6 (SPPC). In one embodiment, the nucleic acid encodes a precursor polypeptide comprising a hinge region having a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the nucleic acid encodes a precursor polypeptide that lacks a hinge region. In one embodiment, the nucleic acid encodes a precursor polypeptide comprising a transmembrane region having the amino acid sequence of SEQ ID NO:36 (from CD28), SEQ ID NO:37 (from CD8), SEQ ID NO:38 (from 4-1BB), or SEQ ID NO:39 (from CD3). In one embodiment, the nucleic acid encodes a precursor polypeptide comprising an intracellular JAK-STAT signaling region having an IL2Rβ intracellular region with Box 1 and Box 2 motifs (SEQ ID NO:43). In one embodiment, the nucleic acid encodes a precursor polypeptide comprising an intracellular JAK-STAT signaling region having the amino acid sequence from at least one CD3ζ ITAM domain which includes a heterologous STAT binding motif, for example: CD3ζ ITAM 1 (SEQ ID NO:52, 53 or 54); CD3ζ ITAM 2; CD3ζ ITAM 3 (SEQ ID NO:49, 58 or 60); CD3ζ ITAM3 having a deleted portion (designated ITAM d3, SEQ ID NO:51 or 55); CD3ζ ITAM 1 and 2; CD3ζ ITAM 1, 2 and 3 (SEQ ID NO:47); CD3ζ ITAM 1 and 3 (SEQ ID NO:49); CD3ζ ITAM 2 and 3; or CD3ζ ITAM 1 and ITAM d3 (SEQ ID NO:51 or 56). In one embodiment, the STAT binding motif in the CD3ζ ITAM domain comprises the amino acid sequence YXXQ (SEQ ID NO:70), or can be substituted with YXXF (SEQ ID NO:68) or YXXL (SEQ ID NO:69), wherein “X” is any amino acid. In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the nucleic acid encodes a precursor polypeptide comprising an intracellular JAK-STAT signaling region which optionally includes a costimulatory region having an intracellular region from CD28 (SEQ ID NO:41), 4-1BB (SEQ ID NO:40) or OX40 (SEQ ID NO:42). In one embodiment, the nucleic acid encodes a precursor polypeptide comprising a self-cleaving sequence having the sequence of a T2A, P2A, E2A, or F2A sequence (SEQ ID NO:91, 92, 93 and 94, respectively). In one embodiment, the nucleic acid encodes a precursor polypeptide comprising a light chain leader sequence comprising the amino acid sequence of SEQ ID NO:89 or 90, or the precursor polypeptide lacks a light chain leader sequence. In one embodiment, the nucleic acid encodes a precursor polypeptide comprising an antibody light chain variable region having an amino acid sequence having at least 95% sequence identity to the amino acid sequence of any one of SEQ ID NO: 10, 12, 14, 16, 18, 20, 22, 24, 26, 28, 30 or 32. In one embodiment, the nucleic acid encodes a precursor polypeptide comprising an antibody light chain constant region having a sequence from a human light chain constant region. In one embodiment, the nucleic acid encodes a precursor polypeptide comprising an antibody light chain constant region having a sequence from a kappa or lambda light chain constant region. In one embodiment, the nucleic acid encodes a precursor polypeptide comprising an antibody light chain constant region having an amino acid sequence having at least 95% sequence identity to the amino acid sequence of SEQ ID NO:7 (lambda) or 8 (kappa). In one embodiment, the nucleic acid encodes a precursor polypeptide (e.g., FIG. 3A) comprising the amino acid sequence of SEQ ID NO: 95, 98, 101, 113, 116, 119, 122, 125 or 128.

The present disclosure provides a nucleic acid encoding an mDAR precursor polypeptide comprising a plurality of polypeptide regions, the plurality comprising, in order from the amino terminus to the carboxyl terminus: (i) a light chain leader sequence, (ii) an antibody light chain variable region, (iii) an antibody light chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40), (vii) a self-cleaving sequence, (viii) a heavy chain leader sequence, (ix) an antibody heavy chain variable region, and (x) an antibody heavy chain constant region, wherein the self-cleaving sequence permits cleaving of the precursor polypeptide and release of the encoded first and second polypeptide chains (FIG. 3B).

The present disclosure provides a nucleic acid encoding an mDAR precursor polypeptide (FIG. 3B) comprising a light chain leader sequence having the amino acid sequence of SEQ ID NO:89 or 90, or lacks a light chain leader sequence. In one embodiment, the nucleic acid encodes a precursor polypeptide comprising an antibody light chain variable region having an amino acid sequence having at least 95% sequence identity to the amino acid sequence of any one of SEQ ID NO: 10, 12, 14, 16, 18, 20, 22, 24, 26, 28, 30 or 32. In one embodiment, the nucleic acid encodes a precursor polypeptide comprising an antibody light chain constant region having a sequence from a human light chain constant region. In one embodiment, the nucleic acid encodes a precursor polypeptide comprising an antibody light chain constant region having a sequence from a kappa or lambda light chain constant region. In one embodiment, the nucleic acid encodes a precursor polypeptide comprising an antibody light chain constant region having an amino acid sequence having at least 95% sequence identity to the amino acid sequence of SEQ ID NO:7 (lambda) or 8 (kappa). In one embodiment, the nucleic acid encodes a precursor polypeptide comprising a hinge region having a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the precursor polypeptides lacks a hinge region. In one embodiment, the nucleic acid encodes a precursor polypeptide comprising a transmembrane region having the amino acid sequence of SEQ ID NO:36 (from CD28), SEQ ID NO:37 (from CD8), SEQ ID NO:38 (from 4-1BB), or SEQ ID NO:39 (from CD3). In one embodiment, the nucleic acid encodes a precursor polypeptide comprising an intracellular JAK-STAT signaling region having an IL2Rβ intracellular region (SEQ ID NO:43) with Box 1 and Box 2 motifs. In one embodiment, the nucleic acid encodes a precursor polypeptide comprising an intracellular JAK-STAT signaling region having the amino acid sequence from at least one CD3ζ ITAM domain which includes a heterologous STAT binding motif, for example: CD3ζ ITAM domain which includes a heterologous STAT binding motif, for example: CD3ζ ITAM 1 (SEQ ID NO:52, 53 or 54); CD3ζ ITAM 2; CD3ζ ITAM 3 (SEQ ID NO:49, 58 or 60); CD3ζ ITAM3 having a deleted portion (designated ITAM d3, SEQ ID NO:51 or 55); CD3ζ ITAM 1 and 2; CD3ζ ITAM 1, 2 and 3 (SEQ ID NO:47); CD3ζ ITAM 1 and 3 (SEQ ID NO:49); CD3ζ ITAM 2 and 3; or CD3ζ ITAM 1 and ITAM d3 (SEQ ID NO:51 or 56). In one embodiment, the STAT binding motif in the CD3ζ ITAM domain comprises the amino acid sequence YXXQ (SEQ ID NO:70), or can be substituted with YXXF (SEQ ID NO:68) or YXXL (SEQ ID NO:69), wherein “X” is any amino acid. In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the nucleic acid encodes a precursor polypeptide comprising an intracellular JAK-STAT signaling region which optionally includes a costimulatory region having an intracellular region from CD28 (SEQ ID NO:41), 4-1BB (SEQ ID NO:40) or OX40 (SEQ ID NO:42). In one embodiment, the nucleic acid encodes a precursor polypeptide comprising a self-cleaving sequence having the sequence of a T2A, P2A, E2A, or F2A sequence (SEQ ID NO:91, 92, 93 and 94, respectively). In one embodiment, the nucleic acid encodes a precursor polypeptide comprising a heavy chain leader sequence comprising the amino acid sequence of SEQ ID NO:88 or 90, or the precursor polypeptide lacks a heavy chain leader sequence. In one embodiment, the nucleic acid encodes a precursor polypeptide comprising an antibody heavy chain variable region having an amino acid sequence having at least 95% sequence identity to the amino acid sequence of any one of SEQ ID NOS: 9, 11, 13, 15, 17, 19, 21, 23, 25, 27, 29 or 31. In one embodiment, the nucleic acid encodes a precursor polypeptide comprising an antibody heavy chain constant region having sequences derived from a human antibody constant region, e.g., a human CH1 domain. In one embodiment, the nucleic acid encodes a precursor polypeptide comprising an antibody heavy chain constant region which is derived from an IgM, IgA, IgG, IgE or IgD antibody. In one embodiment, the nucleic acid encodes a precursor polypeptide comprising an antibody heavy chain constant region having the amino acid sequence of SEQ ID NO:5 (CPPC) or 6 (SPPC).

The present disclosure provides a first nucleic acid encoding a first polypeptide chain and a second nucleic acid encoding a second polypeptide chain (FIG. 1A). In one embodiment, the first nucleic acid encoding the first polypeptide chain (FIG. 1A) comprises a heavy chain leader sequence having the amino acid sequence of SEQ ID NO:88 or 90, or lacks a heavy chain leader sequence. In one embodiment, the second nucleic acid encoding the second polypeptide chain (FIG. 1A) comprising a light chain leader sequence comprising the amino acid sequence of SEQ ID NO:89 or 90, or the second polypeptide chain lacks a light chain leader sequence. In one embodiment: (a) the first nucleic acid encodes the first polypeptide chain comprising a plurality of polypeptide regions, the plurality comprising, in order from the amino terminus to the carboxyl terminus: (i) an antibody heavy chain variable region (VH), (ii) an antibody heavy chain constant region (CH), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) the second nucleic acid encodes the second polypeptide chain comprising a plurality of polypeptide regions, the plurality comprising, in order from the amino terminus to the carboxyl terminus: (i) an antibody light chain variable region (VL) (e.g., kappa or lambda), and (ii) an antibody light chain constant region (CL), wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain (FIG. 1A).

In one embodiment, the first nucleic acid encoding the first polypeptide chain (FIG. 1A) comprises a heavy chain leader sequence having the amino acid sequence of SEQ ID NO:88 or 90, or lacks a heavy chain leader sequence. In one embodiment, the first nucleic acid encoding the first polypeptide chain comprising an antibody heavy chain variable region having an amino acid sequence having at least 95% sequence identity to the amino acid sequence of any one of SEQ ID NOS: 9, 11, 13, 15, 17, 19, 21, 23, 25, 27, 29 or 31. In one embodiment, the first nucleic acid encoding the first polypeptide chain comprising an antibody heavy chain constant region having sequences derived from a human antibody constant region, e.g., a human CH1 domain. In one embodiment, the first nucleic acid encoding the first polypeptide chain comprising an antibody heavy chain constant region which is derived from an IgM, IgA, IgG, IgE or IgD antibody. In one embodiment, the first nucleic acid encoding the first polypeptide chain comprising an antibody heavy chain constant region having the amino acid sequence of SEQ ID NO:5 (CPPC) or 6 (SPPC). In one embodiment, the first nucleic acid encoding the first polypeptide chain comprising a hinge region having a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the first nucleic acid encoding the first polypeptide chain that lacks a hinge region. In one embodiment, the first nucleic acid encoding the first polypeptide chain comprising a transmembrane region having the amino acid sequence of SEQ ID NO:36 (from CD28), SEQ ID NO:37 (from CD8), SEQ ID NO:38 (from 4-1BB), or SEQ ID NO:39 (from CD3). In one embodiment, the first nucleic acid encoding the first polypeptide chain comprising an intracellular JAK-STAT signaling region having an IL2Rβ intracellular region (SEQ ID NO:43) with Box 1 and Box 2 motifs. In one embodiment, the first nucleic acid encoding the first polypeptide chain comprising an intracellular JAK-STAT signaling region having the amino acid sequence from at least one CD3ζ ITAM domain which includes a heterologous STAT binding motif, for example: CD3ζ ITAM 1 (SEQ ID NO:52, 53 or 54); CD3ζ ITAM 2; CD3ζ ITAM 3 (SEQ ID NO:49, 58 or 60); CD3ζ ITAM3 having a deleted portion (designated ITAM d3, SEQ ID NO:51 or 55); CD3ζ ITAM 1 and 2; CD3ζ ITAM 1, 2 and 3 (SEQ ID NO:47); CD3ζ ITAM 1 and 3 (SEQ ID NO:49); CD3ζ ITAM 2 and 3; or CD3ζ ITAM 1 and ITAM d3 (SEQ ID NO:51 or 56). In one embodiment, the STAT binding motif in the CD3ζ ITAM domain comprises the amino acid sequence YXXQ (SEQ ID NO:70), or can be substituted with YXXF (SEQ ID NO:68) or YXXL (SEQ ID NO:69), wherein “X” is any amino acid. In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the first nucleic acid encoding the first polypeptide chain comprising an intracellular JAK-STAT signaling region which optionally includes a costimulatory region having an intracellular region from CD28 (SEQ ID NO:41), 4-1BB (SEQ ID NO:40) or OX40 (SEQ ID NO:42). In one embodiment, the first nucleic acid encoding the first polypeptide chain (FIG. 1A) comprising the amino acid sequence of SEQ ID NO:96, 99, 102, 114, 117, 120, 123, 126 or 129 or an amino acid sequence having at least 95%, at least 96%, at least 97%, at least 98%, or at least 99% identity to any of SEQ ID NO:96, 99, 102, 114, 117, 120, 123, 126 or 129.

In one embodiment, the second nucleic acid encoding the second polypeptide chain (FIG. 1A) comprising a light chain leader sequence comprising the amino acid sequence of SEQ ID NO:89 or 90, or the second polypeptide chain lacks a light chain leader sequence. In one embodiment, the second nucleic acid encoding the second polypeptide chain comprising an antibody light chain variable region having an amino acid sequence having at least 95% sequence identity to the amino acid sequence of any one of SEQ ID NO: 10, 12, 14, 16, 18, 20, 22, 24, 26, 28, 30 or 32. In one embodiment, the second nucleic acid encoding the second polypeptide chain comprising an antibody light chain constant region having a sequence from a human light chain constant region. In one embodiment, the second nucleic acid encoding the second polypeptide chain comprising an antibody light chain constant region having a sequence from a kappa or lambda light chain constant region. In one embodiment, the second nucleic acid encoding the second polypeptide chain comprising an antibody light chain constant region having an amino acid sequence having at least 95% sequence identity to the amino acid sequence of SEQ ID NO:7 (lambda) or 8 (kappa). In one embodiment, the second nucleic acid encoding the second polypeptide chain (FIG. 1A) comprises the amino acid sequence of SEQ ID NO:97, 100, 103, 115, 118, 121 124, 127 or 130 or an amino acid sequence having at least 95%, at least 96%, at least 97%, at least 98%, or at least 99% identity to any of SEQ ID NO:97, 100, 103, 115, 118, 121 124, 127 or 130.

The present disclosure provides a first nucleic acid encoding a first polypeptide chain and a second nucleic acid encoding a second polypeptide chain (FIG. 1B). In one embodiment, the first nucleic acid encoding the first polypeptide chain (FIG. 1B) comprises a light chain leader sequence having the amino acid sequence of SEQ ID NO:89 or 90, or lacks a light chain leader sequence. In one embodiment, the second nucleic acid encoding the second polypeptide chain (FIG. 1B) comprising a heavy chain leader sequence comprising the amino acid sequence of SEQ ID NO:88 or 90, or the second polypeptide chain lacks a heavy chain leader sequence. In one embodiment: (a) a first polypeptide chain comprising a plurality of polypeptide regions, the plurality comprising, in order from the amino terminus to the carboxyl terminus: (i) an antibody light chain variable region (VL), (ii) an antibody light chain constant region (CL), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) a second polypeptide chain comprising regions, the plurality comprising, in order from the amino terminus to the carboxyl terminus: (i) an antibody heavy chain variable region (VH), and (ii) an antibody heavy chain constant region (CH), wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain (FIG. 1B).

In one embodiment, the first nucleic acid encoding the first polypeptide chain (FIG. 1B) comprises a light chain leader sequence having the amino acid sequence of SEQ ID NO:89 or 90, or lacks a light chain leader sequence. In one embodiment, the first nucleic acid encoding the first polypeptide chain comprising an antibody light chain variable region comprising an amino acid sequence having at least 95% sequence identity to the amino acid sequence of any one of SEQ ID NO: 10, 12, 14, 16, 18, 20, 22, 24, 26, 28, 30 or 32. In one embodiment, the first nucleic acid encoding the first polypeptide chain comprising the antibody light chain constant region comprises a sequence from a human light chain constant region. In one embodiment, the first nucleic acid encoding the first polypeptide chain comprising the antibody light chain constant region comprises a sequence from a kappa or lambda light chain constant region. In one embodiment, the first nucleic acid encoding the first polypeptide chain comprising the antibody light chain constant region comprises an amino acid sequence having at least 95% sequence identity to the amino acid sequence of SEQ ID NO:7 (lambda) or 8 (kappa). In one embodiment, the first nucleic acid encoding the first polypeptide chain comprising the hinge region comprises a CD28 hinge comprising the amino acid sequence of SEQ ID NO:34, or a CD8 hinge comprising the amino acid sequence of SEQ ID NO:33, or a hinge region comprising a CD28 and CD8 hinge sequences of SEQ ID NO:35 (e.g., long hinge). In one embodiment, the first nucleic acid encoding the first polypeptide chain which lacks a hinge region. In one embodiment, the first nucleic acid encoding the first polypeptide chain comprising the transmembrane region comprises the amino acid sequence of SEQ ID NO:36 (from CD28), SEQ ID NO:37 (from CD8), SEQ ID NO:38 (from 4-1BB), or SEQ ID NO:39 (from CD3). In one embodiment, the first nucleic acid encoding the first polypeptide chain comprising the intracellular JAK-STAT signaling region comprises an IL2Rβ intracellular region (SEQ ID NO:43) having Box 1 and Box 2 motifs. In one embodiment, the first nucleic acid encoding the first polypeptide chain comprising the intracellular JAK-STAT signaling region comprises the amino acid sequence from at least one CD3ζ ITAM domain which includes a heterologous STAT binding motif, for example: CD3ζ ITAM 1 (SEQ ID NO:52, 53 or 54); CD3ζ ITAM 2; CD3ζ ITAM 3 (SEQ ID NO:49, 58 or 60); CD3ζ ITAM3 having a deleted portion (designated ITAM d3, SEQ ID NO:51 or 55); CD3ζ ITAM 1 and 2; CD3ζ ITAM 1, 2 and 3 (SEQ ID NO:47); CD3ζ ITAM 1 and 3 (SEQ ID NO:49); CD3ζ ITAM 2 and 3; or CD3ζ ITAM 1 and ITAM d3 (SEQ ID NO:51 or 56). In one embodiment, the STAT binding motif in the CD3ζ ITAM domain comprises the amino acid sequence YXXQ (SEQ ID NO:70), or can be substituted with YXXF (SEQ ID NO:68) or YXXL (SEQ ID NO:69), wherein “X” is any amino acid. In one embodiment, the STAT binding motif within the CD3ζ ITAM domain comprises YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (SEQ ID NOS:70-87, respectively). In one embodiment, the first nucleic acid encoding the first polypeptide chain comprising the intracellular JAK-STAT signaling region optionally includes a costimulatory region comprising an intracellular region from CD28 (SEQ ID NO:41), 4-1BB (SEQ ID NO:40) or OX40 (SEQ ID NO:42).

In one embodiment, the second nucleic acid encoding the second polypeptide chain (FIG. 1B) comprises a heavy chain leader sequence having the amino acid sequence of SEQ ID NO:88 or 90, or lacks a heavy chain leader sequence. In one embodiment, the second nucleic acid encoding the second polypeptide chain comprising an antibody heavy chain variable region having an amino acid sequence having at least 95% sequence identity to the amino acid sequence of any one of SEQ ID NO: 9, 11, 13, 15, 17, 19, 21, 23, 25, 27, 29 or 31. In one embodiment, the second nucleic acid encoding the second polypeptide chain comprising the antibody heavy chain constant region comprises sequences derived from a human antibody constant region, e.g., a human CH1 domain. In one embodiment, the second nucleic acid encoding the second polypeptide chain comprising the antibody heavy chain constant region can be derived from an IgM, IgA, IgG, IgE or IgD antibody. In one embodiment, the second nucleic acid encoding the second polypeptide chain comprises a heavy chain constant region having the amino acid sequence of SEQ ID NO:5 (CPPC) or 6 (SPPC).

### Vectors

The present disclosure provides vectors operably linked to one or more nucleic acids that encode any of the precursor polypeptides, first polypeptide chains, second polypeptide chains, or first and second polypeptide chains described herein.

The present disclosure provides a vector comprising at least one promoter sequence operably linked to a nucleic acid that encodes an mDAR precursor polypeptide such as any disclosed herein. In some exemplary embodiments, the vector is operably linked to a nucleic acid encoding an mDAR precursor polypeptide (e.g., FIG. 3A) comprising the amino acid sequence of SEQ ID NO:95, 98, 101, 113, 116, 119, 122, 125 or 128, or an amino acid sequence having at least 95%, at least 96%, at least 97%, at least 98%, or at least 99% identity thereto.

The present disclosure also provides a first vector operably linked to a first nucleic acid that encodes an mDAR first polypeptide chain (e.g., FIG. 1A) comprising a plurality of polypeptide regions, the plurality comprising, in order from the amino terminus to the carboxyl terminus: (i) a heavy chain leader sequence, (ii) an antibody heavy chain variable region, (iii) an antibody heavy chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40). In one embodiment, the first vector is operably linked to a first nucleic acid encoding an mDAR first polypeptide chain that lacks a heavy chain leader region.

The present disclosure provides a second vector operably linked to a second nucleic acid that encodes an mDAR second polypeptide chain (e.g., FIG. 1B) comprising a plurality of polypeptide regions, the plurality comprising, in order from the amino terminus to the carboxyl terminus: (i) a light chain leader sequence, (ii) an antibody light chain variable region, and (iii) an antibody light chain constant region. In one embodiment, the second vector is operably linked to a second nucleic acid encoding an mDAR second polypeptide chain that lacks a light chain leader region.

The present disclosure provides a vector (e.g., a single vector) that is operably linked to nucleic acids encoding an mDAR first polypeptide chain and an mDAR second polypeptide chain (FIG. 1A) wherein the first nucleic acid encoding the first polypeptide chain comprises a heavy chain leader sequence having the amino acid sequence of SEQ ID NO:88 or 90, or lacks a heavy chain leader sequence. In one embodiment, the second nucleic acid encoding the second polypeptide chain comprising a light chain leader sequence comprising the amino acid sequence of SEQ ID NO:89 or 90, or the second polypeptide chain lacks a light chain leader sequence. In one embodiment the vector is operably linked to: (a) the first nucleic acid encodes the first polypeptide chain comprising a plurality of polypeptide regions, the plurality comprising, in order from the amino terminus to the carboxyl terminus: (i) an antibody heavy chain variable region (VH), (ii) an antibody heavy chain constant region (CH), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) the second nucleic acid encodes the second polypeptide chain comprising a plurality of polypeptide regions, the plurality comprising, in order from the amino terminus to the carboxyl terminus: (i) an antibody light chain variable region (VL) (e.g., kappa or lambda), and (ii) an antibody light chain constant region (CL), wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain (FIG. 1A).

The present disclosure provides a first vector operably linked to a first nucleic acid that encodes an mDAR first polypeptide chain (e.g., FIG. 1B) comprising a plurality of polypeptide regions, the plurality comprising, in order from the amino terminus to the carboxyl terminus: (i) a light chain leader sequence, (ii) an antibody light chain variable region, (iii) an antibody light chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40). In one embodiment, the first vector is operably linked to a first nucleic acid encoding an mDAR first polypeptide chain that lacks a light chain leader region.

The present disclosure provides a second vector operably linked to a second nucleic acid that encodes an mDAR second polypeptide chain (FIG. 1B) comprising a plurality of polypeptide regions, the plurality comprising, in order from the amino terminus to the carboxyl terminus: (i) a heavy chain leader sequence, (ii) an antibody heavy chain variable region, and (iii) an antibody heavy chain constant region. In one embodiment, the second vector is operably linked to a second nucleic acid encoding an mDAR second polypeptide chain that lacks a heavy chain leader region.

The present disclosure provides a vector (e.g., a single vector) that is operably linked to nucleic acids encoding the mDAR first polypeptide chain and mDAR second polypeptide chain (FIG. 1B) wherein the first nucleic acid encoding the first polypeptide chain (FIG. 1B) comprises a light chain leader sequence having the amino acid sequence of SEQ ID NO:89 or 90, or lacks a light chain leader sequence. In one embodiment, the second nucleic acid encoding the second polypeptide chain (FIG. 1B) comprising a heavy chain leader sequence comprising the amino acid sequence of SEQ ID NO:88 or 90, or the second polypeptide chain lacks a heavy chain leader sequence. In one embodiment: (a) a first polypeptide chain comprising a plurality of polypeptide regions, the plurality comprising, in order from the amino terminus to the carboxyl terminus: (i) an antibody light chain variable region (VL), (ii) an antibody light chain constant region (CL), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) a second polypeptide chain comprising regions, the plurality comprising, in order from the amino terminus to the carboxyl terminus: (i) an antibody heavy chain variable region (VH), and (ii) an antibody heavy chain constant region (CH), wherein the antibody heavy chain constant region and the antibody light chain constant region form a dimerization domain for formation of the memory dimeric antigen receptor (mDAR), and wherein the antibody heavy chain variable region and the antibody light chain variable region form an antigen binding domain (FIG. 1B).

In one embodiment, any of the first vector, second vector or single vector described herein are operably linked to a nucleic acid encoding a heavy chain leader sequence which comprises the amino acid sequence of SEQ ID NO:88 or 90.

In one embodiment, any of the first vector, second vector or single vector described herein are operably linked to a nucleic acid encoding an antibody heavy chain variable region which comprises a CD38 antibody heavy chain variable region comprising an amino acid sequence having at least 95% sequence identity to the amino acid sequence of any one of SEQ ID NOS: 9, 11, 13, 15, 17, 19, 21, 23, 25, 27, 29 or 31.

In one embodiment, any of the first vector, second vector or single vector described herein are operably linked to a nucleic acid encoding an antibody heavy chain constant region comprising the amino acid sequence of SEQ ID NO:5 (CPPC) or 6 (SPPC).

In one embodiment, any of the first vector, second vector or single vector described herein are operably linked to a nucleic acid encoding a hinge region comprises a CD28 hinge region comprising the amino acid sequence of SEQ ID NO:34, a CD8 hinge region comprising the amino acid sequence of SEQ ID NO:33, or a hinge region having CD28 and CD8 comprising the amino acid sequence of SEQ ID NO:35 (e.g., long hinge).

In one embodiment, any of the first vector, second vector or single vector described herein are operably linked to a nucleic acid encoding a transmembrane region comprising the amino acid sequence of SEQ ID NO:36 (from CD28), SEQ ID NO:37 (from CD8), SEQ ID NO:38 (from 4-1BB), or SEQ ID NO:39 (from CD3).

In one embodiment, any of the first vector, second vector or single vector described herein are optionally operably linked to a nucleic acid encoding an intracellular JAK-STAT signaling region comprising any order and any combination of 1-5 intracellular sequences from 4-1BB costimulatory sequence comprising the amino acid sequence of SEQ ID NO:40, CD28 costimulatory sequence comprising the amino acid sequence of SEQ ID NO:41, or OX40 costimulatory sequence comprising the amino acid sequence of SEQ ID NO:42.

### Host Cells

The present disclosure provides a host cell, or a population of host cells, which harbors one or more expression vectors operably linked to a nucleic acid (e.g., transgene) that encodes any of the mDAR first polypeptide chains, second polypeptide chains, first and second polypeptide chains, or precursor polypeptides described herein.

In one embodiment, the host cell or population of host cells are introduced with one or more expression vectors, where the vectors are operably linked to a nucleic acid transgene encoding any of the memory dimeric antigen receptor (mDAR) polypeptides described herein. The host cell or the population of host cells comprise T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

In one embodiment, the host cell or population of host cells are autologous and are derived from the subject to which they are to be administered to receive treatment of host cells expressing memory dimeric antigen receptors (mDAR). In one embodiment, blood (e.g., whole blood) can be obtained from the subject to be treated, the desired cells (e.g., T cells) can be recovered/enriched from the subject's blood, and autologous transgenic cells can be prepared by introducing into the desired cells one or more expression vectors operably linked to nucleic acids encoding any of the precursor polypeptides, or the first polypeptide chain and/or second polypeptide chains described herein. Administering to the subject autologous transgenic T cells expressing a memory dimeric antigen receptor construct greatly reduces graft-versus-host disease in the subject.

In one embodiment, the host cell or population of host cells used to treat the subject are allogeneic and are derived from a different subject (e.g., a donor) from the subject to which they are to be administered, or are derived from multiple donors. In one embodiment, the donor (or the donors) will not receive treatment of host cells expressing memory dimeric antigen receptors (mDAR). Allogeneic cells can be obtained from blood (e.g., whole blood) from at least one donor in a similar manner employed for the autologous cells. In one embodiment, blood (e.g., whole blood) can be obtained from at least one donor, the desired cells can be recovered/enriched from the donor's (or donors') blood, and autologous transgenic cells can be prepared by introducing into the donor's (or donors') desired cells one or more expression vectors operably linked to nucleic acids encoding any of the precursor polypeptides, or the first polypeptide chain and/or second polypeptide chains described herein. Administering to the subject allogeneic transgenic T cells expressing a memory dimeric antigen receptor construct can lead to graft-versus-host disease in the subject.

In one embodiment, the desired cells recovered from the subject's blood, or from the donors' blood, include T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

In one embodiment, the host cell or population of host cells harbor one or more expression vectors that can direct transient introduction of the transgene into the host cells or stable insertion of the transgene into the host cells' genome, where the transgene comprises nucleic acids encoding any of the memory dimeric antigen receptors described herein. The expression vector(s) can direct transcription and/or translation of the transgene in the host cell. The expression vectors can include one or more regulatory sequences, such as inducible and/or constitutive promoters and enhancers. The expression vectors can include ribosomal binding sites and/or polyadenylation sites. In one embodiment, the expression vector, which is operably linked to the nucleic acid encoding any of the precursor polypeptides, or the first polypeptide chain and/or second polypeptide chains described herein, and the expression vector can direct production of the memory dimeric antigen receptor (mDAR) construct which can be embedded in the host cell's membrane and be displayed on the surface of the transgenic host cell or the memory dimeric antigen receptor can be secreted into the cell culture medium. In one embodiment, transgenic host cells can harbor one or more expression vectors operably linked to the nucleic acid transgene that encodes any of the precursor polypeptides, or the first polypeptide chain and/or second polypeptide chains described herein, and the host cells can be cultured in an appropriate culture medium to transiently or stably express a memory dimeric antigen receptor construct.

The expression vector can include nucleic acid backbone sequences derived from a retrovirus, lentivirus or adenovirus. The expression vector can include the transgene encoding the mDAR polypeptide chains and additional sequences for homologous directed repair for use with a CRISPR (cluster regularly interspaced short palindromic repeats) system for insertion or replacement of the transgene into the host cell's genome. In one embodiment, the transgene used in a CRISPR system can be operably joined to a promoter for mediating constitutive or inducible transcription of the mDAR polypeptide chains. In one embodiment, CRISPR includes Cas9 or Cpf1 (Cas12a). In one embodiment, the transgene can be part of a transposon for use with a transposase system. Examples of transposase systems include commercially-available systems such as PIGGYBAC, SUPER PIGGYBAC and SLEEPING BEAUTY (including SB100X).

The present disclosure provides a host cell, or a population of host cells, which harbors an expression vector operably linked to a nucleic acid that encodes an mDAR precursor polypeptide (e.g., FIG. 3A) comprising a plurality of polypeptide regions, the plurality comprising, in order from the amino terminus to the carboxyl terminus: (i) a heavy chain leader sequence; (ii) an antibody heavy chain variable region; (iii) an antibody heavy chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40); (vii) a self-cleaving sequence; (viii) a light chain leader sequence; (ix) an antibody light chain variable region; and (x) an antibody light chain constant region, wherein the self-cleaving sequence permits cleaving of the precursor polypeptide and release of the first and second polypeptide chains. In one embodiment, the host cell, or population of host cells, expresses the precursor polypeptide.

The present disclosure provides a first host cell, or a first population of host cells, which harbors a first expression vector operably linked to a nucleic acid that encodes an mDAR first polypeptide chain (e.g., FIG. 1A) comprising a plurality of polypeptide regions, the plurality comprising, in order from the amino terminus to the carboxyl terminus: (i) a heavy chain leader sequence; (ii) an antibody heavy chain variable region, (iii) an antibody heavy chain constant region, (iv) an optional hinge region, (v) a transmembrane region, (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40). In one embodiment, the first vector is operably linked to a first nucleic acid encoding an mDAR first polypeptide chain that lacks a heavy chain leader region. In one embodiment, the first host cell, or the first population of host cells, expresses the first polypeptide chains.

The present disclosure provides a second host cell, or a second population of host cells, which harbors a second expression vector operably linked to a nucleic acid that encodes an mDAR second polypeptide chain (e.g., FIG. 1A) comprising a plurality of polypeptide regions, the plurality comprising, in order from the amino terminus to the carboxyl terminus: (i) a light chain leader sequence; (ii) an antibody light chain variable region; and (iii) an antibody light chain constant region. In one embodiment, the second vector is operably linked to a second nucleic acid encoding an mDAR second polypeptide chain that lacks a light chain leader region. In one embodiment, the second host cell, or the second population of host cells, expresses the second polypeptide chains.

In one embodiment, the host cell, or the population of host cells, harbors a first expression vector operably linked to a nucleic acid that encodes the first polypeptide chain (FIG. 1A) and harbors a second expression vector operably linked to a nucleic acid that encodes the second polypeptide chain (e.g., FIG. 1A), wherein (a) the first polypeptide chain comprises (i) a heavy chain leader sequence; (ii) an antibody heavy chain variable region; (iii) an antibody heavy chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40); and wherein (b) the second polypeptide chain comprises (i) a light chain leader sequence; (ii) an antibody light chain variable region; and (iii) an antibody light chain constant region. In one embodiment, the first nucleic acid encodes an mDAR first polypeptide chain that lacks a heavy chain leader region and/or lacks a hinge region. In one embodiment, the second nucleic acid encodes an mDAR second polypeptide chain that lacks a light chain leader region. In one embodiment, the host cell, or the population of host cells, expresses the first and second polypeptide chains.

In one embodiment, the host cell, or the population of host cells, harbors an expression vector operably linked to a first nucleic acid that encodes the mDAR first polypeptide chain (e.g., FIG. 1A) and the expression vector is linked to a second nucleic acid that encodes the mDAR second polypeptide chain (e.g., FIG. 1A), wherein: (a) the first nucleic acid encodes the first polypeptide chain comprising a plurality of polypeptide regions, the plurality comprising, in order from the amino terminus to the carboxyl terminus: (i) an antibody heavy chain variable region (VH); (ii) an antibody heavy chain constant region (CH); (iii) an optional hinge region; (iv) a transmembrane region (TM); and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) the second nucleic acid encodes the second polypeptide chain comprising a plurality of polypeptide regions, the plurality comprising, in order from the amino terminus to the carboxyl terminus: (i) an antibody light chain variable region (VL) (e.g., kappa or lambda); and (ii) an antibody light chain constant region (CL). In one embodiment, the first nucleic acid encodes an mDAR first polypeptide chain that lacks a heavy chain leader region and/or lacks a hinge region. In one embodiment, the second nucleic acid encodes an mDAR second polypeptide chain that lacks a light chain leader region. In one embodiment, the host cell, or the population of host cells, expresses the mDAR first and second polypeptide chains.

The present disclosure provides a host cell, or a population of host cells, which harbors an expression vector operably linked to a nucleic acid that encodes an mDAR precursor polypeptide (FIG. 3B) comprising a plurality of polypeptide regions, the plurality comprising, in order from the amino terminus to the carboxyl terminus: (i) a light chain leader sequence; (ii) an antibody light chain variable region; (iii) an antibody light chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40); (vii) a self-cleaving sequence; (viii) a heavy chain leader sequence; (ix) an antibody heavy chain variable region, and (x) an antibody heavy chain constant region, wherein the self-cleaving sequence permits cleaving of the precursor polypeptide and release of the first and second polypeptide chains. In one embodiment, the host cell, or population of host cells, expresses the precursor polypeptide.

The present disclosure provides a first host cell, or a first population of host cells, which harbors a first expression vector operably linked to a nucleic acid that encodes an mDAR first polypeptide chain (FIG. 1B) comprising (i) a light chain leader sequence; (ii) an antibody light chain variable region; (iii) an antibody light chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40). In one embodiment, the first vector is operably linked to a first nucleic acid encoding an mDAR first polypeptide chain that lacks a light chain leader region. In one embodiment, the first host cell, or the first population of host cells, expresses the first polypeptide chains.

The present disclosure provides a second host cell, or a second population of host cells, which harbors a second expression vector operably linked to a nucleic acid that encodes an mDAR second polypeptide chain (FIG. 1B) comprising (i) a heavy chain leader sequence; (ii) an antibody heavy chain variable region; and (iii) an antibody heavy chain constant region. In one embodiment, the second polypeptide chain is exemplified in FIG. 1B. In one embodiment, the second host cell, or the second population of host cells, expresses the second polypeptide chains.

In one embodiment, the host cell, or the population of host cells, harbors a first expression vector operably linked to a nucleic acid that encodes the first polypeptide chain (FIG. 1B) and harbors a second expression vector operably linked to a nucleic acid that encodes the second polypeptide chain (FIG. 1B), wherein (a) the first polypeptide chain comprises (i) a light chain leader sequence; (ii) an antibody light chain variable region; (iii) an antibody light chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a STAT3 binding motif, and (3) an optional intracellular costimulatory region (e.g., CD28 or 4-1BB or OX40); and wherein (b) a second polypeptide chain comprises (i) a heavy chain leader sequence; (ii) an antibody heavy chain variable region; and (iii) an antibody heavy chain constant region. In one embodiment, the first nucleic acid encodes an mDAR first polypeptide chain that lacks a heavy chain leader region and/or lacks a hinge region. In one embodiment, the second nucleic acid encodes an mDAR second polypeptide chain that lacks a light chain leader region. In one embodiment, the host cell, or the population of host cells, expresses the first and second polypeptide chains.

In one embodiment, the host cell, or the population of host cells, harbors an expression vector operably linked to a first nucleic acid that encodes the mDAR first polypeptide chain (e.g., FIG. 1B) and the expression vector is linked to a second nucleic acid that encodes the mDAR second polypeptide chain e.g., (e.g., FIG. 1A), wherein: (a) the first nucleic acid encodes the first polypeptide chain comprising a plurality of polypeptide regions, the plurality comprising, in order from the amino terminus to the carboxyl terminus: (i) an antibody light chain variable region (VL); (ii) an antibody light chain constant region (CL); (iii) an optional hinge region; (iv) a transmembrane region (TM); and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζ ITAM domain with two ITAM motifs and a heterologous STAT3 binding motif, and (3) an optional intracellular costimulatory region (e.g., CD28 or 4-1BB or OX40); and (b) the second nucleic acid encodes the second polypeptide chain comprising a plurality of polypeptide regions, the plurality comprising, in order from the amino terminus to the carboxyl terminus: (i) an antibody heavy chain variable region (VH); and (ii) an antibody heavy chain constant region (CH). In one embodiment, the first nucleic acid encodes an mDAR first polypeptide chain that lacks a light chain leader region and/or lacks a hinge region. In one embodiment, the second nucleic acid encodes an mDAR second polypeptide chain that lacks a heavy chain leader region. In one embodiment, the host cell, or the population of host cells, expresses the mDAR first and second polypeptide chains.

### Methods for Treating

The present disclosure further provides methods for conducting adoptive cell therapy by administering to a subject transgenic host cells (e.g., a population of transgenic host cells) that have been engineered to express the memory dimeric antigen receptor constructs.

The present disclosure further provides a method of treating a subject having a disease, disorder or condition associated with detrimental expression (e.g., elevated expression or over-expression) of a tumor antigen (e.g., CD38 antigen). Such a method comprise administering to the subject an effective amount of one or more populations of host cells as discussed above and/or which harbor at least one expression vector operably linked to one or more nucleic acids encoding any of the first polypeptide chains or second polypeptide chains, or any of the first and second polypeptide chains, or any of the precursor polypeptide chains described herein. In one embodiment, the host cell or the population of host cells express any of the first and second polypeptide chains, or any of the precursor polypeptide chains described herein.

In any of the foregoing embodiments, the subject may have a disease, disorder or condition associated with detrimental expression of a tumor antigen, wherein the disorder is cancer, including, but not limited to hematologic breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma.

In one embodiment, the cancer is a hematologic cancer selected from the group consisting of non-Hodgkin's lymphoma (NHL), Burkitt's lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CIVIL) and multiple myeloma (MM). In one embodiment, the cancer is a CD38-positive cancer, such as a CD38-positive hematologic cancer, e.g., a CD38-positive B-cell hematologic cancer (e.g., lymphoma (such as NHL), leukemia (such as CLL), or myeloma.

CD38 protein-Human (UniProt P28907)
SEQ ID NO: 1
MANCEFSPVSGDKPCCRLSRRAQLCLGVSILVLILVVVLAVVVPRWRQQWSGPGTTKRFP
ETVLARCVKYTEIHPEMRHVDCQSVWDAFKGAFISKHPCNITEEDYQPLMKLGTQTVPCN
KILLWSRIKDLAHQFTQVQRDMFTLEDTLLGYLADDLTWCGEFNTSKINYQSCPDWRKDC
SNNPVSVFWKTVSRRFAEAACDVVHVMLNGSRSKIFDKNSTFGSVEVHNLQPEKVQTLEA
WVIHGGREDSRDLCQDPTIKELESIISKRNIQFSCKNIYRPDKFLQCVKNPEDSSCTSEI
CD38 protein-Cynomolgus monkey (UniProt 05VANO)
SEQ ID NO: 2
MANCEFSPVSGDKPCCRLSRRAQVCLGVCLLVLLILVVVVAVVLPRWRQQWSGSGTTSRF
PETVLARCVKYTEVHPEMRHVDCQSVWDAFKGAFISKYPCNITEEDYQPLVKLGTQTVPC
NKTLLWSRIKDLAHQFTQVQRDMFTLEDMLLGYLADDLTWCGEFNTFEINYQSCPDWRKD
CSNNPVSVFWKTVSRRFAETACGVVHVMLNGSRSKIFDKNSTFGSVEVHNLQPEKVQALE
AWVIHGGREDSRDLCQDPTIKELESIISKRNIRFFCKNIYRPDKFLQCVKNPEDSSCLSG
CD38 protein-Mouse
SEQ ID NO: 3
MANYEFSQVSGDRPGCRLSRKAQIGLGVGLLVLIALVVGIVVILLRPRSLLVWTGEPTTK
HFSDIFLGRCLIYTQILRPEMRDQNCQEILSTFKGAFVSKNPCNITREDYAPLVKLVTQT
IPCNKTLFWSKSKHLAHQYTWIQGKMFTLEDTLLGYIADDLRWCGDPSTSDMNYVSCPHW
SENCPNNPITVFWKVISQKFAEDACGVVQVMLNGSLREPFYKNSTFGSVEVFSLDPNKVH
KLQAWVMHDIEGASSNACSSSSLNELKMIVQKRNMIFACVDNYRPARFLQCVKNPEHPSC
RLNT
CD38 protein-Rat
SEQ ID NO: 4
MANYEFSQVSEDRPGCRLTRKAQIGLGVGLLLLVALVVVVVIVLWPRSPLVWKGKPTTKH
FADIILGRCLIYTQILRPEMRDQDCKKILSTFKRGFISKNPCNITNEDYAPLVKLVTQTI
PCNKTLFWSKSKHLAHQYTWIQGKMFTLEDTLLGYIADDLRWCGDPSTSDMNYDSCPHWS
ENCPNNPVAVFWNVISQKFAEDACGVVQVMLNGSLSEPFYRNSTFGSVEVFNLDPNKVHK
LQAWVMHDIKGTSSNACSSPSINELKSIVNKRNMIFACQDNYRPVRFLQCVKNPEHPSCR
LNV
Anti-CD38 heavy chain constant (IgG1 CPPC)
SEQ ID NO: 5
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHT [symbol] PAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Anti-CD38 heavy chain constant (IgG1 SPPC)
SEQ ID NO: 6
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHT [symbol] PAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Anti-CD38 light chain constant (lambda)
SEQ ID NO: 7
GQPKAAPSVTLFPPSSEELQANKATLVCLISDFYPGAVTVAWKADSSPVKAGVETTTPSKQSNNKYAASS
YLSLTPEQWKSHRSYSCQVTHEGSTVEKTVAPTECS
Anti-CD38 light chain constant (kappa)
SEQ ID NO: 8
RTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Anti-CD38 A2 heavy chain variable region
SEQ ID NO: 9
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREDWGGEFTDWGRGTLVTVSS
Anti-CD38 A2 light chain variable region
SEQ ID NO: 10
QAGLTQPPSASGTSGQRVTISCSGSSSNIGINFVYWYQHLPGTAPKLLIYKNNQRPSGVPDRFSGSKSGN
SASLAISGLRSEDEADYYCAAWDDSLSGYVFGSGTKVTVL
Anti-CD38 A2-SV heavy chain variable region
SEQ ID NO: 11
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREDWGGEFTDWGRGTLVTVSS
Anti-CD38 A2-SV light chain variable region
SEQ ID NO: 12
QSVLTQPPSASGTSGQRVTISCSGSSSNIGINFVYWYQHLPGTAPKLLIYKNNQRPSGVPDRFSGSKSGN
SASLAISGLRSEDEADYYCAAWDDSLSGYVFGSGTKVTVL
Anti-CD38 3H10 heavy chain variable region
SEQ ID NO: 13
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREGWSGEFTDWGQGTLVTVSS
Anti-CD38 3H10 light chain variable region
SEQ ID NO: 14
QAGLTQPPSASGTSGQRVTISCSGSSSNIGINFVYWYQHLPGTAPKLLIYKNNQRPSGVPDRFSGSKSGN
SASLAISGLRSEDEADYYCAAWDDSLSGYVFGSGTKVTVL
Anti-CD38 3H10m1 heavy chain variable region
SEQ ID NO: 15
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREGWSGEFTDWGQGTLVTVSS
Anti-CD38 3H10m1 light chain variable region
SEQ ID NO: 16
QAGLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3H10m1g heavy chain variable region
SEQ ID NO: 17
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREGWSGEFTDWGQGTLVTVSS
Anti-CD38 3H10m1g light chain variable region
SEQ ID NO: 18
QSVLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3G8m1 heavy chain variable region
SEQ ID NO: 19
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREAWGGEFTNWGQGTLVTVSS
Anti-CD38 3G8m1 light chain variable region
SEQ ID NO: 20
QAGLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3E3m1 heavy chain variable region
SEQ ID NO: 21
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREAWGGEFTDWGQGTLVTVSS
Anti-CD38 3E3m1 light chain variable region
SEQ ID NO: 22
QAGLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3G3 heavy chain variable region
SEQ ID NO: 23
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREAWSGEFTDWGQGTLVTVSS
Anti-CD38 3G3 light chain variable region
SEQ ID NO: 24
QAGLTQPPSASGTSGQRVTISCSGSSSNIGINFVYWYQHLPGTAPKLLIYKNNQRPSGVPDRFSGSKSGN
SASLAISGLRSEDEADYYCAAWDDSLSGYVFGSGTKVTVL
Anti-CD38 3E10 heavy chain variable region
SEQ ID NO: 25
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREDWGGEFTDWGRGTLVTVSS
Anti-CD38 3E10 light chain variable region
SEQ ID NO: 26
QAGLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3E10N heavy chain variable region
SEQ ID NO: 27
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREDWGGEFTDWGQGTLVTVSS
Anti-CD38 3E10N light chain variable region
SEQ ID NO: 28
QSVLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3E10NS heavy chain variable region
SEQ ID NO: 29
QVQLVESGGGLVKPGGSLRLSCAASGFTFSDDYMSWIRQAPGKGLEWVASVSSGRPTTYYADSVRGRFTI
SRDNAKNSLYLQMNSLRAEDTAVYYCAREDWGGEFTDWGQGTLVTVSS
Anti-CD38 3E10NS light chain variable region
SEQ ID NO: 30
QSVLTQPPSASGTSGQRVTISCSGSSSNIGFHFVYWYQHLPGTAPKLLIYKNNQRPSGVPDRFSGSKSGN
SASLAISGLRSEDEADYYCAAWDDSLSGYVFGSGTKVTVL
Anti-CD38 3E11 heavy chain variable region
SEQ ID NO: 31
QVQLVESGGGLVKPGGSLRLSCAASGFTFSDDYMSWIRQAPGKGLEWVASVSNGRPTTYYADSVRGRFTI
SRDNAKNSLYLQMNSLRAEDTAVYYCAREGWGGEFTDWGQGTLVTVSS
Anti-CD38 3E11 light chain variable region
SEQ ID NO: 32
QAGLTQPPSASGTSGQRVTISCSGSSSNIGINFVYWYQHLPGTAPKLLIYKNNQRPSGVPDRFSGSKSGN
SASLAISGLRSEDEADYYCAAWDDSLSGYVFGSGTKVTVL
CD8 hinge
SEQ ID NO: 33
AKPTTTPAPRPPTPAPTIASQPLSLRPEACRPAAGGAVHTRGLDFAPR
CD28 hinge
SEQ ID NO: 34
KIEVMYPPPYLDNEKSNGTIIHVKGKHLCPSPLFPGPSKP
CD8 + CD28 hinge (long hinge)
SEQ ID NO: 35
AKPTTTPAPRPPTPAPTIASQPLSLRPEACRPAAGGAVHTRGLDFAPRKIEVMYPPPYLDNEKSNGTIIH
VKGKHLCPSPLFPGPSKP
CD28 transmembrane
SEQ ID NO: 36
FWVLVVVGGVLACYSLLVTVAFIIFWV
CD8 transmembrane
SEQ ID NO: 37
IYIWAPLAGTCGVILLSLVITLY
4-1BB transmembrane
SEQ ID NO: 38
IISFFLALTSTALLFLLFFLTLRFSVV
CD3zeta transmembrane
SEQ ID NO: 39
LCYLLDGILFIYGVILTALFL
4-1BB costimulatory
SEQ ID NO: 40
KRGRKKLLYIFKQPFMRPVQTTQEEDGCSCRFPEEEEGGCEL
CD28 costimulatory
SEQ ID NO: 41
RSKRSRLLHSDYMNMTPRRPGPTRKHYQPYAPPRDFAAYRS
OX40 costimulatory
SEQ ID NO: 42
ALYLLRRDQRLPPDAHKPPGGGSFRTPIQEEQADAHSTLAKI
Human IL2Rbeta intracellular (with Box 1 and Box 2 motifs)
SEQ ID NO: 43
NCRNTGPWLKKVLKCNT [symbol] D [symbol] SKFFSQLSSEHGGDVQKWLSSPFPSSSFSPGGLAPEISPLEVLERDKVTQ
LLPLNTDAYLSLQELQGQDPTHLV
Box 1 from IL2Rbeta intracellular region
SEQ ID NO: 44
LKCNTPDPS
Box 2 from IL2Rbeta intracellular region
SEQ ID NO: 45
PLEVLERDKV
CD3zeta ITAM 1, 2, 3 (w/BRRl, 2, 3; no STAT binding motif YXXO)
SEQ ID NO: 46
RVKFSRSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDA [symbol] ALPPR
CD3zeta ITAM 1, 2, 3 (w/BRRl, 2, 3; w/STAT binding motif YXXO; BRR1, 2, 3)
SEQ ID NO: 47
RVKFSRSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDA [symbol] ALPPR
CD3zeta ITAM 1, 3 (w/BRR1 2, 3; no STAT binding motif YXXO)
SEQ ID NO: 48
RVKFSRSADAPAYQQGQNQLYNELNLGRREEYDVLDKRRGRDPEMGGKPRRKNPQEGLGMKGERRRGKGH
DGLYQGLSTATKDTYDA [symbol] ALPPR
CD3zeta ITAM 1, 3 (w/BRR1 2, 3; w/STAT binding motif YXXO)
SEQ ID NO: 49
RVKFSRSADAPAYQQGQNQLYNELNLGRREEYDVLDKRRGRDPEMGGKPRRKNPQEGLGMKGERRRGKGH
DGLYQGLSTATKDTYDA [symbol] ALPPR
CD3zeta ITAM 1, d3 (w/BRR3; no STAT binding motif YXXO)
SEQ ID NO: 50
RVKFSRSADAPAYQQGQNQLYNELNLGRREEYDVLGMKGERRRGKGHDGLYQGLSTATKDTYDA [symbol] AL
PPR
CD3zeta ITAM 1, d3 (w/BRR3; w/STAT binding motif YXXO)
SEQ ID NO: 51
RVKFSRSADAPAYQQGQNQLYNELNLGRREEYDVLGMKGERRRGKGHDGLYQGLSTATKDTYDA [symbol] AL
PPR
CD3zeta ITAM 1 (no BRR1, 2, 3)
SEQ ID NO: 52
RVKFSRSADAPAYQQGQNQLYNELNLGRREEYDVL
CD3zeta ITAM 1 (w/BRRI)
SEQ ID NO: 53
RVKFSRSADAPAYQQGQNQLYNELNLGRREEYDVLDKRRG
CD3zeta ITAM 1 (w/BRR1 2)
SEQ ID NO: 54
RVKFSRSADAPAYQQGQNQLYNELNLGRREEYDVLDKRRGRDPEMGGKPRRKNPQEGL
CD3zeta ITAM d3 (w/BRR3; no STAT binding motif YXXO)
SEQ ID NO: 55
GMKGERRRGKGHDGLYQGLSTATKDTYDA [symbol] ALPPR
CD3zeta ITAM d3 (w/BRR3; w/STAT binding motif YXXO)
SEQ ID NO: 56
GMKGERRRGKGHDGLYQGLSTATKDTYDA [symbol] ALPPR
CD3zeta ITAM 3 (w/BRR2, 3; no STAT binding motif YXXO)
SEQ ID NO: 57
RGRDPEMGGKPRRKNPQEGLYNELQKDKMAEAYSEIGMKGERRRGKGHDGLYQGLSTATKDTYDA [symbol] A
LPPR
CD3zeta ITAM 3 (w/BRR2, 3; w/STAT binding motif; YXXO)
SEQ ID NO: 58
RGRDPEMGGKPRRKNPQEGLYNELQKDKMAEAYSEIGMKGERRRGKGHDGLYQGLSTATKDTYDA [symbol] A
LPPR
CD3zeta ITAM 3 (w/BRR3; no STAT binding motif YXXO)
SEQ ID NO: 59
AYSEIGMKGERRRGKGHDGLYQGLSTATKDTYD [symbol] ALPPR
CD3zeta ITAM 3 (w/BRR3; w/STAT binding motif YXXO)
SEQ ID NO: 60
AYSEIGMKGERRRGKGHDGLYQGLSTATKDTYDA [symbol] ALPPR
CD3zeta ITAM 1 motif
SEQ ID NO: 61
YNELNLGRREEYDVL
CD3zeta ITAM 2 motif
SEQ ID NO: 62
YNELQKDKMAE
CD3zeta ITAM 3 motif
SEQ ID NO: 63
YQGLSTATKDTYDAL
BRRI motif
SEQ ID NO: 64
DKRRG
BRR2 motif
SEQ ID NO: 65
KPRRK
BRR3 motif
SEQ ID NO: 66
KGERRRGK
STAT binding motif (general sequence)
SEQ ID NO: 67
YXXQ
STAT binding motif (general sequence)
SEQ ID NO: 68
YXXF
STAT binding motif (general sequence)
SEQ ID NO: 69
YXXL
STAT binding motif
SEQ ID NO: 70
YRHQ
STAT binding motif
SEQ ID NO: 71
YLRQ
STAT binding motif
SEQ ID NO: 72
YFKQ
STAT binding motif
SEQ ID NO: 73
YLRQ
STAT binding motif
SEQ ID NO: 74
YMPQ
STAT binding motif
SEQ ID NO: 75
YVLQ
STAT binding motif
SEQ ID NO: 76
YQPQ
STAT binding motif
SEQ ID NO: 77
YKPQ
STAT binding motif
SEQ ID NO: 78
YRPQ
STAT binding motif
SEQ ID NO: 79
YTHQ
STAT binding motif
SEQ ID NO: 80
YLKQ
STAT binding motif
SEQ ID NO: 81
YHNQ
STAT binding motif
SEQ ID NO: 82
YFFF
STAT binding motif
SEQ ID NO: 83
YCTF
STAT binding motif
SEQ ID NO: 84
YLSL
STAT binding motif
SEQ ID NO: 85
YLSLQ
STAT binding motif
SEQ ID NO: 86
YCTFP
STAT binding motif
SEQ ID NO: 87
YFFFH
Heavy chain leader sequence
SEQ ID NO: 88
MEWSWVFLFFLSVTTGVHS
Light chain leader sequence
SEQ ID NO: 89
MSVPTQVLGLLLLWLTDARC
Alternative leader sequence
SEQ ID NO: 90
MALPVTALLLPLALLLHAARP
T2A self-cleaving sequence
SEQ ID NO: 91
GSGEGRGSLLTCGDVEENPGP
P2A self-cleaving sequence
SEQ ID NO: 92
GSGATNFSLLKQAGDVEENPGP
E2A self-cleaving sequence
SEQ ID NO: 93
GSGQCTNYALLKLAGDVESNPGP
F2A self-cleaving sequence
SEQ ID NO: 94
GSGVKQTLNFDLLKLAGDVESNPGP
Precursor memory DAR (V1)
SEQ ID NO: 95
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLVRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKDTYDA [symbol] ALPPR [symbol]
MSVPTQVLGLLLLWLTDARCQSVLTQPPSASGTSGQRVTISCSGSSSNI
GINFVYWYQHLPGTAPKLLIYKNNQRPSGVPDRFSGSKSGNSASLAISGLRSEDEADYYCAAWDDSLSGY
VFGSGTKVTVLGQPKAAPSVTLFPPSSEELQANKATLVCLISDFYPGAVTVAWKADSSPVKAGVETTTPS
KQSNNKYAASSYLSLTPEQWKSHRSYSCQVTHEGSTVEKTVAPTECS
Memory DAR (V1) heavv chain (1st polypeptide)
SEQ ID NO: 96
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLVRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKDTYDA [symbol] ALPPR
Memory DAR (V1) light chain (2nd polypeptide)
SEQ ID NO: 97
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V2)
SEQ ID NO: 98
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLVRVKFSRSADAPAYQQGQNQLYNELNLGRREEYDVLDKRRGRD
PEMGGKPRRKNPQEGLGMKGERRRGKGHDGLYQGLSTATKDTYDA [symbol] ALPPR [symbol] [symbol]
MSVPTQVLGLLLLWLTDARCQSVLTQPPSASGTSGQRVTISCSGSSSNIGINFVYWYQHLPGTAP
KLLIYKNNQRPSGVPDRFSGSKSGNSASLAISGLRSEDEADYYCAAWDDSLSGYVFGSGTKVTVLGQPKA
APSVTLFPPSSEELQANKATLVCLISDFYPGAVTVAWKADSSPVKAGVETTTPSKQSNNKYAASSYLSLT
PEQWKSHRSYSCQVTHEGSTVEKTVAPTECS
Memory DAR (V2) heavy chain (1st polypeptide)
SEQ ID NO: 99
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLVRVKFSRSADAPAYQQGQNQLYNELNLGRREEYDVLDKRRGRD
PEMGGKPRRKNPQEGLGMKGERRRGKGHDGLYQGLSTATKDTYDA [symbol] ALPPR
Memory DAR (V2) light chain (2nd polypeptide)
SEQ ID NO: 100
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V3)
SEQ ID NO: 101
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLVRVKFSRSADAPAYQQGQNQLYNELNLGRREEYDVLGMKGERR
RGKGHDGLYQGLSTATKDTYDA [symbol] ALPPR [symbol] MSVPTQVLGLLLLWLTDA
RCQSVLTQPPSASGTSGQRVTISCSGSSSNIGINFVYWYQHLPGTAPKLLIYKNNQRPSGVPDRFSGSKS
GNSASLAISGLRSEDEADYYCAAWDDSLSGYVFGSGTKVTVLGQPKAAPSVTLFPPSSEELQANKATLVC
LISDFYPGAVTVAWKADSSPVKAGVETTTPSKQSNNKYAASSYLSLTPEQWKSHRSYSCQVTHEGSTVEK
TVAPTECS
Memory DAR (V3) heavy chain (1st polypeptide)
SEQ ID NO: 102
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLVRVKFSRSADAPAYQQGQNQLYNELNLGRREEYDVLGMKGERR
RGKGHDGLYQGLSTATKDTYDA [symbol] ALPPR
Memory DAR (V3) light chain (2nd polypeptide)
SEQ ID NO: 103
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor traditional DAR (V10)
SEQ ID NO: 104
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDA [symbol] ALPPR [symbol] MSV
PTQVLGLLLLWLTDARCQSVLTQPPSASGTSGQRVTISCSGSSSNIGINFVYWYQHLPGTAPKLLIYKNN
QRPSGVPDRFSGSKSGNSASLAISGLRSEDEADYYCAAWDDSLSGYVFGSGTKVTVLGQPKAAPSVTLFP
PSSEELQANKATLVCLISDFYPGAVTVAWKADSSPVKAGVETTTPSKQSNNKYAASSYLSLTPEQWKSHR
SYSCQVTHEGSTVEKTVAPTECS
Traditional anti-CD38 DAR (V10) heavy chain (1st polypeptide)
SEQ ID NO: 105
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDA [symbol] ALPPR
Traditional anti-CD38 DAR (V10) light chain (2nd polypeptide)
SEQ ID NO: 106
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor traditional DAR (V11)
SEQ ID NO: 107
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQEGLGMKGERRR
GKGHDGLYQGLSTATKDTYDA [symbol] ALPPR [symbol] MSVPTQVLGLLLLWLTDAR
CQSVLTQPPSASGTSGQRVTISCSGSSSNIGINFVYWYQHLPGTAPKLLIYKNNQRPSGVPDRFSGSKSG
NSASLAISGLRSEDEADYYCAAWDDSLSGYVFGSGTKVTVLGQPKAAPSVTLFPPSSEELQANKATLVCL
ISDFYPGAVTVAWKADSSPVKAGVETTTPSKQSNNKYAASSYLSLTPEQWKSHRSYSCQVTHEGSTVEKT
VAPTECS
Traditional DAR (V11) heavy chain (1st polypeptide)
SEQ ID NO: 108
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQEGLGMKGERRR
GKGHDGLYQGLSTATKDTYDA [symbol] ALPPR
Traditional DAR (V11) light chain (2nd polypeptide)
SEQ ID NO: 109
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor traditional DAR (V12)
SEQ ID NO: 110
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELRVKFSRSADAPAYQQGQNQLYNELNLGRREEYDVLGMKGERRRGKGHDGLYQGLSTATKDTYDA [symbol] [symbol]
ALPPR [symbol] MSVPTQVLGLLLLWLTDARCQSVLTQPPSASGTSGQRVTISC
SGSSSNIGINFVYWYQHLPGTAPKLLIYKNNQRPSGVPDRFSGSKSGNSASLAISGLRSEDEADYYCAAW
DDSLSGYVFGSGTKVTVLGQPKAAPSVTLFPPSSEELQANKATLVCLISDFYPGAVTVAWKADSSPVKAG
VETTTPSKQSNNKYAASSYLSLTPEQWKSHRSYSCQVTHEGSTVEKTVAPTECS
Traditional DAR (V12) heavy chain (1st polypeptide)
SEQ ID NO: 111
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVKRGRKKLLYIFKQPFMRPVQTTQEEDGCSCRFPEEEEG
GCELRVKFSRSADAPAYQQGQNQLYNELNLGRREEYDVLGMKGERRRGKGHDGLYQGLSTATKDTYDA [symbol] [symbol]
ALPPR
Traditional DAR (V12) light chain (2nd polypeptide)
SEQ ID NO: 112
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V4)
SEQ ID NO: 113
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVNCRNTGPWLKKVLKCNTPDPSKFFSQLSSEHGGDVQKW
LSSPFPSSSFSPGGLAPEISPLEVLERDKVTQLLPLNTDAYLSLQELQGQDPTHLVRVKFSRSADAPAYQ
QGQNQLYNELNLGRREEYDVLDKRRGRDPEMGGKPRRKNPQEGLYNELQKDKMAEAYSEIGMKGERRRGK
GHDGLYQGLSTATKDTYDA [symbol] ALPPR [symbol] MSVPTQVLGLLLLWLTDARCQ
SVLTQPPSASGTSGQRVTISCSGSSSNIGINFVYWYQHLPGTAPKLLIYKNNQRPSGVPDRFSGSKSGNS
ASLAISGLRSEDEADYYCAAWDDSLSGYVFGSGTKVTVLGQPKAAPSVTLFPPSSEELQANKATLVCLIS
DFYPGAVTVAWKADSSPVKAGVETTTPSKQSNNKYAASSYLSLTPEQWKSHRSYSCQVTHEGSTVEKTVA
PTECS
Memory DAR (V4) heavy chain (1st polypeptide)
SEQ ID NO: 114
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVNCRNTGPWLKKVLKCNTPDPSKFFSQLSSEHGGDVQKW
LSSPFPSSSFSPGGLAPEISPLEVLERDKVTQLLPLNTDAYLSLQELQGQDPTHLVRVKFSRSADAPAYQ
QGQNQLYNELNLGRREEYDVLDKRRGRDPEMGGKPRRKNPQEGLYNELQKDKMAEAYSEIGMKGERRRGK
GHDGLYQGLSTATKDTYDA [symbol] ALPPR
Memory DAR (V4) light chain (2nd polypeptide)
SEQ ID NO: 115
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V5)
SEQ ID NO: 116
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVNCRNTGPWLKKVLKCNTPDPSKFFSQLSSEHGGDVQKW
LSSPFPSSSFSPGGLAPEISPLEVLERDKVTQLLPLNTDAYLSLQELQGQDPTHLVRVKFSRSADAPAYQ
QGQNQLYNELNLGRREEYDVLDKRRGRDPEMGGKPRRKNPQEGLGMKGERRRGKGHDGLYQGLSTATKDT
YDA [symbol] ALPPRGSGEGRGSLLTCGDVEENPGPMSVPTQVLGLLLLWLTDARCQSVLTQPPSASGTSGQR
VTISCSGSSSNIGINFVYWYQHLPGTAPKLLIYKNNQRPSGVPDRFSGSKSGNSASLAISGLRSEDEADY
YCAAWDDSLSGYVFGSGTKVTVLGQPKAAPSVTLFPPSSEELQANKATLVCLISDFYPGAVTVAWKADSS
PVKAGVETTTPSKQSNNKYAASSYLSLTPEQWKSHRSYSCQVTHEGSTVEKTVAPTECS
Memory DAR (V5) heavy chain (1st polypeptide)
SEQ ID NO: 117
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVNCRNTGPWLKKVLKCNTPDPSKFFSQLSSEHGGDVQKW
LSSPFPSSSFSPGGLAPEISPLEVLERDKVTQLLPLNTDAYLSLQELQGQDPTHLVRVKFSRSADAPAYQ
QGQNQLYNELNLGRREEYDVLDKRRGRDPEMGGKPRRKNPQEGLGMKGERRRGKGHDGLYQGLSTATKDT
YDA [symbol] ALPPR
Memory DAR (V5) light chain (2nd polypeptide)
SEQ ID NO: 118
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V6)
SEQ ID NO: 119
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLEP
GPSKPFWVLVVVGGVLACYSLLVTVAFIIFWVNCRNTGPWLKKVLKCNTPDPSKFFSQLSSEHGGDVQKW
LSSPFPSSSFSPGGLAPEISPLEVLERDKVTQLLPLNTDAYLSLQELQGQDPTHLVRVKFSRSADAPAYQ
QGQNQLYNELNLGRREEYDVLGMKGERRRGKGHDGLYQGLSTATKDTYDA [symbol] ALPPR [symbol] [symbol]
MSVPTQVLGLLLLWLTDARCQSVLTQPPSASGTSGQRVTISCSGSSSNIGINFVYWYQHL
PGTAPKLLIYKNNQRPSGVPDRFSGSKSGNSASLAISGLRSEDEADYYCAAWDDSLSGYVFGSGTKVTVL
GQPKAAPSVTLFPPSSEELQANKATLVCLISDFYPGAVTVAWKADSSPVKAGVETTTPSKQSNNKYAASS
YLSLTPEQWKSHRSYSCQVTHEGSTVEKTVAPTECS
Memory DAR (V6) heavy chain (1st polypeptide)
SEQ ID NO: 120
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVNCRNTGPWLKKVLKCNTPDPSKFFSQLSSEHGGDVQKW
LSSPFPSSSFSPGGLAPEISPLEVLERDKVTQLLPLNTDAYLSLQELQGQDPTHLVRVKFSRSADAPAYQ
QGQNQLYNELNLGRREEYDVLGMKGERRRGKGHDGLYQGLSTATKDTYDA [symbol] ALPPR
Memory DAR (V6) light chain (2nd polypeptide)
SEQ ID NO: 121
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V7)
SEQ ID NO: 122
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVRVKFSRSADAPAYQQGQNQLYNELNLGRREEYDVLDKR
RGNCRNTGPWLKKVLKCNTPDPSKFFSQLSSEHGGDVQKWLSSPFPSSSFSPGGLAPEISPLEVLERDKV
TQLLPLNTDAYLSLQELQGQDPTHLVRDPEMGGKPRRKNPQEGLGMKGERRRGKGHDGLYQGLSTATKDT
YDA [symbol] ALPPR [symbol] MSVPTQVLGLLLLWLTDARCQSVLTQPPSASGTSGQR
VTISCSGSSSNIGINFVYWYQHLPGTAPKLLIYKNNQRPSGVPDRFSGSKSGNSASLAISGLRSEDEADY
YCAAWDDSLSGYVFGSGTKVTVLGQPKAAPSVTLFPPSSEELQANKATLVCLISDFYPGAVTVAWKADSS
PVKAGVETTTPSKQSNNKYAASSYLSLTPEQWKSHRSYSCQVTHEGSTVEKTVAPTECS
Memory DAR (V7) heavy chain (1st polypeptide)
SEQ ID NO: 123
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVRVKFSRSADAPAYQQGQNQLYNELNLGRREEYDVLDKR
RGNCRNTGPWLKKVLKCNTPDPSKFFSQLSSEHGGDVQKWLSSPFPSSSFSPGGLAPEISPLEVLERDKV
TQLLPLNTDAYLSLQELQGQDPTHLVRDPEMGGKPRRKNPQEGLGMKGERRRGKGHDGLYQGLSTATKDT
YDA [symbol] ALPPR
Memory DAR (V7) light chain (2nd polypeptide)
SEQ ID NO: 124
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDEYPGAVTVAWKADSSPVKAGVETTTPSKQSNNKYAASSYLSLTPEQWK
SHRSYSCQVTHEGSTVEKTVAPTECS
Precursor memory DAR (V8)
SEQ ID NO: 125
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVRVKFSRSADAPAYQQGQNQLYNELNLGRREEYDVLDKR
RGRDPEMGGKPRRKNPQEGLGMKGERRRGKGHDGLYQGLSTATKDTYDA [symbol] ALPPRNCRNTGPWLKKV
LKCNTPDPSKFFSQLSSEHGGDVQKWLSSPFPSSSFSPGGLAPEISPLEVLERDKVTQLLPLNTDAYLSL
QELQGQDPTHLV [symbol] MSVPTQVLGLLLLWLTDARCQSVLTQPPSASGTSGQR
VTISCSGSSSNIGINFVYWYQHLPGTAPKLLIYKNNQRPSGVPDRFSGSKSGNSASLAISGLRSEDEADY
YCAAWDDSLSGYVFGSGTKVTVLGQPKAAPSVTLFPPSSEELQANKATLVCLISDFYPGAVTVAWKADSS
PVKAGVETTTPSKQSNNKYAASSYLSLTPEQWKSHRSYSCQVTHEGSTVEKTVAPTECS
Memory DAR (V8) heavy chain (1st polypeptide)
SEQ ID NO: 126
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVRVKFSRSADAPAYQQGQNQLYNELNLGRREEYDVLDKR
RGRDPEMGGKPRRKNPQEGLGMKGERRRGKGHDGLYQGLSTATKDTYDA [symbol] ALPPRNCRNTGPWLKKV
LKCNTPDPSKFFSQLSSEHGGDVQKWLSSPFPSSSFSPGGLAPEISPLEVLERDKVTQLLPLNTDAYLSL
QELQGQDPTHLV
Memory DAR (V8) light chain (2nd polypeptide)
SEQ ID NO: 127
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Precursor memory DAR (V9)
SEQ ID NO: 128
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVRVKFSRSADAPAYQQGQNQLYNELNLGRREEYDVLDKR
RGRDPEMGGKPRRKNPQEGLYNELQKDKMAEAYSEIGMKGERRRGKGHDGLYQGLSTATKDTYDA [symbol] A
LPPRNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLV [symbol] MSVPTQVLGLLLLWLTDARCQ
SVLTQPPSASGTSGQRVTISCSGSSSNIGINFVYWYQHLPGTAPKLLIYKNNQRPSGVPDRFSGSKSGNS
ASLAISGLRSEDEADYYCAAWDDSLSGYVFGSGTKVTVLGQPKAAPSVTLFPPSSEELQANKATLVCLIS
DFYPGAVTVAWKADSSPVKAGVETTTPSKQSNNKYAASSYLSLTPEQWKSHRSYSCQVTHEGSTVEKTVA
PTECS
Memory DAR (V9) heavy chain (1st polypeptide)
SEQ ID NO: 129
MEWSWVFLFFLSVTTGVHSQVQLVESGGGLVKPGGSLRLSCAASGFTFSDDYMSWIRQAPGKGLEWVASV
SNGRPTTYYADSVRGRFTISRDNAKNSLYLQMNSLRAEDTAVYYCAREDWGGEFTDWGRGTLVTVSSAST
KGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTKIEVMYPPPYLDNEKSNGTIIHVKGKHLCPSPLFP
GPSKPFWVLVVVGGVLACYSLLVTVAFIIFWVRVKFSRSADAPAYQQGQNQLYNELNLGRREEYDVLDKR
RGRDPEMGGKPRRKNPQEGLYNELQKDKMAEAYSEIGMKGERRRGKGHDGLYQGLSTATKDTYDA [symbol] A
LPPRNCRNTGPWLKKVLKCNTPDPSKFFSQLSSEHGGDVQKWLSSPFPSSSFSPGGLAPEISPLEVLERD
KVTQLLPLNTDAYLSLQELQGQDPTHLV
Memory DAR (V9) light chain (2nd polypeptide)
SEQ ID NO: 130
MSVPTQVLGLLLLWLTDARCQSVLTQPPSASGTSGQRVTISCSGSSSNIGINFVYWYQHLPGTAPKLLIY
KNNQRPSGVPDRFSGSKSGNSASLAISGLRSEDEADYYCAAWDDSLSGYVFGSGTKVTVLGQPKAAPSVT
LFPPSSEELQANKATLVCLISDFYPGAVTVAWKADSSPVKAGVETTTPSKQSNNKYAASSYLSLTPEQWK
SHRSYSCQVTHEGSTVEKTVAPTECS
Memory DAR (V1) intracellular region
SEQ ID NO: 131
KRGRKKLLYIFKQPFMRPVQTTQEEDGCSCRFPEEEEGGCELNCRNTGPWLKKVLKCNTPDPSKFFSQLS
SEHGGDVQKWLSSPFPSSSFSPGGLAPEISPLEVLERDKVTQLLPLNTDAYLSLQELQGQDPTHLVRVKF
SRSADAPAYQQGQNQLYNELNLGRREEYDVLDKRRGRDPEMGGKPRRKNPQEGLYNELQKDKMAEAYSEI
GMKGERRRGKGHDGLYQGLSTATKDTYDA [symbol] ALPPR
Memory DAR (V2) intracellular region
SEQ ID NO: 132
KRGRKKLLYIFKQPFMRPVQTTQEEDGCSCRFPEEEEGGCELNCRNTGPWLKKVLKCNTPDPSKFFSQLS
SEHGGDVQKWLSSPFPSSSFSPGGLAPEISPLEVLERDKVTQLLPLNTDAYLSLQELQGQDPTHLVRVKF
SRSADAPAYQQGQNQLYNELNLGRREEYDVLDKRRGRDPEMGGKPRRKNPQEGLGMKGERRRGKGHDGLY
QGLSTATKDTYDA [symbol] ALPPR
Memory DAR (V3) intracellular region
SEQ ID NO: 133
KRGRKKLLYIFKQPFMRPVQTTQEEDGCSCRFPEEEEGGCELNCRNTGPWLKKVLKCNTPDPSKFFSQLS
SEHGGDVQKWLSSPFPSSSFSPGGLAPEISPLEVLERDKVTQLLPLNTDAYLSLQELQGQDPTHLVRVKF
SRSADAPAYQQGQNQLYNELNLGRREEYDVLGMKGERRRGKGHDGLYQGLSTATKDTYDA [symbol] ALPPR
Memory DAR (V4) intracellular region
SEQ ID NO: 134
NCRNTGPWLKKVLKCNTPDPSKFFSQLSSEHGGDVQKWLSSPFPSSSFSPGGLAPEISPLEVLERDKVTQ
LLPLNTDAYLSLQELQGQDPTHLVRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYDA [symbol] ALPPR
Memory DAR (V5) intracellular region
SEQ ID NO: 135
NCRNTGPWLKKVLKCNTPDPSKFFSQLSSEHGGDVQKWLSSPFPSSSFSPGGLAPEISPLEVLERDKVTQ
LLPLNTDAYLSLQELQGQDPTHLVRVKFSRSADAPAYQQGQNQLYNELNLGRREEYDVLDKRRGRDPEMG
GKPRRKNPQEGLGMKGERRRGKGHDGLYQGLSTATKDTYDA [symbol] ALPPR
Memory DAR (V6) intracellular region
SEQ ID NO: 136
NCRNTGPWLKKVLKCNTPDPSKFFSQLSSEHGGDVQKWLSSPFPSSSFSPGGLAPEISPLEVLERDKVTQ
LLPLNTDAYLSLQELQGQDPTHLVRVKFSRSADAPAYQQGQNQLYNELNLGRREEYDVLGMKGERRRGKG
HDGLYQGLSTATKDTYDA [symbol] ALPPR
Memory DAR (V7) intracellular region
SEQ ID NO: 137
RVKFSRSADAPAYQQGQNQLYNELNLGRREEYDVLDKRRGNCRNTGPWLKKVLKCNTPDPSKFFSQLSSE
HGGDVQKWLSSPFPSSSFSPGGLAPEISPLEVLERDKVTQLLPLNTDAYLSLQELQGQDPTHLVRDPEMG
GKPRRKNPQEGLGMKGERRRGKGHDGLYQGLSTATKDTYDA [symbol] ALPPR
Memory DAR (V8) intracellular region
SEQ ID NO: 138
RVKFSRSADAPAYQQGQNQLYNELNLGRREEYDVLDKRRGRDPEMGGKPRRKNPQEGLGMKGERRRGKGH
DGLYQGLSTATKDTYDA [symbol] ALPPRNCRNTGPWLKKVLKCNTPDPSKFFSOLSSEHGGDVOKWLSSPFP
SSSFSPGGLAPEISPLEVLERDKVTQLLPLNTDAYLSLQELQGQDPTHLV
Memory DAR (V9) intracellular region
SEQ ID NO: 139
RVKFSRSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDA [symbol] ALPPRNCRNTGPWLKKVLKCNTPDPSKFFSQLS
SEHGGDVQKWLSSPFPSSSFSPGGLAPEISPLEVLERDKVTQLLPLNTDAYLSLQELQGQDPTHLV
Traditional DAR (V10) intracellular region
SEQ ID NO: 140
KRGRKKLLYIFKQPFMRPVQTTQEEDGCSCRFPEEEEGGCELRVKFSRSADAPAYQQGQNQLYNELNLGR
REEYDVLDKRRGRDPEMGGKPRRKNPQEGLYNELQKDKMAEAYSEIGMKGERRRGKGHDGLYQGLSTATK
DTYDA [symbol] ALPPR
Traditional DAR (V11) intracellular region
SEQ ID NO: 141
KRGRKKLLYIFKQPFMRPVQTTQEEDGCSCRFPEEEEGGCELRVKFSRSADAPAYQQGQNQLYNELNLGR
REEYDVLDKRRGRDPEMGGKPRRKNPQEGLGMKGERRRGKGHDGLYQGLSTATKDTYDA [symbol] ALPPR
Traditional DAR (V12) intracellular region
SEQ ID NO: 142
KRGRKKLLYIFKQPFMRPVQTTQEEDGCSCRFPEEEEGGCELRVKFSRSADAPAYQQGQNQLYNELNLGR
REEYDVLGMKGERRRGKGHDGLYQGLSTATKDTYDA [symbol] ALPPR
Human IL2Rbeta full-length (UniProtKB P14784)
SEQ ID NO: 143
MAAPALSWRLPLLILLLPLATSWASAAVNGTSQFTCFYNSRANISCVWSQDGALQDTSCQVHAWPDRRRW
NQTCELLPVSQASWACNLILGAPDSQKLTTVDIVTLRVLCREGVRWRVMAIQDFKPFENLRLMAPISLQV
VHVETHRCNISWEISQASHYFERHLEFEARTLSPGHTWEEAPLLTLKQKQEWICLETLTPDTQYEFQVRV
KPLQGEFTTWSPWSQPLAFRTKPAALGKDTIPWLGHLLVGLSGAFGFIILVYLLINCRNTGPWLKKVLKC
NTPDPSKFFSQLSSEHGGDVQKWLSSPFPSSSFSPGGLAPEISPLEVLERDKVTQLLLQQDKVPEPASLS
SNHSLTSCFTNQGYFFFHLPDALEIEACQVYFTYDPYSEEDPDEGVAGAPTGSSPQPLQPLSGEDDAYCT
FPSRDDLLLFSPSLLGGPSPPSTAPGGSGAGEERMPPSLQERVPRDWDPQPLGPPTPGVPDLVDFQPPPE
LVLREAGEEVPDAGPREGVSFPWSRPPGQGEFRALNARLPLNTDAYLSLQELQGQDPTHLV
ITAM from human CD3 gamma (UniProtKB P09693)
SEQ ID NO: 144
DKQTLLPNDQLYQPLKDREDDQYSHLQGN
ITAM from human CD3 delta (UniProtKB P04234)
SEQ ID NO: 145
DTQALLRNDQVYQPLRDRDDAQYSHLGGN
ITAM from human CD3 epsilon (UniProtKB P07766)
SEQ ID NO: 146
ERPPPVPNPDYEPIRKGQRDLYSGLNQR

### EXAMPLES

The following examples are meant to be illustrative and can be used to further understand embodiments of the present disclosure and should not be construed as limiting the scope of the present teachings in any way.

### Example 1: Isolation of Human PBMC Cells and Primary T Cells

Primary human T cells were isolated from healthy human donors either from buffy coats (San Diego blood bank), fresh blood or leukapheresis products (StemCell). Peripheral blood mononuclear cells (PBMC) were isolated by density gradient centrifugation.

Preparation of Donor 1 cells: T cells were isolated from PBMCs by magnetic negative selection using EASYSEP Human T Cell Isolation Kit (from STEMCELL Technologies, catalog No. 17951) or positive selection and activation by DYNABEADS Human T-Expander CD3/CD28 (from Thermo Fisher Scientific, catalog No. 11141D) according to manufacturer's instructions. Donor 1 cells were transfected with nucleic acids encoding a CD38 traditional DAR or CD38 mDAR to generate transgenic T cells that expressed the traditional DAR or mDAR constructs.

Preparation of Donor 2 cells: To deplete the monocytes, PBMC were plated in coated cell culture coated flasks for one to two hours. The nonadherent lymphocytes were washed away from the flask and activated with T cell TRANSACT (from Miltenyi, catalog No. 130-111-160) in a new flask according to manufacturer's instructions. Donor 2 cells were transfected with nucleic acids encoding a CD38 traditional DAR or CD38 mDAR to generate transgenic T cells that expressed the traditional DAR or mDAR constructs.

### Example 2: Primary T Cell Culture and Activation

Primary T cells were cultured in CTS OPTMIZER T Cell Expansion SFM supplemented with 5% CTS Immune Cell SR (Thermo Fisher Scientific) with 300 U/mL IL-2 (Proleukin) at a density of 10⁶ cells per mL. Isolated T cells were stimulated after isolation or after thawing frozen cells. Cells were activated with T Cell TRANSACT (Miltenyi) 3 uL/10⁶ cells per mL for two to three days. Following transfection, T cells were cultured in media with IL-2 at 300 U/mL.

### Example 3: Preparation of Traditional DAR and Memory DAR T Cells

Activated T cells (approximately 9×10⁶ cells) were transfected with nucleic acids encoding either a precursor CD38 traditional DAR construct (V10 (SEQ ID NO:104), V11 (SEQ ID NO:107) or V12 (SEQ ID NO:110)) (see FIG. 2D), or a precursor CD38 memory DAR construct (V1 (SEQ ID NO:95), V2 (SEQ ID NO:98) or V3 (SEQ ID NO:101)) (see FIG. 2A). Transfection of the cells with traditional or memory DAR constructs was performed using CRISPR methods as described in US 2020/0224160; WO 2020/176740, and WO 2020/185867, all of which are incorporated by reference herein in their entireties, that included electroporating a Cas9 RNP that included a guide RNA targeting exon 1 of the TRAC locus (gene encoding the T cell receptor alpha chain) and a donor DNA that included the DAR construct flanked by homology regions of the TRAC gene. Integration of the DAR construct of the donor fragment into the host cell genome occurred at the site of the disrupted TRAC gene, resulting in transfected cells (transgenic T cells) that expressed the introduced DAR construct in the absence of endogenous T cell receptor expression. As controls, activated T cells were transfected with the Cas9 RNP targeting the TRAC locus in the absence of a donor fragment encoding a DAR. This resulted in disruption (knockout) of the TRAC locus resulting in no TCR expression, but these control cells, referred to as TRAC KO or TCR KO cells did not express a DAR.

The introduced traditional and memory DAR constructs comprised antigen binding domains that bound the human CD38 protein. The naming designation of the traditional DAR and memory DAR constructs, with their respective intracellular regions is listed in Table 1 below (see also FIGS. 2A and 2D).

TABLE 1

| | | IL2Rβ | CD3ζ ITAMs +/− |
|---|---|---|---|
| | Costimulatory | region w/ | STAT signaling |
| Designation | Domain | Box 1 & 2 | region BRR motifs |
| Traditional | 4-1BB | No | ITAM 1, 2, 3; |
| DAR V10 | | | No STAT signaling region; |
| | | | BRR 1, 2 and 3. |
| Traditional | 4-1BB | No | ITAM 1 and 3; |
| DAR V11 | | | No STAT signaling region; |
| | | | BRR 1, 2 and 3. |
| Traditional | 4-1BB | No | ITAM 1 and d3; |
| DAR V12 | | | No STAT signaling region; |
| | | | BRR 3. |
| Memory DAR | 4-1BB | Yes | ITAM 1, 2, 3; |
| V1 | | | STAT signaling region; |
| | | | BRR 1, 2 and 3. |
| Memory DAR | 4-1BB | Yes | ITAM 1 and 3; |
| V2 | | | STAT signaling region; |
| | | | BRR 1, 2 and 3. |
| Memory DAR | 4-1BB | Yes | ITAM 1 and d3; |
| V3 | | | STAT signaling region; |
| | | | BRR 3. |

### Example 4: Tumor Cell Lines

Cells of multiple myeloma cell line RPMI 8226 were transduced using a lentivirus carrying luciferase and GFP genes. A single cell clone with luciferase and GFP expression was selected (RPMI8226-FLuc). K562/RPE cells were made similarly by transducing the K562 cells with lentivirus carrying RPE genes. Both cell lines were cultured in RPMI1640 medium (ATCC) supplemented with 10% fetal bovine serum (Sigma).

### Example 5: Characterization of Negative Control Cells

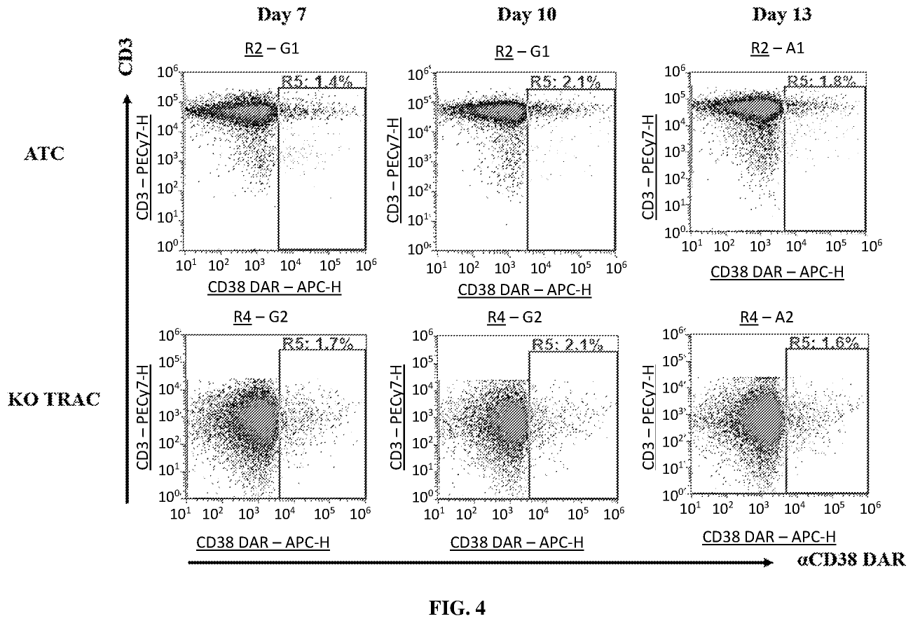

Flow cytometry was used to characterize negative control T cells which included non-transgenic activated T cells (ATC) and T cells having a knocked-out TRAC locus (TRAC KO) but no DAR construct. At day 7, 10, and 13 post-knock-out, the negative control cells were subjected to cell surface staining using PE/Cy7 anti-human CD3 (UCHT1, BioLegend 300420) to detect expression of CD3, PerCP/Cy5.5 anti-human TCRα/β (IP26 BioLegend 306723) to detect expression of TCRα/β, and APC-labeled CD38 Fc fusion protein (BPS Biosciences 71883) to detect expression of a CD38 DAR. Flow cytometry results for the negative control cells stained with PE/Cy7 anti-human CD3 and APC-labeled CD38 Fc is shown in FIG. 4. The non-engineered activated T cells (ATC) show high fluorescence with the anti-CD3 antibody but negligible staining with the CD38 Fc protein, while TCR knockout cells do not demonstrate staining with the TCR antibody, nor do they stain with the CD38 Fc protein.

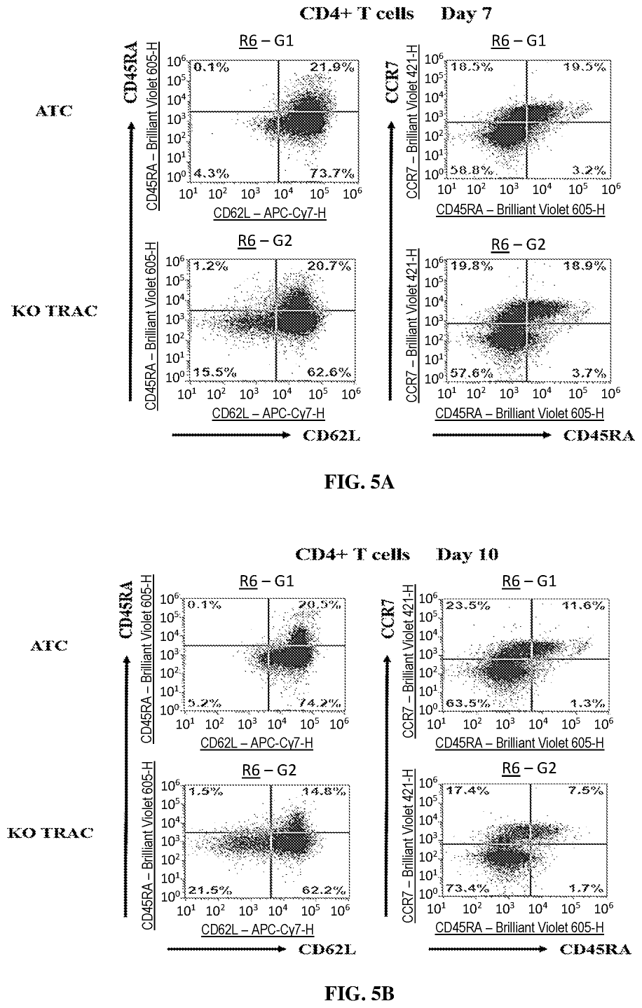

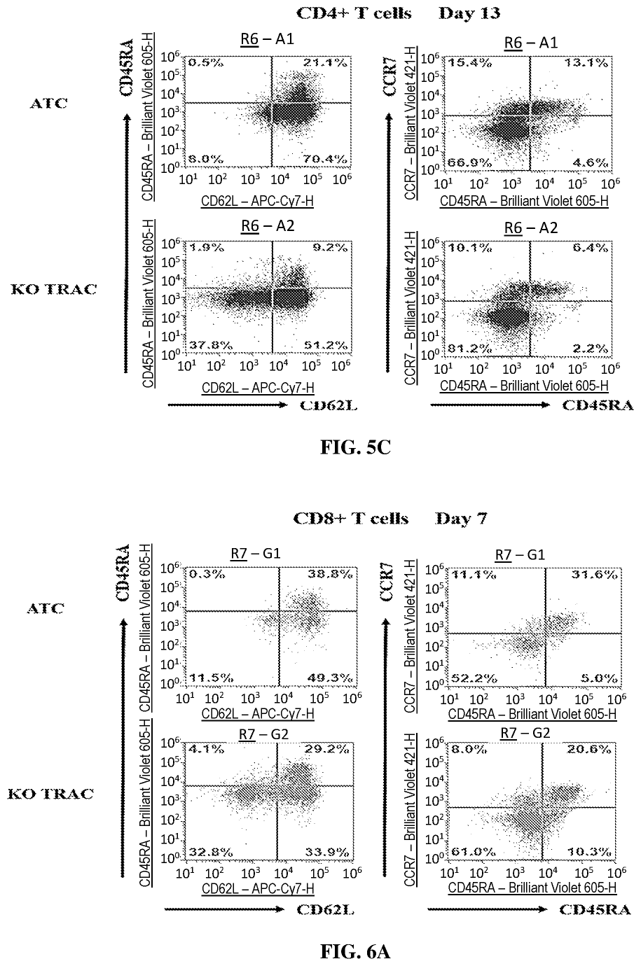

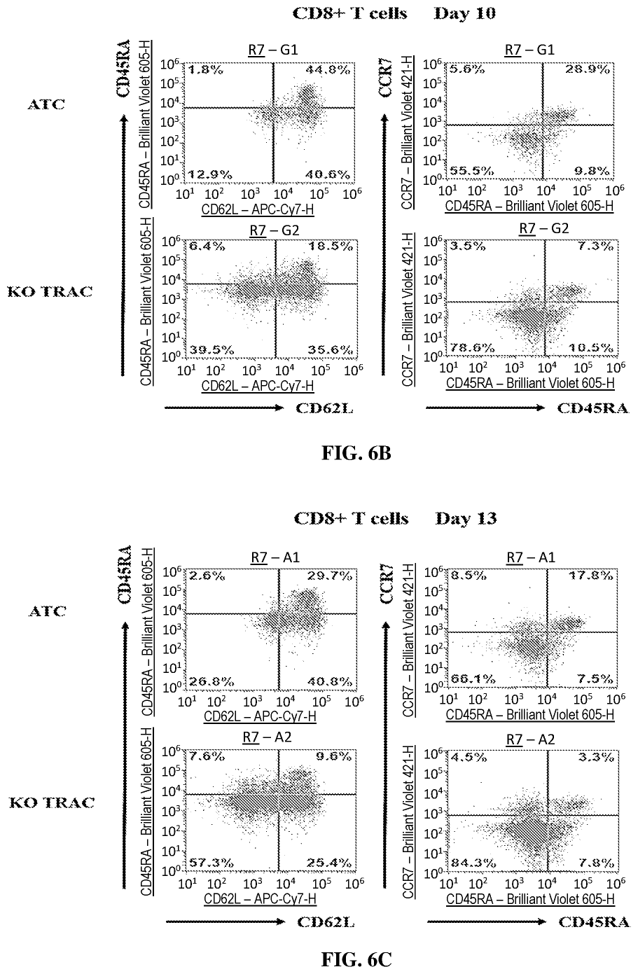

The negative control cells were also subjected to cell surface staining using APC/Cy7 anti-human CD62L (DREG-56 BioLegend 304813), BV605 anti-human CD45RA (HI100 BioLegend 304134), and BV421 anti-human CCR7 (G043H7 BioLegend 353207), to detect expression of memory T cell markers CD62L, CD45RA and/or CCR7 (see for example, Mousset et al. (2019) Cytometry Part A 95A:647-54; Cieri et al. (2013) Blood 1221:573-584). The CD4+ and CD8+ T cells were distinguished by cell surface staining with FITC anti-human CD4 (RPA-T4 BioLegend 300506), or PE anti-human CD8a (RPA-T8 BioLegend, 301051) to detect CD8+ cells. Data for negative control cells that were CD4+ is shown in FIGS. 5A-C and data for negative control cells that were CD8+ is shown in FIGS. 6A-C.

### Example 6: Characterization of Expression of CD38 Traditional DAR and CD38 mDAR in Transgenic T Cells

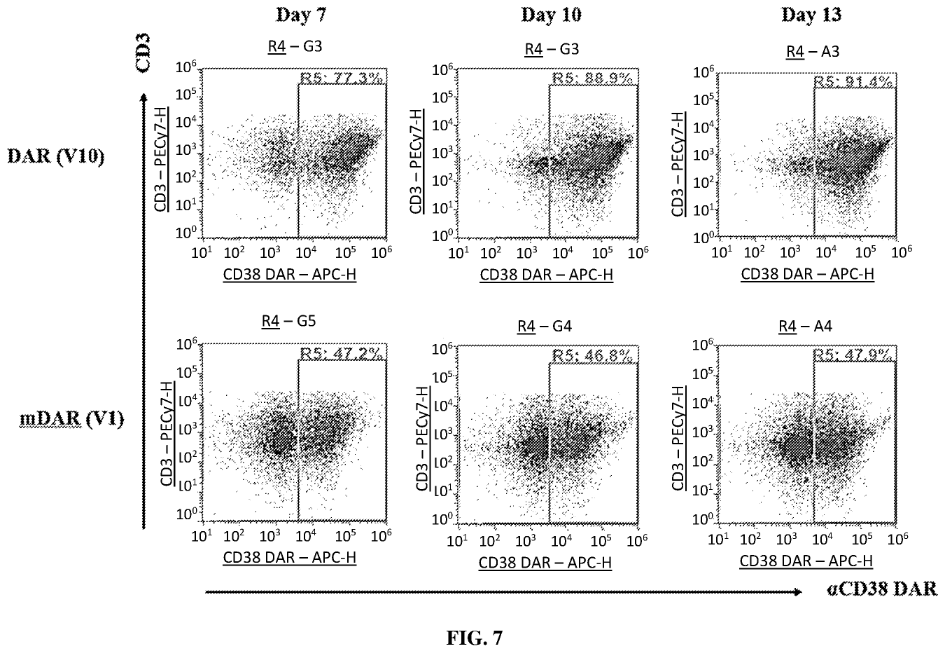

Flow cytometry was used to characterize expression levels of a CD38 traditional DAR (V10) and a CD38 memory DAR (V1) in transgenic T cells, at days 7, 10, and 13 post-transfection. The traditional DAR and mDAR T cells, generated as described in Example 3, carried a knocked-out TRAC locus. The traditional CD38 DAR T cells and CD38 mDAR T cells were subjected to cell surface staining using PE/Cy7 anti-human CD3 (UCHT1, BioLegend 300420) to detect expression of CD3 (to detect the presence of the T cell receptor), PerCP/Cy5.5 anti-human TCRa/b (IP26 BioLegend 306723) to detect expression of TCRa/d, and APC-labeled CD38 Fc fusion protein (BPS Biosciences 71883) to detect expression of a CD38 DAR. Data for expression of CD38 DAR and CD3 (T cell receptor) of the traditional DAR T cells and mDAR T cells is shown in FIG. 7, where nearly 50% of the cell culture transfected with a CD38 mDAR expressed the DAR in the absence of T cell receptor expression, and between 77% and 92% of the cell culture transfected with a CD38 mDAR expressed the DAR in the absence of T cell receptor expression.

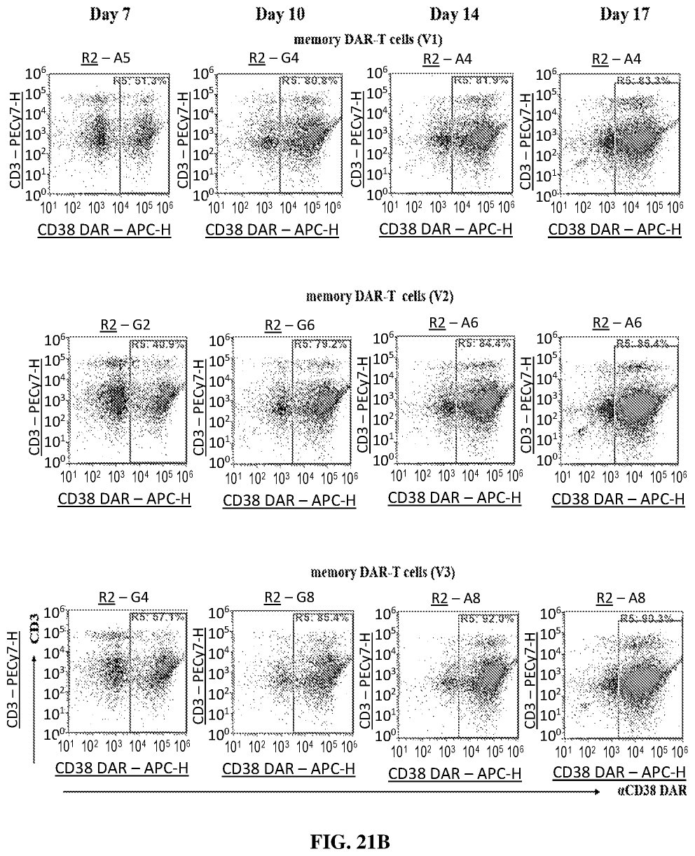

In a similar manner, flow cytometry and cell staining was used to characterize expression levels of CD38 traditional DAR (V10, V11 and V12) and anti-CD37 memory DAR (V1, V2 and V3) in transgenic T cells, at day 7, 10, 14 and 17 post-transfection. The traditional DAR T cells and memory DAR T cells carried a knocked-out TRAC locus and were generated using the method described in Example 3. Data for expression of CD38DAR and CD3 of the traditional DART cells is shown in FIG. 21A and of memory DART cells is shown in FIG. 21B.

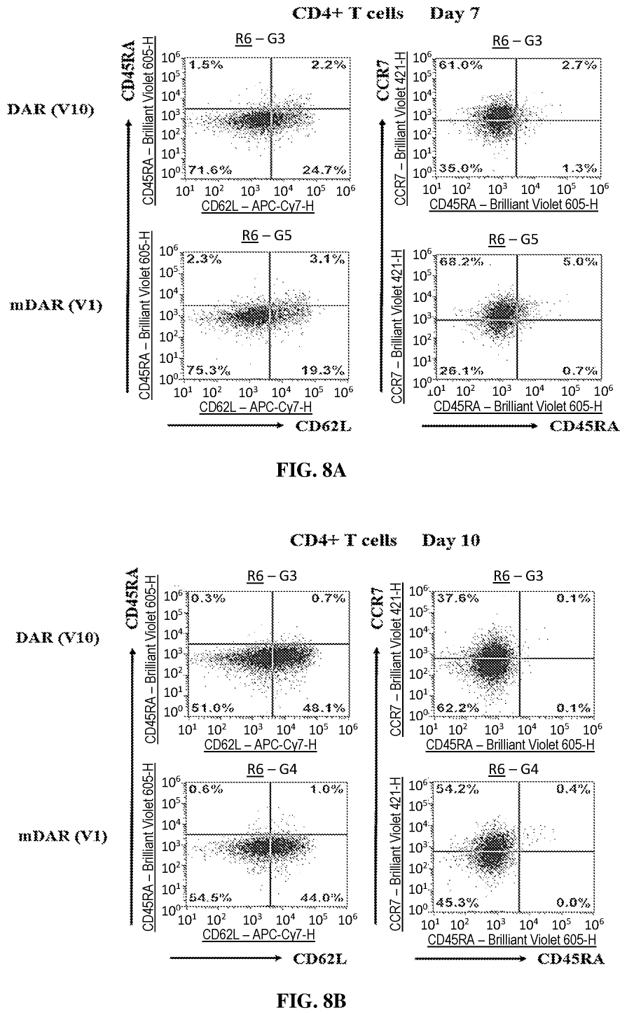

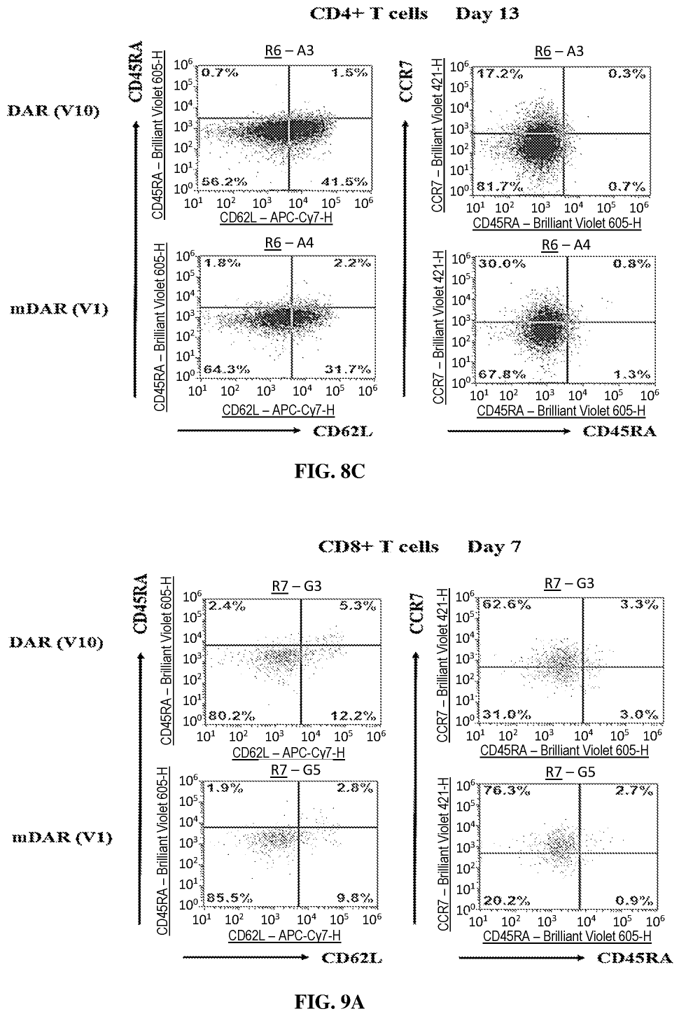

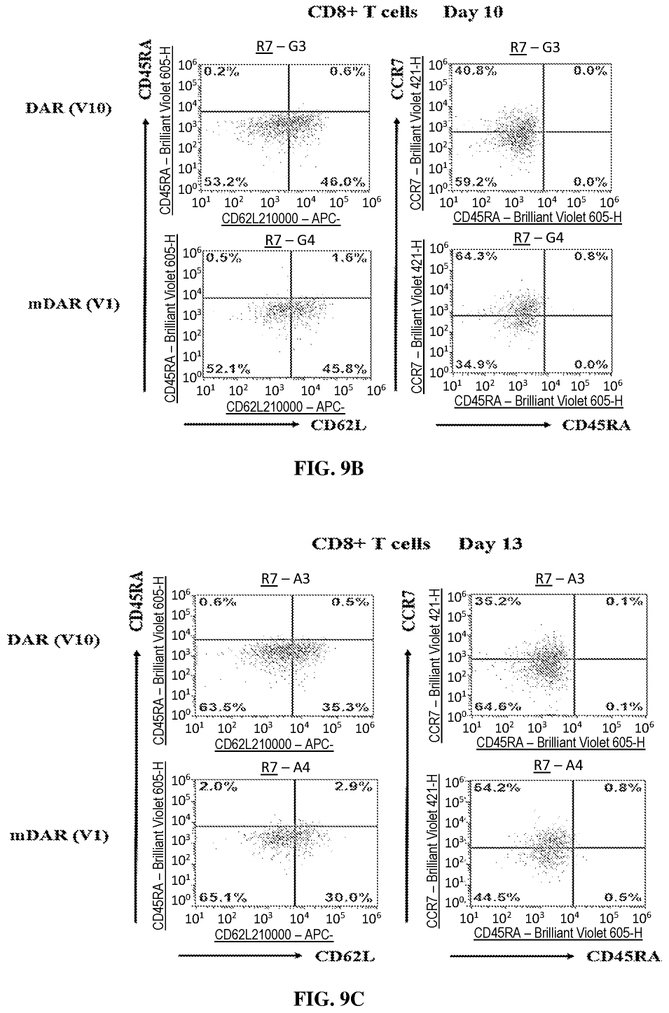

Flow cytometry was used to detect memory T cells in the population of CD38 traditional DAR T cells and CD38 mDAR T cells by cell surface staining using APC/Cy7 anti-human CD62L (DREG-56 BioLegend 304813), BV605 anti-human CD45RA (HI100 BioLegend 304134), and BV421 anti-human CCR7 (G043H7 BioLegend 353207), to detect expression of memory T cells expressing CD62L, CD45RA and/or CCR7. The CD4+ and CD8+ T cells were distinguished by cell surface staining with FITC anti-human CD4 (RPA-T4 BioLegend 300506), or PE anti-human CD8a (RPA-T8 BioLegend, 301051) to detect CD8+ cells. Data for CD4+ memory T cells is shown in FIGS. 8A-C. Data for CD8+ memory T cells is shown in FIGS. 9A-C.

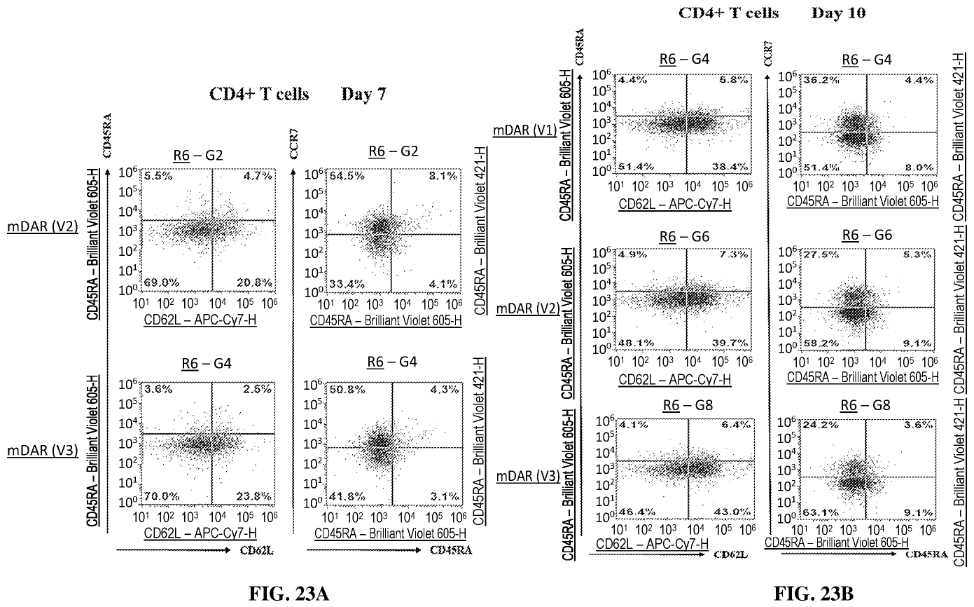

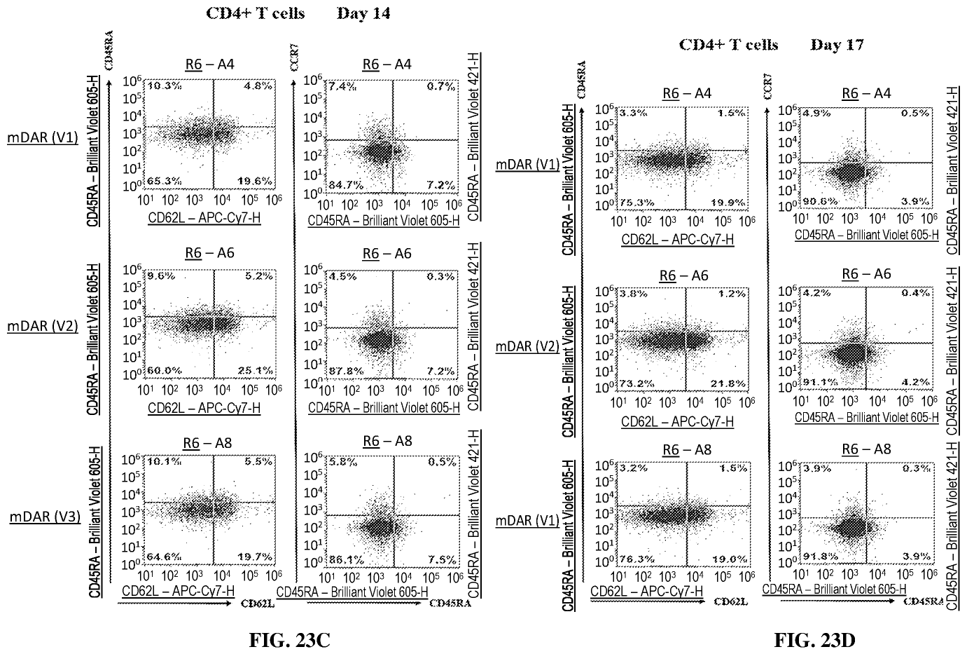

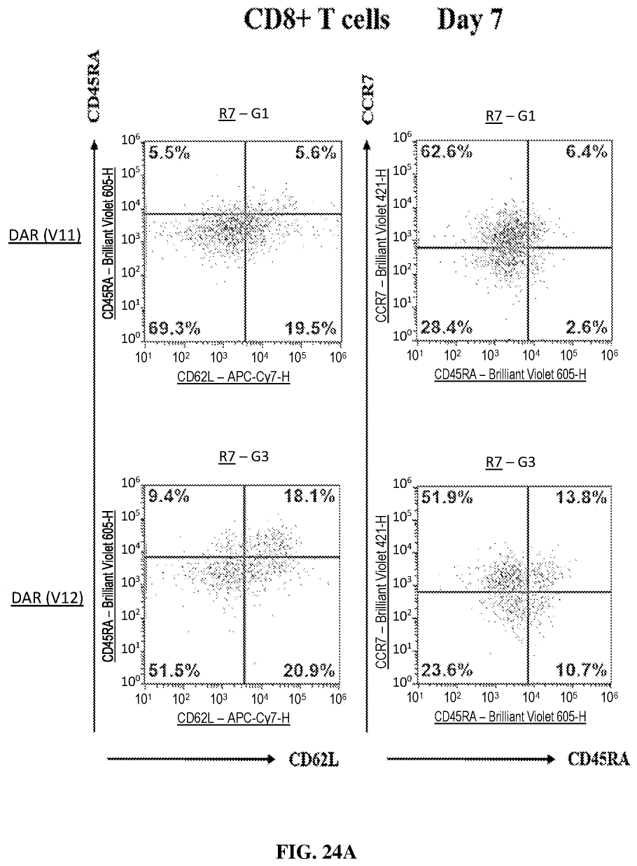

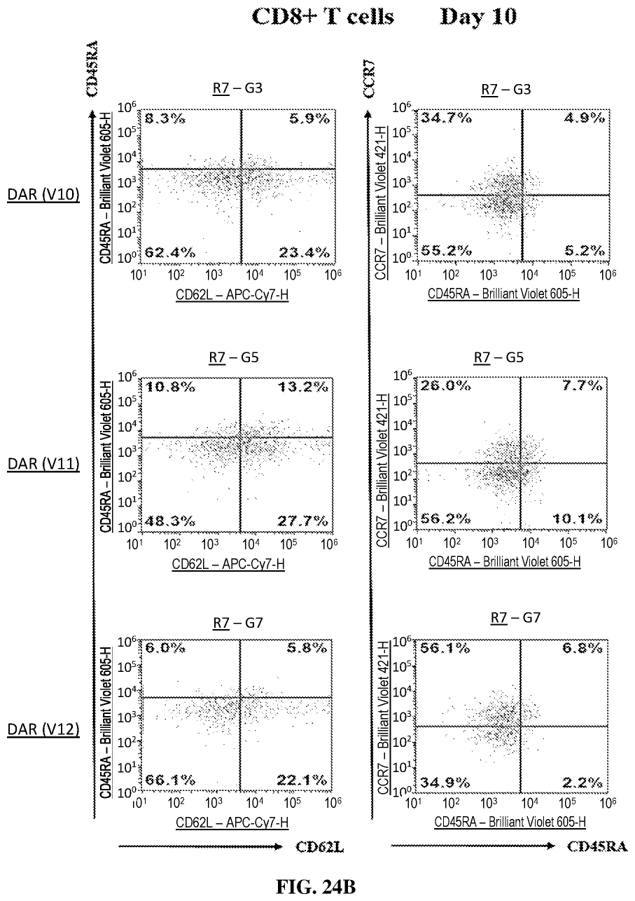

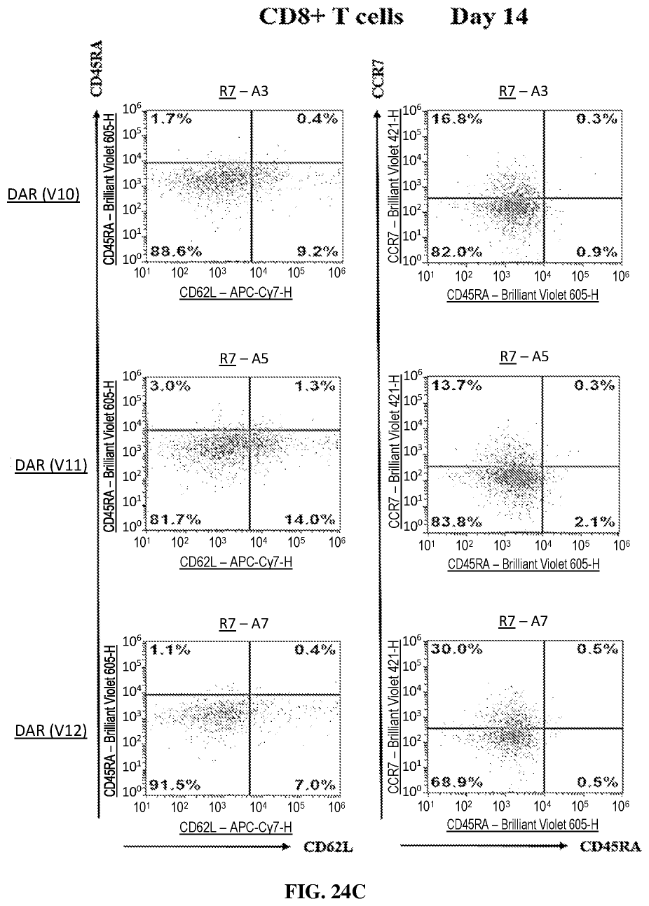

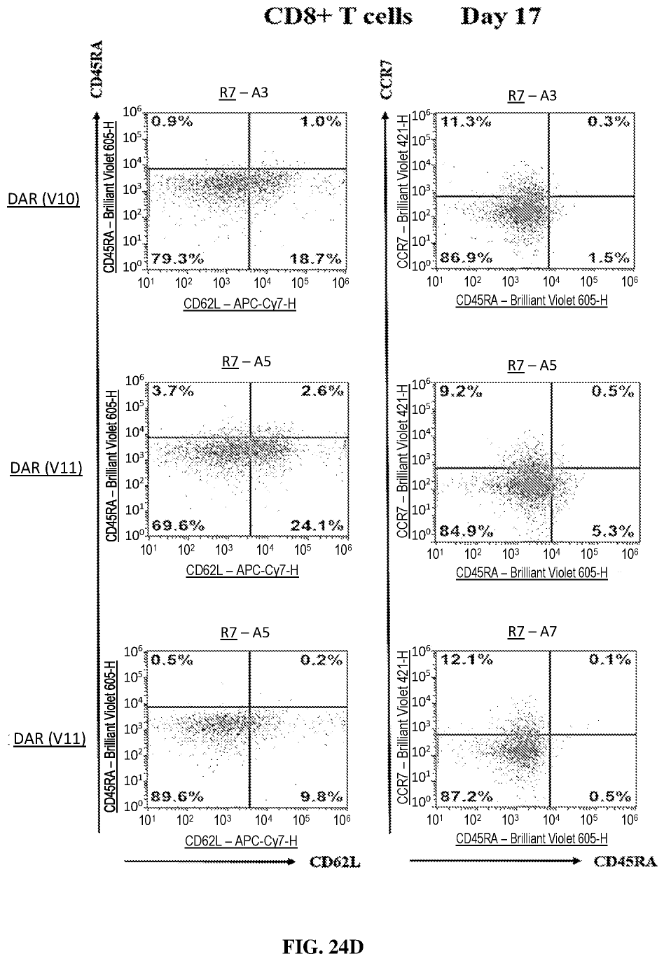

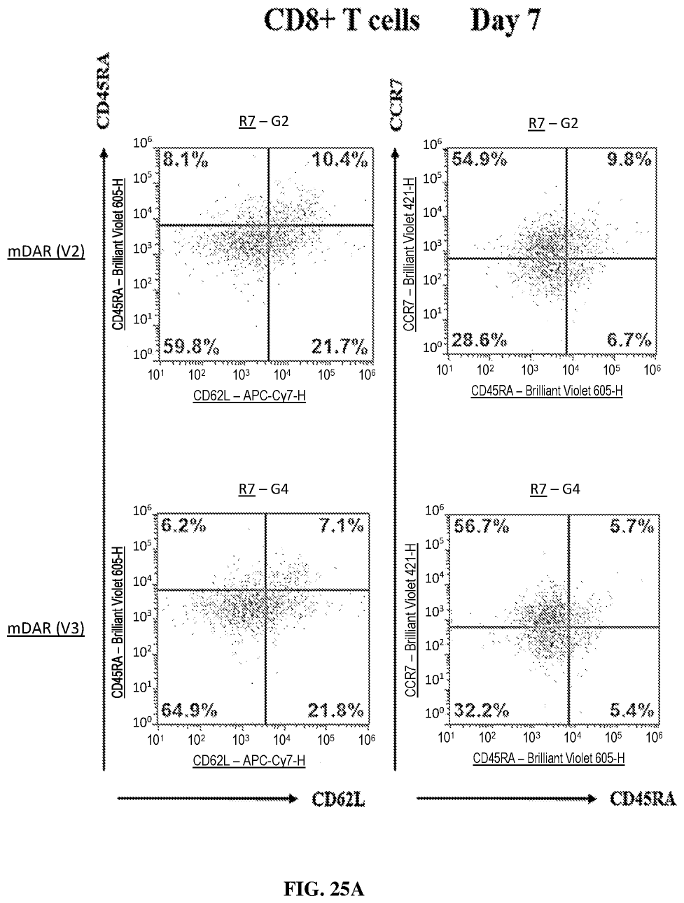

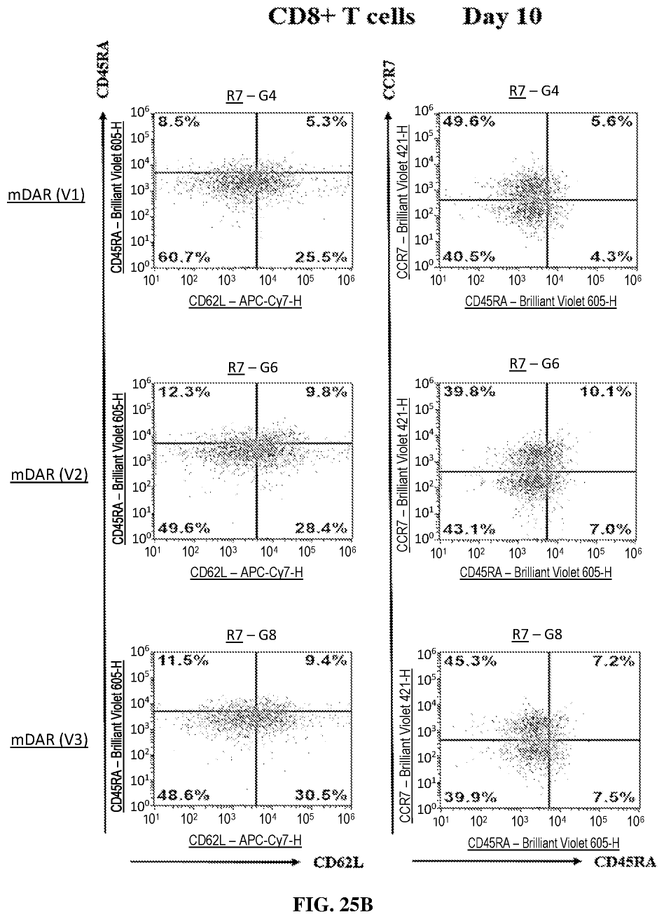

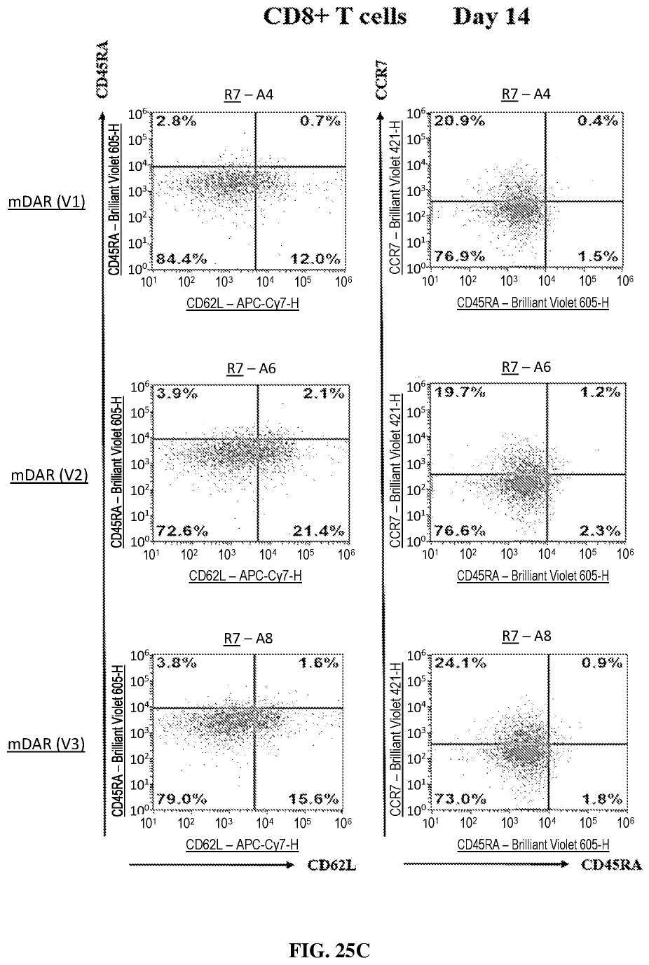

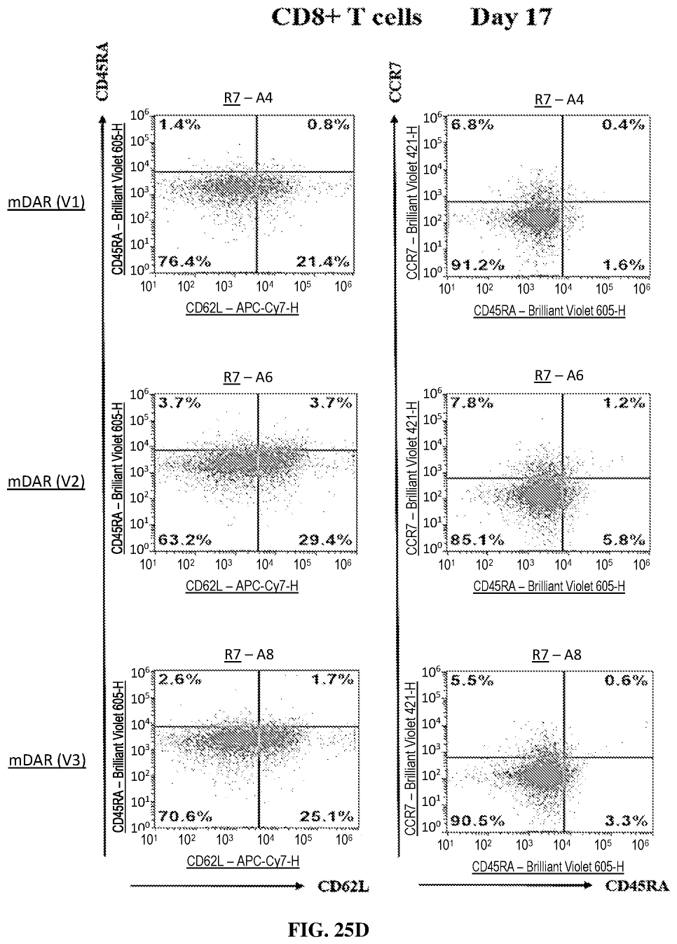

In a similar manner, flow cytometry and cell staining was used to detect memory T cells in the population of CD38 traditional DART cells (expressing V10, V11 or V12) and CD38 mDAR T cells (expressing V1, V2 or V3). Data for CD4+ memory T cells in traditional DAR T cells (V10, V11 and V12) at day 7, 10, 14 and 17 post-transfection is shown in FIGS. 22A-D, respectively. Data for CD4+ memory T cells in memory DAR T cells (V1, V2 and V3) at day 7, 10, 14 and 17 post-transfection is shown in FIGS. 23A-D, respectively. Data for CD8+ memory T cells in traditional DAR T cells (V10, V11 and V12) at day 7, 10, 14 and 17 post-transfection is shown in FIGS. 24A-D, respectively. Data for CD8+ memory T cells in memory DAR T cells (V1, V2 and V3) at day 7, 10, 14 and 17 post-transfection is shown in FIGS. 25A-D, respectively.

### Example 7: Cell Staining for Comparison of Cell Viability of CD38 Traditional DAR T Cells with CD38 mDAR T Cells

Transgenic T cells expressing either CD38 traditional DAR (V10) or memory DAR (V1) were stained with LIVE/DEAD Fixable Yellow Dead Cell stain dye (1:1000 in PBS) (Thermo Fisher, catalog No. L34959) for 30 minutes at 4° C., followed by surface staining of anti-human CD3 and APC-labeled CD38 Fc fusion protein. The transgenic T cells were stained at day 23 post-transfection. The data is shown in FIG. 10.

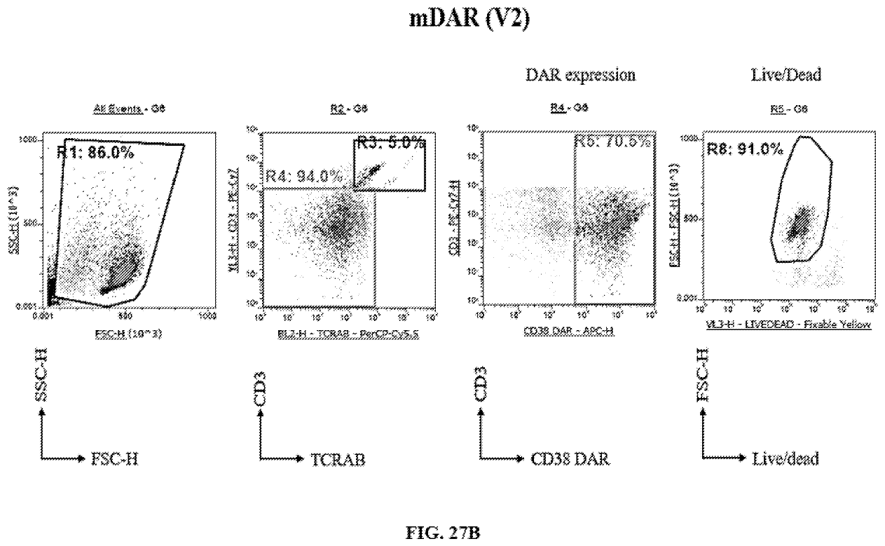

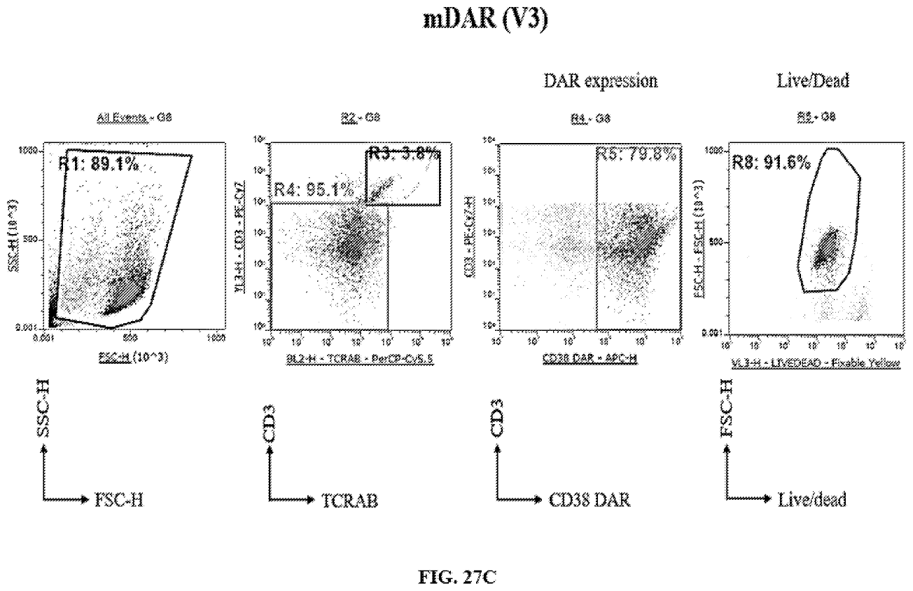

In a similar manner, transgenic T cells expressing either CD38 traditional DAR (V10, V11 or V12) or memory DAR (V1, V2 or V3) were stained for detection of TCRab and CD3, and for CD38 DAR and CD3, as described in Example 6. The transgenic cells were then stained with LIVE/DEAD Fixable Yellow Dead Cell stain dye as described herein at Example 7 above. The transgenic T cells were stained at day 14 post-transfection. The cell viability data for traditional DART cells (V10, V11 and V12) is shown in FIGS. 26A-C. The cell viability data of memory DAR T cells (V1, V2 and V3) is shown in FIGS. 27A-C.

### Example 8: Comparison of Cell Count and Cell Viability

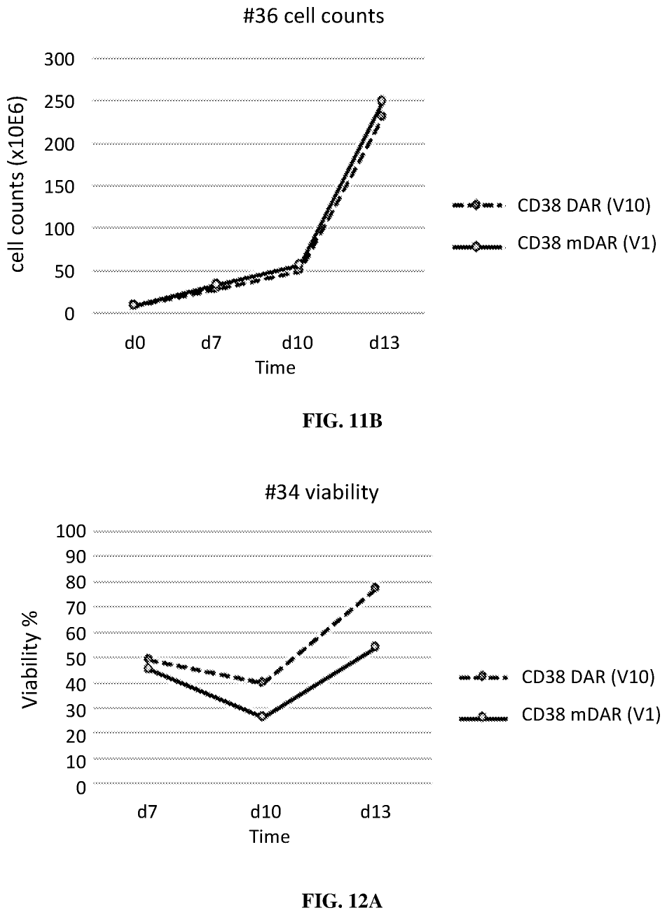

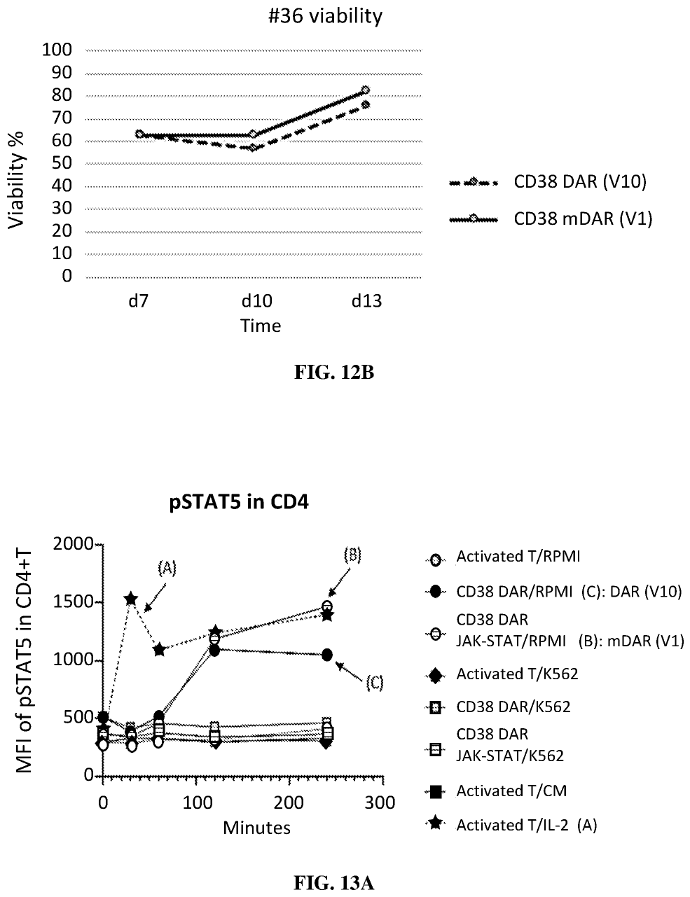

Transgenic T cells expressing either traditional CD38 DAR (V10) or memory CD38 DAR (V1) were generated using the method described in Example 3, using activated T cells from PBMCs from two different donors. Cell counts and cell viability were obtained at day 0, 7, 10 and 13, post-transfection. The cell count and cell viability data for the traditional DAR and mDAR T cells generated from donor 34 is shown in FIGS. 11A and B. The cell count and cell viability data for the traditional DAR and mDAR T cells generated from donor 36 is shown in FIGS. 12A and B.

### Example 9: Detection of Phosphorylated STAT5 and STAT3 In Vitro Activity in Transgenic DAR T Cells

Transgenic T cells expressing either CD38 traditional DAR (V10) or memory DAR (V1) were assayed for JAK-STAT pathway activation. The transgenic DAR T cells were rested in cytokine-free medium (OPTmizer T cell culture medium supplemented with human serum replacement (GIBCO A1048501, Valley Biomedical HP1022HI) overnight and stimulated with CD38+ tumor cell line RPMI8226 or CD38− tumor cell line K562 at an effector-to-target (ET) ratio of 2:1. Non-transgenic control cells were treated with IL-2 (100 IU/mL, GE Healthcare 29062790) or IL-21 (50 ng/mL, PeproTech 20021). After the tumor cells were added to the transgenic DAR T cells, or after the cytokines were added to the non-transgenic control T cells, the cells were fixed with 1.6% formaldehyde, followed by permeabilization with ice-cold methanol at time 0, 30 minutes, 1 hour, 2 hours or 4 hours. The cells were stained with the following dyes to detect phosphorylated STAT5 or STAT3 in CD4+ or CD8+ cells: Alexa Fluor 647 anti-phospho-STAT3 pY705 (BD 557815); PE anti-phospho-STAT5 (pY694) (BD 612567); anti-human CD4 (BioLegend, RPA-T4); and/or anti-human CD8 (BioLegend RPA-T8). Flow cytometry was gated on either CD4+ or CD8+ cells to detect phosphorylated STAT5 and STAT3 cells.

The data for detection of phosphorylated STAT5 in CD4+ cells is shown in FIG. 13A. The detection of phosphorylated STAT5 in CD4+ transgenic mDAR T cells (B) indicates JAK-STAT pathway activation in response to target antigen binding by the mDAR T cells. The data for detection of phosphorylated STAT5 in CD8+ cells is shown in FIG. 13B. The detection of phosphorylated STAT5 in CD8+ transgenic mDAR T cells (B) indicates JAK-STAT pathway activation in response to target antigen binding by the mDAR T cells.

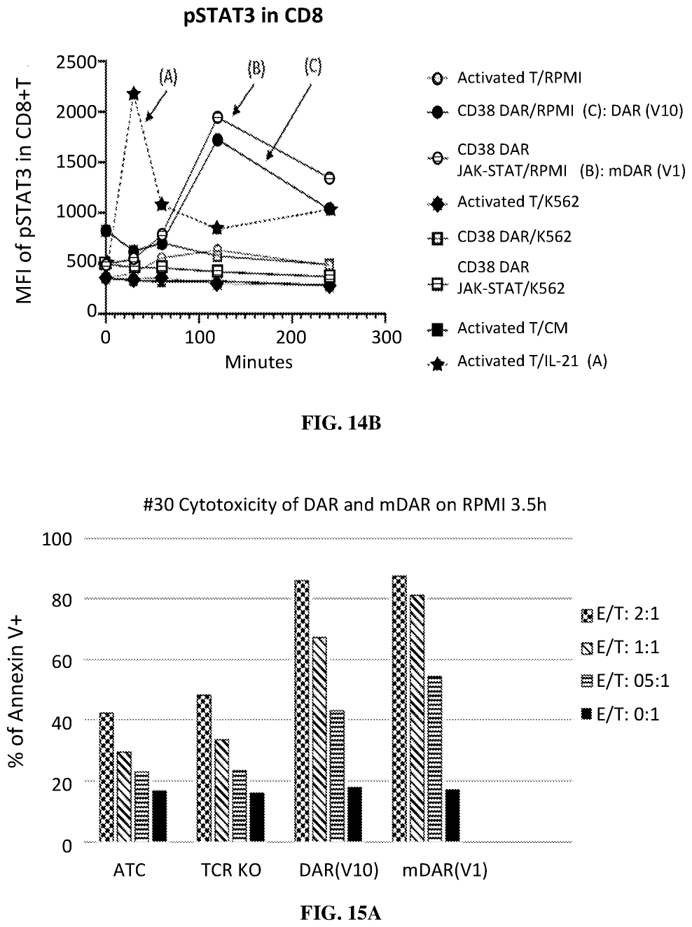

The data for detection of phosphorylated STAT3 in CD4+ cells is shown in FIG. 14A. The detection of phosphorylated STAT3 in CD4+ transgenic mDAR T cells (B) indicates JAK-STAT pathway activation in response to target antigen binding by the mDAR T cells. The data for detection of phosphorylated STAT3 in CD8+ cells is shown in FIG. 14B. The detection of phosphorylated STAT3 in CD8+ transgenic mDAR T cells indicates JAK-STAT pathway activation in response to target antigen binding by the mDAR T cells.

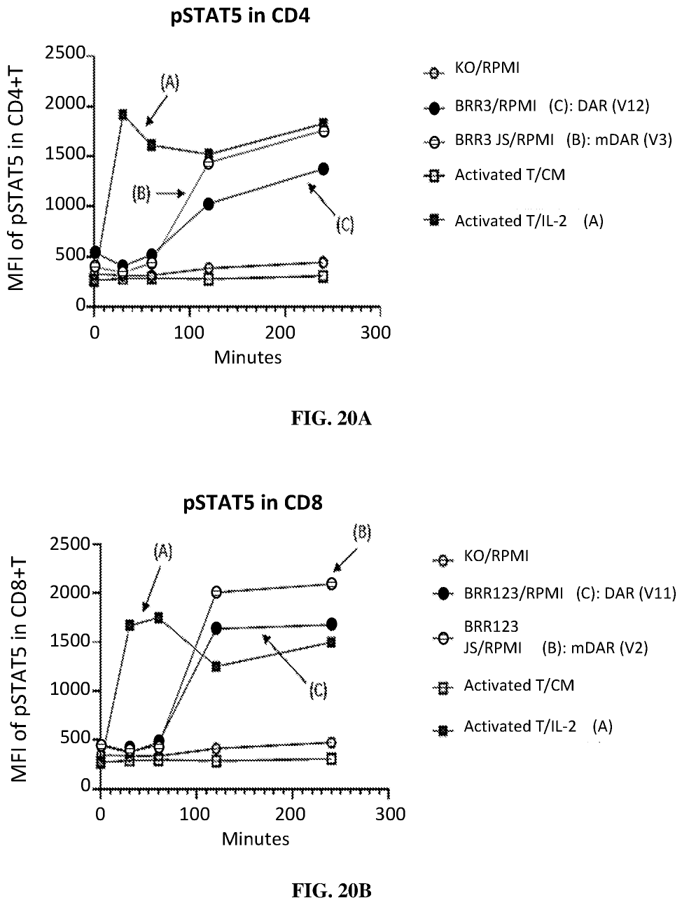

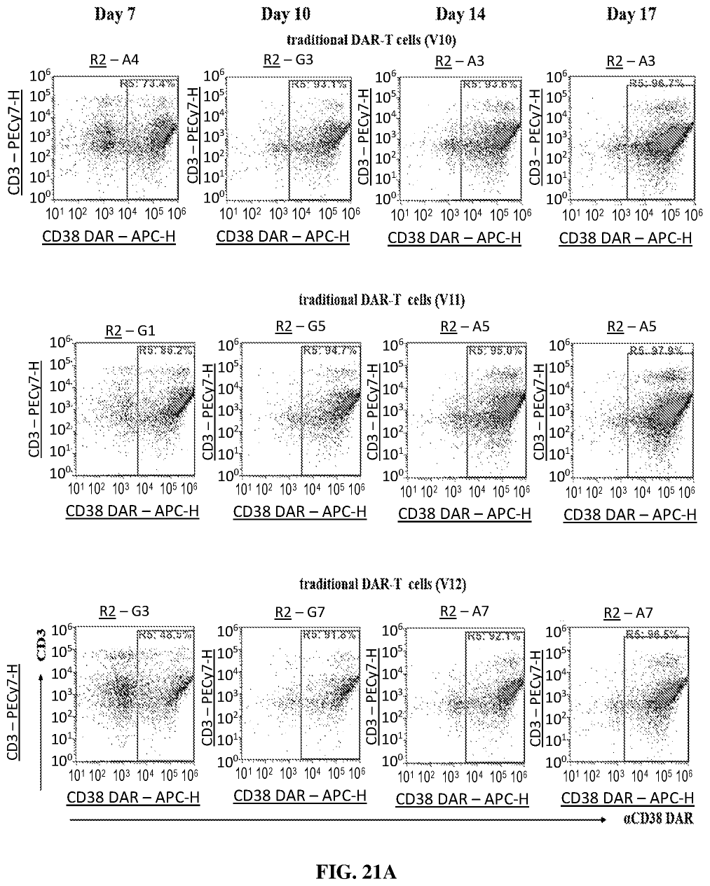

In a similar manner, transgenic T cells expressing either CD38 traditional DAR (V12) or CD38 memory DAR (V3) were assayed for JAK-STAT pathway activation. The data for detection of phosphorylated STAT5 in CD4+ cells is shown in FIG. 20A. The detection of phosphorylated STAT5 in CD4+ transgenic mDAR T cells (B: mDAR (V3)) indicates JAK-STAT pathway activation in response to target antigen binding by the mDAR T cells.

In a similar manner, transgenic T cells expressing either CD38 traditional DAR (V11) or CD38 memory DAR (V2) were assayed for JAK-STAT pathway activation. The data for detection of phosphorylated STAT5 in CD8+ cells is shown in FIG. 20B. The detection of phosphorylated STAT5 in CD8+ transgenic mDAR T cells (B: mDAR (V2)) indicates JAK-STAT pathway activation in response to target antigen binding by the mDAR T cells.

### Example 10: Comparison of In Vitro Cytotoxicity of Transgenic DAR T Cells

Transgenic T cells expressing either CD38 traditional DAR (V10) or memory DAR (V1) were assayed for cytotoxicity activity on RPMI8226, Raji and K562 target cells. Negative control T cells were also assayed. The negative control cells included non-transgenic activated T cells (ATC) and T cells having knocked-out TRAC locus (KO TRAC) but no knocked-in DAR construct. The transgenic DAR T cells were assayed at day 14 post-transfection. The cytotoxicity assays were conducted in micro titer plates. The target cells were prepared at a concentration of 0.5×10⁶ cells/mL, and 100 uL of the target cells were placed in each well. The transgenic DAR T cells were prepared at a concentration of 1×10⁶ cells/mL, and 100 uL, 50 uL or 25 uL were added to the wells containing the target cells to make E/T ratio of 2:1, 1:1 and 0.5:1, respectively. The target cells and transgenic DAR T cells were co-cultured for 3.5 hours (for RMPI target cells) or overnight (for Raji or K562 target cells). The cells were harvested and stained using LIVE/DEAD™ Fixable Yellow Dead Cell Stain Kit for 30 minutes at 4° C. The cells were washed in 100 uL cell staining buffer (BioLegend, San Diego, Calif.) and then resuspended in 100 uL of Annexin V binding buffer containing 2 μL of fluorochrome-conjugated Annexin V. The microtiter plate was incubated 15 minutes at room temperature in the dark. The cells were washed once with 200 μL binding buffer and then analyzed by flow cytometry. Cytotoxicity data of the traditional DAR compared to mDAR T cells on target cells RPMI8226, Raji and K562 cells is shown in FIGS. 15A, 15B and 15C, respectively.

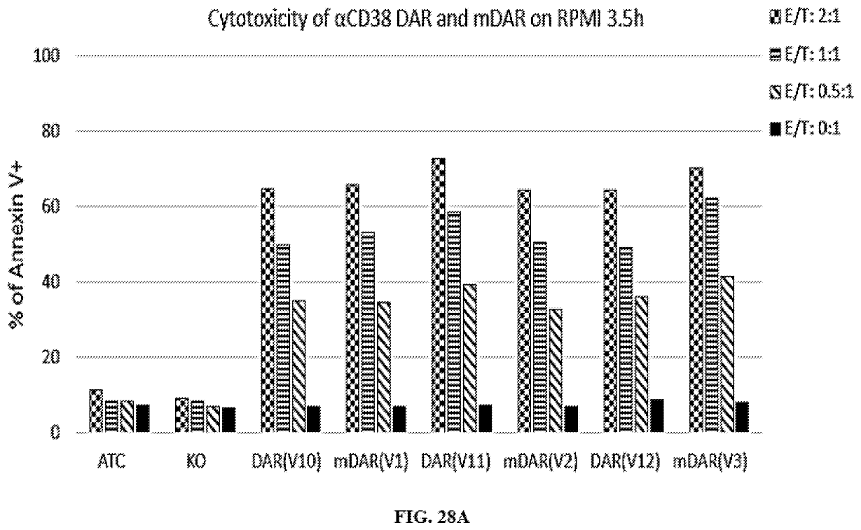

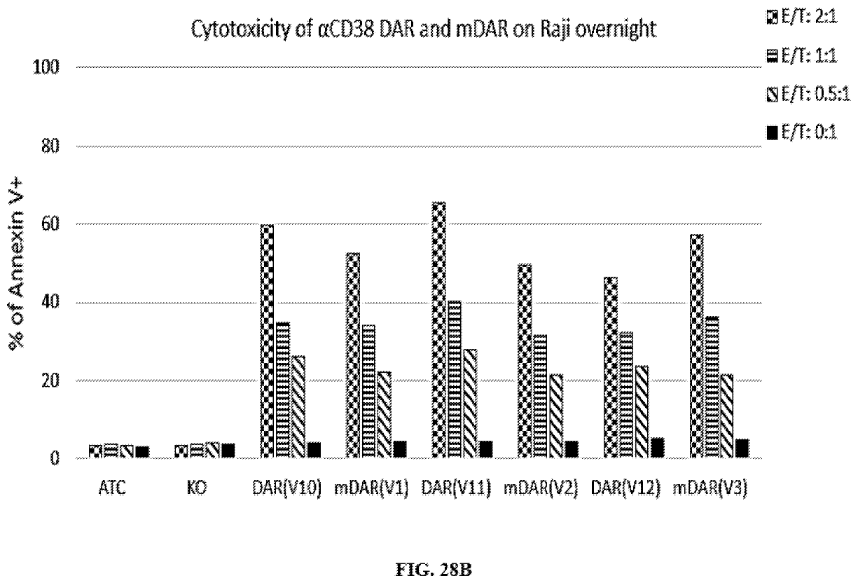

In a similar manner, cytotoxicity activity of transgenic T cells expressing CD38 traditional DAR (V10, V11 or V12) or CD38 memory DAR (V1, V2 or V3), on RPMI or Raji cells, were directly compared. The E/T ratios of 2:1, 1:1, 0.5:1 and 0:1, were tested. The transgenic T cells were assayed at day 15 post-transfection. The cytotoxicity data for RPMI target cells is shown in FIG. 28A, and for Raji target cell in FIG. 28B.

### Example 11: Comparison of In Vitro Cytokine Secretion of Transgenic DART Cells

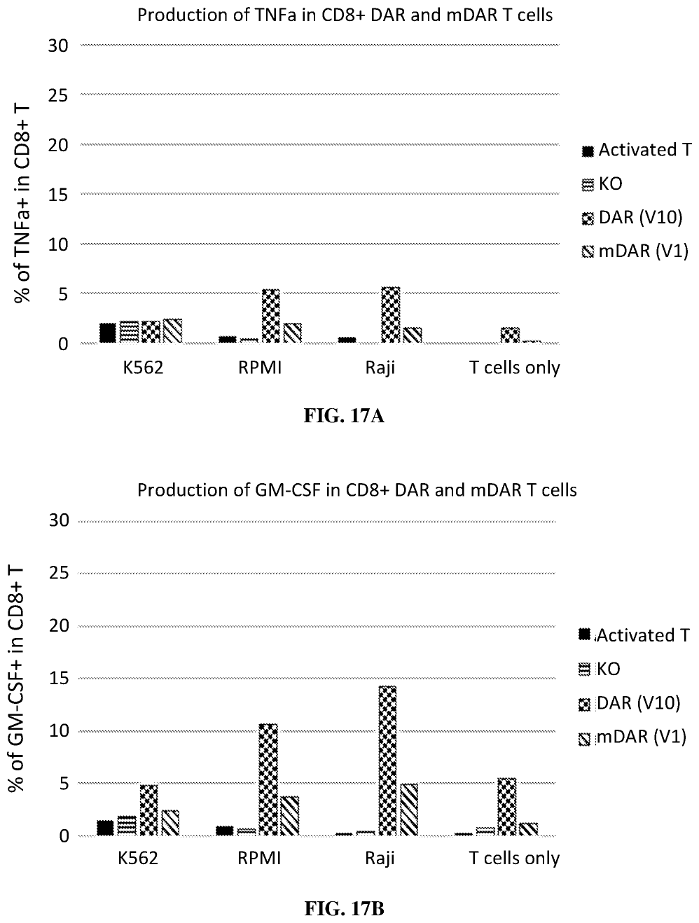

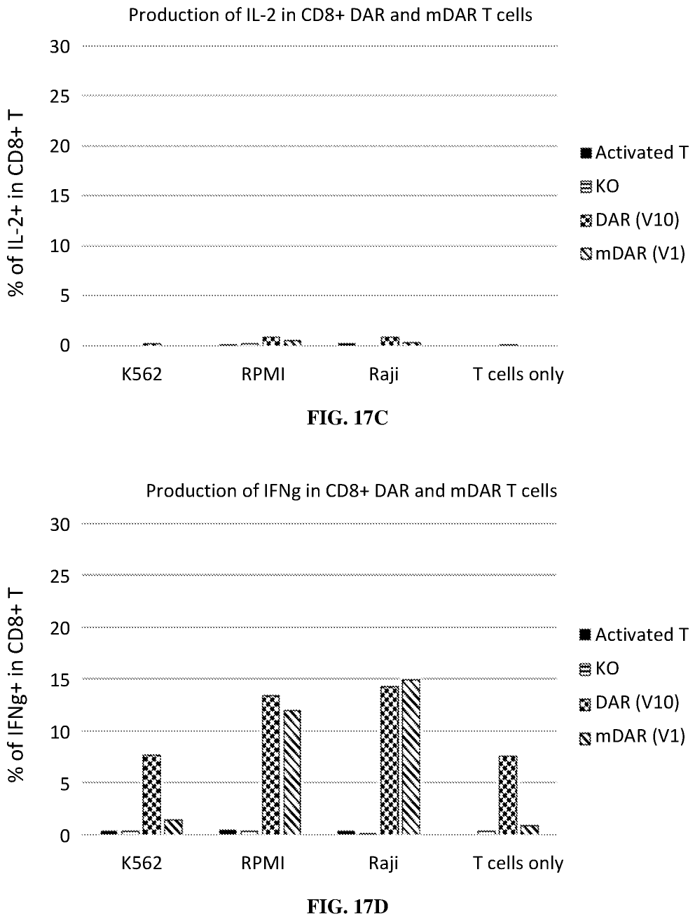

Transgenic T cells expressing either CD38 traditional DAR (V10) or memory DAR (V1) were assayed for their cytokine secretion activity in the presence of RPMI8226, Raji and K562 target cells. Negative control T cells were also assayed. The negative control cells included non-transgenic activated T cells (ATC) and T cells having knocked-out TRAC locus (KO TRAC) but no knocked-in DAR construct. The transgenic DAR T cells were assayed at day 14 post-transfection. The cytokine secretion assays were conducted in micro titer plates. The target cells were prepared at a concentration of 0.5×10⁶ cells/mL, and 100 uL of the target cells were placed in each well. The transgenic DAR T cells were prepared at a concentration of 0.5×10⁶ cells/mL, and 100 uL was added to the wells containing the target cells to make E/T ratio of 1:1. The cells were stained with FITC anti-human CD4 (RPA-T4 BioLegend 300506) or PE anti-human CD8a (RPA-T8 BioLegend, 301051) for detection of CD4+ or CD8+ cells. The cells were fixed and permeabilized using the Cytofix/Cytoperm Kit (BD 554715) and stained with PE anti-human TNFa (MAb11 BioLegend 502909), APC anti-human IFNg (4S.B3 BioLegend 502512), PerCP/Cy5.5 anti-human GM-CSF (BVD2-21C11 BioLegend 502311), or APC/Cy7 anti-human IL-2 (MQ1-17H12 BioLegend 500342). The frequency of cytokine-secreting cells within CD4+ or CD8+ T cell populations was determined by flow cytometry. The percent of CD4+ cells secreting TNFa, GM-CSF, IL-2 or IFNg is shown in FIGS. 16A, B, C and D, respectively. The percent of CD8+ cells secreting TNFa, GM-CSF, IL-2 or IFNg is shown in FIGS. 17A, B, C and D, respectively.

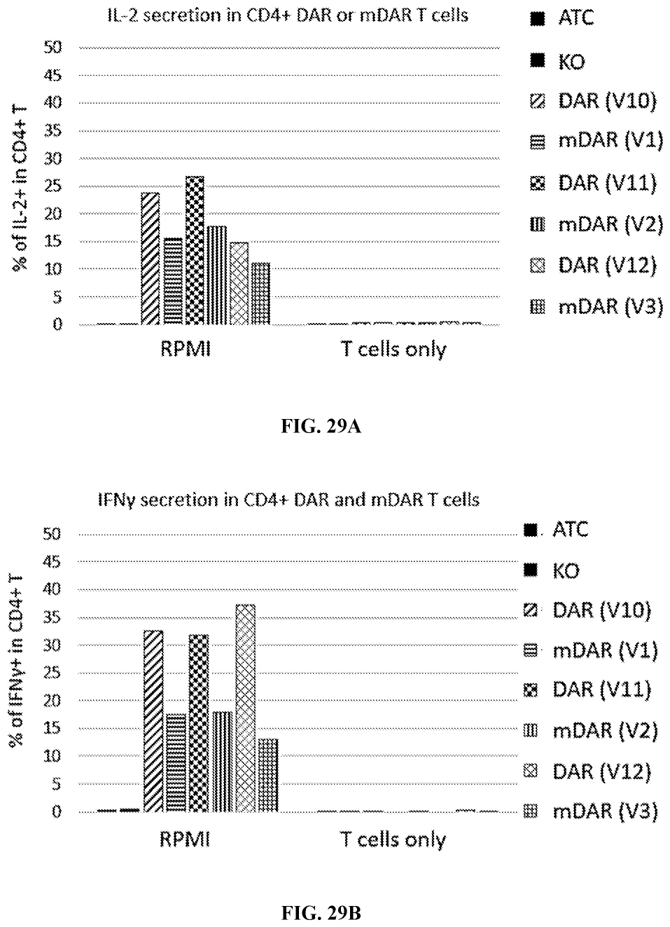

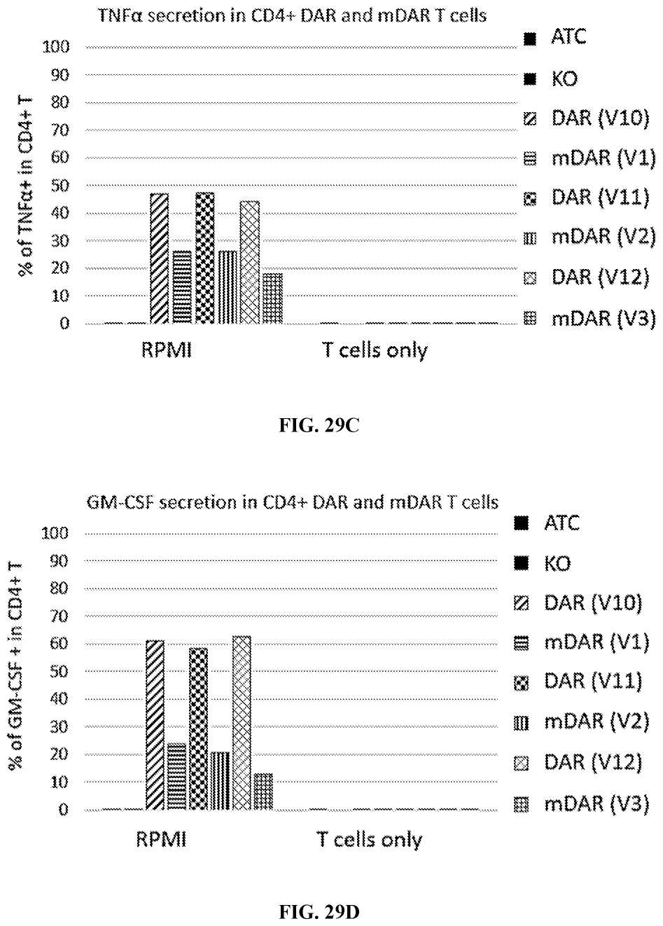

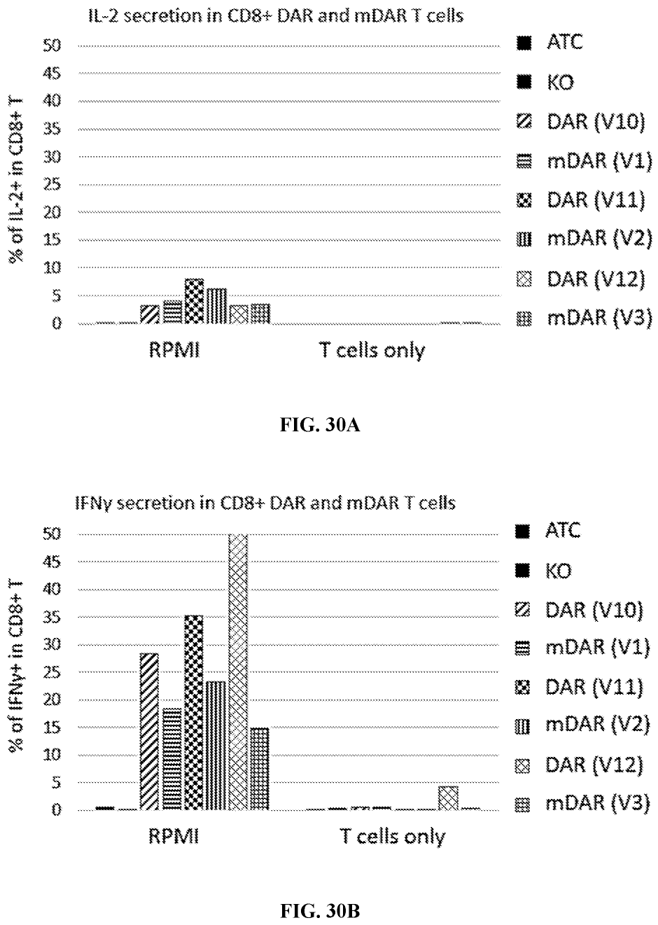

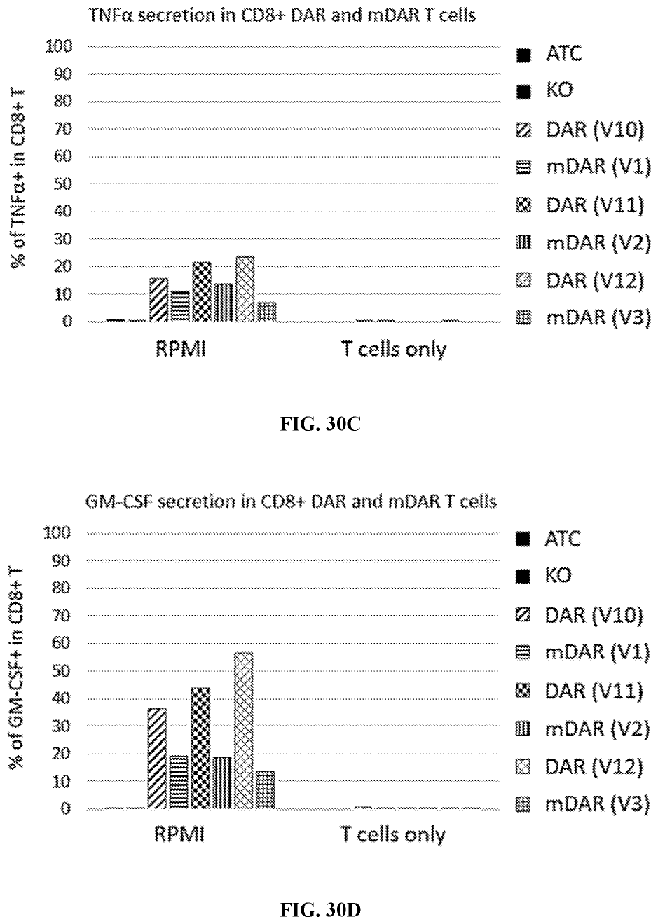

In a similar manner, cytokine secretion activity of CD4+ and CD8+ transgenic T cells expressing CD38 traditional DAR (V10, V11 or V12) or expressing CD38 memory DAR (V1, V2 or V3) were compared. The cytokine secretion assays detected IL-2, IFNg, TNFa and GM-CSF, using transgenic T cells at day 15 post-transfection. The cytokine secretion data of CD4+ transgenic T cells is shown in FIGS. 29A-D, and the data for CD8+ transgenic T cells is shown in FIGS. 30A-D.

### Example 12: In Vivo Tumor Killing in a Mouse Model

Tumoricidal activity of transgenic T cells expressing the CD38 traditional DAR (V1)) was compared to transgenic T cells expressing antiCD38 mDAR (V1) in a RPMI8226 xenograft mouse model. NSG mice were used for the study. Multiple myeloma cell line RPMI8226 transfected with a lentiviral vector to express luciferase and GFP genes were prepared (RPMI8226-FLuc). A total 6×10⁷ cells of RPMI8226-Fluc were suspended in PBS and then injected intravenously into the tail vein of each mouse.

Tumor growth was monitored by measuring total photon flux with an IVIS Lumina III In Vivo Imaging System (Perkin Elmer Health Sciences, Inc) on the dorsal side of each mouse weekly after tumor cell inoculation.

At day 27 after inoculation with the RPMI8226-Fluc tumor cells, a single treatment of 2×10⁶ cells of the transgenic T cells expressing either the traditional DAR (V10) or mDAR (V1) was administered to the mice via the tail vein in PBS.

The total flux of the mice were monitored on day 1 post treatment and weekly thereafter. Blood samples were obtained via the tail vein from each animal at day 1 post-treatment and weekly thereafter. The blood samples were analyzed via flow cytometry for the number of circulating CD45+ human T cells (an indicator of T cell proliferation) in the treated mice. The data shown in FIG. 18A shows that mDAR T cells have more potent anti-tumor effect in the mouse compared to the mouse treated with traditional DAR T cells. The data in FIG. 18B shows that the mouse treated with mDAR T cells has a robust T cell proliferation response which peaks at approximately day 14 post-treatment, compared to the mouse treated with traditional DAR T cells.

On day 40 after inoculation with the RPMI8226-Fluc tumor cells, and after the single treatment with either the traditional DAR (V10) T cells or mDAR (V1) T cells, the mice were re-challenged with 1×10⁷ of the RPMI8226-Fluc tumor cells via inoculation through the tail vein.

The total flux of the mice were monitored on day 1 post treatment and weekly thereafter. Blood samples were obtained via the tail vein from each animal at day 1 post-treatment and weekly thereafter. The blood samples were analyzed via flow cytometry for the number of circulating CD45+ human T cells (an indicator of T cell proliferation) in the treated mice.

The data in FIG. 19A shows that at day 21 post re-challenge, the mouse treated with mDAR T cells are tumor-free while the mice treated with traditional DAR T cells have tumor re-growth. The data in FIG. 19B shows that the mouse treated with mDAR T cells has a robust T cell proliferation response which peaks at approximately day 1 post-re-challenge, compared to the mouse treated with traditional DAR T cells.

A dose study was conducted using the same xenograft mouse model. Mice were inoculated with 6×10⁷ RPMI8226-Fluc tumor cells via inoculation through the tail vein.

At day 27 after inoculation with the RPMI8226-Fluc tumor cells, each animal was administered, via the tail vein, a single treatment of 2×10⁶ cells of the transgenic T cells expressing CD38 memory DAR (V1) at a dose of 1×10⁶, 1×10⁵, 1×10⁴, 1×10³ transgenic T cells, or 1×10⁶ knocked-out control T cells.

The total flux of the mice were monitored one day prior to treatment and then at day 6, 22, 26 and 33 post-treatment. Blood samples were obtained via the tail vein from each animal at day 7, 13, 19, 25 and 33 post-treatment. The blood samples were analyzed via flow cytometry for the number of circulating CD45+CD3− human T cells (an indicator of T cell proliferation) in the treated mice. The data shown in FIG. 19C shows that the higher doses of 1×10⁶ and 1×10⁵ mDAR (V1) T cells have more potent anti-tumor effect in the mice. The data in FIG. 19D shows that the mouse treated with 1×10⁵ mDAR T cells has a robust T cell proliferation response which peaks at approximately day 25 post-treatment.

### Example 13: In Vitro Tumor Cell Re-Challenge Assay

Transgenic T cells expressing either CD38 traditional DAR (V10) or CD38 memory DAR (V1) were generated using the method described in Example 3, using activated T cells from PBMCs. Transgenic T cells at day 18 post-transfection were used for this re-challenge assay.

Challenge: On the first day (e.g., Day 1) 0.3×10⁶ of the transgenic T cells were co-cultured in wells with either RPMI8226 or Raji cells, in cytokine-free medium, at an E/T ratio of 1:1. On the second, third, fourth and fifth day, RPMI8226 or Raji cells (0.3×10⁶ cells) were added to their respective wells.

Re-challenge: Beginning on Day six, and every other day until day twenty-four, 0.6×10⁶ tumor cells (either RPMI8226 or Raji) were added to their respective wells.

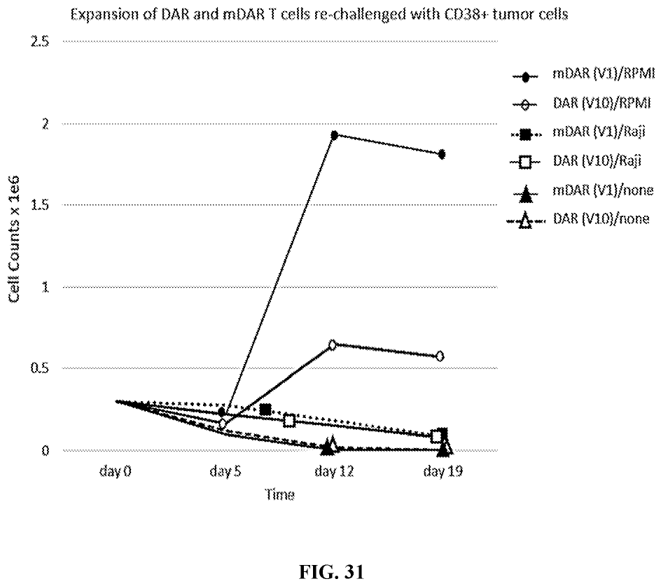

Negative control wells contained transgenic T cells expressing either CD38 traditional DAR (V10) or CD38 memory DAR (V1) but were not challenged or re-challenged with RPMMI8226 or Raji tumor cells. Cell expansion capabilities were determined by obtaining cell counts starting at Day 1 through Day 24. The cell count data is shown in FIG. 31.

### Example 14: Characterization of the Ratio of CD8+/CD4+ Transgenic T Cells

The frequency of CD4+ or CD8+ T cells in a population of transgenic T cells expressing one of three traditional DAR (V10, V11 or V12), or expressing one of three mDAR (V1, V2 or V3) constructs, was determined by flow cytometry. Transgenic T cells were prepared using PBMCs from two different donors (36 and 37) according to the protocol described in Example 3 above. CD4+ or CD8+ T cells were detected at day 7, 10, 14, 17, 28 and 35 post-transfection. The CD4+ and CD8+ T cells were distinguished by cell surface staining with FITC anti-human CD4 (RPA-T4 BioLegend 300506), or PE anti-human CD8a (RPA-T8 BioLegend, 301051) to detect CD8+ cells.

The ratios of CD8+/CD4+ T cells for transgenic T cells prepared from donor 36 is shown in FIG. 32A and for transgenic T cells prepared from donor 37 is shown in FIG. 32B.

### Example 15: In Vivo Study Comparing Treatment of RPMI8826 Tumor-Bearing Mice with mDAR (V1)-T Cells and CD38 DAR (V10)-T Cells

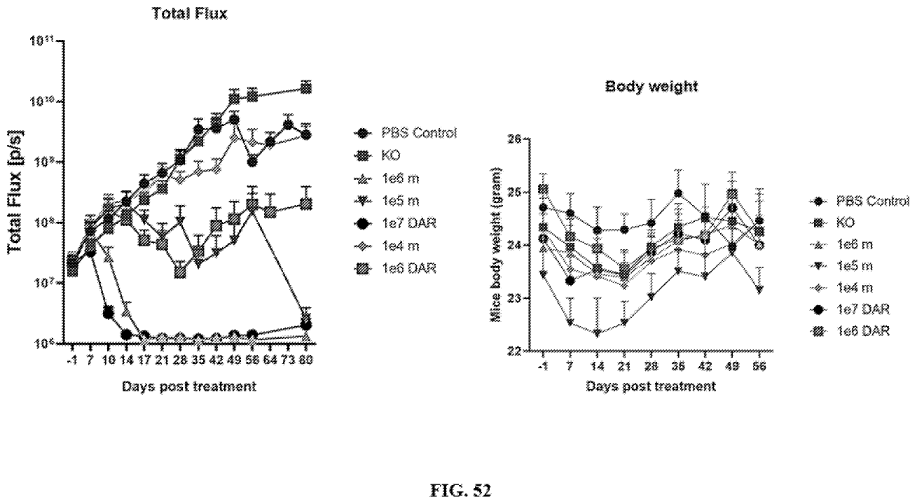

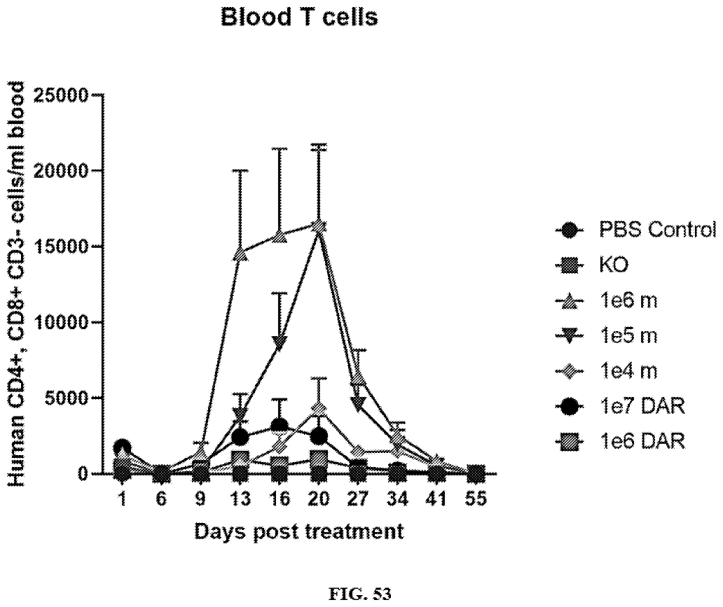

An in vivo study was performed to compare the effects of treating RPMI8226 xenograft tumors in mice with transgenic T cells expressing the CD38 mDAR (V1) and transgenic T cells expressing the CD38 traditional DAR (V10). The T cells expressing the CD38 mDAR (V1) had a construct encoding the precursor V1 mDAR of SEQ ID NO:95 inserted into the TRAC locus, inactivating the T cell receptor gene. The T cells expressing the CD38 DAR (V10) had a construct encoding the precursor V10 DAR of SEQ ID NO:104 inserted into the TRAC locus, inactivating the T cell receptor gene. In this study, mice were injected with 10⁷ RPMI-Fluc cells, and 29 days later the mice were treated by intravenous administration of PBS, TRAC KO cells (10⁶ cells), V1 CD38 mDAR-T cells (either 10⁶, 10⁵, or 10⁴ cells), or V10 CD38 traditional DAR-T cells (10⁷ or 10⁶ cells), with ten mice per treatment group. In vivo imaging was performed at least weekly beginning one week before treatment, and blood was drawn weekly from the tail vein beginning one day after T cell treatment. Body weight was also monitored weekly. FIG. 51 shows the IVIS images of the mice up to eight weeks after treatment, where PBS and TCR KO mice should steady tumor progression, while mice treated with 10⁶ mDAR V1 T cells showing comparable results to mice treated with 10⁷ mDAR V1 T cells, with all ten of the mice of each group demonstrating tumor eradication by Day 17. FIG. 52A provides the total flux from the tumors over time and FIG. 52B provides body weights over the course of the study. FIG. 53, a graph of the number of human T cells found in the peripheral blood of the mice of the DAR treatment groups as well as of the control groups shows that the mDAR cells, when administered in doses of 10⁶ and 10⁵ cells, proliferated in the mice for nearly four weeks after treatment to a much greater extent than did traditional DAR cells, which only showed increased levels at the highest cell dose (10⁷ cells).

### Example 16: Anti-Tumor Efficacy of CD38 DAR, and V1, V2, and V3 CD38 mDARs in an RPMI8226 Xenograft Mouse Model

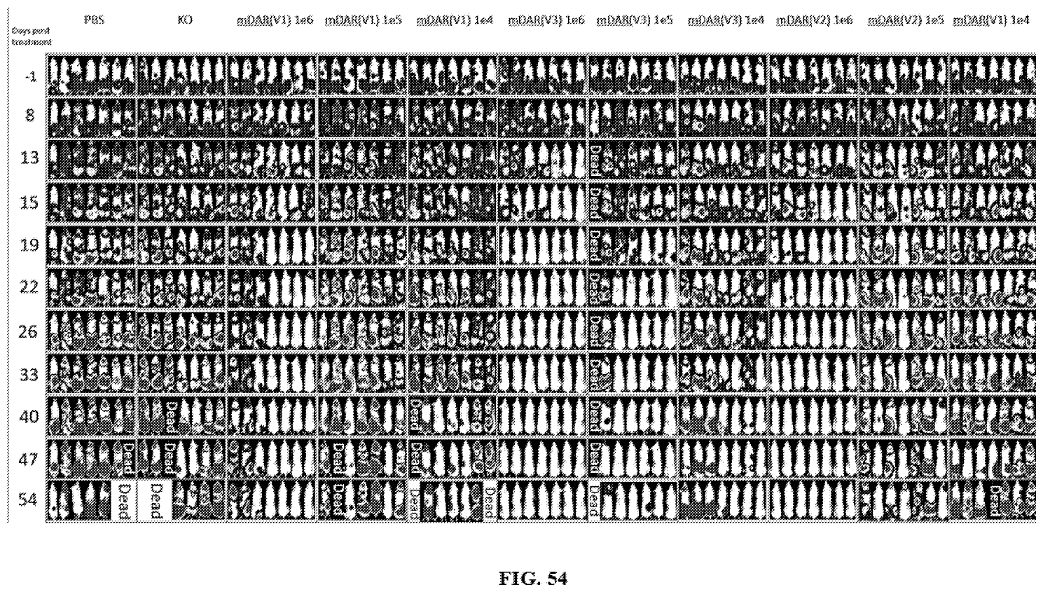

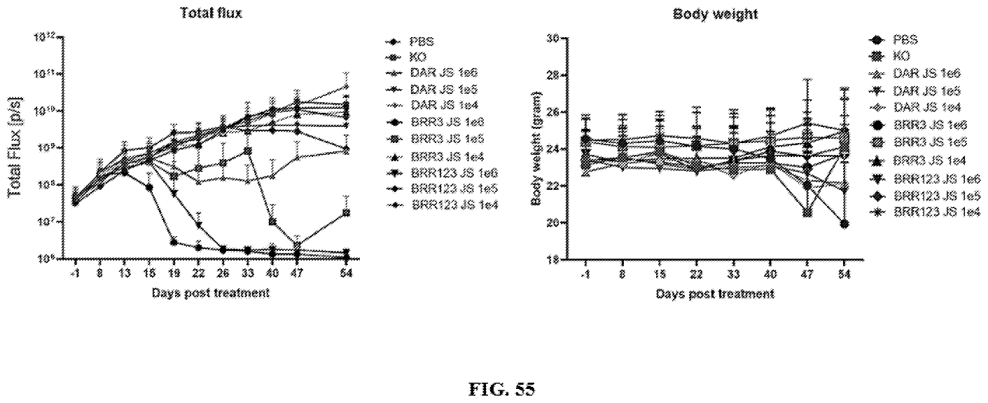

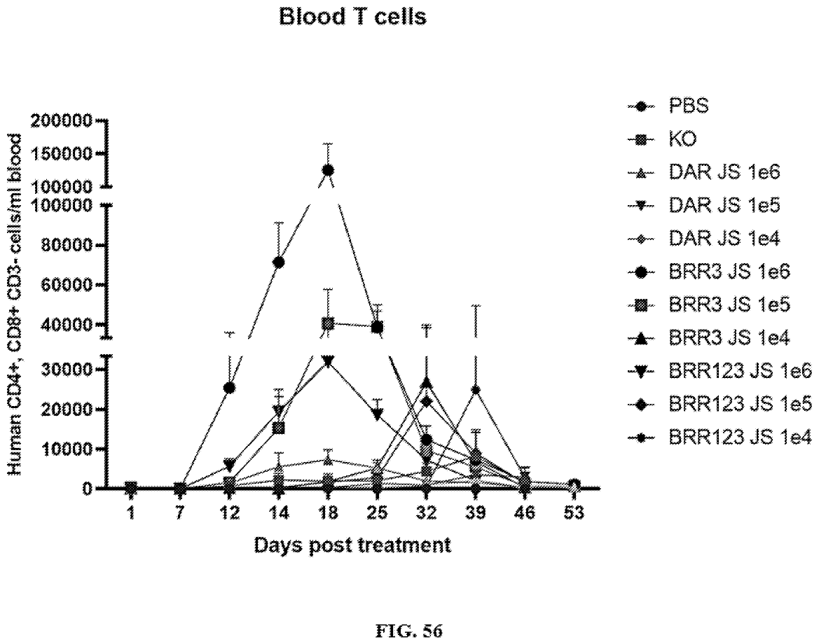

Mice were inoculated with 10⁷ tumor cells and treated as described in Example 15, except that treatments were initiated 27 days later with three different transgenic T cell populations generated as described in Example 3 expressing three different CD38 mDARs: CD38 mDAR(V1), CD38 mDAR(V2), and CD38 mDAR(V3). The engineered T cells expressing the CD38 mDAR (V1) had a construct encoding the precursor V1 mDAR of SEQ ID NO:95 inserted into the TRAC locus, the engineered T cells expressing the CD38 mDAR (V2) had a construct encoding the precursor V2 mDAR of SEQ ID NO:98 inserted into the TRAC locus, and the engineered T cells expressing the CD38 mDAR (V3) had a construct encoding the precursor V3 mDAR of SEQ ID NO:101 inserted into the TRAC locus inactivating the T cell receptor gene. Each mDAR-T cell population was administered at three different doses of 10⁶, 10⁵, and 10⁴ cells in a 200 μl volume. There were seven mice in each treatment group. Control groups were infused with PBS alone and TRAC KO T cells (10⁶ cells). IVIS, performed at least weekly, showed that mDAR V2 and mDAR V3 were highly effective in eradicating tumor, where all seven mice of both the 10⁶ CD38 mDAR V2-T cell and 10⁶ CD38 mDAR V3-T cell treatment groups survived out to Day 55 when the study terminated with no apparent tumor (FIG. 54). In contrast three of the seven 10⁶ CD38 mDAR V1-T cell treatment group had significant tumor burden by Day 54. FIG. 55A shows the average total flux from the labeled tumor for all 11 treatment groups over the course of the study, demonstrating treatment with mDAR(V2) and mDAR(V3)-T cells was highly successful and had higher efficacy with respect to treatment with mDAR(V1)-T cells. FIG. 55B shows the body weight of treated animals over the same time frame. Analysis of blood samples (FIG. 56), drawn weekly throughout the study, showed that mDAR(V3)-T cells, and to a somewhat lesser degree mDAR(V2)-T cells, proliferated in the blood of treated mice to a much greater degree than V1 mDAR-T cells, showing the high in vivo expansion capability of mDAR-T cells.

### Example 17: Anti-Tumor Efficacy of V3 CD38 mDAR in a Daudi Xenograft Mouse Model

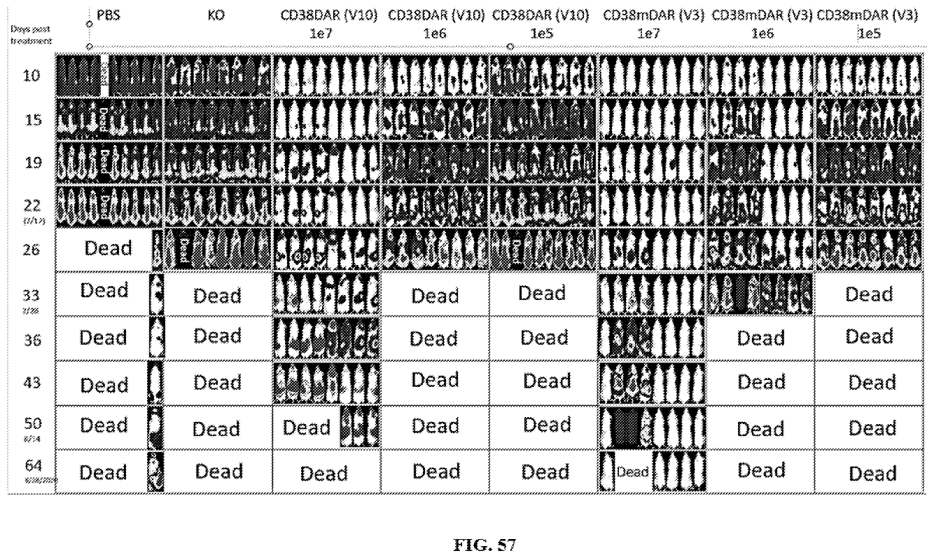

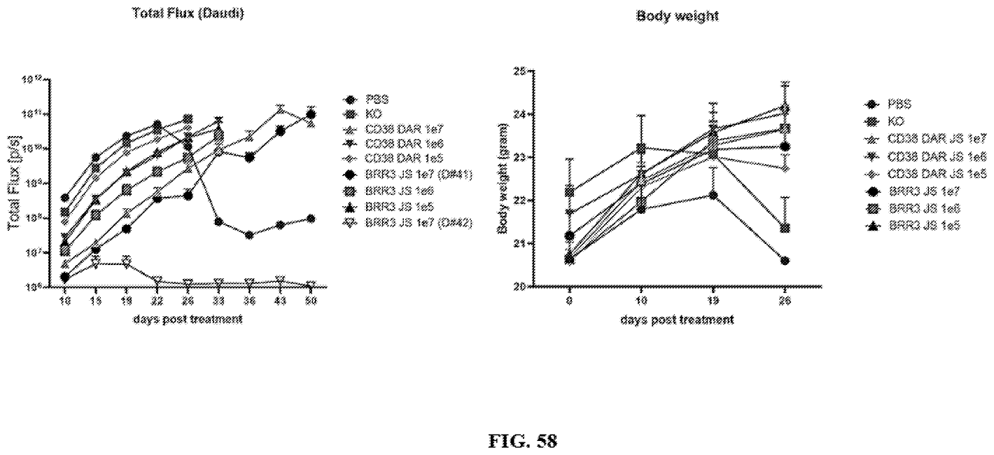

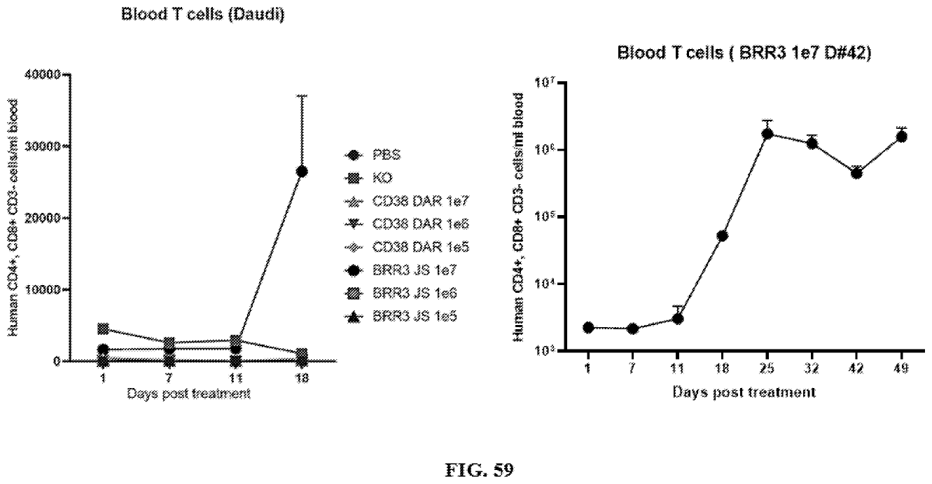

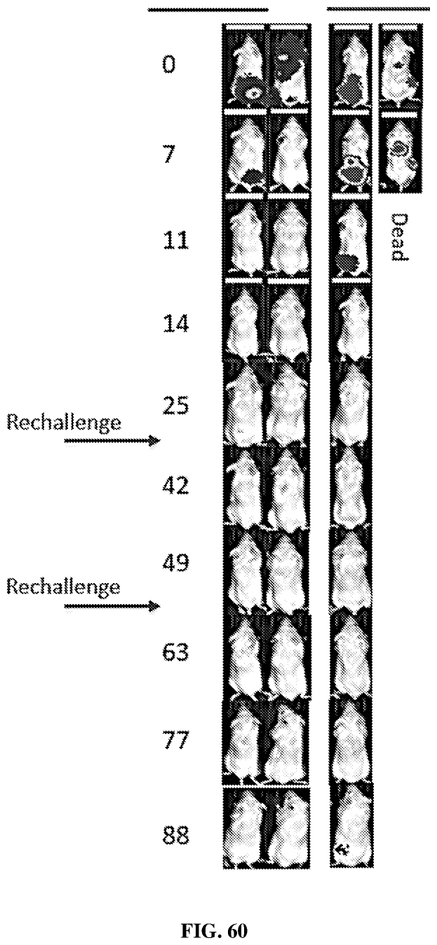

A further study compared treatment of mice inoculated with Daudi tumor cells with transgenic T cells expressing a traditional V10 CD38 DAR (V10) and transgenic T cells expressing a V3 CD38 mDAR, where the T cells expressing the CD38 DAR (V10) had a construct encoding the precursor V10 DAR of SEQ ID NO:104 inserted into the TRAC locus, and the engineered T cells expressing the CD38 mDAR (V3) had a construct encoding the precursor V3 mDAR of SEQ ID NO:101 inserted into the TRAC locus. In this study, groups of mice were treated with 10⁷, 10⁶, and 10⁵ cells expressing CD38 DAR(V10) and 10⁷, 10⁶, and 10⁵ cells expressing a V3 CD38 mDAR, with eight mice in each treatment group. FIG. 57 shows the dramatic difference in survivorship among the groups of mice, where 5 mice of the 10⁷V3 CD38 mDAR-T cell group survived, with no evident tumor burden, at the end of the study at day 64, yet no mice of the groups treated with fewer V3 CD38 mDAR-T cells (i.e., 10⁶ and 10⁵ cells) were alive at Day 36 and all mice of the CD38 CAR(V10) (traditional DAR construct) survived to Day 64. FIG. 58A provides a graph of the total flux from the mice over time, again showing the dramatic difference in tumor burden and survival for mice treated with 10⁷V3 CD38 mDAR-T cells and groups treated with a traditional DAR. FIG. 58B provides the body weights of the mice over the course of the study. FIG. 59A shows that the V3 CD38 mDAR-T cells proliferated extensively in mice treated with 10⁷ cells, and FIG. 59B shows the in vivo proliferation of the introduced mDAR-T cells in mice treated with 10⁷ cells extended for at least 7 weeks post-treatment.

### Example 18: Multiple Rechallenge Study in RPMI Tumor Model

Mice were inoculated intravenously with 10⁸ RPMI-8226 cells and after 64 days were treated with 3.5×10⁶ T cells expressing either CD38 DAR (V10) or CD38 mDAR(V3). At Day 27, when tumor had been eradicated from CD38 mDAR(V3)-T cell-treated mice and nearly eradicated from CD38 DAR(V10)-T cell treated mice, the mice received another inoculum of 10⁸ RPMI-8226 tumor cells. While there was some tumor progression in CD38 DAR(V10)-T cell treated mice, there was no evidence of any tumor becoming established in CD38 mDAR(V3)-T cell-treated mice. The inoculation of tumor was repeated at Day 56, after which CD38 DAR(V10)-T cell treated mice developed tumors, while CD38 mDAR(V3)-T cell-treated mice remained tumor-free (FIG. 60).

We claim:

1. A precursor polypeptide comprising a plurality of polypeptide regions, the plurality comprising, in order from the amino terminus to the carboxyl terminus: (i) a heavy chain leader sequence; (ii) an antibody heavy chain variable region; (iii) an antibody heavy chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having a STAT binding motif, and (3) an optional intracellular costimulatory region; (vii) a self-cleaving sequence; (viii) a light chain leader sequence; (ix) an antibody light chain variable region; and (x) an antibody light chain constant region, wherein the self-cleaving sequence permits cleaving of the precursor polypeptide into a first and second polypeptide chain.

2. The precursor polypeptide of claim 1, wherein the cleaving of the self-cleaving sequence generates (a) the first polypeptide chain, comprising (i) the heavy chain leader sequence, (ii) the antibody heavy chain variable region, (iii) the antibody heavy chain constant region, (iv) the optional hinge region, (v) the transmembrane region, (vi) the intracellular JAK-STAT signaling region, and (b) the second polypeptide chain, comprising (vii) a light chain leader sequence; (viii) an antibody light chain variable region; and (ix) an antibody light chain constant region, and wherein the heavy chain variable region and the light chain variable region are capable of forming an antigen-binding domain that binds a target protein/antigen.

3. A precursor polypeptide comprising a plurality of regions, the plurality comprising, in order from the amino terminus to the carboxyl terminus: (i) a light chain leader sequence; (ii) an antibody light chain variable region; (iii) an antibody light chain constant region; (iv) an optional hinge region; (v) a transmembrane region; (vi) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having a STAT binding motif, and (3) an optional intracellular costimulatory region; (vii) a self-cleaving sequence; (viii) a heavy chain leader sequence; (ix) an antibody heavy chain variable region; and (x) an antibody heavy chain constant region, wherein the self-cleaving sequence permits cleaving the of the precursor polypeptide into a first and second polypeptide chain.

4. The precursor polypeptide of claim 3, wherein the cleaving of the self-cleaving sequence generates (a) the first polypeptide chain, comprising (i) the light chain leader sequence, (ii) the antibody light chain variable region, (iii) the antibody light chain constant region, (iv) the optional hinge region, (v) the transmembrane region, (vi) the intracellular JAK-STAT signaling region, and (b) the second polypeptide chain, comprising (vii) a heavy chain leader sequence; (viii) an antibody heavy chain variable region; and (ix) an antibody heavy chain constant region, and wherein the heavy chain variable region and the light chain variable region are capable of forming an antigen-binding domain that binds a target protein/antigen.

5. The precursor polypeptide of any one of the preceding claims, wherein the cytokine receptor intracellular domain comprises an IL2Rβ intracellular domain having the amino acid sequence of SEQ ID NO:43.

6. The precursor polypeptide of any one of the preceding claims, wherein the CD3ζ intracellular signaling region comprises: CD3ζITAM 1 (SEQ ID NO:52, 53 or 54); CD3ζ ITAM 2; CD3ζITAM 3 (SEQ ID NO:49, 58 or 60); CD3ζITAM3 having a deleted portion (designated ITAM d3, SEQ ID NO:51 or 55); CD3ζITAM 1 and 2; CD3ζITAM 1, 2 and 3 (SEQ ID NO:47); CD3ζITAM 1 and 3 (SEQ ID NO:49); CD3ζITAM 2 and 3; or CD3ζITAM 1 and ITAM d3 (SEQ ID NO:51 or 56).

7. The precursor polypeptide of any one of the preceding claims, wherein the STAT binding motif comprises the amino acid sequence of YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (any one of SEQ ID NO:70-87).

8. The precursor polypeptide of any one of the preceding claims, wherein the optional intracellular costimulatory sequence is present and comprises a costimulatory region comprising an intracellular region from CD28 (SEQ ID NO:41), 4-1BB (SEQ ID NO:40) or OX40 (SEQ ID NO:42).

9. The precursor polypeptide of any one of the preceding claims, wherein the intracellular JAK-STAT signaling region comprises the amino acid sequence of any one of SEQ ID NOS:131-139.

10. The precursor polypeptide of any one of the preceding claims, wherein the antibody heavy chain variable region comprises the heavy chain CDR1, CDR2, and CDR3 of a heavy chain variable region comprising the sequence of any one of SEQ ID NO: 9, 11, 13, 15, 17, 19, 21, 23, 25, 27, 29 or 31.

11. The precursor polypeptide of any one of the preceding claims, wherein the antibody light chain variable region comprises the light chain CDR1, CDR2, and CDR3 of a light chain variable region comprising the sequence of any one of SEQ ID NO: 10, 12, 14, 16, 18, 20, 22, 24, 26, 28, 30 or 32.

12. The precursor polypeptide of any one of the preceding claims, wherein the antibody heavy chain variable region comprises the heavy chain CDR1, CDR2, and CDR3 of a heavy chain variable region and the antibody light chain variable region comprises the light chain CDR1, CDR2, and CDR3 of a light chain variable region, and the heavy and light chain regions comprise the sequence of SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

13. The precursor polypeptide of any one of the preceding claims, wherein the antibody heavy chain variable region comprises a sequence having at least 95%, 97%, 98%, or 99% identity to the sequence of any one of SEQ ID NO: 9, 11, 13, 15, 17, 19, 21, 23, 25, 27, 29 or 31.

14. The precursor polypeptide of any one of the preceding claims, wherein the antibody light chain variable region comprises a sequence having at least 95%, 97%, 98%, or 99% identity to the sequence of any one of SEQ ID NO: 10, 12, 14, 16, 18, 20, 22, 24, 26, 28, 30 or 32.

15. The precursor polypeptide of any one of the preceding claims, wherein the antibody heavy chain variable region comprises a sequence having at least 95%, 97%, 98%, or 99% identity to a first sequence and the antibody light chain variable region comprises a sequence having at least 95%, 97%, 98%, or 99% identity to a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

16. The precursor polypeptide of any one of the preceding claims, wherein the antibody heavy chain variable region comprises the amino acid sequence of SEQ ID NO: 9, 11, 13, 15, 17, 19, 21, 23, 25, 27, 29 or 31.

17. The precursor polypeptide of any one of the preceding claims, wherein the antibody light chain variable region comprises the amino acid sequence of SEQ ID NO: 10, 12, 14, 16, 18, 20, 22, 24, 26, 28, 30 or 32.

18. The precursor polypeptide of any one of the preceding claims, wherein the antibody heavy chain variable region comprises a first sequence and the antibody light chain variable region comprises a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

19. The precursor polypeptide of any one of the preceding claims, wherein the optional hinge region is present and comprises a CD8 hinge region (SEQ ID NO:33), a CD28 hinge region (SEQ ID NO:34), or a hinge comprising CD8 and CD28 hinge regions (SEQ ID NO:35).

20. The precursor polypeptide of any one of the preceding claims, wherein the transmembrane region comprises a CD28 transmembrane region (SEQ ID NO:36), a CD8 transmembrane region (SEQ ID NO:37), a 4-1BB transmembrane region (SEQ ID NO:38), or a CD3ζ transmembrane region (SEQ ID NO:39).

21. The precursor polypeptide of any one of the preceding claims, wherein the self-cleaving sequence comprises the amino acid sequence of T2A (SEQ ID NO:91), P2A (SEQ ID NO:92), E2A (SEQ ID NO:93), or F2A (SEQ ID NO:94).

22. The precursor polypeptide of claim 1, comprising the amino acid sequence of SEQ ID NO: 95, 98, 101, 113, 116, 119, 122, 125 or 128.

23. A memory dimeric antigen receptor (mDAR) construct, comprising:
a) a first polypeptide chain comprising a first plurality of polypeptide regions, the first plurality comprising, in order from the amino terminus to the carboxyl terminus: (i) an antibody heavy chain variable region (VH), (ii) an antibody heavy chain constant region (CH), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζITAM domain with two ITAM motifs and a heterologous STAT3 binding motif, and (3) an optional intracellular costimulatory region (e.g., CD28 or 4-1BB or OX40); and
b) a second polypeptide chain comprising a second plurality of polypeptide regions, the second plurality comprising, in order from the amino terminus to the carboxyl terminus: (vi) an antibody light chain variable region (VL) (e.g., kappa or lambda), and (vii) an antibody light chain constant region (CL);
wherein the antibody heavy chain constant region and the antibody light chain constant region are dimerization domains for formation of the memory dimeric antigen receptor (mDAR); and
the antibody heavy chain variable region and the antibody light chain variable region form an antigen binding domain.

24. A memory dimeric antigen receptor (mDAR) construct, comprising:
a) a first polypeptide chain comprising a first plurality of polypeptide regions, the first plurality comprising, in order from the amino terminus to the carboxyl terminus: (i) an antibody light chain variable region (VL), (ii) an antibody light chain constant region (CL), (iii) an optional hinge region, (iv) a transmembrane region (TM), and (v) an intracellular JAK-STAT signaling region which includes in any order (1) a cytokine receptor intracellular region having a Box 1 motif and a Box 2 motif that bind a Janus kinase (JAK), (2) a CD3ζ intracellular signaling region having at least one CD3ζITAM domain with two ITAM motifs and a heterologous STAT3 binding motif, and (3) an optional intracellular costimulatory region (e.g., CD28 or 4-1BB or OX40); and
b) a second polypeptide chain comprising a second plurality of polypeptide regions, the second plurality comprising, in order from the amino terminus to the carboxyl terminus: (vi) an antibody heavy chain variable region (VH), and (vii) an antibody heavy chain constant region (CH);
wherein the antibody heavy chain constant region and the antibody light chain constant region are dimerization domains for formation of the memory dimeric antigen receptor (mDAR); and
the antibody heavy chain variable region and the antibody light chain variable region form an antigen binding domain.

25. The memory dimeric antigen receptor (mDAR) construct of claim 23 or 24, which binds a CD38 target antigen.

26. The memory dimeric antigen receptor (mDAR) construct of any one of claims 23-, wherein the cytokine receptor intracellular domain comprises an IL2Rβ intracellular domain having the amino acid sequence of SEQ ID NO:43.

27. The memory dimeric antigen receptor (mDAR) construct of any one of claims 23-26, wherein the CD3ζ intracellular signaling region comprises: CD3ζITAM 1 (SEQ ID NO:52, 53 or 54); CD3ζITAM 2; CD3ζITAM 3 (SEQ ID NO:49, 58 or 60); CD3ζITAM3 having a deleted portion (designated ITAM d3, SEQ ID NO:51 or 55); CD3ζITAM 1 and 2; CD3ζITAM 1, 2 and 3 (SEQ ID NO:47); CD3ζITAM 1 and 3 (SEQ ID NO:49); CD3ζITAM 2 and 3; or CD3ζITAM 1 and ITAM d3 (SEQ ID NO:51 or 56).

28. The memory dimeric antigen receptor (mDAR) construct of any one of claims 23-27, wherein the STAT binding motif comprises the amino acid sequence of YRHQ, YLRQ, YFKQ, YLPQ, YMPQ, YVLQ, YQPQ, YKPQ, YRPQ, YTHQ, YLKQ, YHNQ, YFFF, YCTF, YLSL, YLSLQ, YCTFP or YFFFH (any one of SEQ ID NO:70-87).

29. The memory dimeric antigen receptor (mDAR) construct of any one of claims 23-28, wherein the optional intracellular costimulatory sequence comprises a costimulatory region comprising an intracellular region from CD28 (SEQ ID NO:41), 4-1BB (SEQ ID NO:40) or OX40 (SEQ ID NO:42).

30. The memory dimeric antigen receptor (mDAR) construct of any one of claims 23-29, wherein the intracellular JAK-STAT signaling region comprises the amino acid sequence of any one of SEQ ID NOS:131-139.

31. The memory dimeric antigen receptor (mDAR) construct of any one of claims 23-30, wherein the antibody heavy chain variable region comprises the heavy chain CDR1, CDR2, and CDR3 of a heavy chain variable region comprising the sequence of any one of SEQ ID NO: 9, 11, 13, 15, 17, 19, 21, 23, 25, 27, 29 or 31.

32. The memory dimeric antigen receptor (mDAR) construct of any one of claims 23-31, wherein the antibody light chain variable region comprises the light chain CDR1, CDR2, and CDR3 of a light chain variable region comprising the sequence of any one of SEQ ID NO: 10, 12, 14, 16, 18, 20, 22, 24, 26, 28, 30 or 32.

33. The memory dimeric antigen receptor (mDAR) construct of any one of claims 23-32, wherein the antibody heavy chain variable region comprises the heavy chain CDR1, CDR2, and CDR3 of a heavy chain variable region and the antibody light chain variable region comprises the light chain CDR1, CDR2, and CDR3 of a light chain variable region, and the heavy and light chain regions comprise the sequence of SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

34. The memory dimeric antigen receptor (mDAR) construct of any one of claims 23-33, wherein the antibody heavy chain variable region comprises a sequence having at least 95%, 97%, 98%, or 99% identity to the sequence of any one of SEQ ID NO: 9, 11, 13, 15, 17, 19, 21, 23, 25, 27, 29 or 31.

35. The memory dimeric antigen receptor (mDAR) construct of any one of claims 23-34, wherein the antibody light chain variable region comprises a sequence having at least 95%, 97%, 98%, or 99% identity to the sequence of any one of SEQ ID NO: 10, 12, 14, 16, 18, 20, 22, 24, 26, 28, 30 or 32.

36. The memory dimeric antigen receptor (mDAR) construct of any one of claims 23-35, wherein the antibody heavy chain variable region comprises a sequence having at least 95%, 97%, 98%, or 99% identity to a first sequence and the antibody light chain variable region comprises a sequence having at least 95%, 97%, 98%, or 99% identity to a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

37. The memory dimeric antigen receptor (mDAR) construct of any one of claims 23-36, wherein the antibody heavy chain variable region comprises the amino acid sequence of SEQ ID NO: 9, 11, 13, 15, 17, 19, 21, 23, 25, 27, 29 or 31.

38. The memory dimeric antigen receptor (mDAR) construct of any one of claims 23-37, wherein the antibody light chain variable region comprises the amino acid sequence of SEQ ID NO: 10, 12, 14, 16, 18, 20, 22, 24, 26, 28, 30 or 32.

39. The memory dimeric antigen receptor (mDAR) construct of any one of claims 23-38, wherein the antibody heavy chain variable region comprises the amino acid sequence of SEQ ID NO: 9, 11, 13, 15, 17, 19, 21, 23, 25, 27, 29 or 31.

40. The memory dimeric antigen receptor (mDAR) construct of any one of claims 23-39, wherein the antibody light chain variable region comprises the amino acid sequence of SEQ ID NO: 10, 12, 14, 16, 18, 20, 22, 24, 26, 28, 30 or 32.

41. The memory dimeric antigen receptor (mDAR) construct of any one of claims 23-40, wherein the antibody heavy chain variable region comprises a first sequence and the antibody light chain variable region comprises a a second sequence, and the first and second sequences are SEQ ID NOs: 9 and 10; 11 and 12; 13 and 14; 15 and 16; 17 and 18; 19 and 20; 21 and 22; 23 and 24; 25 and 26; 27 and 28; 29 and 30; or 31 and 32, respectively.

42. The memory dimeric antigen receptor (mDAR) construct of any one of claims 23-41, wherein the optional hinge region comprises a CD8 hinge region (SEQ ID NO:33), a CD28 hinge region (SEQ ID NO:34), or a hinge comprising CD8 and CD28 hinge regions (SEQ ID NO:35).

43. The memory dimeric antigen receptor (mDAR) construct of any one of claims 23-42, wherein the transmembrane region comprises a CD28 transmembrane region (SEQ ID NO:36), a CD8 transmembrane region (SEQ ID NO:37), a 4-1BB transmembrane region (SEQ ID NO:38), or a CD3ζ transmembrane region (SEQ ID NO:39).

44. The memory dimeric antigen receptor (mDAR) construct of any one of claims 23-43, wherein the first polypeptide chain comprises the amino acid sequence of SEQ ID NO: 96, 99, 102, 114, 117, 120, 123, 126 or 129.

45. The memory dimeric antigen receptor (mDAR) construct of any one of claims 23-44, wherein the second polypeptide chain comprises the amino acid sequence of SEQ ID NO: 97, 100, 103, 115, 118, 121 124, 127 or 130.

46. A nucleic acid encoding the precursor polypeptide of any one of claims 1-22.

47. An expression vector comprising the nucleic acid of claim 46 operably linked to a promoter.

48. A host cell, or a population of host cells, harboring the nucleic acid of claim 46 operably linked to a promoter, optionally wherein the nucleic acid is present in an expression vector.

49. The host cell or the population of host cells of claim 48, wherein the host cell is a T lymphocyte (e.g., regulatory T cell, gamma-delta T cell or cytotoxic T cell), a NK (natural killer) cell, a macrophages, a dendritic cell, a mast cell, an eosinophil, a B lymphocyte, or a monocyte, or the population of host cells comprises T lymphocytes (e.g., regulatory T cells, gamma-delta T cells or cytotoxic T cells), NK (natural killer) cells, macrophages, dendritic cells, mast cells, eosinophils, B lymphocytes or monocytes.

50. A method for preparing a population of host cells expressing a plurality of a memory dimeric antigen receptor (mDAR), comprising: culturing the population of host cells of any one of claims 32-35 under conditions suitable for expressing a plurality of the precursor polypeptide by the population of host cells, and suitable for processing the plurality of precursor polypeptides into a plurality of memory dimeric antigen receptors (mDARs) by the population of host cells, wherein the processing by the population of host cells comprises cleaving the plurality of precursor polypeptide into a plurality of first and second polypeptide chains, assembling the plurality of first and second polypeptide chains to form a plurality of memory dimeric antigen receptors (mDARs), and anchoring the plurality of memory dimeric antigen receptors (mDARs) in the cellular membrane of the population of host cells.

51. The method of claim 50, wherein the expression vector is transiently introduced into the host cell or the population of host cells.

52. The method of claim 50, wherein the nucleic acid encoding the precursor polypeptide is stably inserted into the host cells' genome.

53. A population of host cells comprising a plurality of memory dimeric antigen receptors (mDARs) anchored in the cellular membrane of the population of host cells prepared by the method of any one of claims 50-52.

54. A population of host cells comprising a plurality of memory dimeric antigen receptors (mDARs) anchored in the cellular membrane of the population of host cells, wherein the mDARs are mDARs according to any one of claims 23-45.

55. A pharmaceutical composition comprising the population of host cells of claim 49, 53, or 54 and a pharmaceutically-acceptable excipient.

56. A method for treating a subject having a disease, disorder or condition associated with expression of a tumor antigen in the subject, comprising: administering to the subject the population of host cells of claim 48, 49, 53, or 54 or the pharmaceutical composition of claim 55.

57. The method of claim 56, wherein the disease is a CD38-positive cancer and/or a hematologic cancer.

58. The method of claim 56, wherein the hematologic cancer is non-Hodgkin's lymphoma (NHL), Burkitt's lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CIVIL), or multiple myeloma (MM).

59. The method of any one of claims 56-58, wherein the tumor antigen is CD38.

60. The method of any one of claims 56-59, wherein the host cells comprise autologous host cells.

61. The method of any one of claims 56-60, wherein the host cells comprise allogeneic host cells.
